(19) United States
(12) Patent Application Publication
(10) Pub. No.: US 2020/0289609 A1
(43) Pub. Date: Sep. 17, 2020

(54) PEPTIDOMIMETIC MACROCYCLES AND USES THEREOF

(71) Applicant: Aileron Therapeutics, Inc., Watertown, MA (US)

(72) Inventors: Vojislav VUKOVIC, Arlington, MA (US); Luis CARVAJAL, Acton, MA (US); David Allen ANNIS, Cambridge, MA (US)

(21) Appl. No.: 16/818,284

(22) Filed: Mar. 13, 2020

Related U.S. Application Data
(60) Provisional application No. 62/926,018, filed on Oct. 25, 2019.
(60) Provisional application No. 62/819,195, filed on Mar. 15, 2019.

(51) Int. Cl. A61K 38/12; A61K 31/337; A61P 35/00

(52) U.S. Cl. CPC A61K 38/12; A61P 35/00; A61K 31/337

(57) ABSTRACT

The present disclosure describes methods of using peptidomimetic macrocycles in combination with an additional therapy to treat a condition, for example, cancer. In some embodiments, the peptidomimetic macrocycle can mitigate a side effect (e.g., mucositis, neutropenia, or thrombocytopenia) of the additional therapy.

### CROSS REFERENCE

This application claims the benefit of U.S. Provisional Application No. 62/819,195 filed Mar. 15, 2019, and U.S. Provisional Application No. 62/926,018, filed Oct. 25, 2019, each of which is incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 26, 2020, is named 35224-834_201_SL.txt and is 1,600,680 bytes in size.

### BACKGROUND

Side effects that can result from anticancer therapies include myelosuppression and mucositis. Myelosuppression relates to the destruction of bone marrow, while mucositis involves inflammation and ulceration of mucous membranes of the digestive tract. Side effects such as myelosuppression and mucositis can limit the dose of an anticancer therapy that can be safely administered to a patient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the disclosure provides a method of treating a tumor in a subject in need thereof, the method comprising administering to the subject a therapeutically effective amount of a peptidomimetic macrocycle and a therapeutically effective amount of a first additional pharmaceutically-active agent, wherein the administration of the peptidomimetic macrocycle induces cell cycle arrest in a non-cancerous tissue in the subject, the administration of the peptidomimetic macrocycle does not induce cell cycle arrest in the tumor; and the administration of the peptidomimetic macrocycle does not induce apoptosis in the tumor.

In some embodiments, the disclosure provides a method of treating a cancer in a subject in need thereof, the method comprising administering to the subject a therapeutically effective amount of a peptidomimetic macrocycle and a therapeutically effective amount of a first additional pharmaceutically-active agent, wherein the cancer has a p53 deactivating mutation; a non-cancerous tissue of the subject comprises a functional p53 protein; and the non-cancerous tissue is bone marrow or digestive tract tissue.

In some embodiments, the disclosure provides a method of treating a tumor in a subject in need thereof, the method comprising administering to the subject a therapeutically effective amount of a peptidomimetic macrocycle and a therapeutically effective amount of a first additional pharmaceutically-active agent, wherein the administration of the peptidomimetic macrocycle does not induce cell cycle arrest in the tumor; the administration of the peptidomimetic macrocycle does not induce apoptosis in the tumor; the therapeutically effective amount of the first additional pharmaceutically-active agent is associated with a side effect; and the administration of the peptidomimetic macrocycle reduces a likelihood of the subject developing the side effect.

### BRIEF DESCRIPTION OF THE DRAWINGS

FIG. 1 shows the human wild type p53 protein sequence (SEQ ID NO: 1949).

FIG. 2 presents a schematic showing dose dependent effects of peptidomimetic macrocycles on cell cycle arrest and cell death.

FIG. 3 presents a schematic showing the mechanism of how a combination treatment of chemotherapy and the peptidomimetic macrocycle AP-1 can treat p53 mutant tumors and prevent myelosuppression.

FIG. 4 presents data that shows the effect of AP-1 in p53 wild type cells vs. p53 mutant cells.

FIG. 5 presents data on the effect of AP-1 on apoptosis (top panel) and cell cycle arrest (bottom panel). For each group of bar graphs shown in the top panel, the following dose levels of AP-1 are represented in order from left to right: vehicle control, 1 μM, 5 μM, and 10 μM. For each group of bar graphs shown in the bottom panel, the following dose levels of AP-1 are represented in order from left to right: vehicle control, 1 μM, and 2.5 μM.

FIG. 6 shows the effect of AP-1 on DNA-synthesis and cell cycle arrest in CD34⁺ bone marrow cells.

FIG. 7 shows the percentage of 5-ethynyl-2′-deoxyuridine (EdU) positive CD34⁺ bone marrow cells (which is indicative of cells in S-phase) at two different time points following treatment with vehicle or AP-1.

FIG. 8 shows the effect of pretreatment with AP-1 on topotecan induced DNA damage as measured by γH2AX incorporation.

FIG. 9 shows mRNA expression levels of p21, p53 upregulated modulator of apoptosis (PUMA), and Noxa in mouse bone marrow cells following in vivo treatment with a single dose of AP-1.

FIG. 10 shows EdU incorporation in hematopoietic stem and progenitor cells following in vivo treatment with a single dose of AP-1 at 10 mg/kg.

FIG. 11 shows EdU incorporation in lineage negative, Kit positive hematopoietic stem and progenitor cells following in vivo treatment with a single dose of AP-1.

FIG. 12 shows serum levels of macrophage inhibitory cytokine-1 (MIC-1) in mice following treatment with a single dose of AP-1.

FIG. 13 shows p53, p21, and bromodeoxyuridine (BrdU) staining in MCF-7 mouse tumors following treatment with 20 mg/kg AP-1.

FIG. 14 shows quantification of p53, p21, poly(ADP-ribose) polymerase (PARP), and BrdU staining in MCF-7 tumor tissue following treatment with 20 mg/kg AP-1.

FIG. 15 shows MCF-7.1 tumor volume in mice following treatment with vehicle, AP-1, Abraxane®, or AP-1 in combination with Abraxane®.

FIG. 16A and FIG. 16B show neutrophil levels is mice following treatment with vehicle, AP-1, topotecan, or a combination of AP-1 and topotecan.

FIG. 16C shows neutrophil levels in mice of two different treatment groups.

FIG. 16D shows neutrophil levels in mice of two different treatment groups.

FIG. 17A shows median tumor volume and mouse survival in a MC38 tumor mouse cancer model following treatment with vehicle, AP-1, topotecan, or a combination of AP-1 and topotecan.

FIG. 17B shows median tumor volume and mouse survival in a H69 tumor mouse cancer model following treatment with vehicle, AP-1, topotecan, or a combination of AP-1 and topotecan.

FIG. 17C shows median tumor volume and mouse survival in a H211 tumor mouse cancer model following treatment with vehicle, AP-1, topotecan, or a combination of AP-1 and topotecan.

FIG. 18A shows neutrophil levels in mice following treatment with vehicle, AP-1, carboplatin and paclitaxel, or carboplatin and paclitaxel in combination with AP-1.

FIG. 18B shows neutrophil levels in mice of two separate treatment groups.

FIG. 19 shows neutrophil levels in mice following treatment with vehicle, AP-1, docetaxel, or AP-1 in combination with docetaxel.

FIG. 20 shows a schematic of the study design for a small cell lung cancer Phase 1b dose optimization study (RP2D=recommended Phase 2 dose).

FIG. 21 shows a schematic of the study design for a small cell lung cancer Phase 1b dose expansion study.

FIG. 22 shows a schematic of the study design for a small cell lung cancer Phase 2 trial (RP2D=recommended Phase 2 dose).

FIG. 23 shows histological sections of gut tissue from mice treated with topotecan alone or topotecan in combination with AP-1.

FIG. 24A shows histological scores of hypertrophy/hyperplasia in intestinal tissue of mice following treatment with vehicle, AP-1, topotecan, or a combination of AP-1 and topotecan.

FIG. 24B shows histological scores of hypertrophy/hyperplasia in intestinal tissue of mice in two separate treatment groups.

### DETAILED DESCRIPTION

Anticancer therapies such as, for example, chemotherapeutic agents can have dose limiting side effects that can limit the efficacy of such therapies. Dose limiting side effects can include, for example, myelosuppression and mucositis. Mucositis can lead to painful inflammation and ulceration of mucous membranes lining the digestive tract. Ulcerations can lead to weight loss, infection, and/or sepsis. Myelosuppression can lead to a decrease in the production of cells responsible for providing immunity (leukocytes), carrying oxygen (erythrocytes), and the mediation of blood clotting (thrombocytes). Myelosuppression can have serious consequences for a subject and can result in a weakened immune system, anemia, neutropenia, thrombocytopenia, and/or spontaneous and severe bleeding. Mucositis and myelosuppression can result from anticancer therapy-induced cytotoxicity in cells of the digestive tract and bone marrow, respectively. In some instances, inducing cell cycle arrest in cells can protect the cells from the cytotoxic effects of anticancer therapies (e.g., chemotherapeutic agents).

Cell cycle arrest can be induced via activation of the human transcription factor protein p53, which is encoded by the TP53 gene. The E3 ubiquitin ligase MDM2, also known as HDM2, negatively regulates p53 function through a direct binding interaction that neutralizes the p53 transactivation activity. Neutralization of p53 transactivation activity leads to export from the nucleus of p53 protein, which targets p53 for degradation via the ubiquitylation-proteasomal pathway.

MDMX (MDM4) is a negative regulator of p53, and significant structural homology exists between the p53 binding interfaces of MDM2 and MDMX. The p53-MDM2 and p53-MDMX protein-protein interactions are mediated by the same 15-residue alpha-helical transactivation domain of p53, which inserts into hydrophobic clefts on the surface of MDM2 and MDMX. Three residues within this domain of p53 (F19, W23, and L26) are essential for binding to MDM2 and MDMX.

Described herein are p53-based peptidomimetic macrocycles that modulate an activity of p53. Peptidomimetic macrocycles of the disclosure can modulate p53 activity by, for example, inhibiting the interactions between p53 and MDM2 and/or p53 and MDMX proteins. Also provided herein are uses of p53-based peptidomimetic macrocycles and an additional pharmaceutically-active agent for the treatment of a condition, for example, cancer or another hyperproliferative disease. Further, provided herein are p53-based peptidomimetic macrocycles that can be used to mitigate a side effect (e.g., myelosuppression or mucositis) caused by a second pharmaceutically-active agent. For example, a method disclosed herein can comprise treating cancer in subject in need thereof by administering a peptidomimetic macrocycles in combination with a second pharmaceutically-active agent (e.g., a chemotherapeutic agent). In some instances, the peptidomimetic macrocycle can induce cell cycle arrest in the bone marrow and/or digestive tract tissue of the subject and mitigate a myelosuppression related side effect (e.g., neutropenia or thrombocytopenia) and/or mucositis caused by the second pharmaceutically-active agent.

### Definitions

As used herein, the term “macrocycle” refers to a molecule having a chemical structure including a ring or cycle formed by at least 9 covalently bonded atoms.

As used herein, the term “peptidomimetic macrocycle” or “crosslinked polypeptide” refers to a compound comprising a plurality of amino acid residues joined by a plurality of peptide bonds and at least one macrocycle-forming linker which forms a macrocycle between a first naturally-occurring or non-naturally-occurring amino acid residue (or analogue) and a second naturally-occurring or non-naturally-occurring amino acid residue (or analogue) within the same molecule. Peptidomimetic macrocycle include embodiments where the macrocycle-forming linker connects the α-carbon of the first amino acid residue (or analogue) to the α-carbon of the second amino acid residue (or analogue). The peptidomimetic macrocycles optionally include one or more non-peptide bonds between one or more amino acid residues and/or amino acid analogue residues, and optionally include one or more non-naturally-occurring amino acid residues or amino acid analogue residues in addition to any which form the macrocycle. A “corresponding uncrosslinked polypeptide” when referred to in the context of a peptidomimetic macrocycle is understood to relate to a polypeptide of the same length as the macrocycle and comprising the equivalent natural amino acids of the wild-type sequence corresponding to the macrocycle.

AP-1 is an alpha helical hydrocarbon crosslinked polypeptide macrocycle with an amino acid sequence less than 20 amino acids long that is derived from the transactivation domain of wild type human p53 protein. AP-1 contains a phenylalanine, a tryptophan and a leucine amino acid in the same positions relative to each other as in the transactivation domain of wild type human p53 protein. AP-1 has a single cross link spanning amino acids in the i to the i+7 position of the amino acid sequence and has more than three amino acids between the i+7 position and the carboxyl terminus. AP-1 binds to human MDM2 and MDM4 and has an observed mass of 950-975 m/e as measured by electrospray ionization-mass spectrometry.

As used herein, the term “stability” refers to the maintenance of a defined secondary structure in solution by a peptidomimetic macrocycle as measured by circular dichroism, NMR or another biophysical measure, or resistance to proteolytic degradation in vitro or in vivo. Non-limiting examples of secondary structures contemplated herein are α-helices, 3₁₀ helices, β-turns, and β-pleated sheets.

As used herein, the term “helical stability” refers to the maintenance of an α-helical structure by a peptidomimetic macrocycle as measured by circular dichroism or NMR. In some embodiments, a peptidomimetic macrocycle can exhibit at least a 1.25, 1.5, 1.75, or 2-fold increase in α-helicity as determined by circular dichroism compared to a corresponding uncrosslinked macrocycle.

The term “amino acid” refers to a molecule containing both an amino group and a carboxyl group. Suitable amino acids include, without limitation, both the D- and L-isomers of the naturally-occurring amino acids, as well as non-naturally-occurring amino acids prepared by organic synthesis or other metabolic routes. The term amino acid, as used herein, includes, without limitation, α-amino acids, natural amino acids, non-natural amino acids, and amino acid analogues.

The term “α-amino acid” refers to a molecule containing both an amino group and a carboxyl group bound to a carbon which is designated the α-carbon.

The term “μ-amino acid” refers to a molecule containing both an amino group and a carboxyl group in a β configuration.

The term “naturally-occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | | | | Side-chain | |
|---|---|---|---|---|---|
| | 3-Letter | 1-Letter | Side-chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | Positive | −3.2 |
| | | | | (10%) | |
| | | | | Neutral | |
| | | | | (90%) | |
| Isoleucine | ILe | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acids” are glycine, alanine, proline, and analogues thereof. “Large hydrophobic amino acids” are valine, leucine, isoleucine, phenylalanine, methionine, tryptophan, and analogues thereof. “Polar amino acids” are serine, threonine, asparagine, glutamine, cysteine, tyrosine, and analogues thereof. “Charged amino acids” are lysine, arginine, histidine, aspartate, glutamate, and analogues thereof.

The term “amino acid analogue” refers to a molecule which is structurally similar to an amino acid and which can be substituted for an amino acid in the formation of a peptidomimetic macrocycle. Amino acid analogues include, without limitation, β-amino acids and amino acids wherein the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogues include, without limitation, structures according to the following:

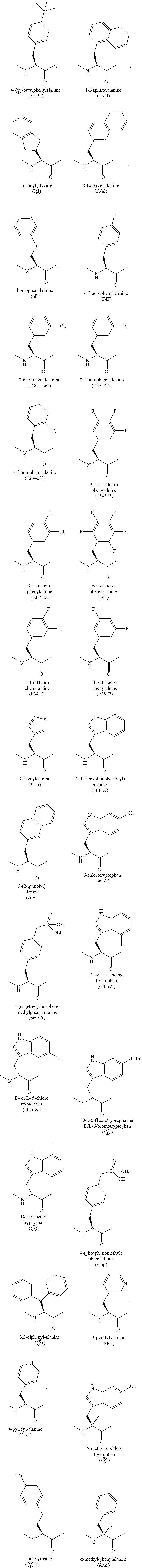

[chemical structure]

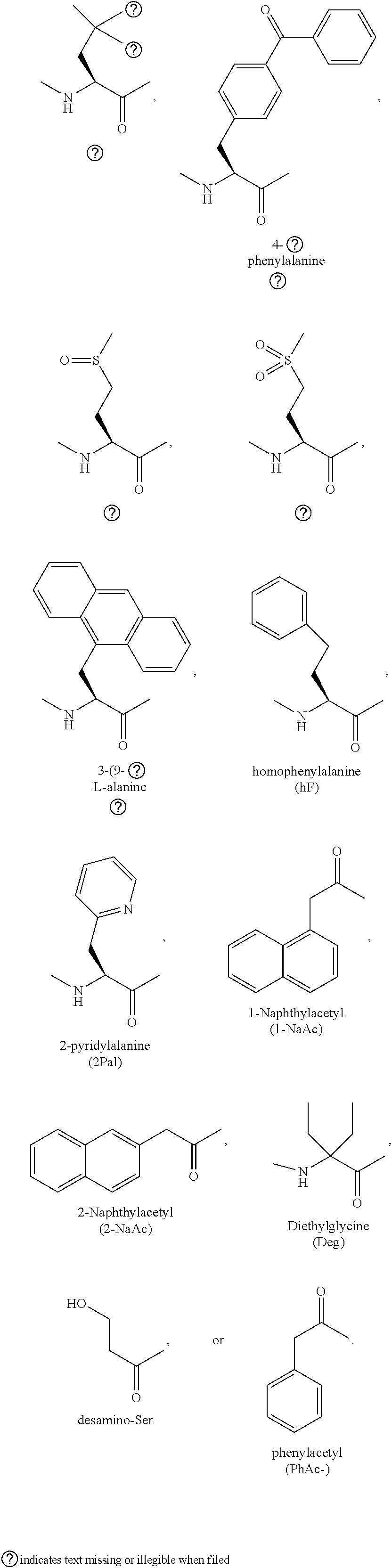

Amino acid analogues include β-amino acid analogues. Examples of β-amino acid analogues include, but are not limited to, the following: cyclic β-amino acid analogues; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogues include analogues of alanine, valine, glycine or leucine. Examples of amino acid analogues of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexyl ammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexyl ammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH,dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine.dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogues include analogues of arginine or lysine. Examples of amino acid analogues of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-omithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-omithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)₂-OH (symmetrical); Lys(ivDde)-OH; Lys(Me)₂-OH.HCl; Lys(Me₃)-OH chloride; Nω-nitro-D-arginine; and Nω-nitro-L-arginine.

Amino acid analogues include analogues of aspartic or glutamic acids. Examples of amino acid analogues of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-3-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogues include analogues of cysteine and methionine. Examples of amino acid analogues of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogues include analogues of phenylalanine and tyrosine. Examples of amino acid analogues of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogues include analogues of proline. Examples of amino acid analogues of proline include, but are not limited to, 3,4-dehydro-proline, 4-fluoro-proline, cis-4-hydroxy-proline, thiazolidine-2-carboxylic acid, and trans-4-fluoro-proline.

Amino acid analogues include analogues of serine and threonine. Examples of amino acid analogues of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogues include analogues of tryptophan. Examples of amino acid analogues of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogues are racemic. In some embodiments, the D isomer of the amino acid analogue is used. In some embodiments, the L isomer of the amino acid analogue is used. In other embodiments, the amino acid analogue comprises chiral centers that are in the R or S configuration. In still other embodiments, the amino group(s) of a β-amino acid analogue is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analogue is protected, e.g., as its ester derivative. In some embodiments the salt of the amino acid analogue is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, e.g., is replaced with another amino acid residue from the same side chain family. Other examples of acceptable substitutions are substitutions based on isosteric considerations (e.g., norleucine for methionine) or other properties (e.g., 2-thienylalanine for phenylalanine, or 6-Cl-tryptophan for tryptophan).

The term “capping group” refers to the chemical moiety occurring at either the carboxy or amino terminus of the polypeptide chain of the subject peptidomimetic macrocycle. The capping group of a carboxy terminus includes an unmodified carboxylic acid (i.e. —COOH) or a carboxylic acid with a substituent. For example, the carboxy terminus can be substituted with an amino group to yield a carboxamide at the C-terminus. Various substituents include but are not limited to primary, secondary, and tertiary amines, including pegylated secondary amines. Representative secondary amine capping groups for the C-terminus include:

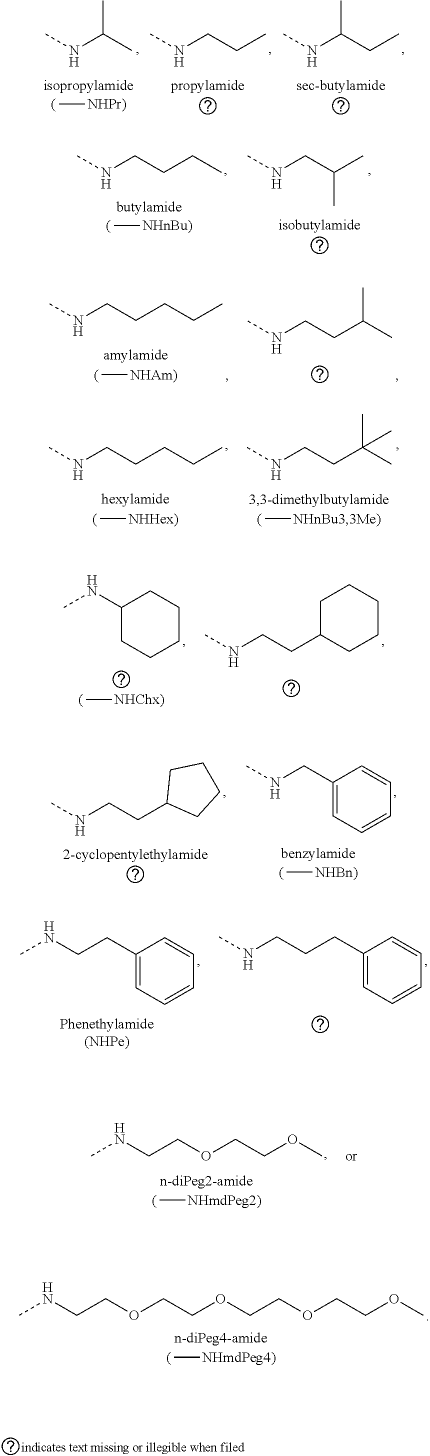

The capping group of an amino terminus includes an unmodified amine (i.e. —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates. Representative capping groups for the N-terminus include, but are not limited to, 4-FBzl (4-fluoro-benzyl) and the following:

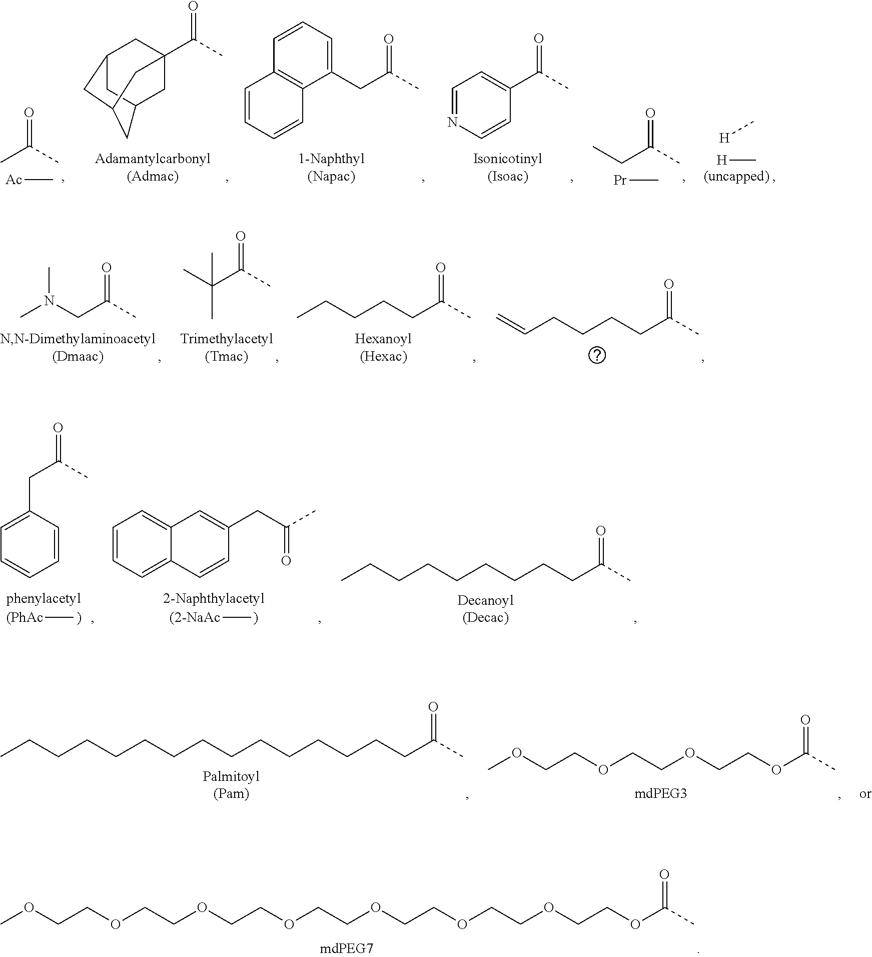

The term “member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen or fluoro substituents or methyl side chains do not participate in forming the macrocycle.

The symbol “ [symbol] ” when used as part of a molecular structure refers to a single bond or a trans or cis double bond.

The term “amino acid side chain” refers to a moiety attached to the α-carbon (or another backbone atom) in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally-occurring amino acid side chains are also included, for example, those that occur in nature (e.g., an amino acid metabolite) or those that are made synthetically (e.g., an α,α di-substituted amino acid).

The term “α,α di-substituted amino” acid refers to a molecule or moiety containing both an amino group and a carboxyl group bound to a carbon (the α-carbon) that is attached to two natural or non-natural amino acid side chains.

The term “polypeptide” encompasses two or more naturally- or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “first C-terminal amino acid” refers to the amino acid which is closest to the C-terminus. The term “second C-terminal amino acid” refers to the amino acid attached at the N-terminus of the first C-terminal amino acid.

The term “macrocyclization reagent” or “macrocycle-forming reagent” as used herein refers to any reagent which can be used to prepare a peptidomimetic macrocycle by mediating the reaction between two reactive groups. Reactive groups can be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents can additionally include, for example, Ru reagents known in the art such as Cp*RuCl(PPh₃)₂, [Cp*RuCl]₄ or other Ru reagents which can provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. In other examples, catalysts have W or Mo centers. In some embodiments, the reactive groups are thiol groups. In some embodiments, the macrocyclization reagent is, for example, a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1 to 10 (inclusive) carbon atoms in it. In the absence of any numerical designation, “alkyl” is a chain (straight or branched) having 1 to 20 (inclusive) carbon atoms.

The term “alkylene” refers to a divalent alkyl (i.e., —R—).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2 to 10 (inclusive) carbon atoms. The term “lower alkenyl” refers to a C₂-C₆ alkenyl chain. In the absence of any numerical designation, “alkenyl” is a chain (straight or branched) having 2 to 20 (inclusive) carbon atoms.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2 to 10 (inclusive) carbon atoms. The term “lower alkynyl” refers to a C₂-C₆ alkynyl chain. In the absence of any numerical designation, “alkynyl” is a chain (straight or branched) having 2 to 20 (inclusive) carbon atoms.

The term “aryl” refers to a 6-carbon monocyclic or 10-carbon bicyclic aromatic ring system wherein 0, 1, 2, 3, or 4 atoms of each ring are substituted by a substituent. Examples of aryl groups include phenyl, naphthyl and the like. The term “arylalkoxy” refers to an alkoxy substituted with aryl.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH₂-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl.

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)₂CH₂C(O)NH₂, —CH₂—CH₂—NH—C(O)—CH₃, —CH₂—CH₂—NH—C(O)—CH₃—CH₃, and —CH₂—CH₂—NH—C(O)—CH═CH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂ CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, the compounds disclosed herein contain one or more asymmetric centers and thus occur as racemates and racemic mixtures, single enantiomers, individual diastereomers and diastereomeric mixtures. All such isomeric forms of these compounds are included unless expressly provided otherwise. In some embodiments, the compounds disclosed herein are also represented in multiple tautomeric forms, in such instances, the compounds include all tautomeric forms of the compounds described herein (e.g., if alkylation of a ring system results in alkylation at multiple sites, the disclosure includes all such reaction products). All such isomeric forms of such compounds are included unless expressly provided otherwise. All crystal forms of the compounds described herein are included unless expressly provided otherwise.

As used herein, the terms “increase” and “decrease” mean, respectively, to cause a statistically significantly (i.e., p<0.1) increase or decrease of at least 5%.

As used herein, the recitation of a numerical range for a variable is intended to convey that the variable is equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values ≥0 and ≤2 if the variable is inherently continuous.

As used herein, unless specifically indicated otherwise, the word “or” is used in the inclusive sense of “and/or” and not the exclusive sense of “either/or”.

The term “on average” represents the mean value derived from performing at least three independent replicates for each data point.

The term “biological activity” encompasses structural and functional properties of a macrocycle. Biological activity is, for example, structural stability, alpha-helicity, affinity for a target, resistance to proteolytic degradation, cell penetrability, intracellular stability, in vivo stability, or any combination thereof.

The term “binding affinity” refers to the strength of a binding interaction, for example between a peptidomimetic macrocycle and a target. Binding affinity can be expressed, for example, as equilibrium dissociation constant (“K_{D}”), which is expressed in units which are a measure of concentration (e.g. M, mM, μM, nM etc). Numerically, binding affinity and K_{D} values vary inversely, such that a lower binding affinity corresponds to a higher K_{D} value, while a higher binding affinity corresponds to a lower K_{D} value. Where high binding affinity is desirable, “improved” binding affinity refers to higher binding affinity and therefore lower K_{D} values.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The terms “combination therapy” or “combined treatment” or in “combination” as used herein denotes any form of concurrent or parallel treatment with at least two distinct therapeutic agents.

The term “in vitro efficacy” refers to the extent to which a test compound, such as a peptidomimetic macrocycle, produces a beneficial result in an in vitro test system or assay. In vitro efficacy can be measured, for example, as an “IC₅₀” or “EC₅₀” value, which represents the concentration of the test compound which produces 50% of the maximal effect in the test system.

The term “ratio of in vitro efficacies” or “in vitro efficacy ratio” refers to the ratio of IC₅₀ or EC₅₀ values from a first assay (the numerator) versus a second assay (the denominator). Consequently, an improved in vitro efficacy ratio for Assay 1 versus Assay 2 refers to a lower value for the ratio expressed as IC₅₀(Assay 1)/IC₅₀(Assay 2) or alternatively as EC₅₀ (Assay 1)/EC₅₀ (Assay 2). This concept can also be characterized as “improved selectivity” in Assay 1 versus Assay 2, which can be due either to a decrease in the IC₅₀ or EC₅₀ value for Target 1 or an increase in the value for the IC₅₀ or EC₅₀ value for Target 2.

As used in the present application, “biological sample” means any fluid or other material derived from the body of a normal or diseased subject, such as blood, bone marrow, serum, plasma, lymph, urine, saliva, tears, cerebrospinal fluid, milk, amniotic fluid, bile, ascites fluid, pus, and the like. Also included within the meaning of the term “biological sample” is an organ or tissue extract and culture fluid in which any cells or tissue preparation from a subject has been incubated. The biological samples can be any samples from which genetic material can be obtained. Biological samples can also include solid or liquid cancer cell samples or specimens. The cancer cell sample can be a cancer cell tissue sample. In some embodiments, the cancer cell tissue sample can be obtained from surgically excised tissue. Non-limiting examples of sources of biological samples include fine needle aspiration, core needle biopsy, vacuum assisted biopsy, incisional biopsy, excisional biopsy, punch biopsy, shave biopsy or skin biopsy. In some cases, the biological samples comprise fine needle aspiration samples. In some embodiments, the biological samples comprise tissue samples, including, for example, excisional biopsy, incisional biopsy, or other biopsy. The biological samples can comprise a mixture of two or more sources; for example, fine needle aspirates and tissue samples. Tissue samples and cellular samples can also be obtained without invasive surgery, for example by punctuating the chest wall or the abdominal wall or from masses of breast, thyroid or other sites with a fine needle and withdrawing cellular material (fine needle aspiration biopsy). In some embodiments, a biological sample is a bone marrow aspirate sample. A biological sample can be obtained by methods known in the art such as the biopsy methods provided herein, swabbing, scraping, phlebotomy, or any other suitable method.

The term “solid tumor” or “solid cancer” as used herein refers to tumors that usually do not contain cysts or liquid areas. Solid tumors as used herein include sarcomas, carcinomas and lymphomas. In various embodiments, leukemia (cancer of blood) is not solid tumor.

The term “liquid cancer” as used herein refers to cancer cells that are present in body fluids, such as blood, lymph and bone marrow. Liquid cancers include leukemia, myeloma and liquid lymphomas. Liquid lymphomas include lymphomas that contain cysts or liquid areas. Liquid cancers as used herein do not include solid tumors, such as sarcomas and carcinomas or solid lymphomas that do not contain cysts or liquid areas.

### Treatment of Cancer

A method described herein can be used to treat cancer. Types of cancer that can be treated with a method of the disclosure include, without limitation, solid tumor cancers and liquid cancers. In some embodiments, a method of treating cancer described herein comprises administration of a peptidomimetic macrocycle in combination with a second pharmaceutically-active agent.

Solid tumor cancers that can be treated by the methods provided herein include, but are not limited to, sarcomas, carcinomas, and lymphomas. In specific embodiments, solid tumors that can be treated in accordance with the methods described include, but are not limited to, cancer of the breast, liver, neuroblastoma, head, neck, eye, mouth, throat, esophagus, esophagus, chest, bone, lung, kidney, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. Solid tumors that can be treated by the instant methods include tumors and/or metastasis (wherever located) other than lymphatic cancer, for example brain and other central nervous system tumors (including but not limited to tumors of the meninges, brain, spinal cord, cranial nerves and other parts of central nervous system, e.g. glioblastomas or medulloblastomas); head and/or neck cancer; breast tumors; circulatory system tumors (including but not limited to heart, mediastinum and pleura, and other intrathoracic organs, vascular tumors and tumor-associated vascular tissue); excretory system tumors (including but not limited to tumors of kidney, renal pelvis, ureter, bladder, other and unspecified urinary organs); gastrointestinal tract tumors (including but not limited to tumors of the esophagus, stomach, small intestine, colon, colorectal, rectosigmoid junction, rectum, anus and anal canal, tumors involving the liver and intrahepatic bile ducts, gall bladder, other and unspecified parts of biliary tract, pancreas, other and digestive organs); oral cavity tumors (including but not limited to tumors of lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (including but not limited to tumors of vulva, vagina, Cervix uteri, Corpus uteri, uterus, ovary, and other sites associated with female genital organs, placenta, penis, prostate, testis, and other sites associated with male genital organs); respiratory tract tumors (including but not limited to tumors of nasal cavity and middle ear, accessory sinuses, larynx, trachea, bronchus and lung, e.g. small cell lung cancer or non-small cell lung cancer); skeletal system tumors (including but not limited to tumors of bone and articular cartilage of limbs, bone articular cartilage and other sites); skin tumors (including but not limited to malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

In some examples, the solid tumor treated by the methods of the instant disclosure is pancreatic cancer, bladder cancer, colon cancer, liver cancer, colorectal cancer (colon cancer or rectal cancer), breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, skin cancer, ocular tumor, choriocarcinoma (tumor of the placenta), sarcoma or soft tissue cancer.

In some examples, the solid tumor to be treated by the methods of the instant disclosure is selected bladder cancer, bone cancer, breast cancer, cervical cancer, CNS cancer, colon cancer, ocular tumor, renal cancer, liver cancer, lung cancer, pancreatic cancer, choriocarcinoma (tumor of the placenta), prostate cancer, sarcoma, skin cancer, soft tissue cancer or gastric cancer.

In some examples, the solid tumor treated by the methods of the instant disclosure is breast cancer. Non limiting examples of breast cancer that can be treated by the instant methods include ductal carcinoma in situ (DCIS or intraductal carcinoma), lobular carcinoma in situ (LCIS), invasive (or infiltrating) ductal carcinoma, invasive (or infiltrating) lobular carcinoma, inflammatory breast cancer, triple-negative breast cancer, paget disease of the nipple, phyllodes tumor (phyllodes tumor or cystosarcoma phyllodes), angiosarcoma, adenoid cystic (or adenocystic) carcinoma, low-grade adenosquamous carcinoma, medullary carcinoma, papillary carcinoma, tubular carcinoma, metaplastic carcinoma, micropapillary carcinoma, and mixed carcinoma.

In some examples, the solid tumor treated by the methods of the instant disclosure is bone cancer. Non limiting examples of bone cancer that can be treated by the instant methods include osteosarcoma, chondrosarcoma, the Ewing Sarcoma Family of Tumors (ESFTs).

In some examples, the solid tumor treated by the methods of the instant disclosure is skin cancer. Non limiting examples of skin cancer that can be treated by the instant methods include melanoma, basal cell skin cancer, and squamous cell skin cancer.

In some examples, the solid tumor treated by the methods of the instant disclosure is ocular tumor. Non limiting examples of ocular tumor that can be treated by the methods of the instant disclosure include ocular tumor is choroidal nevus, choroidal melanoma, choroidal metastasis, choroidal hemangioma, choroidal osteoma, iris melanoma, uveal melanoma, intraocular lymphoma, melanocytoma, metastasis retinal capillary hemangiomas, congenital hypertrophy of the RPE, RPE adenoma or retinoblastoma.

Liquid cancer cancers that can be treated by the methods provided herein include, but are not limited to, leukemias, myelomas, and liquid lymphomas. In specific embodiments, liquid cancers that can be treated in accordance with the methods described include, but are not limited to, liquid lymphomas, leukemias, and myelomas. Non-limiting examples of liquid lymphomas and leukemias that can be treated in accordance with the methods described include chronic lymphocytic leukemia/small lymphocytic lymphoma, B-cell prolymphocytic leukemia, lymphoplasmacytic lymphoma (such as Waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma.

Examples of liquid cancers include cancers involving hyperplastic/neoplastic cells of hematopoietic origin, e.g., arising from myeloid, lymphoid or erythroid lineages, or precursor cells thereof. Non-limiting examples of disorders include: acute leukemias, e.g., erythroblastic leukemia, and acute megakaryoblastic leukemia. Additional non-limiting examples of myeloid disorders include, but are not limited to, acute promyeloid leukemia (APML), acute myelogenous leukemia (AML), and chronic myelogenous leukemia (CML); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), multiple myeloma, hairy cell leukemia (HLL), and Waldenstrom's macroglobulinemia (WM). Additional forms of malignant liquid lymphomas include, but are not limited to non-Hodgkin lymphoma and variants thereof, adult T cell leukemia/lymphoma (ATL), cutaneous T-cell lymphoma (CTCL), peripheral T-cell lymphoma (PTCL), large granular lymphocytic leukemia (LGF), Hodgkin's disease, and Reed-Sternberg disease. For example, liquid cancers include, but are not limited to, acute lymphocytic leukemia (ALL); T-cell acute lymphocytic leukemia (T-ALL); anaplastic large cell lymphoma (ALCL); chronic myelogenous leukemia (CML); acute myeloid leukemia (AML); chronic lymphocytic leukemia (CLL); B-cell chronic lymphocytic leukemia (B-CLL); diffuse large B-cell lymphomas (DLBCL); hyper eosinophilia/chronic eosinophilia; and Burkitt's lymphoma.

In some embodiments, the cancer comprises an acute lymphoblastic leukemia; acute myeloid leukemia; AIDS-related cancers; AIDS-related lymphoma; chronic lymphocytic leukemia; chronic myelogenous leukemia; chronic myeloproliferative disorders; adult T cell leukemia/lymphoma (ATL); cutaneous T-cell lymphoma (CTCL); peripheral T-cell lymphoma (PTCL); Hodgkin lymphoma; multiple myeloma; multiple myeloma/plasma cell neoplasm; Non-Hodgkin lymphoma; or primary central nervous system (CNS) lymphoma. In some embodiments, the liquid cancer can be B-cell chronic lymphocytic leukemia, B-cell lymphoma-DLBCL, B-cell lymphoma-DLBCL-germinal center-like, B-cell lymphoma-DLBCL-activated B-cell-like, or Burkitt's lymphoma.

In some embodiments, a subject treated in accordance with the methods provided herein is a human who has or is diagnosed with cancer with a p53 deactivating mutation and/or lacking active p53. In some embodiments, a subject treated for cancer in accordance with the methods provided herein is a human predisposed or susceptible to cancer with a p53 deactivating mutation and/or lacking active p53. In some embodiments, a subject treated for cancer in accordance with the methods provided herein is a human at risk of developing cancer with a p53 deactivating mutation and/or lacking active p53. A p53 deactivating mutation in some examples can be a mutation in the DNA-binding domain of the p53 protein. In some examples, the p53 deactivating mutation can be a missense mutation. In various examples, the cancer can be determined to have one or more p53 deactivating mutations selected from mutations at one or more of residues R175, G245, R248, R249, R273, and R282. The presence of a p53 deactivating mutation and/or the lack of wild type p53 in the cancer can be determined by any suitable method known in art, for example by sequencing, array-based testing, RNA analysis and amplifications methods like PCR.

In certain embodiments, the human subject is refractory and/or intolerant to one or more other treatments of the cancer. In some embodiments, the human subject has had at least one unsuccessful prior treatment and/or therapy of the cancer.

In some embodiments, a subject treated for cancer in accordance with the methods provided herein is a human, who has or is diagnosed with a cancer. In other embodiments, a subject treated for cancer in accordance with the methods provided herein is a human, predisposed or susceptible to a cancer. In some embodiments, a subject treated for cancer in accordance with the methods provided herein is a human, at risk of developing a cancer.

In some embodiments, a subject treated for a cancer in accordance with the methods provided herein is a human, who has or is diagnosed with a cancer, determined to have a p53 deactivating mutation and/or lack wild type p53. In some embodiments, a subject treated for cancer in accordance with the methods provided herein is a human, predisposed or susceptible to a cancer, determined to have a p53 deactivating mutation and/or lack wild type p53. In some embodiments, a subject treated for cancer in accordance with the methods provided herein is a human, at risk of developing a tumor, determined to have a p53 deactivating mutation and/or expressing wild type p53. In some embodiments, a p53 deactivating mutation can lead to loss of (or a decrease in) the in vitro apoptotic activity of p53. Non-limiting examples of p53 deactivating mutations are shown in the following table:

| Mutation at position | Amino acid change |
|---|---|
| 62 | E62_W91del |
| 122 | V122X |
| 135 | C135S |
| 143 | V143A |
| 144 | Q144P |
| 146 | W146X |
| 157 | V157F |
| 158 | R158H |
| 163 | Y163N |
| 168 | H168Y |
| 173 | V173L |
| 175 | R175H |
| 175 | R175P |
| 175 | R175Q |
| 175 | R175S |
| 219 | P219H |
| 234 | Y234C |
| 234 | Y234H |
| 237 | M237I |
| 240 | S240R |
| 245 | G245C |
| 245 | G245S |
| 246 | M246I |
| 248 | R248Q |
| 248 | R248W |
| 249 | R249S |
| 272 | V272M |
| 273 | R273H |
| 274 | V274F |
| 279 | G279E |
| 280 | R280K |
| 281 | D281H |
| 282 | R282W |
| 306 | R306P |
| 308 | P300_L308del |
| 327 | P300_Y327del |
| 332 | D324_I332del |
| 337 | R337C |
| 344 | L344P |

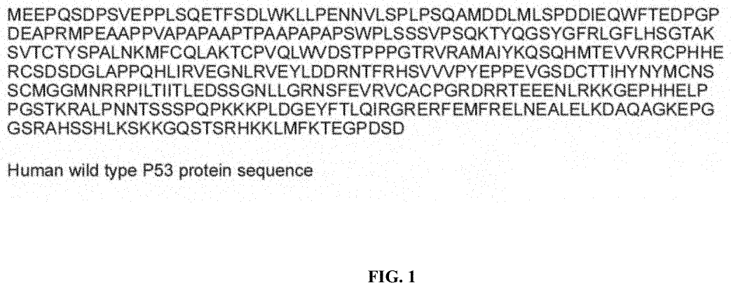

The table above refers to the sequence of p53 shown in FIG. 1. Amino acid changes are reported as: the amino acid being substituted followed by the position of the amino acid being substituted in the wild type p53 sequence, followed by the amino acid used for substitution. For example, L344P, indicates that the lysine (K) at the 344 position in the wild type sequence is replaced by a proline (P).

In some embodiments, the subject treated for cancer in accordance with the methods provided herein is a human, who has or is diagnosed with a tumor that is p53 negative. In some embodiments, a subject treated for cancer in accordance with the methods provided herein is a human, predisposed or susceptible to a cancer that is p53 negative. In some embodiments, a subject treated for cancer in accordance with the methods provided herein is a human, at risk of developing a cancer that is p53 negative.

In some embodiments, the subject treated for cancer in accordance with the methods provided herein is a human, who has or is diagnosed with a tumor that expresses p53 with a partial loss of function mutation. In other embodiments, a subject treated for cancer in accordance with the methods provided herein is a human, predisposed or susceptible to a cancer that expresses p53 with partial loss of function mutation. In some embodiments, a subject treated for tumor in accordance with the methods provided herein is a human, at risk of developing a cancer that expresses p53 with partial loss of function mutation. In some embodiments, a partial loss of p53 function mutation can cause the mutant p53 to exhibit some level of function of normal p53, but to a lesser or slower extent. For example, a partial loss of p53 function can mean that the cells become arrested in cell division to a lesser or slower extent.

In some embodiments, the subject treated for cancer in accordance with the methods provided herein is a human, who has or is diagnosed with a tumor that expresses p53 with a copy loss mutation and a deactivating mutation. In some embodiments, a subject treated for cancer in accordance with the methods provided herein is a human, predisposed or susceptible to a tumor that expresses p53 with a copy loss mutation and a deactivating mutation. In some embodiments, a subject treated for cancer in accordance with the methods provided herein is a human, at risk of developing a tumor that expresses p53 with a copy loss mutation and a deactivating mutation.

In some embodiments, the subject treated for cancer in accordance with the methods provided herein is a human, who has or is diagnosed with a cancer that expresses p53 with a copy loss mutation. In other embodiments, a subject treated for cancer in accordance with the methods provided herein is a human, predisposed or susceptible to a tumor that expresses p53 with a copy loss mutation. In some embodiments, a subject treated for cancer in accordance with the methods provided herein is a human, at risk of developing a cancer that expresses p53 with a copy loss mutation.

In some embodiments, a subject treated for cancer in accordance with the methods provided herein is a human, who has or is diagnosed with a tumor, determined to have a dominant p53 deactivating mutation. Dominant p53 deactivating mutation or dominant negative mutation, as used herein, is a mutation wherein the mutated p53 inhibits or disrupt the activity of the wild-type p53 gene.

In some embodiments, a subject treated for cancer in accordance with the methods provided herein is a human with non-cancerous tissue comprising a functional p53 protein. In some embodiments, the non-cancerous tissue comprising a functional p53 protein is bone marrow or tissue of the digestive tract (i.e., digestive tract tissue). In some embodiments, the subject is a human lacking a p53 deactivating mutation and/or expressing wild type p53. A p53 deactivating mutation in some examples can be a mutation in a DNA-binding domain of the p53 protein. In some embodiments, the p53 deactivating mutation can be a missense mutation. In some embodiments, the bone marrow of the subject can be determined to lack one or more p53 deactivating mutations selected from mutations at one or more of residues R175, G245, R248, R249, R273, and R282. The lack of a p53 deactivating mutation and/or the presence of wild type p53 in a non-cancerous tissue of the subject can be determined by any suitable method, for example by sequencing, array-based testing, RNA analysis and amplifications methods such as PCR.

In some embodiments, the subject treated for cancer in accordance with the methods provided herein is a human with non-cancerous tissue (e.g., bone marrow or tissue of the digestive tract) that expresses p53 with one or more silent mutations. Silent mutations can be mutations that cause no change in the encoded p53 amino acid sequence.

In some embodiments, a subject treated for cancer in accordance with the methods provided herein is a human with non-cancerous tissue (e.g., bone marrow or tissue of the digestive tract) determined to lack a dominant p53 deactivating mutation.

In some embodiments, the subject treated for cancer in accordance with the methods provided herein is a human with non-cancerous tissue (e.g., bone marrow or tissue of the digestive tract) that expresses p53 with a partial loss of function mutation.

Methods of Detecting Wild Type p53 and/or p53 Mutations

In some embodiments, a subject with a cancer having a p53-deactivating mutations and non-cancerous tissue comprising functional p53 protein is a candidate for cancer treatment with a method disclosed herein. Cancer cells and/or non-cancerous tissue from a subject can be assayed in order to determine the presence or absence of p53-deactivating mutations and/or the expression of wild type p53 in cancer/non-cancerous tissue prior to treatment with a compound of the disclosure. In some embodiments, the non-cancerous tissue is bone marrow. In some embodiments, the non-cancerous tissue is tissue of the digestive tract.

The activity of the p53 pathway can be determined by the mutational status of genes involved in the p53 pathways, including, for example, AKT1, AKT2, AKT3, ALK, BRAF, CDK4, CDKN2A, DDR2, EGFR, ERBB2 (HER2), FGFR1, FGFR3, GNA11, GNQ, GNAS, KDR, KIT, KRAS, MAP2K1 (MEK1), MET, HRAS, NOTCH1, NRAS, NTRK2, PIK3CA, NF1, PTEN, RAC1, RB1, NTRK3, STK11, PIK3R1, TSC1, TSC2, RET, TP53, and VHL. Genes that modulate the activity of p53 can also be assessed, including, for example, kinases: ABL1, JAK1, JAAK2, JAK3; receptor tyrosine kinases: FLT3 and KIT; receptors: CSF3R, IL7R, MPL, and NOTCH1; transcription factors: BCOR, CEBPA, CREBBP, ETV6, GATA1, GATA2. MLL, KZF1, PAX5, RUNX1, STAT3, WT1, and TP53; epigenetic factors: ASXL1, DNMT3A, EZH2, KDM6A (UTX), SUZ12, TET2, PTPN11, SF3B1, SRSF2, U2AF35, and ZRSR2; RAS proteins: HRAS, KRAS, and NRAS; adaptors CBL and CBL-B; FBXW7, IDH1, IDH2, and NPM1.

Cancer cell samples can be obtained, for example, from solid or liquid tumors via primary or metastatic tumor resection (e.g. pneumonectomy, lobetomy, wedge resection, and craniotomy) primary or metastatic disease biopsy (e.g. transbronchial or needle core), pleural or ascites fluid (e.g. FFPE cell pellet), or macro-dissection of tumor rich areas (solid tumors).

To detect the p53 wild type gene and/or lack of p53 deactivation mutation in a cancerous tissue, cancerous tissue can be isolated from surrounding normal tissues. For example, the tissue can be isolated from paraffin or cryostat sections. Cancer cells can also be separated from normal cells by flow cytometry.

Non-cancerous tissue samples can be obtained, for example, from bone marrow, bone marrow aspirate, bone marrow clot, a bone marrow biopsy, digestive tract tissues such as intestinal lining, stomach lining, and mucous membranes; liver, spleen pancreas, skin, lungs, heart, kidney, gall bladder, appendix, brain, mouth, tongue, throat, ocular tissue, fat, muscle, and lymph nodes.

Various methods and assays for analyzing wild type p53 and/or p53 mutations are suitable for use in the methods of the disclosure. Non-limiting examples of assays include polymerase chain reaction (PCR), restriction fragment length polymorphism (RFLP), microarray, Southern blot, northern blot, western blot, eastern blot, hematoxylin and eosin (H&E) staining, microscopic assessment of tumors, DNA sequencing, RNA sequencing, next-generation DNA sequencing (NGS) (e.g. extraction, purification, quantification, and amplification of DNA, library preparation) immunohistochemistry, and fluorescent in situ hybridization (FISH).

A microarray allows a researcher to investigate multiple DNA sequences attached to a surface, for example, a DNA chip made of glass or silicon, or a polymeric bead or resin. The DNA sequences are hybridized with fluorescent or luminescent probes. The microarray can indicate the presence of oligonucleotide sequences in a sample based on hybridization of sample sequences to the probes, followed by washing and subsequent detection of the probes. Quantification of the fluorescent or luminescent signal indicates the presence of known oligonucleotide sequences in the sample.

A microarray allows a researcher to investigate multiple DNA sequences attached to a surface, for example, a DNA chip made of glass or silicon, or a polymeric bead or resin. The DNA sequences are hybridized with fluorescent or luminescent probes. The microarray can indicate the presence of oligonucleotide sequences in a sample based on hybridization of sample sequences to the probes, followed by washing and subsequent detection of the probes. Quantification of the fluorescent or luminescent signal indicates the presence of known oligonucleotide sequences in the sample.

In some embodiments, an assay comprises amplifying a biomolecule from a biological sample such as a bone marrow or cancer sample. The biomolecule can be a nucleic acid molecule, such as DNA or RNA. In some embodiments, the assay comprises circularization of a nucleic acid molecule, followed by digestion of the circularized nucleic acid molecule.

In some embodiments, the assay comprises contacting an organism, or a biochemical sample collected from an organism, such as a nucleic acid sample, with a library of oligonucleotides, such as PCR primers. The library can contain any number of oligonucleotide molecules. The oligonucleotide molecules can bind individual DNA or RNA motifs, or any combination of motifs described herein. The motifs can be any distance apart, and the distance can be known or unknown. In some embodiments, two or more oligonucleotides in the same library bind motifs a known distance apart in a parent nucleic acid sequence. Binding of the primers to the parent sequence can take place based on the complementarity of the primers to the parent sequence. Binding can take place, for example, under annealing, or under stringent conditions.

In some embodiments, the results of an assay are used to design a new oligonucleotide sequence for future use. In some embodiments, the results of an assay are used to design a new oligonucleotide library for future use. In some embodiments, the results of an assay are used to revise, refine, or update an existing oligonucleotide library for future use. For example, an assay can reveal that a previously-undocumented nucleic acid sequence is associated with the presence of a target material. This information can be used to design or redesign nucleic acid molecules and libraries.

In some embodiments, one or more nucleic acid molecules in a library comprise a barcode tag. In some embodiments, one or more of the nucleic acid molecules in a library comprise type I or type II restriction sites suitable for circularization and cutting an amplified sample nucleic acid sequence. Such primers can be used to circularize a PCR product and cut the PCR product to provide a product nucleic acid sequence with a sequence that is organized differently from the nucleic acid sequence native to the sample organism.

After a PCR experiment, the presence of an amplified sequence can be verified. Non-limiting examples of methods for finding an amplified sequence include DNA sequencing, whole transcriptome shotgun sequencing (WTSS, or RNA-seq), mass spectrometry (MS), microarray, pyrosequencing, column purification analysis, polyacrylamide gel electrophoresis, and index tag sequencing of a PCR product generated from an index-tagged primer.

In some embodiments, more than one nucleic acid sequence in the sample organism is amplified. Non-limiting examples of methods of separating different nucleic acid sequences in a PCR product mixture include column purification, high performance liquid chromatography (HPLC), HPLC/MS, polyacrylamide gel electrophoresis, size exclusion chromatography.

The amplified nucleic acid molecules can be identified by sequencing. Nucleic acid sequencing can be done on automated instrumentation. Sequencing experiments can be done in parallel to analyze tens, hundreds, or thousands of sequences simultaneously. Non-limiting examples of sequencing techniques follow.

In pyrosequencing, DNA is amplified within a water droplet containing a single DNA template bound to a primer-coated bead in an oil solution. Nucleotides are added to a growing sequence, and the addition of each base is evidenced by visual light.

Ion semiconductor sequencing detects the addition of a nucleic acid residue as an electrical signal associated with a hydrogen ion liberated during synthesis. A reaction well containing a template is flooded with the four types of nucleotide building blocks, one at a time. The timing of the electrical signal identifies which building block was added and identifies the corresponding residue in the template.

DNA nanoball uses rolling circle replication to amplify DNA into nanoballs. Unchained sequencing by ligation of the nanoballs reveals the DNA sequence.

In a reversible dyes approach, nucleic acid molecules are annealed to primers on a slide and amplified. Four types of fluorescent dye residues, each complementary to a native nucleobase, are added, the residue complementary to the next base in the nucleic acid sequence is added, and unincorporated dyes are rinsed from the slide. Four types of reversible terminator bases (RT-bases) are added, and non-incorporated nucleotides are washed away. Fluorescence indicates the addition of a dye residue, thus identifying the complementary base in the template sequence. The dye residue is chemically removed, and the cycle repeats.

Detection of the presence or absence of point mutations can be accomplished by molecular cloning of the p53 allele(s) present in the cancer cell tissue and sequencing that allele(s). Alternatively, the polymerase chain reaction can be used to amplify p53 gene sequences directly from a genomic DNA preparation from a biological sample such as bone marrow, tissue of the digestive tract, cancer cells, or cancer tissue. The DNA sequence of the amplified sequences can then be determined. Specific deletions of p53 genes can also be detected. For example, restriction fragment length polymorphism (RFLP) probes for the p53 gene or surrounding marker genes can be used to score loss of a p53 allele.

Loss of wild type p53 genes can also be detected on the basis of the loss of a wild type expression product of the p53 gene. Such expression products include both the mRNA as well as the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via polymerase chain reaction (PCR).

Alternatively, mismatch detection can be used to detect the presence or absence of point mutations in the p53 gene or the mRNA product. The method can involve the use of a labeled riboprobe that is complementary to the human wild type p53 gene. The riboprobe and either mRNA or DNA isolated from the cancer cell tissue are annealed (hybridized) together and subsequently digested with the enzyme RNase A, which is able to detect some mismatches in a duplex RNA structure. If a mismatch is detected by RNase A, the enzyme cleaves at the site of the mismatch. Thus, when the annealed RNA preparation is separated on an electrophoretic gel matrix, if a mismatch has been detected and cleaved by RNase A, a RNA product is seen that is smaller than is the full-length duplex RNA for the riboprobe and the p53 mRNA or DNA. The riboprobe need not be the full length of the p53 mRNA or gene but can be a segment of either. If the riboprobe comprises only a segment of the p53 mRNA, then a number of these probes can be used to screen the whole mRNA sequence for mismatches.

In similar fashion, DNA probes can be used to detect the presence or absence mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA, which might contain a mutation, can be amplified using PCR before hybridization.

DNA sequences of the p53 gene from a biological sample such as bone marrow, tissue of the digestive tract, cancer cells, or cancerous tissue, which have been amplified by use of polymerase chain reaction, can also be screened using allele-specific probes. These probes are nucleic acid oligomers, each of which contains a region of the p53 gene sequence harboring a known mutation. For example, one oligomer can be about 30 nucleotides in length, corresponding to a portion of the p53 gene sequence. At the position coding for the 175th codon of the p53 gene, the oligomer encodes an alanine, rather than the wild type codon valine. By use of a battery of such allele-specific probes, the PCR amplification products can be screened to identify the presence of a previously-identified mutation in the p53 gene. Hybridization of allele-specific probes with amplified p53 sequences can be performed, for example, on a nylon filter. Hybridization to a particular probe indicates the presence of the same mutation in the biological sample as in the allele-specific probe.

The identification of p53 gene structural changes in a biological sample such as bone marrow, tissue of the digestive tract, cancer cells, or cancerous tissue can be facilitated through the application of a diverse series of high resolution, high throughput microarray platforms. Essentially two types of array include those that carry PCR products from cloned nucleic acids (e.g. cDNA, BACs, cosmids) and those that use oligonucleotides. The methods can provide a way to survey genome wide DNA copy number abnormalities and expression levels to allow correlations between losses, gains and amplifications in cancer cells with genes that are over- and under-expressed in the same samples. The gene expression arrays that provide estimates of mRNA levels in biological samples have given rise to exon-specific arrays that can identify both gene expression levels, alternative splicing events and mRNA processing alterations.

Oligonucleotide arrays can be used to interrogate single nucleotide polymorphisms (SNPs) throughout the genome for linkage and association studies and these have been adapted to quantify copy number abnormalities and loss of heterozygosity events. DNA sequencing arrays can allow resequencing of chromosome regions, exomes, and whole genomes.

Single nucleotide polymorphism (SNP)-based arrays or other gene arrays or chips can determine the presence or absence of wild type p53 allele and the structure of mutations. SNPs can be synonymous or nonsynonymous substitutions. Synonymous SNP substitutions do not result in a change of amino acid in the protein due to the degeneracy of the genetic code, but can affect function in other ways. For example, a seemingly silent mutation in a gene that codes for a membrane transport protein can slow down translation, allowing the peptide chain to misfold, and produce a less functional mutant membrane transport protein. Nonsynonymous SNP substitutions can be missense substitutions or nonsense substitutions. Missense substitutions occur when a single base change results in change in amino acid sequence of the protein and malfunction thereof leads to disease. Nonsense substitutions occur when a point mutation results in a premature stop codon, or a nonsense codon in the transcribed mRNA, which results in a truncated and usually, nonfunctional, protein product. As SNPs are highly conserved throughout evolution and within a population, the map of SNPs serves as an excellent genotypic marker for research. A SNP array can be a useful tool to study the whole genome.

In addition, SNP-based arrays can be used for studying the Loss Of Heterozygosity (LOH). LOH is a form of allelic imbalance that can result from the complete loss of an allele or from an increase in copy number of one allele relative to the other. While other chip-based methods (e.g., comparative genomic hybridization) can detect only genomic gains or deletions, SNP-based arrays have the additional advantage of detecting copy number neutral LOH due to uniparental disomy (UPD). In UPD, one allele or whole chromosome from one parent are missing, and the other parental allele is reduplicated (uni-parental=from one parent, disomy=duplicated). In a disease setting, this occurrence can be pathologic when the wild type allele (e.g., from the mother) is missing and instead two copies of the heterozygous allele (e.g., from the father) are present. This usage of SNP-based arrays has a huge potential in cancer diagnostics as LOH is a prominent characteristic of most human cancers. SNP-based array technologies have shown that cancers (e.g. gastric cancer, liver cancer, etc.) and hematologic malignancies (ALL, MDS, CML etc) have a high rate of LOH due to genomic deletions or UPD and genomic gains. In the present disclosure, using high density SNP-based arrays to detect LOH can allow for the identification of pattern of allelic imbalance to determine the presence of wild type p53 allele.

Mutations of wild type p53 genes can also be detected on the basis of the mutation of a wild type expression product of the p53 gene. Such expression products include both the mRNA and the p53 protein product itself. Point mutations can be detected by sequencing the mRNA directly or via molecular cloning of cDNA made from the mRNA. The sequence of the cloned cDNA can be determined using DNA sequencing techniques. The cDNA can also be sequenced via the polymerase chain reaction (PCR). A panel of monoclonal antibodies can be used in which each of the epitopes involved in p53 functions are represented by a monoclonal antibody. Loss or perturbation of binding of a monoclonal antibody in the panel can indicate mutational alteration of the p53 protein and thus of the p53 gene itself. Mutant p53 genes or gene products can also be detected in body samples, including, for example, bone marrow, tissue of the digestive tract, cancer cells, cancerous tissues, serum, stool, urine, and sputum. The same techniques discussed above for detection of mutant p53 genes or gene products in tissues can be applied to other body samples.

Loss of wild type p53 genes can also be detected by screening for loss of wild type p53 protein function. Protein p53 binds to the SV40 large T antigen as well as to the adenovirus E1B antigen. Loss of the ability of the p53 protein to bind to either or both of these antigens indicates a mutational alteration in the protein and reflects a mutational alteration of the gene. Alternatively, a panel of monoclonal antibodies can be used in which each of the epitopes involved in p53 functions is represented by a monoclonal antibody. Loss or perturbation of binding of a monoclonal antibody in the panel would indicate mutational alteration of the p53 protein and thus of the p53 gene. Any method for detecting an altered p53 protein can be used to detect loss of wild type p53 genes.

Wild type p53 and/or p53 mutations in cancerous or non-cancerous tissue can be detected any time before, during, or after the administration of a peptidomimetic macrocycle and/or another pharmaceutically-active agent. In some embodiments, the detection is performed before administration of a peptidomimetic macrocycle or other pharmaceutically-active agent, for example about 5 years-1 month, 4 years-1 month, 3 years-1 month, 2 years-1 month, 1 years-1 month, 5 years-1 week, 4 years-1 week, 3 years-1 month, 2 years-1 week, 1 year-1 week, 5 years-1 day, 4 years-1 day, 3 years-1 days, 2 years-1 day, 1 year-1 day, 15 months-1 month, 15 months-1 week, 15 months-1 day, 12 months-1 month, 12 months-1 week, 12 months-1 day, 6 months-1 month, 6 months-1 week, 6 months-1 day, 3 months-1 month, 3 months-1 week, or 3 months-1 day prior to the first administration of the peptidomimetic macrocycle or other pharmaceutically-active agent. In some examples, wild type p53 and/or p53 mutations are detected up to 6 years, up to 5 years, up to 4 years, up to 3 years, up to 24 months, up to 23 months, up to 22 months, up to 21 months, up to 20 months, up to 19 months, up to 18 months, up to 17 months, up to 16 months, up to 15 months, up to 14 months, up to 13 months, up to 12 months, up to 11 months, up to 10 months, up to 9 months, up to 8 months, up to 7 months, up to 6 months, up to 5 months, up to 4 months, up to 3 months, up to 2 months, up to 1 months, up to 4 weeks (28 days), up to 3 weeks (21 days), up to 2 weeks (14 days), up to 1 week (7 days), up to 6 days, up to 5 days, up to 4 days, up to 3 days, up to 2 days or up to 1 day before the first administration of the peptidomimetic macrocycle or other pharmaceutically-active agent to the subject.

### Myelopreservation Agents

A method disclosed herein can comprise administration of a peptidomimetic macrocycle in combination with a second pharmaceutically-active agent. In some instances, the peptidomimetic macrocycle can serve as a myelopreservation agent. A myelopreservation agent can prevent, reduce, or reduce a likelihood of myelosuppressive side effects of a pharmaceutically-active agent. Myelosuppressive side effects can be due to cytotoxic effects of a pharmaceutically-active agent on bone marrow cells. Non-limiting examples of myelosuppressive side effects include anemia, leukopenia, neutropenia, thrombocytopenia, and pancytopenia. In some instances, cells undergoing cell cycle arrest are resistant to the cytotoxic effects of a pharmaceutically-active agent. Cell cycle arrest can be induced by, for example, activation of p53. In some embodiments, a method of treating cancer disclosed herein comprises inducing cell cycle arrest in bone marrow via p53 activation in order to reduce the myelosuppressive side effects of a pharmaceutically-active agent. p53 activation can be induced by, for example, inhibition of MDM2 and/or MDMX proteins via administration of a peptidomimetic macrocycle disclosed herein. In some embodiments, the peptidomimetic macrocycle binds to MDM2 and/or MDMX proteins. In some embodiments, the peptidomimetic macrocycle is administered at a dose that is less than a dose needed to induce apoptosis in a tissue such as bone marrow.

### Protection Against Drug-Induced Mucositis

In some embodiments, a peptidomimetic macrocycle disclosed herein can prevent, reduce, or reduce a likelihood of mucositis caused by a second pharmaceutically-active agent. Mucositis can be due to cytotoxic effects of a pharmaceutically-active agent on the cells lining the digestive tract. Cell death along the digestive tract can lead to thinning of the epithelium, resulting in mucosal destruction. In some instances, cells of the digestive tract undergoing cell cycle arrest are resistant to the cytotoxic effects of a pharmaceutically active agent (e.g., a chemotherapeutic agent). Cell cycle arrest can be induced by, for example, activation of p53. In some embodiments, a method of treating cancer disclosed herein comprises inducing cell cycle arrest in the digestive tract via p53 activation in order to reduce mucositis caused by a pharmaceutically-active agent. p53 activation can be induced by, for example, inhibition of MDM2 and/or MDMX proteins via administration of a peptidomimetic macrocycle. In some embodiments, the peptidomimetic macrocycle binds to MDM2 and/or MDMX proteins. In some embodiments, the peptidomimetic macrocycle is administered at a dose that is less than a dose needed to induce apoptosis in tissue such as digestive tract tissue.

### Peptidomimetic Macrocycles

In some embodiments, a peptidomimetic macrocycle has the Formula (I):

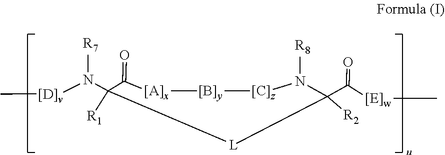

wherein:
each A, C, D, and E is independently a natural or non-natural amino acid or an amino acid analog, and each terminal D and E independently optionally includes a capping group;
each B is independently a natural or non-natural amino acid, an amino acid analog,

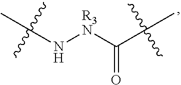

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]n, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000, for example 1-500, 1-200, 1-100, 1-50, 1-30, 1-20, or 1-10;
u is an integer from 1-10, for example 1-5, 1-3 or 1-2;
each x, y, and z is independently an integer from 0-10, for example the sum of x+y+z is 2, 3, or 6; and
n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In an embodiment of any of the Formulas described herein, L₁ and L₂, either alone or in combination, do not form a triazole or a thioether.

In one example, at least one of R₁ and R₂ is alkyl that is unsubstituted or substituted with halo-. In another example, both R₁ and R₂ are independently alkyl that is unsubstituted or substituted with halo-. In some embodiments, at least one of R₁ and R₂ is methyl. In other embodiments, R₁ and R₂ are methyl.

In some embodiments, x+y+z is at least 3. In other embodiments, x+y+z is 1, 2, 3, 4, 5, 6, 7, 8, 9 or 10. In some embodiments, the sum of x+y+z is 3 or 6. In some embodiments, the sum of x+y+z is 3. In other embodiments, the sum of x+y+z is 6. Each occurrence of A, B, C, D or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is an α-helix and R₈ is —H, allowing for intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

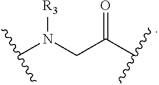

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, peptidomimetic macrocycles are also provided of the formula:

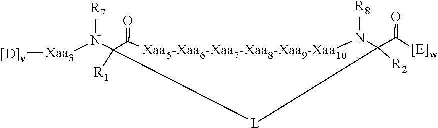

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 1945), wherein each X is an amino acid;
each D and E is independently a natural or non-natural amino acid or an amino acid analog;
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000, for example 1-500, 1-200, 1-100, 1-50, 1-30, 1-20 or 1-10;
w is an integer from 3-1000, for example 3-500, 3-200, 3-100, 3-50, 3-30, 3-20, or 3-10; and
n is an integer from 1-5.

In some embodiments, v and w are integers from 1-30. In some embodiments, w is an integer from 3-1000, for example 3-500, 3-200, 3-100, 3-50, 3-30, 3-20, or 3-10. In some embodiments, the sum of x+y+z is 3 or 6. In some embodiments, the sum of x+y+z is 3. In other embodiments, the sum of x+y+z is 6.

In some embodiments of any of the Formulas described herein, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-His5-Tyr₆-Trp₇-Ala₈-Gln₉-Leu₁₀-X₁-Ser₁₂ (SEQ ID NO: 1945). In other embodiments, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 1945). In other embodiments, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-His₅-Tyr₆-Trp₇-Ala₈-Gln₉-Leu₁₀-X₁₁-Ser₁₂ (SEQ ID NO: 1945). In other embodiments, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-His5-Tyr₆-Trp₇-Ala₈-Gln₉-Leu₁₀-X₁₁-Ser₁₂ (SEQ ID NO: 1945). In other embodiments, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-His5-Tyr₆-Trp₇-Ala₈-Gln₉-Leu₁₀-X₁₁-Ser₁₂ (SEQ ID NO: 1945).

In some embodiments, a peptidomimetic macrocycle has the Formula:

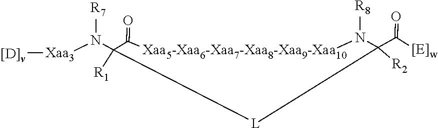

wherein:
each of Xaa₃, Xaa₅, Xaa₆, Xaa₇, Xaa₈, Xaa₉, and Xaa₁₀ is individually an amino acid, wherein at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 1946), wherein each X is an amino acid;
each D is independently a natural or non-natural amino acid or an amino acid analog;
each E is independently a natural or non-natural amino acid or an amino acid analog, for example an amino acid selected from Ala (alanine), D-Ala (D-alanine), Aib (α-aminoisobutyric acid), Sar (N-methyl glycine), and Ser (serine);
R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each L and L′ is independently a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄-]n, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v is an integer from 1-1000, for example 1-500, 1-200, 1-100, 1-50, 1-30, 1-20, or 1-10;
w is an integer from 3-1000, for example 3-500, 3-200, 3-100, 3-50, 3-30, 3-20, or 3-10; and
n is an integer from 1-5.

In some embodiments of the above Formula, at least three of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 1946). In other embodiments of the above Formula, at least four of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 1946). In other embodiments of the above Formula, at least five of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X₁₁-Ala₁₂ (SEQ ID NO: 1946). In other embodiments of the above Formula, at least six of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X11-Ala₁₂ (SEQ ID NO: 1946). In other embodiments of the above Formula, at least seven of Xaa₃, Xaa₅, Xaa₆, Xaa₇, Xaa₈, Xaa₉, and Xaa₁₀ are the same amino acid as the amino acid at the corresponding position of the sequence Phe₃-X₄-Glu₅-Tyr₆-Trp₇-Ala₈-Gln₉-Leu₁₀/Cba₁₀-X11-Ala₁₂ (SEQ ID NO: 1946).

In some embodiments, w is an integer from 3-10, for example 3-6, 3-8, 6-8, or 6-10. In some embodiments, w is 3. In other embodiments, w is 6. In some embodiments, v is an integer from 1-10. In some embodiments, v is 2.

In an embodiment of any of the Formulas described herein, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 3. In other embodiments, x+y+z is 1, 2, 3, 4, 5, 6, 7, 8, 9 or 10. In some embodiments, the sum of x+y+z is 3 or 6. In some embodiments, the sum of x+y+z is 3. In other embodiments, the sum of x+y+z is 6. Each occurrence of A, B, C, D or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is an α-helix and R₈ is —H, allowing intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

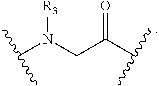

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, a peptidomimetic macrocycle of Formula (I) has Formula (Ia):

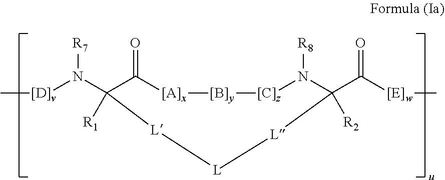

wherein:
each A, C, D, and E is independently a natural or non-natural amino acid or an amino acid analog;
each B is independently a natural or non-natural amino acid, amino acid analog,

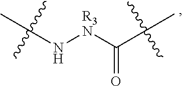

[—NH-L₃-CO—], [—NH-L₃-SO₂—], or [—NH-L₃-];
each L is independently a macrocycle-forming linker;
each L′ is independently alkylene, alkenylene, alkynylene, heteroalkylene, cycloalkylene, heterocycloalkylene, arylene, or heteroarylene, each being optionally substituted with R₅, or a bond, or together with R₁ and the atom to which both R₁ and L′ are bound forms a ring;
each L″ is independently alkylene, alkenylene, alkynylene, heteroalkylene, cycloalkylene, heterocycloalkylene, arylene, or heteroarylene, each being optionally substituted with R₅, or a bond, or together with R₂ and the atom to which both R₂ and L″ are bound forms a ring;
each R₁ is independently —H, alkyl, alkenyl, alkynyl, arylalkyl, cycloalkyl, cycloalkylalkyl, heteroalkyl, or heterocycloalkyl, unsubstituted or substituted with halo-, or together with L′ and the atom to which both R₁ and L′ are bound forms a ring;
each R₂ is independently —H, alkyl, alkenyl, alkynyl, arylalkyl, cycloalkyl, cycloalkylalkyl, heteroalkyl, or heterocycloalkyl, unsubstituted or substituted with halo-, or together with L″ and the atom to which both R₂ and L″ are bound forms a ring;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
n is an integer from 1-5;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000, for example 1-500, 1-200, 1-100, 1-50, 1-40, 1-25, 1-20, 1-15, or 1-10;
each x, y and z is independently an integer from 0-10, for example x+y+z is 2, 3, or 6; and
u is an integer from 1-10, for example 1-5, 1-3, or 1-2.

In some embodiments, L is a macrocycle-forming linker of the formula -L₁-L₂-. In some embodiments, each L₁ and L₂ is independently alkylene, alkenylene, alkynylene, heteroalkylene, cycloalkylene, heterocycloalkylene, arylene, heteroarylene, or [—R₄—K—R₄—]ₙ, each being optionally substituted with R₅; each R₄ is independently alkylene, alkenylene, alkynylene, heteroalkylene, cycloalkylene, heterocycloalkylene, arylene, or heteroarylene; each K is independently O, S, SO, SO₂, CO, CO₂, or CONR₃; and n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 2. In other embodiments, x+y+z is 1, 2, 3, 4, 5, 6, 7, 8, 9, or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g. Gln-Asp-Ala as well as embodiments wherein the amino acids are identical, e.g. Gln-Gln-Gln. This applies for any value of x, y, or z in the indicated ranges. Similarly, when u is greater than 1, each compound can encompass peptidomimetic macrocycles which are the same or different. For example, a compound can comprise peptidomimetic macrocycles comprising different linker lengths or chemical compositions.

In some embodiments, the peptidomimetic macrocycle comprises a secondary structure which is a helix and R₈ is —H, allowing intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

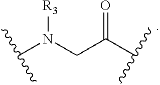

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Cα.

In one embodiment, the peptidomimetic macrocycle of Formula (I) is:

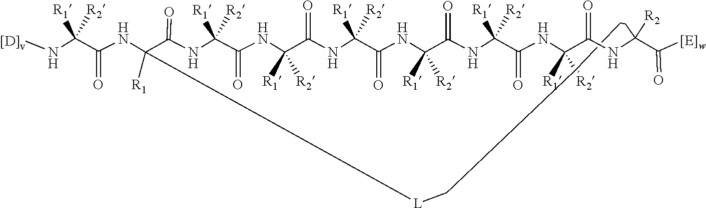

wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle of Formula (I) is:

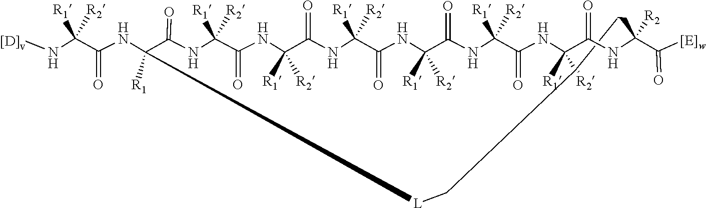

wherein each R₁′ and R₂′ is independently an amino acid.

In other embodiments, the peptidomimetic macrocycle of Formula (I) is a compound of any of the formulas shown below:

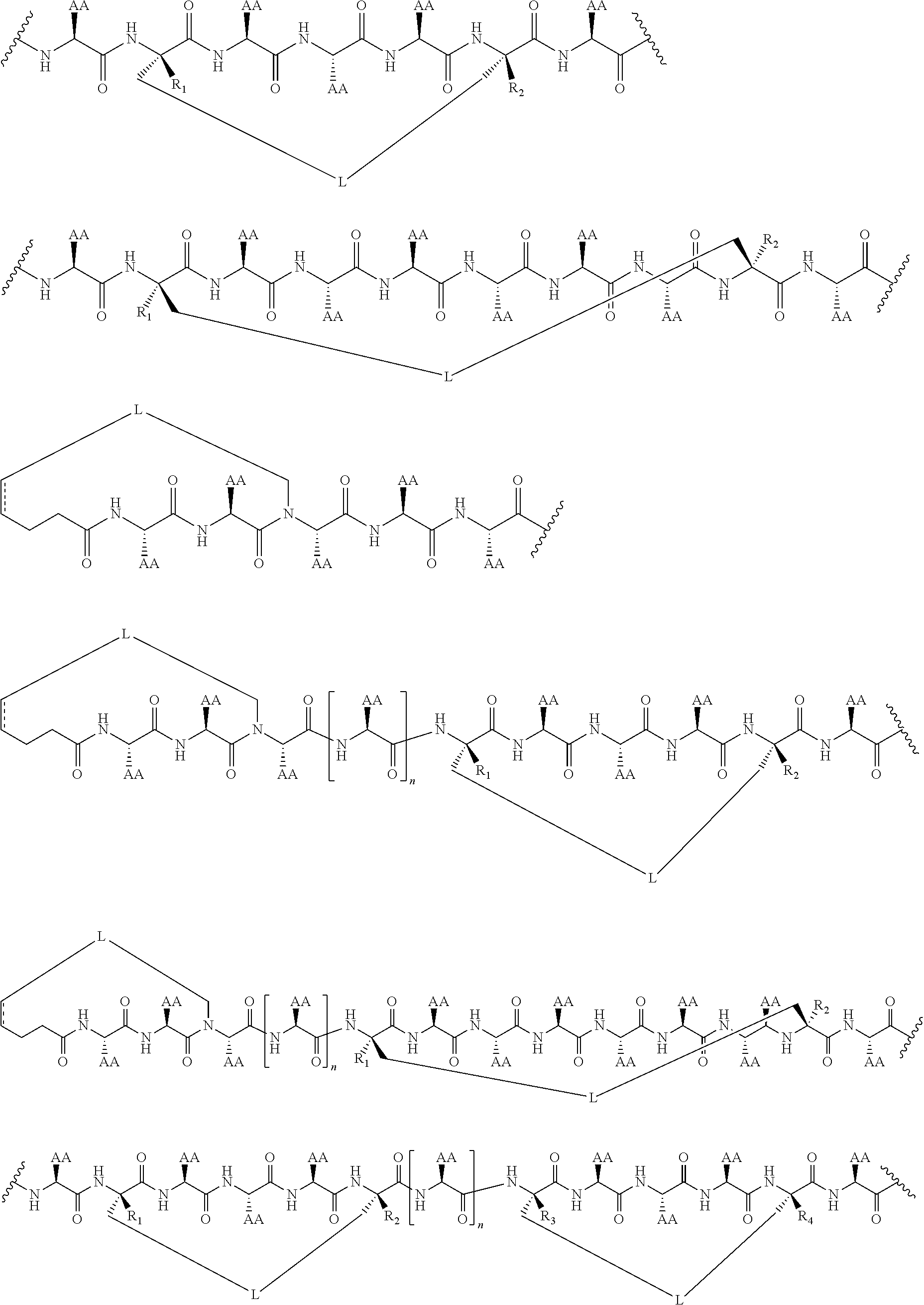

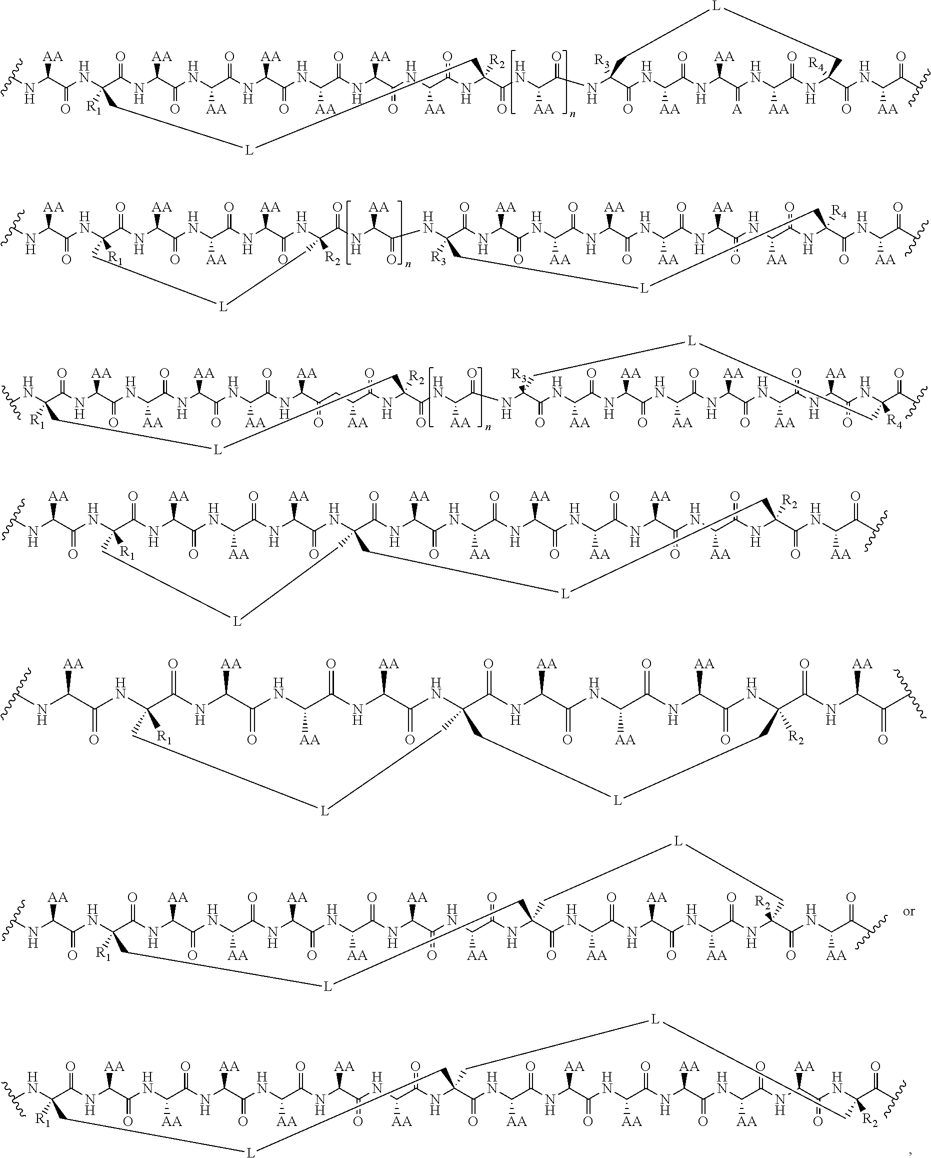

wherein “AA” represents any natural or non-natural amino acid side chain and “ [symbol] ” is [D]ᵥ, [E]_{w} as defined above, and n is an integer between 0 and 20, 50, 100, 200, 300, 400 or 500. In some embodiments, n is 0. In other embodiments, n is less than 50.

Non-limiting examples of embodiments of the macrocycle-forming linker L are shown below.

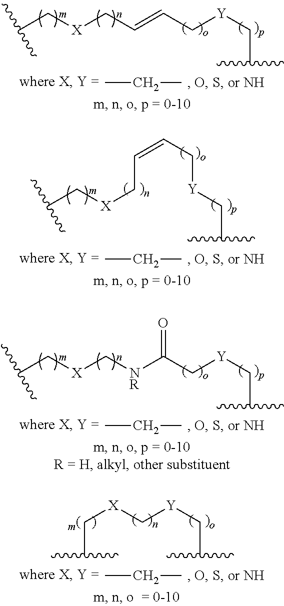

In other embodiments, D and/or E in the compound of Formula I are further modified to facilitate cellular uptake. In some embodiments, lipidating or PEGylating a peptidomimetic macrocycle facilitates cellular uptake, increases bioavailability, increases blood circulation, alters pharmacokinetics, decreases immunogenicity and/or decreases the needed frequency of administration.

In other embodiments, at least one of [D] and [E] in the compound of Formula I represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers. In an embodiment, u is 2.

In some embodiments, the peptidomimetic macrocycles have the Formula (I):

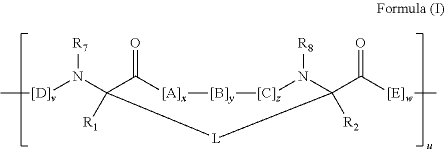

wherein:
each A, C, D, and E is independently a natural or non-natural amino acid or an amino acid analog;
each B is independently a natural or non-natural amino acid, amino acid analog,

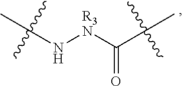

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently macrocycle-forming linker of the formula

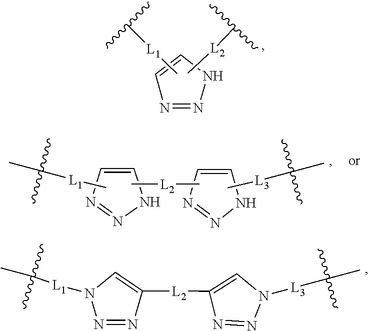

wherein each L₁, L₂ and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent; each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 1-1000;
each x, y and z is independently an integer from 0-10;
us is an integer from 1-10; and
n is an integer from 1-5.

In one example, at least one of R₁ and R₂ is alkyl that is unsubstituted or substituted with halo-. In another example, both R₁ and R₂ are independently alkyl that are unsubstituted or substituted with halo-. In some embodiments, at least one of R₁ and R₂ is methyl. In other embodiments, R₁ and R₂ are methyl.

In some embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g. Gln-Asp-Ala as well as embodiments wherein the amino acids are identical, e.g. Gln-Gln-Gln. This applies for any value of x, y, or z in the indicated ranges.

In some embodiments, each of the first two amino acid represented by E comprises an uncharged side chain or a negatively charged side chain. In some embodiments, each of the first three amino acid represented by E comprises an uncharged side chain or a negatively charged side chain. In some embodiments, each of the first four amino acid represented by E comprises an uncharged side chain or a negatively charged side chain. In some embodiments, one or more or each of the amino acid that is i+1, i+2, i+3, i+4, i+5, and/or i+6 with respect to Xaa₁₃ represented by E comprises an uncharged side chain or a negatively charged side chain.

In some embodiments, the first C-terminal amino acid and/or the second C-terminal amino acid represented by E comprise a hydrophobic side chain. For example, the first C-terminal amino acid and/or the second C-terminal amino acid represented by E comprises a hydrophobic side chain, for example a small hydrophobic side chain. In some embodiments, the first C-terminal amino acid, the second C-terminal amino acid, and/or the third C-terminal amino acid represented by E comprise a hydrophobic side chain. For example, the first C-terminal amino acid, the second C-terminal amino acid, and/or the third C-terminal amino acid represented by E comprises a hydrophobic side chain, for example a small hydrophobic side chain. In some embodiments, one or more or each of the amino acid that is i+1, i+2, i+3, i+4, i+5, and/or i+6 with respect to Xaa₁₃ represented by E comprises an uncharged side chain or a negatively charged side chain.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, the peptidomimetic macrocycle comprises a secondary structure which is a helix and R₈ is —H, allowing intra-helical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

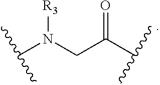

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Cα.

In some embodiments, L is a macrocycle-forming linker of the formula

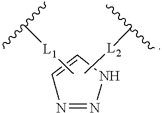

In some embodiments, L is a macrocycle-forming linker of the formula

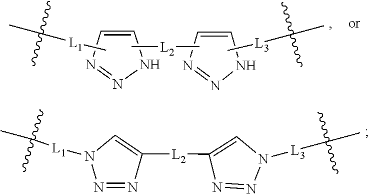

or a tautomer thereof.

Non-limiting examples of embodiments of the macrocycle-forming linker L are shown below:

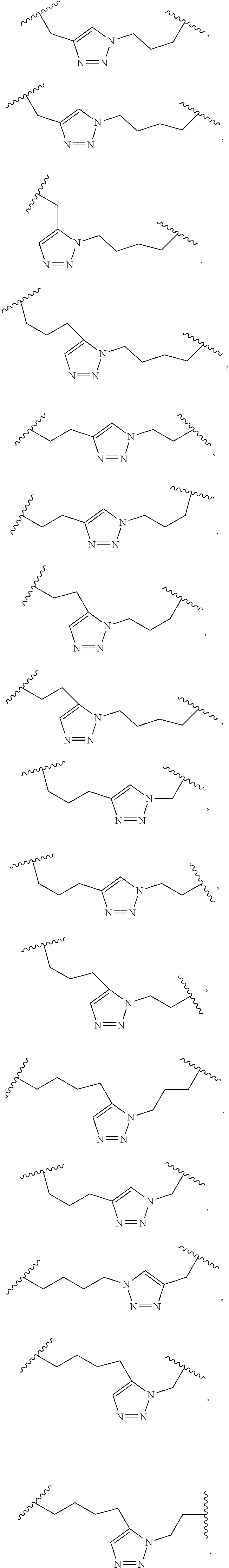

[chemical structure]

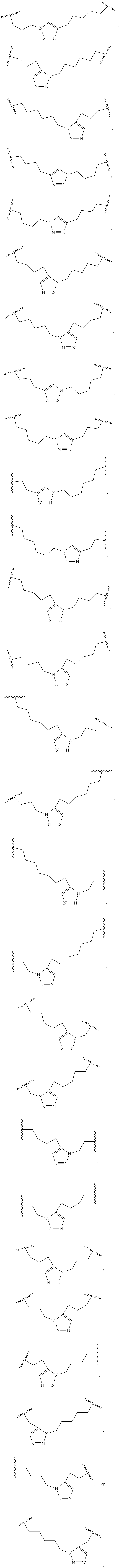

Amino acids which are used in the formation of triazole crosslinkers are represented according to the legend indicated below. Stereochemistry at the alpha position of each amino acid is S unless otherwise indicated. For azide amino acids, the number of carbon atoms indicated refers to the number of methylene units between the alpha carbon and the terminal azide. For alkyne amino acids, the number of carbon atoms indicated is the number of methylene units between the alpha position and the triazole moiety plus the two carbon atoms within the triazole group derived from the alkyne.

| | |
|---|---|
| $5a5 | Alpha-Me alkyne 1,5 triazole (5 carbon) |
| $5n3 | Alpha-Me azide 1,5 triazole (3 carbon) |
| $4rn6 | Alpha-Me R-azide 1,4 triazole (6 carbon) |
| $4a5 | Alpha-Me alkyne 1,4 triazole (5 carbon) |

In some embodiments, any of the macrocycle-forming linkers described herein can be used in any combination with any of the sequences shown in Table 1, Table 1a, Table 1b, Table 1c, Table 2a, Table 2b, Table 3, or Table 3a and also with any of the R— substituents indicated herein.

In some embodiments, the peptidomimetic macrocycle comprises at least one α-helix motif. For example, A, B and/or C in the compound of Formula I include one or more α-helices. As a general matter, α-helices include between 3 and 4 amino acid residues per turn. In some embodiments, the α-helix of the peptidomimetic macrocycle includes 1 to 5 turns and, therefore, 3 to 20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix.

In some embodiments, the macrocycle-forming linker spans from 1 turn to 5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5 Å to 9 Å per turn of the α-helix, or approximately 6 Å to 8 Å per turn of the α-helix.

Where the macrocycle-forming linker spans approximately 1 turn of an α-helix, the length is equal to approximately 5 carbon-carbon bonds to 13 carbon-carbon bonds, approximately 7 carbon-carbon bonds to 11 carbon-carbon bonds, or approximately 9 carbon-carbon bonds. Where the macrocycle-forming linker spans approximately 2 turns of an α-helix, the length is equal to approximately 8 carbon-carbon bonds to 16 carbon-carbon bonds, approximately 10 carbon-carbon bonds to 14 carbon-carbon bonds, or approximately 12 carbon-carbon bonds. Where the macrocycle-forming linker spans approximately 3 turns of an α-helix, the length is equal to approximately 14 carbon-carbon bonds to 22 carbon-carbon bonds, approximately 16 carbon-carbon bonds to 20 carbon-carbon bonds, or approximately 18 carbon-carbon bonds. Where the macrocycle-forming linker spans approximately 4 turns of an α-helix, the length is equal to approximately 20 carbon-carbon bonds to 28 carbon-carbon bonds, approximately 22 carbon-carbon bonds to 26 carbon-carbon bonds, or approximately 24 carbon-carbon bonds. Where the macrocycle-forming linker spans approximately 5 turns of an α-helix, the length is equal to approximately 26 carbon-carbon bonds to 34 carbon-carbon bonds, approximately 28 carbon-carbon bonds to 32 carbon-carbon bonds, or approximately 30 carbon-carbon bonds. Where the macrocycle-forming linker spans approximately 1 turn of an α-helix, the linkage contains approximately 4 atoms to 12 atoms, approximately 6 atoms to 10 atoms, or approximately 8 atoms. Where the macrocycle-forming linker spans approximately 2 turns of the α-helix, the linkage contains approximately 7 atoms to 15 atoms, approximately 9 atoms to 13 atoms, or approximately 11 atoms. Where the macrocycle-forming linker spans approximately 3 turns of the α-helix, the linkage contains approximately 13 atoms to 21 atoms, approximately 15 atoms to 19 atoms, or approximately 17 atoms. Where the macrocycle-forming linker spans approximately 4 turns of the α-helix, the linkage contains approximately 19 atoms to 27 atoms, approximately 21 atoms to 25 atoms, or approximately 23 atoms. Where the macrocycle-forming linker spans approximately 5 turns of the α-helix, the linkage contains approximately 25 atoms to 33 atoms, approximately 27 atoms to 31 atoms, or approximately 29 atoms.

Where the macrocycle-forming linker spans approximately 1 turn of the α-helix, the resulting macrocycle forms a ring containing approximately 17 members to 25 members, approximately 19 members to 23 members, or approximately 21 members. Where the macrocycle-forming linker spans approximately 2 turns of the α-helix, the resulting macrocycle forms a ring containing approximately 29 members to 37 members, approximately 31 members to 35 members, or approximately 33 members. Where the macrocycle-forming linker spans approximately 3 turns of the α-helix, the resulting macrocycle forms a ring containing approximately 44 members to 52 members, approximately 46 members to 50 members, or approximately 48 members. Where the macrocycle-forming linker spans approximately 4 turns of the α-helix, the resulting macrocycle forms a ring containing approximately 59 members to 67 members, approximately 61 members to 65 members, or approximately 63 members. Where the macrocycle-forming linker spans approximately 5 turns of the α-helix, the resulting macrocycle forms a ring containing approximately 74 members to 82 members, approximately 76 members to 80 members, or approximately 78 members.

In other embodiments, provided are peptidomimetic macrocycles of Formula (II) or (IIa):

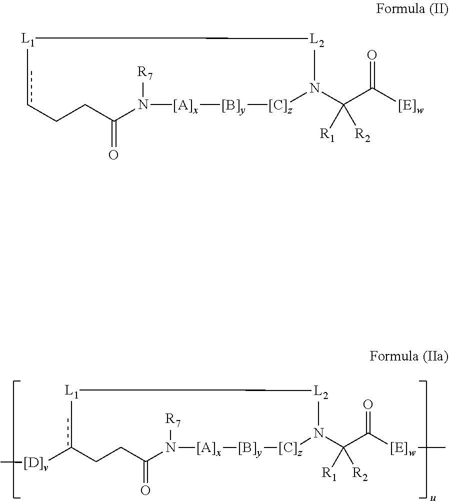

wherein:
each A, C, D, and E is independently a natural or non-natural amino acid or an amino acid analog, and the terminal D and E independently optionally include a capping group;
each B is independently a natural or non-natural amino acid, amino acid analog,

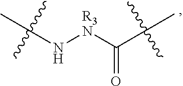

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅;
each v and w is independently an integer from 1-1000;
u is an integer from 1-10;
each x, y, and z is independently integers from 0-10; and
n is an integer from 1-5.

In one example, L₁ and L₂, either alone or in combination, do not form a triazole or a thioether.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, x+y+z is at least 1. In other embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments wherein the amino acids are not identical, e.g. Gln-Asp-Ala as well as embodiments wherein the amino acids are identical, e.g. Gln-Gln-Gln. This applies for any value of x, y, or z in the indicated ranges.

In some embodiments, the peptidomimetic macrocycle comprises a secondary structure which is an α-helix and R₈ is —H, allowing intra-helical hydrogen bonding. In some embodiments, at least one of A, B, C, D or E is an α,α-disubstituted amino acid. In one example, B is an α,α-disubstituted amino acid. For example, at least one of A, B, C, D or E is 2-aminoisobutyric acid. In other embodiments, at least one of A, B, C, D or E is

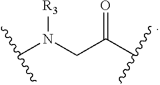

In other embodiments, the length of the macrocycle-forming linker L as measured from a first Cα to a second Cα is selected to stabilize a desired secondary peptide structure, such as an α-helix formed by residues of the peptidomimetic macrocycle including, but not necessarily limited to, those between the first Cα to a second Cα.

Non-limiting examples of embodiments of the macrocycle-forming linker -L₁-L₂- are shown below.

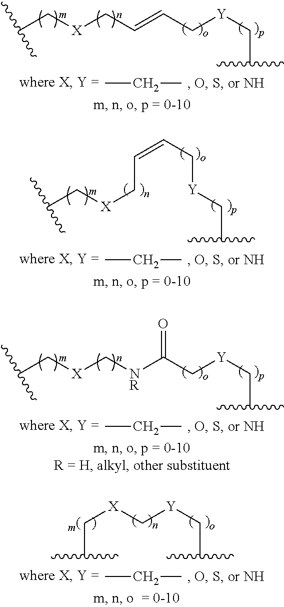

In some embodiments, the peptidomimetic macrocycle has the Formula (III) or Formula (IIIa):

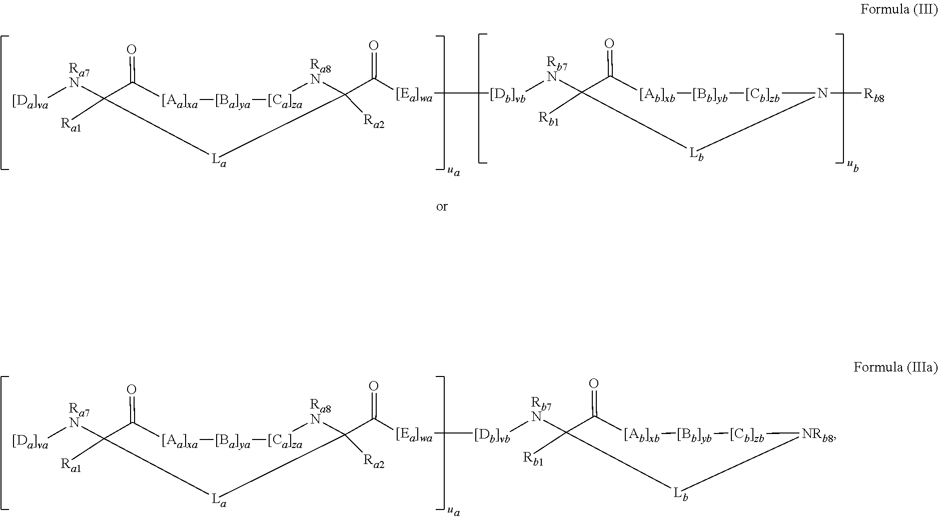

wherein:
each Aₐ, Cₐ, Dₐ, Eₐ, A_{b}, C_{b}, and D_{b} is independently a natural or non-natural amino acid or an amino acid analog;
each Bₐ and B_{b} is independently a natural or non-natural amino acid, amino acid analog,

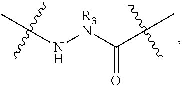

[—NH-L₄-CO—], [—NH-L₄-SO₂—], or [—NH-L₄-];
each Rₐ₁ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₁ forms a macrocycle-forming linker L′ connected to the alpha position of one of the Dₐ or Eₐ amino acids; or together with Lₐ forms a ring that is unsubstituted or substituted;
each Rₐ₂ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₂ forms a macrocycle-forming linker L′ connected to the alpha position of one of the Dₐ or Eₐ amino acids; or together with Lₐ forms a ring that is unsubstituted or substituted;
each R_{b1} is independently alkyl, alkenyl, alkynyl, arylalkyl, cycloalkyl, cycloalkylalkyl, heteroalkyl, or heterocycloalkyl, any of which is unsubstituted or substituted; or H; or R_{b1} forms a macrocycle-forming linker L′ connected to the alpha position of one of the D_{b} amino acids; or together with L_{b} forms a ring that is unsubstituted or substituted;
each R₃ is independently alkyl, alkenyl, alkynyl, arylalkyl, heteroalkyl, cycloalkyl, heterocycloalkyl, cycloalkylalkyl, cycloaryl, or heterocycloaryl, any of which is unsubstituted or substituted, or H;
each Lₐ is independently a macrocycle-forming linker, and optionally forms a ring with Rₐ₁ or Rₐ₂ that is unsubstituted or substituted;
each L_{b} is independently a macrocycle-forming linker, and optionally forms a ring with R_{b1} that is unsubstituted or substituted;
each L′ is independently a macrocycle-forming linker;
each L₄ is independently alkylene, alkenylene, alkynylene, heteroalkylene, cycloalkylene, heterocycloalkylene, cycloarylene, heterocycloarylene, or [—R₄—K—R₄-]ₙ, any of which is unsubstituted or substituted;
each R₄ is independently alkylene, alkenylene, alkynylene, heteroalkylene, cycloalkylene, heterocycloalkylene, arylene, or heteroarylene, any of which is unsubstituted or substituted;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
Rₐ₇ is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with a Dₐ amino acid;
R_{b7} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with a D_{b} amino acid;
Rₐ₈ is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or part of a cyclic structure with an Eₐ amino acid;
R_{b8} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted; or H; or an amino acid sequence of 1-1000 amino acid residues;
each va and vb is independently an integer from 0-1000;
each wa and wb is independently an integer from 0-1000;
each uₐ and u_{b} is independently 0, 1, 2, 3, 4, 5, 6, 7, 8, 9, or 10, wherein ua+ub is at least 1;
each xa and xb is independently 0, 1, 2, 3, 4, 5, 6, 7, 8, 9, or 10;
each ya and yb is independently 0, 1, 2, 3, 4, 5, 6, 7, 8, 9, or 10;
each za and zb is independently 0, 1, 2, 3, 4, 5, 6, 7, 8, 9, or 10; and
each n is independently 1, 2, 3, 4, or 5,
   or a pharmaceutically-acceptable salt thereof.

In some embodiments, the peptidomimetic macrocycle has the Formula (III) or Formula (IIIa):

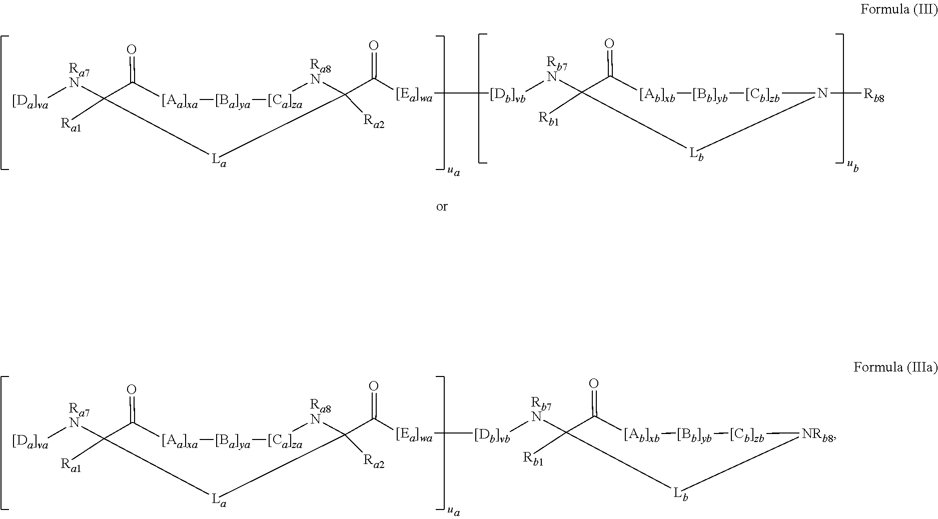

wherein:
each Aₐ, Cₐ, Dₐ, Eₐ, A_{b}, C_{b}, and D_{b} is independently a natural or non-natural amino acid or an amino acid analogue;
each Bₐ and B_{b} is independently a natural or non-natural amino acid, amino acid analog,

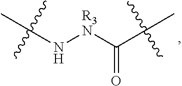

[—NH-L₄-CO—], [—NH-L₄-SO₂—], or [—NH-L₄-];
each Rₐ₁ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₁ forms a macrocycle-forming linker L′ connected to the alpha position of one of the Dₐ or Eₐ amino acids; or together with Lₐ forms a ring that is unsubstituted or substituted;
each Rₐ₂ is independently alkyl, alkenyl, alkynyl, arylalkyl, cycloalkyl, cycloalkylalkyl, heteroalkyl, or heterocycloalkyl, any of which is unsubstituted or substituted; or H; or Rₐ₂ forms a macrocycle-forming linker L′ connected to the alpha position of one of the Dₐ or Eₐ amino acids; or together with Lₐ forms a ring that is unsubstituted or substituted;
each R_{b1} is independently alkyl, alkenyl, alkynyl, arylalkyl, cycloalkyl, cycloalkylalkyl, heteroalkyl, or heterocycloalkyl, any of which is unsubstituted or substituted; or H; or R_{b1} forms a macrocycle-forming linker L′ connected to the alpha position of one of the D_{b} amino acids; or together with L_{b} forms a ring that is unsubstituted or substituted;
each R₃ is independently alkyl, alkenyl, alkynyl, arylalkyl, heteroalkyl, cycloalkyl, heterocycloalkyl, cycloalkylalkyl, cycloaryl, or heterocycloaryl, any of which is unsubstituted or substituted with R₅, or H;
each Lₐ is independently a macrocycle-forming linker, and optionally forms a ring with Rₐ₁ or Rₐ₂ that is unsubstituted or substituted;
each L_{b} is independently a macrocycle-forming linker, and optionally forms a ring with R_{b1} that is unsubstituted or substituted;
each L′ is independently a macrocycle-forming linker;
each L₄ is independently alkylene, alkenylene, alkynylene, heteroalkylene, cycloalkylene, heterocycloalkylene, cycloarylene, heterocycloarylene, or [—R₄—K—R₄—]ₙ, any of which is unsubstituted or substituted with R₅;
each R₄ is independently alkylene, alkenylene, alkynylene, heteroalkylene, cycloalkylene, heterocycloalkylene, arylene, or heteroarylene, any of which is unsubstituted or substituted with R₅;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent;
each R₆ is independently H, alkyl, alkenyl, alkynyl, arylalkyl, cycloalkylalkyl, heterocycloalkyl, a fluorescent moiety, a radioisotope or a therapeutic agent;
each Rₐ₇ is independently alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with a Dₐ amino acid;
R_{b7} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with a D_{b} amino acid;
each Rₐ₈ is independently alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or part of a cyclic structure with an Eₐ amino acid;
R_{b8} is alkyl, alkenyl, alkynyl, arylalkyl, cycloalkyl, heteroalkyl, cycloalkylalkyl, heterocycloalkyl, cycloaryl, or heterocycloaryl, any of which is unsubstituted or substituted with R₅; or H; or an amino acid sequence of 1-1000 amino acid residues;
each va and vb is independently an integer from 0-1000;
each wa and wb is independently an integer from 0-1000;
each uₐ and u_{b} is independently 0, 1, 2, 3, 4, 5, 6, 7, 8, 9, or 10, wherein uₐ+u_{b} is at least 1;
each xa and xb is independently 0, 1, 2, 3, 4, 5, 6, 7, 8, 9, or 10;
each ya and yb is independently 0, 1, 2, 3, 4, 5, 6, 7, 8, 9, or 10;
each za and zb is independently 0, 1, 2, 3, 4, 5, 6, 7, 8, 9, or 10; and
each n is independently 1, 2, 3, 4, or 5,
   or a pharmaceutically-acceptable salt thereof.

In some embodiments, the peptidomimetic macrocycle of the invention has the formula defined above, wherein:
each Lₐ is independently a macrocycle-forming linker of the formula -L₁-L₂-, and optionally forms a ring with Rₐ₁ or Rₐ₂ that is unsubstituted or substituted;
each L_{b} is independently a macrocycle-forming linker of the formula -L₁-L₂-, and optionally forms a ring with R_{b1} that is unsubstituted or substituted;
each L′ is independently a macrocycle-forming linker of the formula -L₁-L₂-;
each L₁ and L₂ is independently alkylene, alkenylene, alkynylene, heteroalkylene, cycloalkylene, heterocycloalkylene, cycloarylene, heterocycloarylene, or [—R₄—K—R₄-]ₙ, any of which is unsubstituted or substituted with R₅;
each R₄ is independently alkylene, alkenylene, alkynylene, heteroalkylene, cycloalkylene, heterocycloalkylene, arylene, or heteroarylene, any of which is unsubstituted or substituted with R₅;
each K is independently O, S, SO, SO₂, CO, CO₂, OCO₂, NR₃, CONR₃, OCONR₃, OSO₂NR₃, NR_{3q}, CONR_{3q}, OCONR_{3q}, or OSO₂NR_{3q}, wherein each R_{3q} is independently a point of attachment to Rₐ₁, Rₐ₂, or R_{b1};
each R₅ is independently halogen, alkyl, —OR₆, —N(R₆)₂, —SR₆, —SOR₆, —SO₂R₆, —CO₂R₆, a fluorescent moiety, a radioisotope, or a therapeutic agent; and
each R₆ is independently H, alkyl, alkenyl, alkynyl, arylalkyl, cycloalkylalkyl, heterocycloalkyl, a fluorescent moiety, a radioisotope or a therapeutic agent,
   or a pharmaceutically-acceptable salt thereof.

In some embodiments, the peptidomimetic macrocycle has the formula defined above wherein one of Lₐ and L_{b} is a bis-thioether-containing macrocycle-forming linker. In some embodiments, one of Lₐ and L_{b} is a macrocycle-forming linker of the formula -L₁-S-L₂-S-L₃-.

In some embodiments, the peptidomimetic macrocycle has the formula defined above wherein one of Lₐ and L_{b} is a bis-sulfone-containing macrocycle-forming linker. In some embodiments, one of Lₐ and L_{b} is a macrocycle-forming linker of the formula -L₁-SO₂-L₂-SO₂-L₃-.

In some embodiments, the peptidomimetic macrocycle has the formula defined above wherein one of Lₐ and L_{b} is a bis-sulfoxide-containing macrocycle-forming linker. In some embodiments, one of Lₐ and L_{b} is a macrocycle-forming linker of the formula -L₁-S(O)-L₂-S(O)-L₃-.

In some embodiments, a peptidomimetic macrocycle of the invention comprises one or more secondary structures. In some embodiments, the peptidomimetic macrocycle comprises a secondary structure that is an α-helix. In some embodiments, the peptidomimetic macrocycle comprises a secondary structure that is a β-hairpin turn.

In some embodiments, uₐ is 0. In some embodiments, uₐ is 0, and L_{b} is a macrocycle-forming linker that crosslinks an α-helical secondary structure. In some embodiments, uₐ is 0, and L_{b} is a macrocycle-forming linker that crosslinks a β-hairpin secondary structure. In some embodiments, uₐ is 0, and L_{b} is a hydrocarbon-containing macrocycle-forming linker that crosslinks an α-helical secondary structure. In some embodiments, uₐ is 0, and L_{b} is a hydrocarbon-containing macrocycle-forming linker that crosslinks a β-hairpin secondary structure.

In some embodiments, u_{b} is 0. In some embodiments, u_{b} is 0, and Lₐ is a macrocycle-forming linker that crosslinks an α-helical secondary structure. In some embodiments, u_{b} is 0, and Lₐ is a macrocycle-forming linker that crosslinks a β-hairpin secondary structure. In some embodiments, u_{b} is 0, and Lₐ is a hydrocarbon-containing macrocycle-forming linker that crosslinks an α-helical secondary structure. In some embodiments, u_{b} is 0, and Lₐ is a hydrocarbon-containing macrocycle-forming linker that crosslinks a β-hairpin secondary structure.

In some embodiments, the peptidomimetic macrocycle comprises only α-helical secondary structures. In other embodiments, the peptidomimetic macrocycle comprises only β-hairpin secondary structures.

In other embodiments, the peptidomimetic macrocycle comprises a combination of secondary structures, wherein the secondary structures are α-helical and β-hairpin structures. In some embodiments, Lₐ and L_{b} are a combination of hydrocarbon-, triazole, or sulfur-containing macrocycle-forming linkers. In some embodiments, the peptidomimetic macrocycle comprises Lₐ and L_{b}, wherein Lₐ is a hydrocarbon-containing macrocycle-forming linker that crosslinks a β-hairpin structure, and L_{b} is a triazole-containing macrocycle-forming linker that crosslinks an α-helical structure. In some embodiments, the peptidomimetic macrocycle comprises Lₐ and L_{b}, wherein Lₐ is a hydrocarbon-containing macrocycle-forming linker that crosslinks an α-helical structure, and L_{b} is a triazole-containing macrocycle-forming linker that crosslinks a β-hairpin structure. In some embodiments, the peptidomimetic macrocycle comprises Lₐ and L_{b}, wherein Lₐ is a triazole-containing macrocycle-forming linker that crosslinks an α-helical structure, and L_{b} is a hydrocarbon-containing macrocycle-forming linker that crosslinks a (β-hairpin structure. In some embodiments, the peptidomimetic macrocycle comprises Lₐ and L_{b}, wherein Lₐ is a triazole-containing macrocycle-forming linker that crosslinks a (β-hairpin structure, and L_{b} is a hydrocarbon-containing macrocycle-forming linker that crosslinks an α-helical structure.

In some embodiments, uₐ+u_{b} is at least 1. In some embodiments, uₐ+u_{b}=2.

In some embodiments, uₐ is 1, u_{b} is 1, Lₐ is a triazole-containing macrocycle-forming linker that crosslinks an α-helical secondary structure, and L_{b} is a hydrocarbon-containing macrocycle-forming linker that crosslinks an α-helical structure. In some embodiments, uₐ is 1, u_{b} is 1, Lₐ is a triazole-containing macrocycle-forming linker that crosslinks an α-helical secondary structure, and L_{b} is a hydrocarbon-containing macrocycle-forming linker that crosslinks a β-hairpin structure. In some embodiments, uₐ is 1, u_{b} is 1, Lₐ is a triazole-containing macrocycle-forming linker that crosslinks a β-hairpin secondary structure, and L_{b} is a hydrocarbon-containing macrocycle-forming linker that crosslinks an α-helical structure. In some embodiments, uₐ is 1, u_{b} is 1, Lₐ is a triazole-containing macrocycle-forming linker that crosslinks a β-hairpin secondary structure, and L_{b} is a hydrocarbon-containing macrocycle-forming linker that crosslinks a β-hairpin structure.

In some embodiments, uₐ is 1, u_{b} is 1, Lₐ is a hydrocarbon-containing macrocycle-forming linker that crosslinks an α-helical secondary structure, and L_{b} is a triazole-containing macrocycle-forming linker that crosslinks an α-helical secondary structure. In some embodiments, uₐ is 1, u_{b} is 1, Lₐ is a hydrocarbon-containing macrocycle-forming linker that crosslinks an α-helical secondary structure, and L_{b} is a triazole-containing macrocycle-forming linker that crosslinks a β-hairpin secondary structure. In some embodiments, uₐ is 1, u_{b} is 1, Lₐ is a hydrocarbon-containing macrocycle-forming linker that crosslinks a β-hairpin secondary structure, and L_{b} is a triazole-containing macrocycle-forming linker that crosslinks an α-helical secondary structure. In some embodiments, uₐ is 1, u_{b} is 1, Lₐ is a hydrocarbon-containing macrocycle-forming linker that crosslinks a β-hairpin secondary structure, and L_{b} is a triazole-containing macrocycle-forming linker that crosslinks a β-hairpin secondary structure.

In some embodiments, uₐ is 1, u_{b} is 1, Lₐ is a hydrocarbon-containing macrocycle-forming linker with an α-helical secondary structure, and L_{b} is a sulfur-containing macrocycle-forming linker. In some embodiments, uₐ is 1, u_{b} is 1, Lₐ is a hydrocarbon-containing macrocycle-forming linker with a β-hairpin secondary structure, and L_{b} is a sulfur-containing macrocycle-forming linker.

In some embodiments, uₐ is 1, u_{b} is 1, Lₐ is a sulfur-containing macrocycle-forming linker, and L_{b} is a hydrocarbon-containing macrocycle-forming linker with an α-helical secondary structure. In some embodiments, uₐ is 1, u_{b} is 1, Lₐ is a sulfur-containing macrocycle-forming linker, and L_{b} is a hydrocarbon-containing macrocycle-forming linker with a β-hairpin secondary structure.

In some embodiments, uₐ is 1, u_{b} is 1, Lₐ is a hydrocarbon-containing macrocycle-forming linker that crosslinks an α-helical structure, and L_{b} is a hydrocarbon-containing macrocycle-forming linker that crosslinks an α-helical structure. In some embodiments, uₐ is 1, u_{b} is 1, Lₐ is a hydrocarbon-containing macrocycle-forming linker that crosslinks an α-helical structure, and L_{b} is a hydrocarbon-containing macrocycle-forming linker that crosslinks a 3-hairpin structure. In some embodiments, uₐ is 1, u_{b} is 1, Lₐ is a hydrocarbon-containing macrocycle-forming linker that crosslinks a β-hairpin structure, and L_{b} is a hydrocarbon-containing macrocycle-forming linker that crosslinks an α-helical structure. In some embodiments, uₐ is 1, u_{b} is 1, Lₐ is a hydrocarbon-containing macrocycle-forming linker that crosslinks a β-hairpin structure, and L_{b} is a hydrocarbon-containing macrocycle-forming linker that crosslinks a β-hairpin structure.

In some embodiments, R_{b1} is H.

Unless otherwise stated, any compounds (including peptidomimetic macrocycles, macrocycle precursors, and other compositions) are also meant to encompass compounds which differ only in the presence of one or more isotopically enriched atoms. For example, compounds having the described structures except for the replacement of a hydrogen atom by deuterium or tritium, or the replacement of a carbon atom by ¹³C or ¹⁴C are contemplated.

In some embodiments, the compounds disclosed herein can contain unnatural proportions of atomic isotopes at one or more of atoms that constitute such compounds. For example, the compounds can be radiolabeled with radioactive isotopes, such as for example tritium (³H), iodine-125 (¹²⁵I) or carbon-14 (¹⁴C). In other embodiments, one or more carbon atoms is replaced with a silicon atom. All isotopic variations of the compounds disclosed herein, whether radioactive or not, are contemplated herein.

In some embodiments, the peptidomimetic macrocycle comprises an amino acid sequence that is at least 60%, at least 65%, at least 70%, at least 75%, at least 80%, at least 85%, at least 90%, or at least 95% identical to an amino acid sequence listed in Table 1, Table 1a, Table 1b, Table 1c, Table 2a, Table 2b, Table 3, or Table 3a. In some embodiments, the peptidomimetic macrocycle comprises an amino acid sequence that is at least 60% identical to an amino acid sequence listed in Table 1, Table 1a, Table 1b, Table 1c, Table 2a, Table 2b, Table 3, or Table 3a. In some embodiments, the peptidomimetic macrocycle comprises an amino acid sequence that is at least 65% identical to an amino acid sequence listed in Table 1, Table 1a, Table 1b, Table 1c, Table 2a, Table 2b, Table 3, or Table 3a. In some embodiments, the peptidomimetic macrocycle comprises an amino acid sequence that is at least 70% identical to an amino acid sequence listed in Table 1, Table 1a, Table 1b, Table 1c, Table 2a, Table 2b, Table 3, or Table 3a. In some embodiments, the peptidomimetic macrocycle comprises an amino acid sequence that is at least 75% identical to an amino acid sequence listed in Table 1, Table 1a, Table 1b, Table 1c, Table 2a, Table 2b, Table 3, or Table 3a.

In some embodiments, the peptidomimetic macrocycle is at least 60%, at least 65%, at least 70%, at least 75%, at least 80%, at least 85%, at least 90%, or at least 95% identical to an amino acid sequence listed in Table 1, Table 1a, Table 1b, Table 1c, Table 2a, Table 2b, Table 3, or Table 3a. In some embodiments, the peptidomimetic macrocycle is at least 60% identical to an amino acid sequence listed in Table 1, Table 1a, Table 1b, Table 1c, Table 2a, Table 2b, Table 3, or Table 3a. In some embodiments, the peptidomimetic macrocycle is at least 65% identical to an amino acid sequence listed in Table 1, Table 1a, Table 1b, Table 1c, Table 2a, Table 2b, Table 3, or Table 3a. In some embodiments, the peptidomimetic macrocycle is at least 70% identical to an amino acid sequence listed in Table 1, Table 1a, Table 1b, Table 1c, Table 2a, Table 2b, Table 3, or Table 3a. In some embodiments, the peptidomimetic macrocycle is at least 75% identical to an amino acid sequence listed in Table 1, Table 1a, Table 1b, Table 1c, Table 2a, Table 2b, Table 3, or Table 3a.

In some embodiments, a peptidomimetic macrocycle has an amino acid sequence comprising a carboxy terminus, a cross link spanning amino acids in the i to i+7 position of the amino acid sequence, and at least 1, at least 2, at least 3, at least 4, at least 5, 1-100, 2-100, 3-100, 4-100, 5-100, 1-10, 2-10, 3-10, 4-10, or 5-10 amino acids between the i+7 position of the amino acid sequence and the carboxy terminus.

### Preparation of Peptidomimetic Macrocycles

Peptidomimetic macrocycles can be prepared by any of a variety of methods known in the art. For example, any of the residues indicated by “$” or “$r8” in Table 1, Table 1a, Table 1b, Table 1c, Table 2a, Table 2b, Table 3, or Table 3a can be substituted with a residue capable of forming a crosslinker with a second residue in the same molecule or a precursor of such a residue.

α,α-Disubstituted amino acids and amino acid precursors can be employed in synthesis of the peptidomimetic macrocycle precursor polypeptides. For example, the “S5-olefin amino acid” is (S)-α-(2′-pentenyl) alanine and the “R₈ olefin amino acid” is (R)-α-(2′-octenyl) alanine. Following incorporation of such amino acids into precursor polypeptides, the terminal olefins are reacted with a metathesis catalyst, leading to the formation of the peptidomimetic macrocycle. In various embodiments, the following amino acids can be employed in the synthesis of the peptidomimetic macrocycle:

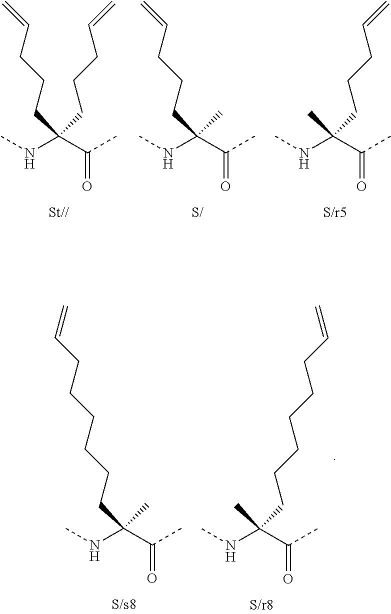

In other embodiments, the peptidomimetic macrocycles are of Formula IV or IVa. In such embodiments, amino acid precursors are used containing an additional substituent R— at the alpha position. Such amino acids are incorporated into the macrocycle precursor at the desired positions, which can be at the positions where the crosslinker is substituted or, alternatively, elsewhere in the sequence of the macrocycle precursor. Cyclization of the precursor is then effected according to the indicated method.

### Pharmaceutically-Acceptable Salts

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Purity of Compounds of the Invention

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutical Compositions

Pharmaceutical compositions disclosed herein include peptidomimetic macrocycles and pharmaceutically-acceptable derivatives or prodrugs thereof. A “pharmaceutically-acceptable derivative” means any pharmaceutically-acceptable salt, ester, salt of an ester, pro-drug or other derivative of a compound disclosed herein which, upon administration to a recipient, is capable of providing (directly or indirectly) a compound disclosed herein. Particularly favored pharmaceutically-acceptable derivatives are those that increase the bioavailability of the compounds when administered to a mammal (e.g., by increasing absorption into the blood of an orally administered compound) or which increases delivery of the active compound to a biological compartment (e.g., the brain or lymphatic system) relative to the parent species. Some pharmaceutically-acceptable derivatives include a chemical group which increases aqueous solubility or active transport across the gastrointestinal mucosa.

In some embodiments, peptidomimetic macrocycles are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow administration by injection, alter metabolism, and alter rate of excretion.

For preparing pharmaceutical compositions from the compounds disclosed herein, pharmaceutically-acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the crosslinked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation can be in unit dosage form. In such form the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

When one or more compositions disclosed herein comprise a combination of a peptidomimetic macrocycle and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the additional agents are administered separately, as part of a multiple dose regimen, from one or more compounds disclosed herein. Alternatively, those agents are part of a single dosage form, mixed together with the compounds disclosed herein in a single composition.

In some embodiments, a pharmaceutical composition disclosed herein comprises a peptidomimetic macrocycle at a concentration of about 5 mg/mL to about 50 mg/mL. In some embodiments, a pharmaceutical composition disclosed herein comprises a peptidomimetic macrocycle at a concentration of about 5 mg/mL to about 10 mg/mL, about 5 mg/mL to about 15 mg/mL, about 5 mg/mL to about 20 mg/mL, about 5 mg/mL to about 30 mg/mL, about 5 mg/mL to about 40 mg/mL, about 5 mg/mL to about 50 mg/mL, about 10 mg/mL to about 15 mg/mL, about 10 mg/mL to about 20 mg/mL, about 10 mg/mL to about 30 mg/mL, about 10 mg/mL to about 40 mg/mL, about 10 mg/mL to about 50 mg/mL, about 15 mg/mL to about 20 mg/mL, about 15 mg/mL to about 30 mg/mL, about 15 mg/mL to about 40 mg/mL, about 15 mg/mL to about 50 mg/mL, about 20 mg/mL to about 30 mg/mL, about 20 mg/mL to about 40 mg/mL, about 20 mg/mL to about 50 mg/mL, about 30 mg/mL to about 40 mg/mL, about 30 mg/mL to about 50 mg/mL, or about 40 mg/mL to about 50 mg/mL. In some embodiments, a pharmaceutical composition disclosed herein comprises a peptidomimetic macrocycle at a concentration of about 5 mg/mL, about 10 mg/mL, about 15 mg/mL, about 20 mg/mL, about 30 mg/mL, about 40 mg/mL, or about 50 mg/mL. In some embodiments, a pharmaceutical composition disclosed herein comprises a peptidomimetic macrocycle at a concentration of at least about 5 mg/mL, about 10 mg/mL, about 15 mg/mL, about 20 mg/mL, about 30 mg/mL, or about 40 mg/mL. In some embodiments, a pharmaceutical composition disclosed herein comprises a peptidomimetic macrocycle at a concentration of at most about 10 mg/mL, about 15 mg/mL, about 20 mg/mL, about 30 mg/mL, about 40 mg/mL, or about 50 mg/mL.

In some embodiments, a pharmaceutical composition disclosed herein comprises trehalose. In some embodiments, the concentration of trehalose in a pharmaceutical composition disclosed herein is about 10 mg/mL to about 500 mg/mL. In some embodiments, the concentration of trehalose in a pharmaceutical composition disclosed herein is about 10 mg/mL to about 20 mg/mL, about 10 mg/mL to about 30 mg/mL, about 10 mg/mL to about 40 mg/mL, about 10 mg/mL to about 50 mg/mL, about 10 mg/mL to about 60 mg/mL, about 10 mg/mL to about 70 mg/mL, about 10 mg/mL to about 80 mg/mL, about 10 mg/mL to about 90 mg/mL, about 10 mg/mL to about 100 mg/mL, about 10 mg/mL to about 250 mg/mL, about 10 mg/mL to about 500 mg/mL, about 20 mg/mL to about 30 mg/mL, about 20 mg/mL to about 40 mg/mL, about 20 mg/mL to about 50 mg/mL, about 20 mg/mL to about 60 mg/mL, about 20 mg/mL to about 70 mg/mL, about 20 mg/mL to about 80 mg/mL, about 20 mg/mL to about 90 mg/mL, about 20 mg/mL to about 100 mg/mL, about 20 mg/mL to about 250 mg/mL, about 20 mg/mL to about 500 mg/mL, about 30 mg/mL to about 40 mg/mL, about 30 mg/mL to about 50 mg/mL, about 30 mg/mL to about 60 mg/mL, about 30 mg/mL to about 70 mg/mL, about 30 mg/mL to about 80 mg/mL, about 30 mg/mL to about 90 mg/mL, about 30 mg/mL to about 100 mg/mL, about 30 mg/mL to about 250 mg/mL, about 30 mg/mL to about 500 mg/mL, about 40 mg/mL to about 50 mg/mL, about 40 mg/mL to about 60 mg/mL, about 40 mg/mL to about 70 mg/mL, about 40 mg/mL to about 80 mg/mL, about 40 mg/mL to about 90 mg/mL, about 40 mg/mL to about 100 mg/mL, about 40 mg/mL to about 250 mg/mL, about 40 mg/mL to about 500 mg/mL, about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 50 mg/mL to about 250 mg/mL, about 50 mg/mL to about 500 mg/mL, about 60 mg/mL to about 70 mg/mL, about 60 mg/mL to about 80 mg/mL, about 60 mg/mL to about 90 mg/mL, about 60 mg/mL to about 100 mg/mL, about 60 mg/mL to about 250 mg/mL, about 60 mg/mL to about 500 mg/mL, about 70 mg/mL to about 80 mg/mL, about 70 mg/mL to about 90 mg/mL, about 70 mg/mL to about 100 mg/mL, about 70 mg/mL to about 250 mg/mL, about 70 mg/mL to about 500 mg/mL, about 80 mg/mL to about 90 mg/mL, about 80 mg/mL to about 100 mg/mL, about 80 mg/mL to about 250 mg/mL, about 80 mg/mL to about 500 mg/mL, about 90 mg/mL to about 100 mg/mL, about 90 mg/mL to about 250 mg/mL, about 90 mg/mL to about 500 mg/mL, about 100 mg/mL to about 250 mg/mL, about 100 mg/mL to about 500 mg/mL, or about 250 mg/mL to about 500 mg/mL. In some embodiments, the concentration of trehalose in a pharmaceutical composition disclosed herein is about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 250 mg/mL, or about 500 mg/mL. In some embodiments, the concentration of trehalose in a pharmaceutical composition disclosed herein is at least about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, or about 250 mg/mL. In some embodiments, the concentration of trehalose in a pharmaceutical composition disclosed herein is at most about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 250 mg/mL, or about 500 mg/mL.

In some embodiments, the concentration of trehalose in a pharmaceutical composition disclosed herein is about 100 mM to about 500 mM. In some embodiments, the concentration of trehalose in a pharmaceutical composition disclosed herein is about 100 mM to about 200 mM, about 100 mM to about 220 mM, about 100 mM to about 240 mM, about 100 mM to about 260 mM, about 100 mM to about 280 mM, about 100 mM to about 300 mM, about 100 mM to about 350 mM, about 100 mM to about 400 mM, about 100 mM to about 450 mM, about 100 mM to about 500 mM, about 200 mM to about 220 mM, about 200 mM to about 240 mM, about 200 mM to about 260 mM, about 200 mM to about 280 mM, about 200 mM to about 300 mM, about 200 mM to about 350 mM, about 200 mM to about 400 mM, about 200 mM to about 450 mM, about 200 mM to about 500 mM, about 220 mM to about 240 mM, about 220 mM to about 260 mM, about 220 mM to about 280 mM, about 220 mM to about 300 mM, about 220 mM to about 350 mM, about 220 mM to about 400 mM, about 220 mM to about 450 mM, about 220 mM to about 500 mM, about 240 mM to about 260 mM, about 240 mM to about 280 mM, about 240 mM to about 300 mM, about 240 mM to about 350 mM, about 240 mM to about 400 mM, about 240 mM to about 450 mM, about 240 mM to about 500 mM, about 260 mM to about 280 mM, about 260 mM to about 300 mM, about 260 mM to about 350 mM, about 260 mM to about 400 mM, about 260 mM to about 450 mM, about 260 mM to about 500 mM, about 280 mM to about 300 mM, about 280 mM to about 350 mM, about 280 mM to about 400 mM, about 280 mM to about 450 mM, about 280 mM to about 500 mM, about 300 mM to about 350 mM, about 300 mM to about 400 mM, about 300 mM to about 450 mM, about 300 mM to about 500 mM, about 350 mM to about 400 mM, about 350 mM to about 450 mM, about 350 mM to about 500 mM, about 400 mM to about 450 mM, about 400 mM to about 500 mM, or about 450 mM to about 500 mM. In some embodiments, the concentration of trehalose in a pharmaceutical composition disclosed herein is about 100 mM, about 200 mM, about 220 mM, about 240 mM, about 260 mM, about 280 mM, about 300 mM, about 350 mM, about 400 mM, about 450 mM, or about 500 mM. In some embodiments, the concentration of trehalose in a pharmaceutical composition disclosed herein is at least about 100 mM, about 200 mM, about 220 mM, about 240 mM, about 260 mM, about 280 mM, about 300 mM, about 350 mM, about 400 mM, or about 450 mM. In some embodiments, the concentration of trehalose in a pharmaceutical composition disclosed herein is at most about 200 mM, about 220 mM, about 240 mM, about 260 mM, about 280 mM, about 300 mM, about 350 mM, about 400 mM, about 450 mM, or about 500 mM.

In some embodiments, a pharmaceutical composition disclosed herein comprises a tonicity adjusting agent. In some embodiments, the concentration of the tonicity adjusting agent is about 100 mM to about 500 mM. In some embodiments, the concentration of the tonicity adjusting agent is about 100 mM to about 200 mM, about 100 mM to about 220 mM, about 100 mM to about 240 mM, about 100 mM to about 260 mM, about 100 mM to about 280 mM, about 100 mM to about 300 mM, about 100 mM to about 350 mM, about 100 mM to about 400 mM, about 100 mM to about 450 mM, about 100 mM to about 500 mM, about 200 mM to about 220 mM, about 200 mM to about 240 mM, about 200 mM to about 260 mM, about 200 mM to about 280 mM, about 200 mM to about 300 mM, about 200 mM to about 350 mM, about 200 mM to about 400 mM, about 200 mM to about 450 mM, about 200 mM to about 500 mM, about 220 mM to about 240 mM, about 220 mM to about 260 mM, about 220 mM to about 280 mM, about 220 mM to about 300 mM, about 220 mM to about 350 mM, about 220 mM to about 400 mM, about 220 mM to about 450 mM, about 220 mM to about 500 mM, about 240 mM to about 260 mM, about 240 mM to about 280 mM, about 240 mM to about 300 mM, about 240 mM to about 350 mM, about 240 mM to about 400 mM, about 240 mM to about 450 mM, about 240 mM to about 500 mM, about 260 mM to about 280 mM, about 260 mM to about 300 mM, about 260 mM to about 350 mM, about 260 mM to about 400 mM, about 260 mM to about 450 mM, about 260 mM to about 500 mM, about 280 mM to about 300 mM, about 280 mM to about 350 mM, about 280 mM to about 400 mM, about 280 mM to about 450 mM, about 280 mM to about 500 mM, about 300 mM to about 350 mM, about 300 mM to about 400 mM, about 300 mM to about 450 mM, about 300 mM to about 500 mM, about 350 mM to about 400 mM, about 350 mM to about 450 mM, about 350 mM to about 500 mM, about 400 mM to about 450 mM, about 400 mM to about 500 mM, or about 450 mM to about 500 mM. In some embodiments, the concentration of the tonicity adjusting agent is about 100 mM, about 200 mM, about 220 mM, about 240 mM, about 260 mM, about 280 mM, about 300 mM, about 350 mM, about 400 mM, about 450 mM, or about 500 mM. In some embodiments, the concentration of the tonicity adjusting agent is at least about 100 mM, about 200 mM, about 220 mM, about 240 mM, about 260 mM, about 280 mM, about 300 mM, about 350 mM, about 400 mM, or about 450 mM. In some embodiments, the concentration of the tonicity adjusting agent is at most about 200 mM, about 220 mM, about 240 mM, about 260 mM, about 280 mM, about 300 mM, about 350 mM, about 400 mM, about 450 mM, or about 500 mM. In some embodiments, the tonicity adjusting agent is trehalose.

A pharmaceutical composition of the disclosure can comprise polysorbate. In some embodiments, polysorbate acts as a stabilizing agent. In some embodiments, polysorbate is present in a pharmaceutical composition disclosed herein at a concentration of about 50 ppm to about 500 ppm. In some embodiments, polysorbate is present in a pharmaceutical composition disclosed herein at a concentration of about 50 ppm to about 100 ppm, about 50 ppm to about 150 ppm, about 50 ppm to about 200 ppm, about 50 ppm to about 250 ppm, about 50 ppm to about 300 ppm, about 50 ppm to about 350 ppm, about 50 ppm to about 400 ppm, about 50 ppm to about 450 ppm, about 50 ppm to about 500 ppm, about 100 ppm to about 150 ppm, about 100 ppm to about 200 ppm, about 100 ppm to about 250 ppm, about 100 ppm to about 300 ppm, about 100 ppm to about 350 ppm, about 100 ppm to about 400 ppm, about 100 ppm to about 450 ppm, about 100 ppm to about 500 ppm, about 150 ppm to about 200 ppm, about 150 ppm to about 250 ppm, about 150 ppm to about 300 ppm, about 150 ppm to about 350 ppm, about 150 ppm to about 400 ppm, about 150 ppm to about 450 ppm, about 150 ppm to about 500 ppm, about 200 ppm to about 250 ppm, about 200 ppm to about 300 ppm, about 200 ppm to about 350 ppm, about 200 ppm to about 400 ppm, about 200 ppm to about 450 ppm, about 200 ppm to about 500 ppm, about 250 ppm to about 300 ppm, about 250 ppm to about 350 ppm, about 250 ppm to about 400 ppm, about 250 ppm to about 450 ppm, about 250 ppm to about 500 ppm, about 300 ppm to about 350 ppm, about 300 ppm to about 400 ppm, about 300 ppm to about 450 ppm, about 300 ppm to about 500 ppm, about 350 ppm to about 400 ppm, about 350 ppm to about 450 ppm, about 350 ppm to about 500 ppm, about 400 ppm to about 450 ppm, about 400 ppm to about 500 ppm, or about 450 ppm to about 500 ppm. In some embodiments, polysorbate is present in a pharmaceutical composition disclosed herein at a concentration of about 50 ppm, about 100 ppm, about 150 ppm, about 200 ppm, about 250 ppm, about 300 ppm, about 350 ppm, about 400 ppm, about 450 ppm, or about 500 ppm. In some embodiments, polysorbate is present in a pharmaceutical composition disclosed herein at a concentration of at least about 50 ppm, about 100 ppm, about 150 ppm, about 200 ppm, about 250 ppm, about 300 ppm, about 350 ppm, about 400 ppm, or about 450 ppm. In some embodiments, polysorbate is present in a pharmaceutical composition disclosed herein at a concentration of at most about 100 ppm, about 150 ppm, about 200 ppm, about 250 ppm, about 300 ppm, about 350 ppm, about 400 ppm, about 450 ppm, or about 500 ppm. Non-limiting examples of a polysorbate present in a pharmaceutical composition disclosed herein include polysorbate 20, polysorbate 21, polysorbate 40, polysorbate 60, polysorbate 61, polysorbate 65, polysorbate 80, polysorbate 81, polysorbate 85 or polysorbate 120.

In some aspects, the disclosure provides a pharmaceutical formulation comprising a therapeutically-effective amount of one or more of the peptidomimetic macrocycles described above, formulated together with one or more pharmaceutically-acceptable carriers (additives) and/or diluents. In one embodiment, one or more of the peptidomimetic macrocycles described herein are formulated for parenteral administration, one or more peptidomimetic macrocycles disclosed herein can be formulated as aqueous or non-aqueous solutions, dispersions, suspensions or emulsions or sterile powders which can be reconstituted into sterile injectable solutions or dispersions just prior to use. Such formulations can comprise sugars, alcohols, antioxidants, buffers, bacteriostats, solutes which render the formulation isotonic with the blood of the intended recipient or suspending or thickening agents. These compositions can also contain adjuvants such as preservatives, wetting agents, emulsifying agents and dispersing agents. Prevention of the action of microorganisms upon the subject compounds can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin. If desired, the formulation can be diluted prior to use with, for example, an isotonic saline solution or a dextrose solution. In some examples, the peptidomimetic macrocycle is formulated as an aqueous solution and is administered intravenously.

### Mode of Administration

A therapeutically effective amount of a peptidomimetic macrocycle of the disclosure can be administered in either single or multiple doses by any of the accepted modes of administration. In some embodiments, the peptidomimetic macrocycles of the disclosure are administered parenterally, for example, by subcutaneous, intramuscular, intrathecal, intravenous or epidural injection. For example, the peptidomimetic macrocycle is administered intravenously, intra-arterially, subcutaneously or by infusion. In some examples, the peptidomimetic macrocycle is administered intravenously. In some examples, the peptidomimetic macrocycle is administered intra-arterially.

Regardless of the route of administration selected, the peptidomimetic macrocycles of the present disclosure, and/or the pharmaceutical compositions of the present disclosure, are formulated into pharmaceutically-acceptable dosage forms. The peptidomimetic macrocycles according to the disclosure can be formulated for administration in any convenient way for use in human or veterinary medicine, by analogy with other pharmaceuticals.

In some embodiments, a peptidomimetic macrocycle is administered in combination with an additional pharmaceutically-active agent, for example, any additional therapeutic agent described herein. In some embodiments, the additional pharmaceutically-active agent is administered parenterally, for example, by subcutaneous, intramuscular, intrathecal, intravenous or epidural injection. A peptidomimetic macrocycle and an additional pharmaceutically-active agent, for example, any additional therapeutic agent described herein can be administered via the same or different administration routes. For example, it may be advantageous to administer either the peptidomimetic macrocycle or the additional pharmaceutically-active agent, for example, any additional therapeutic agent described herein, intravenously and the other systemically or orally. For example, the peptidomimetic macrocycle can be administered intravenously and the additional pharmaceutically-active agent can be administered orally. In another example both the peptidomimetic macrocycle and the additional pharmaceutically-active agent, for example, any additional therapeutic agent described herein, are administered intravenously.

### Combination Treatment

Provided herein are methods for the treatment of cancer which involve the administration of a peptidomimetic macrocycle disclosed herein in combination with one or more additional therapies to a subject with cancer. In some embodiments, the cancer possesses a p53 deactivating mutation and/or lacks wild type p53. In some embodiments, the cancer is determined to possess a p53 deactivating mutation and/or lacks wild type p53 prior to the beginning of treatment. In some embodiments, the subject possesses wild-type p53 in non-cancerous tissues such as the bone marrow or tissue of the digestive tract. In some embodiments, presented herein are combination therapies for the treatment of cancer which involve the administration of an effective amount of a peptidomimetic macrocycle disclosed herein in combination with an effective amount of an additional pharmaceutically-active agent to a subject with a p53-mutant cancer and bone marrow expressing wild type p53. In some embodiments, presented herein are combination therapies for the treatment of cancer which involve the administration of an effective amount of a peptidomimetic macrocycle disclosed herein in combination with an effective amount of an additional pharmaceutically-active agent to a subject with a cancer lacking wild type p53 and bone marrow expressing wild type p53. In some embodiments, presented herein are combination therapies for the treatment of cancer which involve the administration of an effective amount of a peptidomimetic macrocycle disclosed herein in combination with an effective amount of an additional pharmaceutically-active agent to a subject with a p53-mutant cancer and tissue of the digestive tract expressing wild type p53. In some embodiments, presented herein are combination therapies for the treatment of cancer which involve the administration of an effective amount of a peptidomimetic macrocycle disclosed herein in combination with an effective amount of an additional pharmaceutically-active agent to a subject with a cancer lacking wild type p53 and tissue of the digestive tract expressing wild type p53.

As used herein, the term “in combination,” refers, in the context of the administration of the peptidomimetic macrocycles, to the administration of the peptidomimetic macrocycles prior to, concurrently with, or subsequent to the administration of one or more additional therapies (e.g., pharmaceutically-active agents, surgery, or radiation) for use in treating cancer. The use of the term “in combination” does not restrict the order in which the peptidomimetic macrocycles and one or more additional therapies are administered to a subject.

The peptidomimetic macrocycles or a composition comprising the same and the at least one additional pharmaceutically-active agent, for example, any additional therapeutic agent described herein, or a composition comprising same can be administered simultaneously (i.e., simultaneous administration) and/or sequentially (i.e., sequential administration).

According to certain embodiments, the peptidomimetic macrocycles and the at least one additional pharmaceutically-active agent, for example, any additional therapeutic agent described herein, are administered simultaneously, either in the same composition or in separate compositions. In some embodiments, simultaneous administration of a peptidomimetic macrocycle and at least one additional pharmaceutically-active agent, for example, any additional therapeutic agent described herein, involves administration of the peptidomimetic macrocycle and additional pharmaceutically-active agent with a time separation of no more than a few minutes, for example, less than about 15 minutes, less than about 10, less than about 5, or less than about 1 minute. When the drugs are administered simultaneously, the peptidomimetic macrocycle and the at least one additional pharmaceutically-active agent, for example, any additional therapeutic agent described herein, can be contained in the same composition (e.g., a composition comprising both the peptidomimetic macrocycle and the at least additional pharmaceutically-active agent) or in separate compositions (e.g., the peptidomimetic macrocycle is contained in one composition and the at least additional pharmaceutically-active agent is contained in another composition).

According to other embodiments, the peptidomimetic macrocycles and the at least one additional pharmaceutically-active agent, for example, any additional therapeutic agent described herein, are administered sequentially, i.e., the peptidomimetic macrocycle is administered either prior to or after the administration of the additional pharmaceutically-active agent. The term “sequential administration” as used herein means that the peptidomimetic macrocycle and the additional pharmaceutically-active agent, for example, any additional therapeutic agent described herein, are administered with a time separation of more than a few minutes, for example, more than about 15 minutes, more than about 20 or more minutes, more than about 30 or more minutes, more than about 40 or more minutes, more than about 50 or more minutes, or more than about 60 or more minutes. In some embodiments, the peptidomimetic macrocycle is administered before the additional pharmaceutically-active agent, for example, any additional therapeutic agent described herein. In some embodiments, the pharmaceutically-active agent, for example, any additional therapeutic agent described herein, is administered before the peptidomimetic macrocycle. The peptidomimetic macrocycle and the additional pharmaceutically-active agent, for example, any additional therapeutic agent described herein, can be contained in separate compositions, which may be contained in the same or different packages.

In some embodiments, the administration of the peptidomimetic macrocycles and the additional pharmaceutically-active agent, for example, any additional therapeutic agent described herein, are concurrent, i.e., the administration period of the peptidomimetic macrocycles and that of the agent overlap with each other. In some embodiments, the administration of the peptidomimetic macrocycles and the additional pharmaceutically-active agent, for example, any additional therapeutic agent described herein, are non-concurrent. For example, in some embodiments, the administration of the peptidomimetic macrocycles is terminated before the additional pharmaceutically-active agent, for example, any additional therapeutic agent described herein, is administered. In some embodiments, the administration of the additional pharmaceutically-active agent, for example, any additional therapeutic agent described herein, is terminated before the peptidomimetic macrocycle is administered. The time period between these two non-concurrent administrations can range from being days apart to being weeks apart.

The dosing frequency of the peptidomimetic macrocycle and the at least one additional pharmaceutically-active agent, for example, any additional therapeutic agent described herein, can be adjusted over the course of the treatment, based on the judgment of an administering physician. When administered separately, the peptidomimetic macrocycle and the at least one additional pharmaceutically-active agent, for example, any additional therapeutic agent described herein, can be administered at different dosing frequency or intervals. For example, the peptidomimetic macrocycle can be daily, while the at least one additional pharmaceutically-active agent, for example, any additional therapeutic agent described herein, can be administered more or less frequently. Or, both the peptidomimetic and the at least one additional pharmaceutically-active agent, for example, any additional therapeutic agent described herein, can be administered daily. In some embodiments, treatment with the peptidomimetic macrocycle can begin 1, 2, 3, 4, 5, 6, 7, 8, 9, or 10 days prior to treatment with the additional pharmaceutically-active agent. In addition, the peptidomimetic macrocycle and the at least one additional pharmaceutically-active agent, for example, any additional therapeutic agent described herein, can be administered using the same route of administration or using different routes of administration.

The combination of the peptidomimetic macrocycles and one or more additional therapies can be administered to a subject in the same pharmaceutical composition. Alternatively, the peptidomimetic macrocycles and one or more additional therapies can be administered concurrently to a subject in separate pharmaceutical compositions. The peptidomimetic macrocycles and one or more additional therapies can be administered sequentially to a subject in separate pharmaceutical compositions. Pharmaceutical compositions containing peptidomimetic macrocycles or one or more additional therapies can be administered to a subject by the same or different routes of administration.

The combination therapies provided herein can involve administering to a subject to in need thereof the peptidomimetic macrocycles in combination with other therapies for treating cancer. Other therapies for cancer or a condition associated therewith can be aimed at controlling or relieving one or more symptoms. Accordingly, in some embodiments, the combination therapies provided herein involve administering to a subject to in need thereof a pain reliever, or other therapies aimed at alleviating or controlling one or more symptoms associated with or a condition associated therewith.

### Dosing Regimen of Combination Treatment

Combination treatments disclosed herein can be administered with a variety of dosing regimens. The timing and selected dosage level of administration of a peptidomimetic macrocycle or an additional pharmaceutically-active agent can depend on a variety of factors including the activity of the particular peptidomimetic macrocycle employed, the route of administration, the rate of excretion or metabolism of the particular peptidomimetic macrocycle being employed, the duration of the treatment, the particular pharmaceutically-active agent used in combination with the particular peptidomimetic macrocycle employed, the age, sex, weight, condition, general health and prior medical history of the patient being treated, and other factors. The dosage values can also vary with the severity of the condition to be alleviated. For any particular subject, specific dosage regimens can be adjusted over time according to the individual need and the professional judgment of the person administering or supervising the administration of the compositions.

A medical professional, such as a physician or veterinarian, can readily determine and prescribe the effective amount of the pharmaceutical composition required. For example, a physician or veterinarian could start doses of the compounds of the disclosure employed in the pharmaceutical composition at levels lower than that required in order to achieve the desired therapeutic effect and gradually increase the dosage until the desired effect is achieved.

In some embodiments, a suitable daily dose of a peptidomimetic macrocycle of the disclosure can be an amount of the peptidomimetic macrocycle which is the lowest dose effective to induce cell cycle arrest in a tissue with a functional p53 protein. In some embodiments, a suitable dose or a peptidomimetic macrocycle of the disclosure is less than an amount of the peptidomimetic macrocycle that is needed to induce apoptosis in a tissue with a functional p53 protein. Such an effective dose can depend upon the factors described above. The precise time of administration and amount of any particular peptidomimetic macrocycle or other pharmaceutically-active agent that will yield the most effective treatment in a given patient can, in some instances, depend upon the activity, pharmacokinetics, and bioavailability of a particular peptidomimetic macrocycle, physiological condition of the patient (including age, sex, disease type and stage, general physical condition, responsiveness to a given dosage and type of medication), route of administration, and the like.

Dosage can be based on the amount of the peptidomimetic macrocycle per kg body weight of the patient. Other amounts are known to those of skill in the art and readily determined. Alternatively, the dosage of the subject disclosure can be determined by reference to the plasma concentrations of the peptidomimetic macrocycle. For example, the maximum plasma concentration (Cmax) and the area under the plasma concentration-time curve from time 0 to infinity (AUC) can be used.

In some embodiments, the subject is a human subject. Doses of a peptidomimetic macrocycle and/or additional pharmaceutically-active agent disclosed herein can be in the range of about 0.01 mg/kg to about 1000 mg/kg per day (e.g., about 0.01 mg/kg to about 100 mg/kg per day, about 0.01 mg/kg to about 10 mg/kg per day, about 0.01 mg/kg to about 3.2 mg/kg per day, about 0.1 mg/kg to about 100 mg/kg per day, about 0.1 mg/kg to about 50 mg/kg per day, about 0.1 mg/kg to about 10 mg/kg per day, about 0.1 mg/kg to about 3.2 mg/kg per day, at most about 3.2 mg/kg per day) of one or each component of the combinations described herein. In some embodiments, doses of a peptidomimetic macrocycle employed for human treatment are in the range of about 0.01 mg/kg to about 100 mg/kg per day (e.g., about 0.01 mg/kg to about 10 mg/kg per day, about 0.1 mg/kg to about 100 mg/kg per day, about 0.1 mg/kg to about 50 mg/kg per day, about 0.1 mg/kg to about 10 mg/kg per day, about 1 mg/kg per day). In some embodiments, doses of the additional pharmaceutically-active agent, for example, any additional therapeutic agent described herein, employed for human treatment can be in the range of about 0.01 mg/kg to about 100 mg/kg per day (e.g., about 0.1 mg/kg to about 100 mg/kg per day, about 0.1 mg/kg to about 50 mg/kg per day, about 10 mg/kg per day or about 30 mg/kg per day). The desired dose may be conveniently administered in a single dose, or as multiple doses administered at appropriate intervals, for example as two, three, four or more sub-doses per day.

In some embodiments, such as when given in combination with the at least one additional pharmaceutically active agent, for example, any additional therapeutic agent described herein, the dosage of a peptidomimetic macrocycle may be given at relatively lower dosages. In some embodiments, the dosage of a peptidomimetic macrocycle may be from about 1 ng/kg to about 100 mg/kg. The dosage of a peptidomimetic macrocycle may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 1.5 mg/kg, 2.0 mg/kg, 2.5 mg/kg, 3 mg/kg, 3.1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

In some embodiments, the dosage of the additional pharmaceutically-active agent, for example, any additional therapeutic agent described herein, may be from about 1 ng/kg to about 100 mg/kg. The dosage of the additional pharmaceutically-active agent may be at any dosage including, but not limited to, about 1 μg/kg, 25 μg/kg, 50 μg/kg, 75 μg/kg, 100 μμg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 g/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

In some embodiments, the dosage of the additional pharmaceutically-active agent, for example, any additional therapeutic agent described herein, is provided as an amount of agent per body surface area of a subject (e.g. mg/m²). In some embodiments, the dosage of the additional pharmaceutically-active agent, for example, any additional therapeutic agent described herein, is about 0.1 mg/m² to about 100 mg/m². In some embodiments, the dosage of the additional pharmaceutically-active agent, for example, any additional therapeutic agent described herein, is about 0.1 mg/m² to about 0.25 mg/m², about 0.1 mg/m² to about 0.5 mg/m², about 0.1 mg/m² to about 0.75 mg/m², about 0.1 mg/m² to about 1 mg/m², about 0.1 mg/m² to about 1.5 mg/m², about 0.1 mg/m² to about 2 mg/m², about 0.1 mg/m² to about 2.5 mg/m², about 0.1 mg/m² to about 5 mg/m², about 0.1 mg/m² to about 10 mg/m², about 0.1 mg/m² to about 75 mg/m², about 0.1 mg/m² to about 100 mg/m², about 0.25 mg/m² to about 0.5 mg/m², about 0.25 mg/m² to about 0.75 mg/m², about 0.25 mg/m² to about 1 mg/m², about 0.25 mg/m² to about 1.5 mg/m², about 0.25 mg/m² to about 2 mg/m², about 0.25 mg/m² to about 2.5 mg/m², about 0.25 mg/m² to about 5 mg/m², about 0.25 mg/m² to about 10 mg/m², about 0.25 mg/m² to about 75 mg/m², about 0.25 mg/m² to about 100 mg/m², about 0.5 mg/m² to about 0.75 mg/m², about 0.5 mg/m² to about 1 mg/m², about 0.5 mg/m² to about 1.5 mg/m², about 0.5 mg/m² to about 2 mg/m², about 0.5 mg/m² to about 2.5 mg/m², about 0.5 mg/m² to about 5 mg/m², about 0.5 mg/m² to about 10 mg/m², about 0.5 mg/m² to about 75 mg/m², about 0.5 mg/m² to about 100 mg/m², about 0.75 mg/m² to about 1 mg/m², about 0.75 mg/m² to about 1.5 mg/m², about 0.75 mg/m² to about 2 mg/m², about 0.75 mg/m² to about 2.5 mg/m², about 0.75 mg/m² to about 5 mg/m², about 0.75 mg/m² to about 10 mg/m², about 1 mg/m² to about 1.5 mg/m², about 1 mg/m² to about 2 mg/m², about 1 mg/m² to about 2.5 mg/m², about 1 mg/m² to about 5 mg/m², about 1 mg/m² to about 10 mg/m², about 1 mg/m² to about 75 mg/m², about 1 mg/m² to about 100 mg/m², about 1.5 mg/m² to about 2 mg/m², about 1.5 mg/m² to about 2.5 mg/m², about 1.5 mg/m² to about 5 mg/m², about 1.5 mg/m² to about 10 mg/m², about 1.5 mg/m² to about 75 mg/m², about 1.5 mg/m² to about 100 mg/m², about 2 mg/m² to about 2.5 mg/m², about 2 mg/m² to about 5 mg/m², about 2 mg/m² to about 10 mg/m², about 2 mg/m² to about 75 mg/m², about 2 mg/m² to about 100 mg/m², about 2.5 mg/m² to about 5 mg/m², about 2.5 mg/m² to about 10 mg/m², about 2.5 mg/m² to about 75 mg/m², about 2.5 mg/m² to about 100 mg/m², about 5 mg/m² to about 10 mg/m², 5 mg/m² to about 75 mg/m², about 5 mg/m² to about 100 mg/m², about 10 mg/m² to about 75 mg/m², about 10 mg/m² to about 100 mg/m², or about 75 mg/m² to about 100 mg/m². In some embodiments, the dosage of the additional pharmaceutically-active agent, for example, any additional therapeutic agent described herein, is about 0.1 mg/m², about 0.25 mg/m², about 0.5 mg/m², about 0.75 mg/m², about 1 mg/m², about 1.5 mg/m², about 2 mg/m², about 2.3 mg/m², about 2.5 mg/m², about 5 mg/m², about 10 mg/m², about 75 mg/m², or about 100 mg/m². In some embodiments, the dosage of the additional pharmaceutically-active agent, for example, any additional therapeutic agent described herein, is at least about 0.1 mg/m², about 0.25 mg/m², about 0.5 mg/m², about 0.75 mg/m², about 1 mg/m², about 1.5 mg/m², about 2 mg/m², about 2.5 mg/m², or about 5 mg/m². In some embodiments, the dosage of the additional pharmaceutically-active agent, for example, any additional therapeutic agent described herein, is at most about 0.25 mg/m², about 0.5 mg/m², about 0.75 mg/m², about 1 mg/m², about 1.5 mg/m², about 2 mg/m², about 2.5 mg/m², about 5 mg/m², about 10 mg/m², or about 75 mg/m².

In some embodiments, the dosage of the additional pharmaceutically-active agent is the approved dosage from the label of the additional pharmaceutically-active agent. In some embodiments, the dosage of the additional pharmaceutically-active agent is 1.5 mg/m² or 2.3 mg/m² topotecan or a pharmaceutically acceptable salt thereof. In some embodiments, the dosage of the additional pharmaceutically-active agent is 75 mg/m² topotecan or a pharmaceutically-acceptable salt thereof. In some embodiments, the approved dosages of the additional pharmaceutically-active agents can be reduced to address adverse side effects such as renal impairment or liver impairment.

The peptidomimetic macrocycle and the additional pharmaceutically-active agent, for example, any additional therapeutic agent described herein, can be provided in a single unit dosage form for being taken together or as separate entities (e.g. in separate containers). The peptidomimetic macrocycle and the additional pharmaceutically-active agent, for example, any additional therapeutic agent described herein, can be administered simultaneously or with a certain time difference. This time difference can be, for example, between about 0.1 hours to about 1 week. In some embodiments, the time difference is about 0.1 hours to about 6 days, about 0.1 hours to about 5 days, about 0.1 hours to about 4 days, about 0.1 hours to about 3 days, about 0.1 hours to about 48 hours, about 0.1 hours to about 36 hours, about 0.1 hours to about 24 hours, about 0.1 hours to about 12 hours, about 0.1 hours to about 6 hours, about 0.1 hours to about 1 hour, about 0.1 hours to about 0.5 hours, about 0.5 hours to about 1 week, about 0.5 hours to about 6 days, about 0.5 hours to about 5 days, about 0.5 hours to about 4 days, about 0.5 hours to about 3 days, about 0.5 hours to about 48 hours, about 0.5 hours to about 36 hours, about 0.5 hours to about 24 hours, about 0.5 hours to about 12 hours, about 0.5 hours to about 6 hours, about 0.5 hours to about 1 hour, about 1 hour to about 1 week, about 1 hour to about 6 days, about 1 hour to about 5 days, about 1 hour to about 4 days, about 1 hour to about 3 days, about 1 hour to about 48 hours, about 1 hour to about 36 hours, about 1 hour to about 24 hours, about 1 hour to about 12 hours, about 1 hour to about 6 hours, about 6 hours to about 12 hours, about 6 hours to about 1 week, about 6 hours to about 6 days, about 6 hours to about 5 days, about 6 hours to about 4 days, about 6 hours to about 3 days, about 6 hours to about 48 hours, about 6 hours to about 36 hours, about 6 hours to about 24 hours, about 6 hours to about 12 hours, about 12 hours to about 1 week, about 12 hours to about 6 days, about 12 hours to about 5 days, about 12 hours to about 4 days, about 12 hours to about 3 days, about 12 hours to about 48 hours, about 12 hours to about 36 hours, about 12 hours to about 24 hours, about 24 hours to about 1 week, about 24 hours to about 6 days, about 24 hours to about 5 days, about 24 hours to about 4 days, about 24 hours to about 3 days, about 24 hours to about 48 hours, about 24 hours to about 36 hours, about 36 hours to about 1 week, about 36 hours to about 6 days, about 36 hours to about 5 days, about 36 hours to about 4 days, about 36 hours to about 3 days, about 36 hours to about 48 hours, or about 48 hours to about 1 week. In some embodiments, the time period is about 1 week, about 6 days, about 5 days, about 4 days, about 3 days, about 48 hours, about 36 hours, about 12 hours, about 6 hours, about 0.5 hours, or about 0.1 hours. In some embodiments, the time period is at most about 1 week, at most about 6 days, at most about 5 days, at most about 4 days, at most about 3 days, at most about 48 hours, at most about 36 hours, at most about 12 hours, at most about 6 hours, or at most about 0.5 hours. In some embodiments, the time period is at least about 6 days, at least about 5 days, at least about 4 days, at least about 3 days, at least about 48 hours, at least about 36 hours, at least about 12 hours, at least about 6 hours, at least about 0.5 hours, or at least about 0.1 hours. In some embodiments, the peptidomimetic macrocycle is administered first, followed by the time difference, followed by administration of the additional pharmaceutically-active agent, for example, any additional therapeutic agent described herein. In some embodiments, the additional pharmaceutically-active agent, for example, any additional therapeutic agent described herein is administered, followed by the time difference, followed by administration of a peptidomimetic macrocycle.

In some embodiments, the peptidomimetic macrocycle is administered about 0.1 hours, about 0.2 hours, about 0.3 hours, about 0.4 hours, about 0.5 hours, about 1 hour, about 6 hours, about 12 hours, about 24 hours, about 36 hours, about 48 hours, about 3 days, about 4 days, about 5 days, about 6 days, or about 1 week before the additional pharmaceutically-active agent, for example, any additional therapeutic agent described herein, is administered. In some embodiments, the peptidomimetic macrocycle is administered about 1 day before the additional pharmaceutically-active agent, for example, any additional therapeutic agent described herein, is administered.

In some embodiments, the peptidomimetic macrocycle is administered about 0.1 hours, about 0.2 hours, about 0.3 hours, about 0.4 hours, about 0.5 hours, about 1 hour, about 6 hours, about 12 hours, about 24 hours, about 36 hours, about 48 hours, about 3 days, about 4 days, about 5 days, about 6 days, or about 1 week after the additional pharmaceutically-active agent, for example, any additional therapeutic agent described herein, is administered. In some embodiments, the peptidomimetic macrocycle is administered about 6 hours after the additional pharmaceutically-active agent, for example, any additional therapeutic agent described herein, is administered.

In some embodiments, the peptidomimetic macrocycle is administered chronologically before the additional pharmaceutically-active agent, for example, any additional therapeutic agent described herein. In some embodiments, the peptidomimetic macrocycle is administered from about 0.1 hours to about 1 week, about 0.1 hours to about 6 days, about 0.1 hours to about 5 days, about 0.1 hours to about 4 days, about 0.1 hours to about 3 days, about 0.1 hours to about 48 hours, about 0.1 hours to about 36 hours, about 0.1 hours to about 24 hours, about 0.1 hours to about 12 hours, about 0.1 hours to about 6 hours, about 0.1 hours to about 1 hour, about 0.1 hours to about 0.5 hours, about 0.5 hours to about 1 week, about 0.5 hours to about 6 days, about 0.5 hours to about 5 days, about 0.5 hours to about 4 days, about 0.5 hours to about 3 days, about 0.5 hours to about 48 hours, about 0.5 hours to about 36 hours, about 0.5 hours to about 24 hours, about 0.5 hours to about 12 hours, about 0.5 hours to about 6 hours, about 0.5 hours to about 1 hour, about 1 hour to about 1 week, about 1 hour to about 6 days, about 1 hour to about 5 days, about 1 hour to about 4 days, about 1 hour to about 3 days, about 1 hour to about 48 hours, about 1 hour to about 36 hours, about 1 hour to about 24 hours, about 1 hour to about 12 hours, about 1 hour to about 6 hours, about 6 hours to about 12 hours, about 6 hours to about 1 week, about 6 hours to about 6 days, about 6 hours to about 5 days, about 6 hours to about 4 days, about 6 hours to about 3 days, about 6 hours to about 48 hours, about 6 hours to about 36 hours, about 6 hours to about 24 hours, about 6 hours to about 12 hours, about 12 hours to about 1 week, about 12 hours to about 6 days, about 12 hours to about 5 days, about 12 hours to about 4 days, about 12 hours to about 3 days, about 12 hours to about 48 hours, about 12 hours to about 36 hours, about 12 hours to about 24 hours, about 24 hours to about 1 week, about 24 hours to about 6 days, about 24 hours to about 5 days, about 24 hours to about 4 days, about 24 hours to about 3 days, about 24 hours to about 48 hours, about 24 hours to about 36 hours, about 36 hours to about 1 week, about 36 hours to about 6 days, about 36 hours to about 5 days, about 36 hours to about 4 days, about 36 hours to about 3 days, about 36 hours to about 48 hours, about 48 hours to about 1 week or any combination thereof, before the additional pharmaceutically-active agent, for example, any additional therapeutic agent described herein, is administered. In some embodiments, the peptidomimetic macrocycle is administered at least about 6 days, at least about 5 days, at least about 4 days, at least about 3 days, at least about 48 hours, at least about 36 hours, at least about 12 hours, at least about 6 hours, at least about 0.5 hours, at least about 0.1 hours or any combination thereof, before the additional pharmaceutically-active agent, for example, any additional therapeutic agent described herein, is administered. For example, the peptidomimetic macrocycle can be administered at least 1 day before a topoisomerase inhibitor (e.g., topotecan) is administered.

In some embodiments, the peptidomimetic macrocycle is administered at most about 1 week, at most about 6 days, at most about 5 days, at most about 4 days, at most about 3 days, at most about 48 hours, at most about 36 hours, at most about 12 hours, at most about 6 hours, at most about 0.5 hours or any combination thereof, before the additional pharmaceutically-active agent is administered. For example, the peptidomimetic macrocycle can be administered at most about 1 week, at most about 6 days, at most about 5 days, at most about 4 days, at most about 3 days, at most about 48 hours, at most about 36 hours, at most about 12 hours, at most about 6 hours, at most about 0.5 hours or any combination thereof, before a topoisomerase inhibitor (e.g. topotecan) is administered.

In some embodiments, the peptidomimetic macrocycle is administered about 1 week, about 6 days, about 5 days, about 4 days, about 3 days, about 48 hours, about 36 hours, about 24 hours, about 12 hours, about 8 hours, about 6 hours, about 0.5 hours, about 0.1 hours, or any combination thereof, before the additional pharmaceutically-active agent, for example, any additional therapeutic agent described herein, is administered. For example, the peptidomimetic macrocycle can be administered about 1 week, about 6 days, about 5 days, about 4 days, about 3 days, about 48 hours, about 36 hours, about 12 hours, about 6 hours, about 0.5 hours, about 0.1 hours, or any combination thereof, before a topoisomerase inhibitor (e.g. topotecan) is administered.

In some embodiments, the peptidomimetic macrocycle is administered chronologically at the same time as the at least one additional pharmaceutically active agent, for example, any additional therapeutic agent described herein.

In some embodiments, the peptidomimetic macrocycle is administered chronologically after the additional pharmaceutically-active agent, for example, any additional therapeutic agent described herein. In some embodiments, the additional pharmaceutically-active agent, for example, any additional therapeutic agent described herein, is administered from 0.1 hours to about 1 week, about 0.1 hours to about 6 days, about 0.1 hours to about 5 days, about 0.1 hours to about 4 days, about 0.1 hours to about 3 days, about 0.1 hours to about 48 hours, about 0.1 hours to about 36 hours, about 0.1 hours to about 24 hours, about 0.1 hours to about 12 hours, about 0.1 hours to about 6 hours, about 0.1 hours to about 1 hour, about 0.1 hours to about 0.5 hours, about 0.5 hours to about 1 week, about 0.5 hours to about 6 days, about 0.5 hours to about 5 days, about 0.5 hours to about 4 days, about 0.5 hours to about 3 days, about 0.5 hours to about 48 hours, about 0.5 hours to about 36 hours, about 0.5 hours to about 24 hours, about 0.5 hours to about 12 hours, about 0.5 hours to about 6 hours, about 0.5 hours to about 1 hour, about 1 hour to about 1 week, about 1 hour to about 6 days, about 1 hour to about 5 days, about 1 hour to about 4 days, about 1 hour to about 3 days, about 1 hour to about 48 hours, about 1 hour to about 36 hours, about 1 hour to about 24 hours, about 1 hour to about 12 hours, about 1 hour to about 6 hours, about 6 hours to about 12 hours, about 6 hours to about 1 week, about 6 hours to about 6 days, about 6 hours to about 5 days, about 6 hours to about 4 days, about 6 hours to about 3 days, about 6 hours to about 48 hours, about 6 hours to about 36 hours, about 6 hours to about 24 hours, about 6 hours to about 12 hours, about 12 hours to about 1 week, about 12 hours to about 6 days, about 12 hours to about 5 days, about 12 hours to about 4 days, about 12 hours to about 3 days, about 12 hours to about 48 hours, about 12 hours to about 36 hours, about 12 hours to about 24 hours, about 24 hours to about 1 week, about 24 hours to about 6 days, about 24 hours to about 5 days, about 24 hours to about 4 days, about 24 hours to about 3 days, about 24 hours to about 48 hours, about 24 hours to about 36 hours, about 36 hours to about 1 week, about 36 hours to about 6 days, about 36 hours to about 5 days, about 36 hours to about 4 days, about 36 hours to about 3 days, about 36 hours to about 48 hours, about 48 hours to about 1 week, about 7-30 days, or any combination thereof, after the peptidomimetic macrocycle is administered. In some embodiments the additional pharmaceutically-active agent is administered at least about 0.1 hours, at least about 0.5 hours at least about 1 hour, at least about 6 hours, at least about 12 hours, at least about 24 hours, at least about 36 hours, at least about 48 hours, at least about 3 days, at least about 4 days, at least about 5 days, at least about 6 days, at least about 1 week, at least about 2 weeks, at least about 3 weeks, at least about 4 weeks, at least about 1 month, or any combination thereof, after the peptidomimetic macrocycle is administered.

In some embodiments, a topoisomerase inhibitor (e.g. topotecan) is administered at most about 0.1 hours, at most about 0.5 hours, at most about 1 hour, at most about 6 hours, at most about 12 hours, at most about 24 hours, at most about 36 hours, at most about 48 hours, at most about 3 days, at most about 4 days, at most about 5 days, at most about 6 days, at most about 1 week, at most about 2 weeks, at most about 3 weeks, at most about 4 weeks, at most about 1 month, or any combination thereof, after the peptidomimetic macrocycle is administered. For example, topotecan can be administered at most about 0.1 hours, at most about 0.5 hours at most about 1 hour at most about 6 hours, at most about 12 hours, at most about 24 hours, at most about 36 hours, at most about 48 hours, at most about 3 days, at most about 4 days, at most about 5 days, at most about 6 days, at most about 1 week, at most about 2 weeks, at most about 3 weeks, at most about 4 weeks, at most about 1 month, or any combination thereof, after the peptidomimetic macrocycle is administered.

In some embodiments, a pharmaceutically active agent (e.g. topotecan, docetaxel, carboplatin, paclitaxel, or any combination thereof) is administered about 0.1 hours, about 0.5 hours, about 1 hour, about 6 hours, about 8 hours about 12 hours, about 24 hours, about 36 hours, about 48 hours, about 3 days, about 4 days, about 5 days, about 6 days, about 1 week, about 2 weeks, about 3 weeks, about 4 weeks, about 1 month, or any combination thereof, after the peptidomimetic macrocycle is administered.

Also, contemplated herein is a drug holiday utilized among the administration of a peptidomimetic macrocycle and an additional pharmaceutically-active agent, for example, any additional therapeutic agent described herein. A drug holiday can be a period of days after the administration of the additional pharmaceutically-active agent, for example, any additional therapeutic agent described herein, and before the administration of a peptidomimetic macrocycle. A drug holiday can be a period of days after the administration of a peptidomimetic macrocycle and before the administration of the additional pharmaceutically-active agent, for example, any additional therapeutic agent described herein. A drug holiday can be a period of days after the sequential administration of one or more of a peptidomimetic macrocycle and an additional pharmaceutically-active agent, for example, any additional therapeutic agent described herein, and before the administration of the peptidomimetic macrocycle, the additional pharmaceutically-active agent, or another therapeutic agent. For example, a drug holiday can be a period of days after the sequential administration of a peptidomimetic macrocycle first, followed administration of an additional pharmaceutically-active agent, for example, any additional therapeutic agent described herein, and before the administration of the peptidomimetic macrocycle again. For example, a drug holiday can be a period of days after the sequential administration of an additional pharmaceutically-active agent first, followed administration of a peptidomimetic macrocycle and before the administration of the additional pharmaceutically-active agent, for example, any additional therapeutic agent described herein.

Suitably the drug holiday can be a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days; or from 1-24, 2-24, 3-24, 4-24, 5-24, 6-24, 7-24, 8-24, 9-24, 10-24, 11-24, or 12-24 hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 days, 1-4, 2-4,or 3-4 weeks; or from 1-12, 2-12, 3-12, 4-12, 5-12, 6-12, 7-12, 8-12, 9-12, 10-12, or 11-12 months.

In some embodiments, an additional pharmaceutically-active agent, for example, any additional therapeutic agent described herein, will be administered first in the sequence, followed by an optional drug holiday, followed by administration of a peptidomimetic macrocycle. In some embodiments, an additional pharmaceutically-active agent, for example, any additional therapeutic agent described herein, will be administered first in the sequence, followed by administration of a peptidomimetic macrocycle, followed by an optional drug holiday, followed by administration of an additional pharmaceutically-active agent.

In some embodiments, an additional pharmaceutically-active agent, for example, any additional therapeutic agent described herein, is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months. For example, a topoisomerase inhibitor is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by a drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months.

In some embodiments, an additional pharmaceutically-active agent, for example, any additional therapeutic agent described herein, is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of an additional pharmaceutically-active agent. For example, a topoisomerase inhibitor is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by administration of a peptidomimetic macrocycle for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by administration of a topoisomerase inhibitor.

In some embodiments, a peptidomimetic macrocycle will be administered first in the sequence, followed by an optional drug holiday, followed by administration of an additional pharmaceutically-active agent, for example, any additional therapeutic agent described herein. In some embodiments, a peptidomimetic macrocycle will be administered first in the sequence, followed by administration of an additional pharmaceutically-active agent, for example, any additional therapeutic agent described herein, followed by an optional drug holiday, followed by administration of a peptidomimetic macrocycle.

In some embodiments, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of an additional pharmaceutically-active agent, for example, any additional therapeutic agent described herein, for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months. For example, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by a drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by administration of a topoisomerase inhibitor for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months.

In some embodiments, a peptidomimetic macrocycle is administered from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by administration of an additional pharmaceutically-active agent, for example, any additional therapeutic agent described herein, for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday; followed by administration of a peptidomimetic macrocycle. For example, a peptidomimetic macrocycle is administered for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by administration of a topoisomerase inhibitor for from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months, followed by an optional drug holiday of from 1-24, 2-24, 3-24, 4-24, 5-24, 6-24, 7-24, 8-24, 9-24, 10-24, 11-24, or 12-24 consecutive hours; from 1-30, 2-30, 3-30, 4-30, 5-30, 6-30, 7-30, 8-30, 9, −30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 consecutive days, 1-4, 2-4,or 3-4 consecutive weeks; or from 1-12, 2-12, 3-12, 4-12, 5-12, 6-12, 7-12, 8-12, 9-12, 10-12, or 11-12 consecutive months; followed by administration of a peptidomimetic macrocycle.

In some embodiments, an additional pharmaceutically-active agent, for example, any additional therapeutic agent described herein, will be administered first in the sequence, followed by an optional drug holiday, followed by administration of a peptidomimetic macrocycle. In some embodiments, a topoisomerase inhibitor will be administered first in the sequence, followed by an optional drug holiday, followed by administration of a peptidomimetic macrocycle, followed by an optional drug holiday, followed by administration of a topoisomerase inhibitor.

In some embodiments, an additional pharmaceutically-active agent, for example, any additional therapeutic agent described herein, is administered for from 1 to 30 consecutive days, followed by an optional drug holiday, followed by administration of peptidomimetic macrocycle for from 1 to 30 consecutive days. In some embodiments, an additional pharmaceutically-active agent, for example, any additional therapeutic agent described herein, is administered for from 1 to 21 consecutive days, followed by an optional drug holiday, followed by administration of a peptidomimetic macrocycle for from 1 to 21 consecutive days. In some embodiments, an additional pharmaceutically-active agent, for example, any additional therapeutic agent described herein, is administered for from 1 to 14 consecutive days, followed by an optional drug holiday, followed by administration of a peptidomimetic macrocycle for from 1 to 14 consecutive days. In some embodiments, an additional pharmaceutically-active agent, for example, any additional therapeutic agent described herein, is administered for 14 consecutive days, followed by an optional drug holiday, followed by administration of a peptidomimetic macrocycle for 7 consecutive days. In some embodiments, an additional pharmaceutically-active agent, for example, any additional therapeutic agent described herein, is administered for 7 consecutive days, followed by an optional drug holiday, followed by administration of a peptidomimetic macrocycle for 7 consecutive days.

In some embodiments, a peptidomimetic macrocycle is administered for from 1 to 30 consecutive days, followed by an optional drug holiday, followed by administration of an additional pharmaceutically-active agent, for example, any additional therapeutic agent described herein, for from 1 to 30 consecutive days. In some embodiments, a peptidomimetic macrocycle is administered for from 1 to 21 consecutive days, followed by an optional drug holiday, followed by administration of an additional pharmaceutically-active agent, for example, any additional therapeutic agent described herein, for from 1 to 21 consecutive days. In some embodiments, a peptidomimetic macrocycle is administered for from 1 to 14 consecutive days, followed by an optional drug holiday, followed by administration of an additional pharmaceutically-active agent, for example, any additional therapeutic agent described herein, for from 1 to 14 consecutive days. In some embodiments, a peptidomimetic macrocycle is administered for 14 consecutive days, followed by an optional drug holiday, followed by administration of an additional pharmaceutically-active agent, for example, any additional therapeutic agent described herein, for 14 consecutive days. In some embodiments, a peptidomimetic macrocycle is administered for 7 consecutive days, followed by an optional drug holiday, followed by administration of an additional pharmaceutically-active agent, for example, any additional therapeutic agent described herein, for 7 consecutive days.

In some embodiments, one of a peptidomimetic macrocycle and an additional pharmaceutically-active agent, for example, any additional therapeutic agent described herein, is administered for from 2 to 30 consecutive days, followed by an optional drug holiday, followed by administration of the other of a peptidomimetic macrocycle and an additional pharmaceutically-active agent from between 2 to 30 consecutive days. In some embodiments, one of a peptidomimetic macrocycle and an additional pharmaceutically-active agent, for example, any additional therapeutic agent described herein, is administered for from 2 to 21 consecutive days, followed by an optional drug holiday, followed by administration of the other of a peptidomimetic macrocycle and an additional pharmaceutically-active agent for from 2 to 21 consecutive days. In some embodiments, one of a peptidomimetic macrocycle and an additional pharmaceutically-active agent, for example, any additional therapeutic agent described herein, is administered for from 2 to 14 consecutive days, followed by a drug holiday of from 1 to 14 days, followed by administration of the other of a peptidomimetic macrocycle and an additional pharmaceutically-active agent for from 2 to 14 consecutive days. In some embodiments, one of a peptidomimetic macrocycle and an additional pharmaceutically-active agent, for example, any additional therapeutic agent described herein, is administered for from 3 to 7 consecutive days, followed by a drug holiday of from 3 to 10 days, followed by administration of the other of a peptidomimetic macrocycle and an additional pharmaceutically-active agent for from 3 to 7 consecutive days.

In some embodiments, a peptidomimetic macrocycle is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the peptidomimetic macrocycle/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day cycle; and the additional pharmaceutically-active agent, for example, any additional therapeutic agent described herein, is administered prior to, concomitantly with, or subsequent to administration of the peptidomimetic macrocycle on one or more days (e.g., on day 1 of cycle 1). In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, 28, or more days. In some embodiments, the combination therapy is administered for 1 to 12 or 13 cycles of 28 days (e.g., about 12 months).

In some embodiments, the components of the combination therapies described herein (e.g., a peptidomimetic macrocycle and any additional therapeutic agent disclosed herein) are cyclically administered to a patient. In some embodiments, a secondary active agent is co-administered in a cyclic administration with the combination therapies provided herein. Cycling therapy involves the administration of an active agent for a period of time, followed by a rest for a period of time, and repeating this sequential administration. Cycling therapy can be performed independently for each active agent (e.g., a peptidomimetic macrocycle and an additional pharmaceutically-active agent, and/or a secondary agent) over a prescribed duration of time. In some embodiments, the cyclic administration of each active agent is dependent upon one or more of the active agents administered to the subject. In some embodiments, administration of a peptidomimetic macrocycle or an additional pharmaceutically-active agent, for example, any therapeutic agent disclosed herein, fixes the day(s) or duration of administration of the peptidomimetic macrocycle and the additional therapeutically-active agent.

In some embodiments, the frequency of administration is in the range of about a daily dose to about a monthly dose. In some embodiments, administration is once a day, twice a day, three times a day, four times a day, once every other day, twice a week, once every week, once every two weeks, once every three weeks, or once every four weeks. In some embodiments, a compound for use in combination therapies described herein is administered once a day. In some embodiments, a compound for use in combination therapies described herein is administered twice a day. In some embodiments, a compound for use in combination therapies described herein is administered three times a day. In some embodiments, a compound for use in combination therapies described herein is administered four times a day.

In some embodiments, the frequency of administration of a peptidomimetic macrocycle is in the range of about a daily dose to about a monthly dose. In some embodiments, administration of a peptidomimetic macrocycle is once a day, twice a day, three times a day, four times a day, once every other day, twice a week, once every week, once every two weeks, once every three weeks, or once every four weeks. In some embodiments, a peptidomimetic macrocycle for use in combination therapies described herein is administered once a day. In some embodiments, a peptidomimetic macrocycle for use in combination therapies described herein is administered twice a day. In some embodiments, a peptidomimetic macrocycle for use in combination therapies described herein is administered three times a day. In some embodiments, a peptidomimetic macrocycle for use in combination therapies described herein is administered four times a day.

In some embodiments, the frequency of administration of an additional pharmaceutically-active agent, for example, any additional therapeutic agent described herein, is in the range of about a daily dose to about a monthly dose. In some embodiments, administration of an additional pharmaceutically-active agent, for example, any additional therapeutic agent described herein, is once a day, twice a day, three times a day, four times a day, once every other day, twice a week, once every week, once every two weeks, once every three weeks, or once every four weeks.

In some embodiments, an additional pharmaceutically-active agent, for example, any additional therapeutic agent described herein, for use in combination therapies described herein is administered once a day. In some embodiments, an additional pharmaceutically-active agent, for example, any additional therapeutic agent described herein, for use in combination therapies described herein is administered twice a day. In some embodiments, an additional pharmaceutically-active agent, for example, any additional therapeutic agent described herein, for use in combination therapies described herein is administered three times a day. In some embodiments, an additional pharmaceutically-active agent, for example, any additional therapeutic agent described herein, for use in combination therapies described herein is administered four times a day.

In some embodiments, a compound for use in combination therapies described herein is administered once per day from one day to six months, from one week to three months, from one week to four weeks, from one week to three weeks, or from one week to two weeks. In some embodiments, a compound for use in combination therapies described herein is administered once per day for one week, two weeks, three weeks, or four weeks. In some embodiments, a compound for use in combination therapies described herein is administered once per day for one week. In some embodiments, a compound for use in combination therapies described herein is administered once per day for two weeks. In some embodiments, a compound for use in combination therapies described herein is administered once per day for three weeks. In some embodiments, a compound for use in combination therapies described herein is administered once per day for four weeks.

Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

In some embodiments, the periodic administration of a peptidomimetic macrocycle and/or the additional pharmaceutically-active agent, for example, any additional therapeutic agent described herein, is affected daily. In some embodiments, the periodic administration of a peptidomimetic macrocycle and/or the additional pharmaceutically-active agent, for example, any additional therapeutic agent described herein, is affected twice daily at one half the amount.

In some embodiments, the periodic administration of a peptidomimetic macrocycle and/or the additional pharmaceutically-active agent, for example, any additional therapeutic agent described herein, is affected once every 3 to 11 days; or once every 5 to 9 days; or once every 7 days; or once every 24 hours. In some embodiments, the periodic administration of a peptidomimetic macrocycle and/or the additional pharmaceutically-active agent, for example, any additional therapeutic agent described herein, is effected once every 2 days, 3 days, 4 days, 5 days, 6 days, 7 days, 8 days, 9 days, 10 days, 11 days, 12 days, 13 days, 14 days, 15 days, 6 days, 16 days, 17 days, 18 days, 19 days, 20 days, 21 days, 22 days, 23 days, 24 days, 25 days, 26 days, 27 days, 28 days, 29 days, or 30 days.

In some embodiments, the periodic administration of a peptidomimetic macrocycle and/or additional pharmaceutically-active agent is affected one, twice, or thrice daily.

For each administration schedule of a peptidomimetic macrocycle, the periodic administration of the additional pharmaceutically-active agent, for example, any additional therapeutic agent described herein, may be affected once every 16-32 hours; or once every 18-30 hours; or once every 20-28 hours; or once every 22-26 hours. In some embodiments, the administration of a peptidomimetic macrocycle substantially precedes the additional pharmaceutically-active agent, for example, any additional therapeutic agent described herein. In some embodiments, the administration of the additional pharmaceutically-active agent, for example, any additional therapeutic agent described herein, substantially precedes the administration of a peptidomimetic macrocycle.

In some embodiments, a peptidomimetic macrocycle and the additional pharmaceutically-active agent, for example, any additional therapeutic agent described herein, may be administered for a period of time of at least 4 days. In some embodiments, the period of time may be 5 days to 5 years; or 10 days to 3 years; or 2 weeks to 1 year; or 1 month to 6 months; or 3 months to 4 months. In some embodiments, a peptidomimetic macrocycle and the additional pharmaceutically-active agent, for example, any additional therapeutic agent described herein, may be administered for the lifetime of the subject.

In some embodiments, a peptidomimetic macrocycle and the additional pharmaceutically-active agent, for example, any additional therapeutic agent described herein, are administered during a treatment period. A treatment period disclosed herein can be, for example, a 1, 2, 3, 4, 5, 6, 7, 8, 9, 10, 12, 13, 14, 15, 16, 17, 18, 19, 20, 21, 22, 23, 24, 25, 26, 27, 28, 29, or 30-day treatment period. The first day of a treatment period can be denoted as, for example, day 0 or day 1. For example, a 22-day treatment period can begin on day 0 and end on day 21. In another example, a 22-day treatment period can begin on day 1 and end on day 22. A peptidomimetic macrocycle and/or an additional pharmaceutically-active agent, for example, any additional therapeutic agent described herein can be administered on any of Days 0, 1, 2, 3, 4, 5, 6, 7, 8, 9, 10, 12, 13, 14, 15, 16, 17, 18, 19, 20, 21, 22, 23, 24, 25, 26, 27, 28, 29, and/or 30 of a treatment period. A peptidomimetic macrocycle and/or an additional pharmaceutically-active agent, for example, any additional therapeutic agent described herein can be administered on one or more days of a treatment period. In some instances, neither a peptidomimetic macrocycle or an additional pharmaceutically-active agent is administered on one or more days of a treatment period. In some instances, the peptidomimetic macrocycle and the additional pharmaceutically-active agent, for example, any additional therapeutic agent described herein are both administered on some days of a treatment period while on other days of the treatment period only one of the macrocycle or the additional pharmaceutically-active agent is administered. For example, a peptidomimetic macrocycle can be administered on Days 1, 2, 3, 4, and 5 of a treatment period and an additional pharmaceutically-active agent (e.g., any additional therapeutic agent described herein) can be administered on Days 2, 3, 4, 5, and 6 of the treatment period. In some embodiments, a peptidomimetic macrocycle can be administered on Days 0, 1, 2, 3, and 4 of a treatment period and an additional pharmaceutically-active agent (e.g., any additional therapeutic agent described herein) can be administered on Days 1, 2, 3, 4, and 5 of the treatment period. In some embodiments, a peptidomimetic macrocycle can be administered on Days 0, 1, and 2 of a treatment period and an additional pharmaceutically-active agent (e.g., any additional therapeutic agent described herein) can be administered on Day 1 of the treatment period. In some embodiments, a peptidomimetic macrocycle can be administered on Days 1, 2, and 3 of a treatment period and an additional pharmaceutically-active agent (e.g., any additional therapeutic agent described herein) can be administered on Day 2 of the treatment period.

In some embodiments, a treatment period is part of a treatment cycle. For example, during a cycle denoted to begin on Day 1 and end on Day 22, a peptidomimetic macrocycle can be administered on days 1, 2, 3, 4, and 5 of the cycle, an additional pharmaceutically-active agent (e.g., any additional therapeutic agent described herein) can be administered on days 2, 3, 4, 5, and 6 of the cycle, and neither the macrocycle nor the additional agent can be administered on days 7-22 of the cycle.

In another example, a peptidomimetic macrocycle can be administered on days 1, 2, and 3 of a cycle denoted to begin on Day 1 and end on Day 22, an additional pharmaceutically-active agent can be administered on day 2 of the cycle, and neither the macrocycle nor the additional agent can be administered on days 4-22 of the cycle.

In some embodiments, multiple treatment cycles can be administered. For example, method disclosed herein can comprise administering 1, 2, 3, 4, 5, 6, 7, 8, 9, 10, 11, 12, 13, 14, 15 or more treatment cycles. In some embodiments, a method disclosed herein comprises administering at least 1, at least 2, at least 3, at least 4, at least 5, at least 6, at least 7, at least 8, at least 9, at least 10, at least 11, at least 12, at least 13, at least 14, or at least 15 treatment cycles. In some embodiments, a method of the disclosure comprises administering a peptidomimetic macrocycle in combination with topotecan according to the example treatment schedule shown, below, with the number of administered treatment cycles varying:

| | | | | | Days without |
|---|---|---|---|---|---|
| | | | Macrocycle | Topotecan | Administration |
| Treatment | Cycle | Cycle | Administration | Administration | of Macrocycle |
| Cycle Number | Start* | End | Days | Days | or Topotecan |
| 1 | Day 1 | Day 22 | Days 1-5 | Days 2-6 | Days 7-22 |
| 2 | Day 23 | Day 44 | Days 23-27 | Days 24-28 | Days 29-44 |
| 3 | Day 45 | Day 66 | Days 45-49 | Days 46-50 | Days 51-66 |
| 4 | Day 67 | Day 88 | Days 67-71 | Days 68-72 | Days 73-88 |
| 5 | Day 89 | Day 110 | Days 89-93 | Days 90-94 | Days 95-110 |
| 6 | Day 111 | Day 132 | Days 111-115 | Days 112-116 | Days 117-132 |
| 7 | Day 133 | Day 154 | Days 133-137 | Days 134-138 | Days 139-154 |
| 8 | Day 155 | Day 176 | Days 155-159 | Days 156-160 | Days 161-176 |
| 9 | Day 177 | Day 198 | Days 177-181 | Days 178-182 | Days 183-198 |
| 10 | Day 199 | Day 220 | Days 199-203 | Days 200-204 | Days 205-220 |
| 11 | Day 221 | Day 242 | Days 221-225 | Days 222-226 | Days 227-242 |
| 12 | Day 243 | Day 264 | Days 243-247 | Days 244-248 | Days 249-264 |
| 13 | Day 265 | Day 286 | Days 265-269 | Days 266-270 | Days 271-286 |
| 14 | Day 287 | Day 308 | Days 287-291 | Days 288-292 | Days 293-308 |
| 15 | Day 309 | Day 330 | Days 309-313 | Days 310-314 | Days 315-330 |

*Day 1 is denoted as the day in which the peptidomimetic macrocycle is first administered to a subject. Each subsequent day in time is then numbered consecutively.

In some embodiments, a method of the disclosure comprises administering a peptidomimetic macrocycle in combination with docetaxel according to the example treatment schedule shown, below, with the number of administered treatment cycles varying:

| | | | | | Days without |
|---|---|---|---|---|---|
| | | | Macrocycle | Docetaxel | Administration |
| Treatment | Cycle | Cycle | Administration | Administration | of Macrocycle |
| Cycle Number | Start* | End | Days | Days | or Docetaxel |
| 1 | Day 1 | Day 22 | Days 1-3 | Day 2 | Days 4-22 |
| 2 | Day 23 | Day 44 | Days 23-25 | Day 24 | Days 26-44 |
| 3 | Day 45 | Day 66 | Days 45-47 | Day 46 | Days 48-66 |
| 4 | Day 67 | Day 88 | Days 67-69 | Day 68 | Days 70-88 |
| 5 | Day 89 | Day 110 | Days 89-91 | Day 90 | Days 92-110 |
| 6 | Day 111 | Day 132 | Days 111-113 | Day 112 | Days 114-132 |
| 7 | Day 133 | Day 154 | Days 133-135 | Day 134 | Days 136-154 |
| 8 | Day 155 | Day 176 | Days 155-157 | Day 156 | Days 158-176 |
| 9 | Day 177 | Day 198 | Days 177-179 | Day 178 | Days 180-198 |
| 10 | Day 199 | Day 220 | Days 199-201 | Day 200 | Days 202-220 |
| 11 | Day 221 | Day 242 | Days 221-223 | Day 222 | Days 224-242 |
| 12 | Day 243 | Day 264 | Days 243-245 | Day 244 | Days 246-264 |
| 13 | Day 265 | Day 286 | Days 265-267 | Day 266 | Days 268-286 |
| 14 | Day 287 | Day 308 | Days 287-289 | Day 288 | Days 290-308 |
| 15 | Day 309 | Day 330 | Days 309-311 | Day 310 | Days 312-330 |

*Day 1 is denoted as the day in which the peptidomimetic macrocycle is first administered to a subject. Each subsequent day in time is then numbered consecutively.

In some embodiments, the peptidomimetic macrocycle and/or additional pharmaceutically-active agent is administered gradually over a period of time. A desired amount of peptidomimetic macrocycle or additional pharmaceutically-active agent can be administered gradually over a period of from about 0.1 h-24 h. In some cases a desired amount of peptidomimetic macrocycle or additional pharmaceutically-active agent is administered gradually over a period of 0.1 h, 0.5 h, 1 h, 1.5 h, 2 h, 2.5 h, 3 h, 3.5 h, 4 h, 4.5 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h. In some examples, a desired amount of peptidomimetic macrocycle or additional pharmaceutically-active agent is administered gradually over a period of 0.25-12 h, for example over a period of 0.25-1 h, 0.25-2 h, 0.25-3 h, 0.25-4 h, 0.25-6 h, 0.25-8 h, 0.25-10 h. In some examples, a desired amount of peptidomimetic macrocycle or additional pharmaceutically-active agent is administered gradually over a period of 0.25-2 h. In some examples, a desired amount of a peptidomimetic macrocycle or additional pharmaceutically-active agent is administered gradually over a period of 0.25-1 h. In some examples, a desired amount of a peptidomimetic macrocycle or additional pharmaceutically-active agent is administered gradually over a period of 0.25 h, 0.3 h, 0.4 h, 0.5 h, 0.6 h, 0.7 h, 0.8 h, 0.9 h, 1.0 h, 1.1 h, 1.2 h, 1.3 h, 1.4 h, 1.5 h, 1.6 h, 1.7 h, 1.8 h, 1.9 h, or 2.0 h. In some examples, a desired amount of a peptidomimetic macrocycle or additional pharmaceutically-active agent is administered gradually over a period of 1 h. In some examples, a desired amount of a peptidomimetic macrocycle or additional pharmaceutically-active agent is administered gradually over a period of 2 h.

Administration of the peptidomimetic macrocycles and/or additional pharmaceutically-active agent can continue as long as necessary to treat a cancer in a subject in need thereof. In some embodiments, one or more peptidomimetic macrocycles of the disclosure or an additional pharmaceutically-active agent is administered for more than 1 day, 1 week, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months. In some embodiments, one or more peptidomimetic macrocycle of the disclosure is administered for less than 1 week, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months.

In some embodiments, one or more peptidomimetic macrocycles of the disclosure and/or an additional pharmaceutically-active agent, for example any additional therapeutic agent disclosed herein, is administered chronically on an ongoing basis. In some embodiments, administration of one or more peptidomimetic macrocycle of the disclosure is continued until documentation of disease progression, unacceptable toxicity, or patient or physician decision to discontinue administration.

### Effects of Combination Treatment

In some embodiments, the administration of the peptidomimetic macrocycles and one or more additional therapies in accordance with the methods presented herein have an additive effect relative the administration of the peptidomimetic macrocycles or said one or more additional therapies alone. In some embodiments, the administration of the peptidomimetic macrocycles and one or more additional therapies in accordance with the methods presented herein have a synergistic effect relative to the administration of the peptidomimetic macrocycles or said one or more additional therapies alone.

In some embodiments, administration of the peptidomimetic macrocycles in combination with one or more additional therapies (e.g., pharmaceutically-active agents) has a synergistic effect. In some embodiments, a synergistic effect of two or more agents (e.g. a peptidomimetic macrocycle and any additional pharmaceutically-active agent disclosed herein) is an effect of the combination of the two or more agents, which effect is greater than the additive effects of the two or more agents. In some embodiments; a synergistic effect of a combination therapy permits the use of lower dosages (e.g., sub-optimal doses) of the peptidomimetic macrocycles or an additional therapy and/or less frequent administration of the peptidomimetic macrocycles or an additional therapy to a subject. In some embodiments, the ability to utilize lower dosages of the peptidomimetic macrocycles or of an additional therapy and/or to administer the peptidomimetic macrocycles or said additional therapy less frequently reduces the toxicity associated with the administration of the peptidomimetic macrocycles or of said additional therapy to a subject without reducing the efficacy of the peptidomimetic macrocycles or of said additional therapy in the treatment of cancer. In some embodiments, a synergistic effect results in improved efficacy of the peptidomimetic macrocycles and each of said additional therapies in treating cancer.

In some embodiments, a synergistic effect of a combination of the peptidomimetic macrocycles and one or more additional therapies (e.g., pharmaceutically-active agents) avoids or reduces adverse or unwanted side effects associated with the use of any single therapy. Non-limiting examples of side effects that can be reduced by a synergistic effect of a combination of a peptidomimetic macrocycle of the disclosure and an additional therapy are mucositis, side effects associated with myelosuppression such as neutropenia and thrombocytopenia; neurotoxicity, diarrhea, hair loss, vomiting, and nausea. In some embodiments, a synergistic effect of a combination of a peptidomimetic macrocycle of the disclosure and one or more additional therapies is a myelopreservative effect. In some embodiments, a reduction in mucositis, neutropenia, thrombocytopenia, or myelosuppression is due to peptidomimetic macrocycle-induced cell cycle arrest in tissues such as, for example, bone marrow and/or digestive tract tissue. In some embodiments, the reduction in side effects caused by a combination of a peptidomimetic macrocycle of the disclosure and an additional therapy allows for an increase in the maximum tolerated dose of the peptidomimetic macrocycle or additional therapy compared to the maximum tolerated dose of the peptidomimetic macrocycle or additional therapy when either the peptidomimetic macrocycle or additional therapy is administered alone.

In some embodiments, administration of a peptidomimetic macrocycle in combination with an additional pharmaceutically-active agent, for example any additional therapeutic agent disclosed herein, does not reduce an effect of the additional pharmaceutically-active agent. For example, expected survival and/or tumor growth inhibition in a subject can be the same following administration of the additional pharmaceutically-active agent alone or following administration of the additional pharmaceutically active agent in combination with a peptidomimetic macrocycle disclosed herein. In some embodiments, administration of a peptidomimetic macrocycle in combination with an additional pharmaceutically-active agent can reduce an effect (e.g. expected survival of a subject or tumor growth inhibition) of the additional pharmaceutically-active agent by less than about 1%, less than about 2%, less than about 3%, less than about 4%, less than about 5%, less than about 6%, less than about 7%, less than about 8%, less than about 9%, or less than about 10% compared to the effect caused by administration of the additional pharmaceutically-active agent alone.

### Additional Pharmaceutically-Active Agents

Additional therapies disclosed herein can include, for example, pharmaceutically-active agents. Non-limiting specific examples of pharmaceutically-active agents that can be used in combination with the peptidomimetic macrocycles include: hormonal agents (e.g., aromatase inhibitor, selective estrogen receptor modulator (SERM), and estrogen receptor antagonist), chemotherapeutic agents (e.g., microtubule disassembly blockers, antimetabolites, topoisomerase inhibitors, and DNA crosslinkers or damaging agents), and anti-antigenic agents (e.g., VEGF antagonists, receptor antagonists, integrin antagonists, vascular targeting agents (VTA)/vascular disrupting agents (VDA)). In some embodiments, an additional therapy disclosed herein is radiation therapy or conventional surgery.

Non-limiting examples of hormonal agents that can be used in combination with the peptidomimetic macrocycles include aromatase inhibitors, SERMs, and estrogen receptor antagonists. Hormonal agents that are aromatase inhibitors can be steroidal or no steroidal. Non-limiting examples of no steroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that can be used in combination with of peptidomimetic macrocycles include microtubule disassembly blockers, antimetabolites, topoisomerase inhibitors, and DNA crosslinkers or damaging agents. Chemotherapeutic agents that are microtubule disassembly blockers include, but are not limited to, taxanes (e.g., paclitaxel (branded/marketed as TAXOL®), docetaxel, eribulin, Abraxane®, larotaxel, ortataxel, and tesetaxel); epothilones (e.g., ixabepilone); and vinca alkaloids (e.g., vinorelbine, vinblastine, vindesine, and vincristine (branded/marketed as ONCOVIN®)).

Chemotherapeutic agents that are antimetabolites include, but are not limited to, folate antimetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine (GEMZAR®), cytarabine, decitabine, floxuridine, tegafur, capecitabine); and deoxyribonucleotide antimetabolites (e.g., hydroxyurea).

Chemotherapeutic agents that are topoisomerase inhibitors include, but are not limited to, class I topoisomerase inhibitors such as topotecan (branded/marketed as HYCAMTIN®) irinotecan, rubitecan, and belotecan; class II topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone, and pixantrone).

Chemotherapeutic agents that are DNA crosslinkers (or DNA damaging agents) include, but are not limited to, alkylating agents (e.g., cyclophosphamide, mechlorethamine, ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethyl enethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

Non-limiting examples of other therapies that can be administered to a subject in combination with the peptidomimetic macrocycles include: (1) a statin such as lovastatin (e.g., branded/marketed as MEVACOR®); (2) an mTOR inhibitor such as sirolimus which is also known as Rapamycin (e.g., branded/marketed as RAPAMUNE®), temsirolimus (e.g., branded/marketed as TORISEL®), evorolimus (e.g., branded/marketed as AFINITOR®), and deforolimus; (3) a farnesyltransferase inhibitor agent such as tipifarnib; (4) an antifibrotic agent such as pirfenidone; (5) a pegylated interferon such as PEG-interferon alfa-2b; (6) a CNS stimulant such as methylphenidate (branded/marketed as RITALIN®); (7) a HER-2 antagonist such as anti-HER-2 antibody (e.g., trastuzumab) and kinase inhibitor (e.g., lapatinib); (8) an IGF-1 antagonist such as an anti-IGF-1 antibody (e.g., AVE1642 and IMC-A11) or an IGF-1 kinase inhibitor; (9) EGFR/HER-1 antagonist such as an anti-EGFR antibody (e.g., cetuximab, panitumamab) or EGFR kinase inhibitor (e.g., erlotinib; gefitinib); (10) SRC antagonist such as bosutinib; (11) cyclin dependent kinase (CDK) inhibitor such as seliciclib; (12) Janus kinase 2 inhibitor such as lestaurtinib; (13) proteasome inhibitor such as bortezomib; (14) phosphodiesterase inhibitor such as anagrelide; (15) inosine monophosphate dehydrogenase inhibitor such as tiazofurine; (16) lipoxygenase inhibitor such as masoprocol; (17) endothelin antagonist; (18) retinoid receptor antagonist such as tretinoin or alitretinoin; (19) immune modulator such as lenalidomide, pomalidomide, or thalidomide; (20) kinase (e.g., tyrosine kinase) inhibitor such as imatinib, dasatinib, erlotinib, nilotinib, gefitinib, sorafenib, sunitinib, lapatinib, or TG100801; (21) non-steroidal anti-inflammatory agent such as celecoxib (branded/marketed as CELEBREX®); (22) human granulocyte colony-stimulating factor (G-CSF) such as filgrastim (branded/marketed as NEUPOGEN®); (23) folinic acid or leucovorin calcium; (24) integrin antagonist such as an integrin a5p31-antagonist (e.g., JSM6427); (25) nuclear factor kappa beta (NF-κβ) antagonist such as OT-551, which is also an anti-oxidant. (26) hedgehog inhibitor such as CUR61414, cyclopamine, GDC-0449, and anti-hedgehog antibody; (27) histone deacetylase (HDAC) inhibitor such as SAHA (also known as vorinostat (branded/marketed as ZOLINZA)), PCI-24781, SB939, CHR-3996, CRA-024781, ITF2357, JNJ-26481585, or PCI-24781; (28) retinoid such as isotretinoin (e.g., branded/marketed as ACCUTANE®); (29) hepatocyte growth factor/scatter factor (HGF/SF) antagonist such as HGF/SF monoclonal antibody (e.g., AMG 102); (30) synthetic chemical such as antineoplaston; (31) anti-diabetic such as rosaiglitazone (e.g., branded/marketed as AVANDIA®); (32) antimalarial and amebicidal drug such as chloroquine (e.g., branded/marketed as ARALEN®); (33) synthetic bradykinin such as RMP-7; (34) platelet-derived growth factor receptor inhibitor such as SU-101; (35) receptor tyrosine kinase inhibitors of Flk-1/KDR/VEGFR2, FGFR1 and PDGFR beta such as SU5416 and SU6668; (36) anti-inflammatory agent such as sulfasalazine (e.g., branded/marketed as AZULFIDINE®); and (37) TGF-beta antisense therapy.

In some embodiments, an antineoplastic agent can be administered to a subject in combination with a peptidomimetic macrocycle. Non-limiting examples of antineoplastic agents can include topoisomerase inhibitors such as, for example, topotecan.

In some embodiments, a peptidomimetic macrocycles disclosed herein can inhibit one or more transporter enzymes (e.g., OATP1B1, OATP1B3, BSEP) at concentrations that can be clinically relevant. Therefore, such a peptidomimetic macrocycles disclosed herein can interact with medications that are predominantly cleared by hepatobiliary transporters. In some embodiments, methotrexate and statins (e.g., atorvastatin, fluvastatin lovastatin, pitavastatin pravastatin, rosuvastatin and simvastatin) are not dosed within 48 hrs, 36 hrs, 24 hrs, or 12 hrs ((for example within 24 hrs) of the administration of such a peptidomimetic macrocycle. Non-limiting examples of medications that can be affected by co-administration of such a peptidomimetic macrocycle disclosed herein are listed in the following table:

| Medication | Therapeutic Area |
|---|---|
| Irinotecan | Oncology |
| Bosentan | Pulmonary artery hypertension |
| Caspofungin | Antifungal |
| Methotrexate | Oncology & rheumatology |
| Repaglinide | Diabetes mellitus |
| Atorvastatin | Hypercholesterolemia |
| Cerivastatin | Hypercholesterolemia |
| Fluvastatin | Hypercholesterolemia |
| Lovastatin | Hypercholesterolemia |
| Pitavastatin | Hypercholesterolemia |
| Pravastatin | Hypercholesterolemia |
| Rosuvastatin | Hypercholesterolemia |
| Simvastatin | Hypercholesterolemia |

In some embodiments, one or more of the medications selected from the table above is not dosed within 48 hrs, 36 hrs, 24 hrs, or 12 hrs (for example within 24 h) of the administration of such a peptidomimetic macrocycle.

### Subject Response to Treatment

The effectiveness and/or response of cancer treatment by the methods disclosed herein can be determined by any suitable method. The response can be a complete response, and which can be an objective response, a clinical response, or a pathological response to treatment. The response can be a duration of survival (or probability of such duration) or progression-free interval. The timing or duration of such events can be determined from about the time of diagnosis, or from about the time treatment is initiated or from about the time treatment is finished (like the final administration of the peptidomimetic macrocycle and/or additional pharmaceutically-active agent). Alternatively, the response can be based upon a reduction in tumor size, tumor volume, or tumor metabolism, or based upon overall tumor burden, or based upon levels of serum markers especially where elevated in the disease state.

The response in individual patients can be characterized as a complete response, a partial response, stable disease, and progressive disease. In some embodiments, the response is complete response (CR). Complete response, in some examples can be defined as disappearance of all target lesions i.e. any pathological lymph nodes (whether target or non-target) must have reduction in short axis to <10 mm. In certain embodiments, the response is a partial response (PR). Partial response can be defined to mean at least 30% decrease in the sum of diameters of target lesions, taking as reference the baseline sum diameters. In some embodiments, the response is progressive disease (PD). Progressive disease can be defined as at least a 20% increase in the sum of diameters of target lesions, taking as reference the smallest sum on study (this includes the baseline sum if that is the smallest) and an absolute increase of at least 5 mm in the sum of diameters of target lesions. The appearance of one or more new lesions can also be considered as progression. In some embodiments, the disease can be stable disease (SD). Stable disease can be characterized by neither sufficient shrinkage to qualify for PR nor sufficient increase to qualify for PD, taking as reference the smallest sum diameters while on study. In certain embodiments, the response is a pathological complete response. A pathological complete response, e.g., as determined by a pathologist following examination of tissue removed at the time of surgery or biopsy, generally refers to an absence of histological evidence of invasive tumor cells in the surgical specimen.

In some embodiments, the effectiveness of methods disclosed herein are assessed by a level of adverse effect reduction seen in a subject or population of subjects. For example, the frequency and/or severity of neutropenia, thrombocytopenia, and/or other hematologic toxicity parameters can be assessed in subjects treated with a combination of a peptidomimetic macrocycle disclosed herein and an additional pharmaceutically-active agent (e.g., any additional therapeutic agent disclosed herein) and compared to the frequency and/or severity of the neutropenia, thrombocytopenia, and/or other hematologic toxicity parameters in subjects treated with the additional pharmaceutically-active agent alone. In some instances, the severity of adverse events is assessed using the Common Terminology Criteria for Adverse Events (CTCAE) grading scale.

### Assays

The properties of peptidomimetic macrocycles are assayed, for example, by using the methods described below. In some embodiments, a peptidomimetic macrocycle has improved biological properties relative to a corresponding polypeptide lacking the substituents described herein.

a. Assays to Determine α-Helicity

In solution, the secondary structure of polypeptides with α-helical domains will reach a dynamic equilibrium between random coil structures and α-helical structures, often expressed as a “percent helicity”. Thus, for example, alpha-helical domains are predominantly random coils in solution, with α-helical content usually under 25%. Peptidomimetic macrocycles with optimized linkers, on the other hand, possess, for example, an alpha-helicity that is at least two-fold greater than that of a corresponding uncrosslinked polypeptide. In some embodiments, macrocycles will possess an alpha-helicity of greater than 50%. To assay the helicity of peptidomimetic macrocycles, the compounds are dissolved in an aqueous solution (e.g. 50 mM potassium phosphate solution at pH 7, or distilled H₂O, to concentrations of 25-50 μM). Circular dichroism (CD) spectra are obtained on a spectropolarimeter using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g. [ΦD]222obs) by the reported value for a model helical decapeptide.

b. Assay to Determine Melting Temperature (Tm)

A peptidomimetic macrocycle comprising a secondary structure such as an α-helix exhibits, for example, a higher melting temperature than a corresponding uncrosslinked polypeptide. Peptidomimetic macrocycles exhibit Tm of >60° C. representing a highly stable structure in aqueous solutions. To assay the effect of macrocycle formation on melting temperature, peptidomimetic macrocycles or unmodified peptides are dissolved in distilled H₂O (e.g. at a final concentration of 50 μM) and the Tm is determined by measuring the change in ellipticity over a temperature range (e.g. 4 to 95° C.) on a spectropolarimeter using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

c. Protease Resistance Assay

The amide bond of the peptide backbone is susceptible to hydrolysis by proteases, thereby rendering peptidic compounds vulnerable to rapid degradation in vivo. Peptide helix formation, however, buries the amide backbone and therefore can shield the amide from proteolytic cleavage. The peptidomimetic macrocycles can be subjected to in vitro trypsin proteolysis to assess for any change in degradation rate compared to a corresponding uncrosslinked polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time (k=−1×slope).

d. Ex Vivo Stability Assay

Peptidomimetic macrocycles with optimized linkers possess, for example, an ex vivo half-life that is at least two-fold greater than that of a corresponding uncrosslinked polypeptide, and possess an ex vivo half-life of 12 hours or more. For ex vivo serum stability studies, a variety of assays can be used. For example, a peptidomimetic macrocycle and a corresponding uncrosslinked polypeptide (2 mcg) are incubated with fresh mouse, rat and/or human serum (2 mL) at 37° C. for 0, 1, 2, 4, 8, and 24 hours. To determine the level of intact compound, the following procedure can be used: The samples are extracted by transferring 100 L of sera to 2 mL centrifuge tubes followed by the addition of 10 μL of 50% formic acid and 500 μL acetonitrile and centrifugation at 14,000 RPM for 10 min at 4+2° C. The supernatants are then transferred to fresh 2 mL tubes and evaporated on Turbovap under N₂<10 psi, 37° C. The samples are reconstituted in 100 μL of 50:50 acetonitrile:water and submitted to LC-MS/MS analysis.

e. In Vitro Binding Assays

To assess the binding and affinity of peptidomimetic macrocycles and peptidomimetic precursors to acceptor proteins, a fluorescence polarization assay (FPA) is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to slower rates of rotation as compared to fluorescent tracers attached to smaller molecules (e.g. FITC-labeled peptides that are free in solution). For example, fluoresceinated peptidomimetic macrocycles (25 nM) are incubated with the acceptor protein (25-1000 nM) in binding buffer (140 mM NaCl, 50 mM Tris-HCL, pH 7.4) for 30 minutes at room temperature. Binding activity is measured, for example, by fluorescence polarization on a luminescence spectrophotometer. K_{d} values can be determined by nonlinear regression analysis using, for example, GraphPad Prism software. A peptidomimetic macrocycle shows, in some embodiments, similar or lower K_{d} than a corresponding uncrosslinked polypeptide.

f. In Vitro Displacement Assays to Characterize Antagonists of Peptide-Protein Interactions

To assess the binding and affinity of compounds that antagonize the interaction between a peptide and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to slower rates of rotation as compared to fluorescent tracers attached to smaller molecules (e.g. FITC-labeled peptides that are free in solution). A compound that antagonizes the interaction between the fluoresceinated peptidomimetic macrocycle and an acceptor protein is detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCL, pH 7.4) for 30 minutes at room temperature. Antagonist binding activity is measured, for example, by fluorescence polarization on a luminescence spectrophotometer. K_{d} values can be determined by nonlinear regression analysis. Any class of molecule, such as small organic molecules, peptides, oligonucleotides or proteins can be examined as putative antagonists in this assay.

g. Assay for Protein-Ligand Binding by Affinity Selection-Mass Spectrometry

To assess the binding and affinity of test compounds for proteins, an affinity-selection mass spectrometry assay is used, for example. Protein-ligand binding experiments are conducted according to the following representative procedure outlined for a system-wide control experiment using 1 μM peptidomimetic macrocycle plus 5 μM hMDM2. A 1 μL DMSO aliquot of a 40 μM stock solution of peptidomimetic macrocycle is dissolved in 19 μL of PBS (50 mM, pH 7.5 Phosphate buffer containing 150 mM NaCl). The resulting solution is mixed by repeated pipetting and clarified by centrifugation at 10,000 g for 10 min. To a 4 μL aliquot of the resulting supernatant is added 4 μL of 10 μM hMDM2 in PBS. Each 8.0 μL experimental sample thus contains 40 pmol (1.5 μg) of protein at 5.0 μM concentration in PBS plus 1 μM peptidomimetic macrocycle and 2.5% DMSO. Duplicate samples thus prepared for each concentration point are incubated for 60 min at room temperature, and then chilled to 4° C. prior to size-exclusion chromatography-LC-MS analysis of 5.0 μL injections. Samples containing a target protein, protein-ligand complexes, and unbound compounds are injected onto an SEC column, where the complexes are separated from non-binding component by a rapid SEC step. The SEC column eluate is monitored using UV detectors to confirm that the early-eluting protein fraction, which elutes in the void volume of the SEC column, is well resolved from unbound components that are retained on the column. After the fraction containing the protein and protein-ligand complexes elute from the primary UV detector, the fraction enters a sample loop where the fraction is excised from the flow stream of the SEC stage and transferred directly to the LC-MS via a valving mechanism. The (M+3H)³⁺ ion of the peptidomimetic macrocycle is observed by ESI-MS at the expected m/z, confirming the detection of the protein-ligand complex.

h. Assay for Protein-Ligand K_{d} Titration Experiments

To assess the binding and affinity of test compounds for proteins, a protein-ligand K_{d} titration experiment is performed, for example. Protein-ligand K_{d} titrations experiments are conducted as follows: 2 μL DMSO aliquots of a serially diluted stock solution of titrant peptidomimetic macrocycle (5, 2.5, . . . , 0.098 mM) are prepared then dissolved in 38 μL of PBS. The resulting solutions are mixed by repeated pipetting and clarified by centrifugation at 10 000 g for 10 min. To 4.0 μL aliquots of the resulting supernatants is added 4.0 μL of 10 μM hMDM2 in PBS. Each 8.0 μL experimental sample thus contains 40 pmol (1.5 μg) of protein at 5.0 μM concentration in PBS, varying concentrations (125, 62.5, . . . , 0.24 μM) of the titrant peptide, and 2.5% DMSO. Duplicate samples thus prepared for each concentration point are incubated at room temperature for 30 min, then chilled to 4° C. prior to SEC-LC-MS analysis of 2.0 μL injections. The (M+H)¹⁺, (M+2H)²⁺, (M+3H)³⁺, and/or (M+Na)¹⁺ ion is observed by ESI-MS; extracted ion chromatograms are quantified, then fit to equations to derive the binding affinity K_{d}.

i. Assay for Competitive Binding Experiments by Affinity Selection-Mass Spectrometry

To determine the ability of test compounds to bind competitively to proteins, an affinity selection mass spectrometry assay is performed, for example. A mixture of ligands at 40 μM per component is prepared by combining 2 μL aliquots of 400 μM stocks of each of the three compounds with 14 μL of DMSO. Then, 1 μL aliquots of this 40 μM per component mixture are combined with 1 μL DMSO aliquots of a serially diluted stock solution of titrant peptidomimetic macrocycle (10, 5, 2.5, . . . , 0.078 mM). These 2 μL samples are dissolved in 38 μL of PBS. The resulting solutions are mixed by repeated pipetting and clarified by centrifugation at 10,000 g for 10 min. To 4.0 μL aliquots of the resulting supernatants is added 4.0 μL of 10 μM hMDM2 protein in PBS. Each 8.0 μL experimental sample thus contains 40 pmol (1.5 μg) of protein at 5.0 μM concentration in PBS plus 0.5 μM ligand, 2.5% DMSO, and varying concentrations (125, 62.5, . . . , 0.98 μM) of the titrant peptidomimetic macrocycle. Duplicate samples thus prepared for each concentration point are incubated at room temperature for 60 min, then chilled to 4° C. prior to SEC-LC-MS analysis of 2.0 μL injections.

j. Binding Assays in Intact Cells

Binding of peptides or peptidomimetic macrocycles to their natural acceptors in intact cells by can be measured immunoprecipitation experiments. For example, intact cells are incubated with fluoresceinated (FITC-labeled) compounds for 4 hrs in the absence of serum, followed by serum replacement and further incubation that ranges from 4-18 hrs. Cells are then pelleted and incubated in lysis buffer (50 mM Tris [pH 7.6], 150 mM NaCl, 1% CHAPS and protease inhibitor cocktail) for 10 minutes at 4° C. Extracts are spun by centrifuge at 14,000 rpm for 15 minutes, and supernatants collected and incubated with 10 μL goat anti-FITC antibody for 2 hrs, rotating at 4° C. followed by further 2 hrs incubation at 4° C. with protein A/G Sepharose (50 L of 50% bead slurry). After quick centrifugation, the pellets are washed in lysis buffer containing increasing salt concentration (e.g., 150, 300, 500 mM). The beads are then re-equilibrated at 150 mM NaCl before addition of SDS-containing sample buffer and boiling. After centrifugation, the supernatants are optionally electrophoresed using 4%-12% gradient Bis-Tris gels followed by transfer into Immobilon-P membranes. After blocking, blots are optionally incubated with an antibody that detects FITC and also with one or more antibodies that detect proteins that bind to the peptidomimetic macrocycle.

k. Cellular Penetrability Assays

A peptidomimetic macrocycle can be, for example, more cell penetrable compared to a corresponding uncrosslinked macrocycle. Peptidomimetic macrocycles with optimized linkers can possess, for example, cell penetrability that is at least two-fold greater than that of a corresponding uncrosslinked macrocycle. Often 20% or more of the applied peptidomimetic macrocycle can be observed to have penetrated the cell after 4 hours. To measure the cell penetrability of peptidomimetic macrocycles and corresponding uncrosslinked macrocycle, intact cells are incubated with fluorescently-labeled (e.g. fluoresceinated) peptidomimetic macrocycles or corresponding uncrosslinked macrocycle (10 μM) for 4 hrs in serum free media at 37° C., washed twice with media and incubated with trypsin (0.25%) for 10 min at 37° C. The cells are washed again and resuspended in PBS. Cellular fluorescence is analyzed.

l. Cellular Efficacy Assays

The efficacy of certain peptidomimetic macrocycles is determined, for example, in cell-based killing assays using a variety of tumorigenic and non-tumorigenic cell lines and primary cells derived from human or mouse cell populations. Cell viability is monitored, for example, over 24-96 hrs of incubation with peptidomimetic macrocycles (0.5 to 50 μM) to identify those that kill at EC₅₀<10 μM. Several standard assays that measure cell viability are commercially available and are optionally used to assess the efficacy of the peptidomimetic macrocycles. Assays that measure Annexin V and caspase activation are optionally used to assess whether the peptidomimetic macrocycles kill cells by activating the apoptotic machinery. For example, the Cell Titer-glo assay is used which determines cell viability as a function of intracellular ATP concentration.

m. In Vivo Stability Assay

To investigate the in vivo stability of the peptidomimetic macrocycles, the compounds are, for example, administered to mice and/or rats by IV, IP, PO or inhalation routes at concentrations ranging from 0.1 to 50 mg/kg and blood specimens withdrawn at 0′, 5′, 15′, 30′, 1 hr, 4 hrs, 8 hrs and 24 hours post-injection. Levels of intact compound in 25 μL of fresh serum are then measured by LC-MS/MS as above.

n. In Vivo Efficacy in Animal Models

To determine the anti-oncogenic activity of peptidomimetic macrocycles in vivo, the compounds are, for example, given alone (IP, IV, PO, by inhalation or nasal routes) or in combination with sub-optimal doses of relevant chemotherapy (e.g., cyclophosphamide, doxorubicin, etoposide). In one example, 5×10⁶ RS4;11 cells (established from the bone marrow of a patient with acute lymphoblastic leukemia) that stably express luciferase are injected by tail vein in NOD-SCID mice 3 hrs after they have been subjected to total body irradiation. If left untreated, this form of leukemia is fatal in 3 weeks in this model. The leukemia is readily monitored, for example, by injecting the mice with D-luciferin (60 mg/kg) and imaging the anesthetized animals. Total body bioluminescence is quantified by integration of photonic flux (photons/sec) by Living Image Software. Peptidomimetic macrocycles alone or in combination with sub-optimal doses of relevant chemotherapeutics agents are, for example, administered to leukemic mice (10 days after injection/day 1 of experiment, in bioluminescence range of 14-16) by tail vein or IP routes at doses ranging from 0.1 mg/kg to 50 mg/kg for 7 to 21 days. Optionally, the mice are imaged throughout the experiment every other day and survival monitored daily for the duration of the experiment. Expired mice are optionally subjected to necropsy at the end of the experiment. Another animal model is implantation into NOD-SCID mice of DoHH2, a cell line derived from human follicular lymphoma that stably expresses luciferase. These in vivo tests optionally generate preliminary pharmacokinetic, pharmacodynamic and toxicology data.

o. In Vivo Gene Expression in Bone Marrow of Mice Treated with a Peptidomimetic Macrocycle

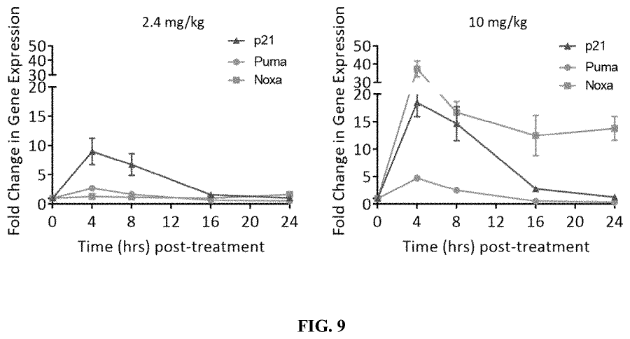

To assess the effects of a peptidomimetic macrocycle of the disclosure on gene expression in bone marrow, a controlled in vivo, preclinical study is performed. A therapeutically-effective amount (e.g. 2.4 mg/kg) of a peptidomimetic macrocycle of the disclosure is administered intravenously to a group of mice. mRNA is extracted from total bone marrow samples of mice at 0, 4, 8, 16, and 24 hours post macrocycle administration. Murine p21 (a downstream mediator of p53 dependent cell cycle arrest), Noxa (an apoptosis marker), and p53 upregulated modulator of apoptosis (PUMA) mRNA expression is assessed by real time polymerase chain reaction (PCR). In some embodiments, average p21 mRNA expression in the bone marrow of mice is increased about 7.5 fold to about 10 fold at about 4 hours after peptidomimetic macrocycle administration, and about five fold to about 7.5 fold at about 8 hours after peptidomimetic macrocycle administration, and returns to about baseline levels by about 16 hours post peptidomimetic macrocycle administration. In some embodiments, average PUMA mRNA expression is increased about 1 fold to about 3 fold at about 4 hours post peptidomimetic macrocycle administration and returns to about baseline levels at about 8 hours post peptidomimetic macrocycle administration. In some embodiments, average Noxa mRNA expression in bone marrow of the mice is unchanged following peptidomimetic macrocycle administration. In some embodiments, changes in average p21 mRNA expression, average PUMA mRNA expression, and/or average Noxa mRNA expression in bone marrow of the group of mice occur with at most a 10%, 20%, 30%, 40%, or 50% deviation from corresponding lines illustrated in FIG. 9.

p. Cell Cycle Arrest in Bone Marrow of a Preclinical Model

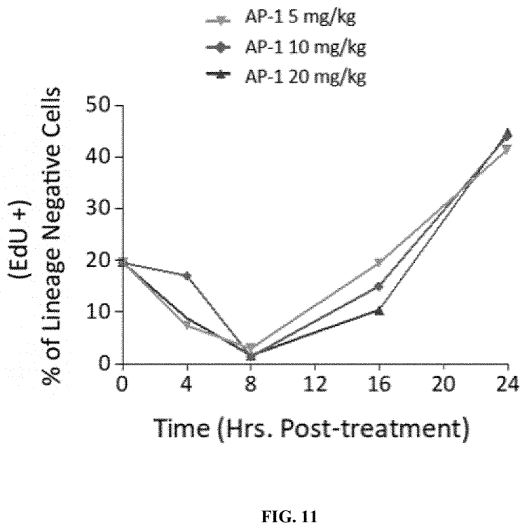

To assess the effects of a peptidomimetic macrocycle of the disclosure on cell cycle arrest in bone marrow a controlled, in vivo study is performed in mice. Mice are treated with 5 mg/kg, 10 mg/kg, or 20 mg/kg of a peptidomimetic macrocycle via intravenous administration. Cell cycle arrest in the bone marrow of mice is then measured by flow cytometry using 5-ethynyl-2′-deoxyuridine (EdU) incorporation in lineage negative, Kit positive, hematopoietic stem and progenitor cells at pre-treatment (0 hours post treatment), and 4 hours, 8 hours, 16 hours, and 24 hours post treatment. In some embodiments, the percentage of EdU+ cells is about 20% pre-treatment, less than about 5% at about 8 hours post treatment, between about 10% to about 25% at about 16 hours post treatment, and between about 40% to about 50% at about 24 hours post treatment. In some embodiments, a change in a percentage of lineage negative, Kit positive, hematopoietic stem and progenitor cells (HSPCs) that are EdU+ occurs with at most a 10%, 20%, 30%, 40%, or 50% deviation from corresponding lines illustrated in FIG. 11.

q. Effect of Peptidomimetic Macrocycle Administration on Topotecan-Induced Neutropenia

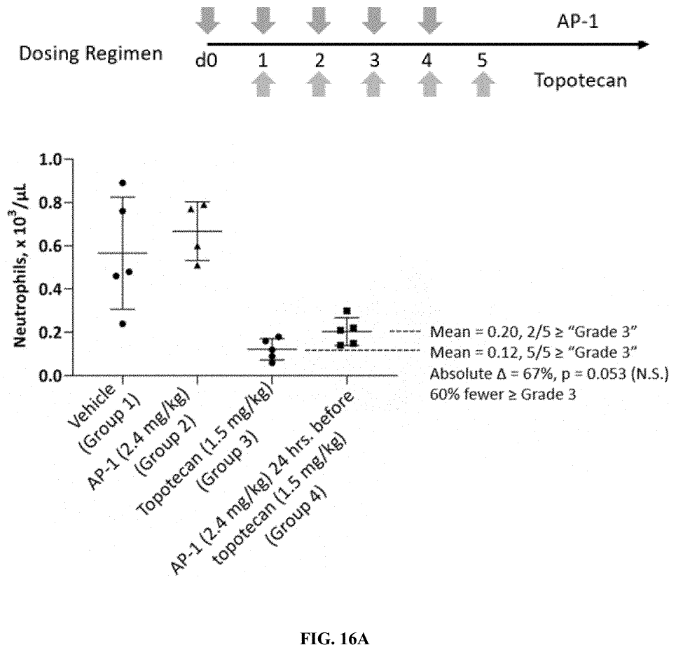

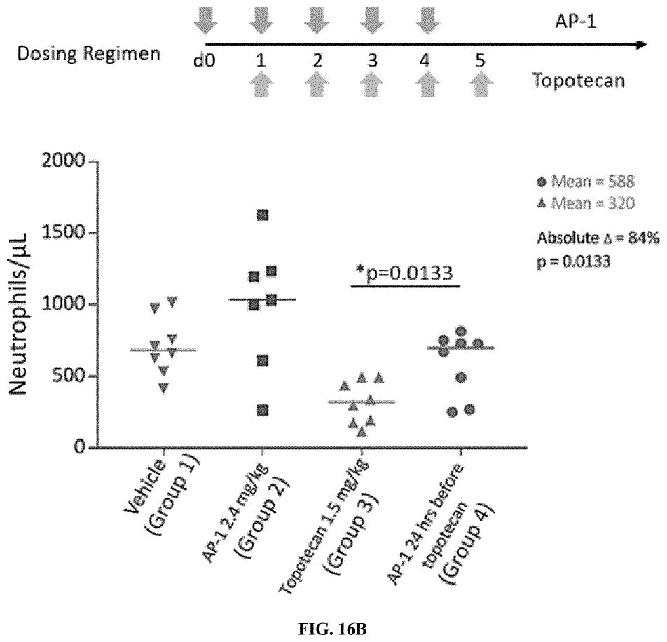

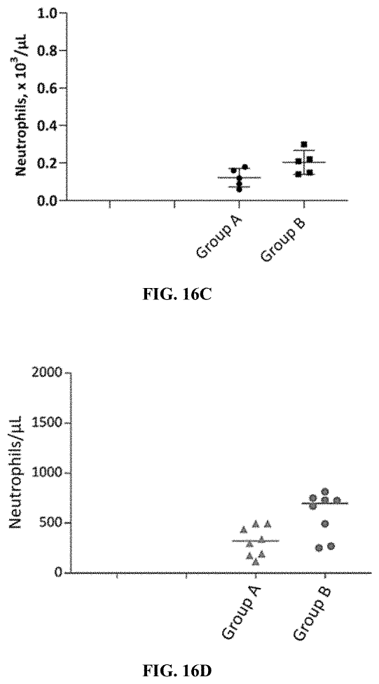

The effect of a peptidomimetic macrocycle of the disclosure on topotecan-induced neutropenia is assessed in a controlled, in vivo study. Four groups of mice are involved in the study, and administration of agents occurs over a 6-day treatment period. The first group of mice (Group 1) is treated with a vehicle control on days 2, 3, 4, 5, and 6 of the treatment period. The second group of mice (Group 2) is treated intravenously with 2.4 mg/kg of a peptidomimetic macrocycle on days 1, 2, 3, 4, and 5 of a 6-day treatment period. The third group of mice (Group 3) is treated with 1.5 mg/kg of topotecan on days 2, 3, 4, 5, and 6 of the 6-day treatment period. The fourth group of mice (Group 4) is treated with 2.4 mg/kg of the peptidomimetic macrocycle on days 1, 2, 3, 4, and 5 of the 6-day treatment period and 1.5 mg/kg of topotecan on days 2, 3, 4, 5, and 6 of the 6-day treatment period. Following the treatment period, complete blood counts are taken to determine the number of neutrophils present per μL of blood. In some embodiments, the mice of Group 4 have an average of about 588 neutrophils per μL of blood. In some embodiments, the number of neutrophils per μL of blood in mice of Group 4 ranges from about 200 to about 1000. In some embodiments, the mice of Group 3 have an average of about 320 neutrophils per μL of blood. In some embodiments, the number of neutrophils per μL of blood in mice of Group 3 ranges from about 10 to about 500. In some embodiments, the mice of Group 2 have a median number of neutrophils per μL of blood of about 1000. In some embodiments, the number of neutrophils per μL of blood in mice of Group 2 ranges from about 200 to about 1800. In some embodiments, the mice of Group 1 have a median number of neutrophils per μL of blood of about 600. In some embodiments, the number of neutrophils per μL of blood in mice of Group 1 ranges from about 450 to about 1000. In some embodiments an average number of neutrophils present per μL of blood in mice of Group 4 is increased by about 20%, 40%, 50%, 60%, 70%, 80%, 100%, 120%, or 140% compared to an average number of neutrophils present per μL of blood in mice of Group 3. In some embodiments, a number of neutrophils present per μL of blood in mice of Group 4 is increased compared to a number of neutrophils present per μL of blood in mice of Group 3 as illustrated in FIG. 16A or FIG. 16B.

r. Effect of Peptidomimetic Macrocycle Administration on Carboplatin and Paclitaxel-Induced Neutropenia

The effect of a peptidomimetic macrocycle of the disclosure on carboplatin and paclitaxel induced neutropenia is assessed in a controlled, in vivo study. Mice are divided into six treatment groups and administered vehicle control, a peptidomimetic macrocycle alone, a combination of carboplatin and paclitaxel, or a combination of a peptidomimetic macrocycle, carboplatin, and paclitaxel. The administration time(s) of the peptidomimetic macrocycle in relation to carboplatin and paclitaxel vary, with the time of carboplatin/paclitaxel administration being denoted as time 0 hours. Positive times (e.g. time +8 hours) indicate peptidomimetic macrocycle treatments that occur after treatment with carboplatin/paclitaxel and negative times (e.g., −1 hour) indicate peptidomimetic macrocycle treatment before carboplatin/paclitaxel administration. AP-1 and paclitaxel are administered intravenously, and carboplatin is administered via intraperitoneal injection.

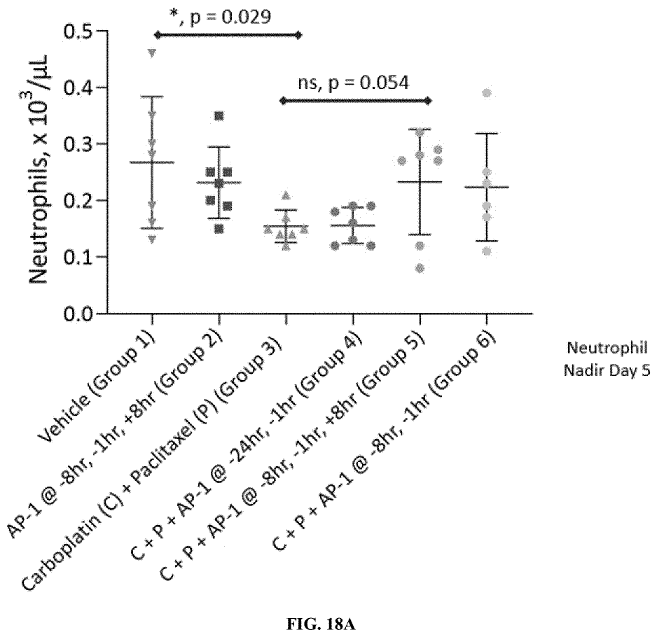

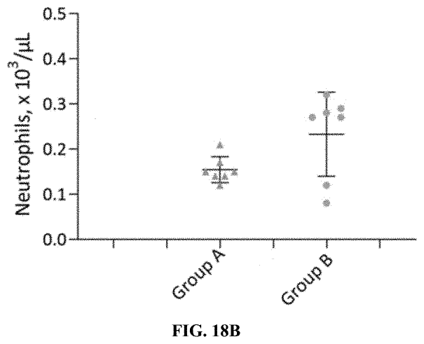

Group 1 is treated with a vehicle control. Group 2 is treated with a peptidomimetic macrocycle (2.4 mg/kg) at times −8 hours, −1 hour and +8 hours. Group 3 is treated with carboplatin (25 mg/kg) and paclitaxel (5 mg/kg) at time 0 hour (C+P). Group 4 is treated with a peptidomimetic macrocycle at times −24 hours and −1 hour, and carboplatin (25 mg/kg) and paclitaxel (5 mg/kg) at time 0 hour. Group 5 is treated with a peptidomimetic macrocycle at times −8 hours, −1 hours and +8 hours, and carboplatin (25 mg/kg) and paclitaxel (5 mg/kg) at time 0 hour. Group 6 is treated with a peptidomimetic macrocycle at times −8 hours and −1 hour, and carboplatin (25 mg/kg) and paclitaxel (5 mg/kg) at time 0 hour. Following treatment, blood is collected from mice and neutrophil levels in blood are determined. In some embodiments, the mice of Group 6 have an average of about 225 neutrophils per μL of blood. In some embodiments, the number of neutrophils per μL of blood in mice of Group 6 ranges from about 100 to about 400. In some embodiments, the mice of Group 5 have an average of about 250 neutrophils per μL of blood. In some embodiments, the number of neutrophils per μL of blood in mice of Group 5 ranges from about 50 to about 350. In some embodiments, the mice of Group 4 have an average of about 150 neutrophils per μL of blood. In some embodiments, the number of neutrophils per μL of blood in mice of Group 4 ranges from about 100 to about 200. In some embodiments, the mice of Group 3 have an average of about 150 neutrophils per μL of blood. In some embodiments, the number of neutrophils per μL of blood in mice of Group 3 ranges from about 100 to about 225. In some embodiments, the mice of Group 2 have an average of about 225 neutrophils per μL of blood. In some embodiments, the number of neutrophils per μL of blood in mice of Group 2 ranges from about 150 to about 375. In some embodiments, the mice of Group 1 have an average of about 275 neutrophils per μL of blood. In some embodiments, the number of neutrophils per μL of blood in mice of Group 1 ranges from about 175 to about 475. In some embodiments, an average number of neutrophils present per μL of blood in mice of Group 5 is increased by about 20%, 40%, 50%, 60%, 70%, 80%, 100%, 120%, or 140% compared to an average number of neutrophils present per mL of blood in mice of Group 3. In some embodiments, a number of neutrophils present per μL of blood in mice of Group 5 is increased compared to a number of neutrophils present per μL of blood in mice of Group 3 as illustrated in FIG. 18A.

s. Effect of Peptidomimetic Macrocycle Administration on Topotecan-Induced Mucositis

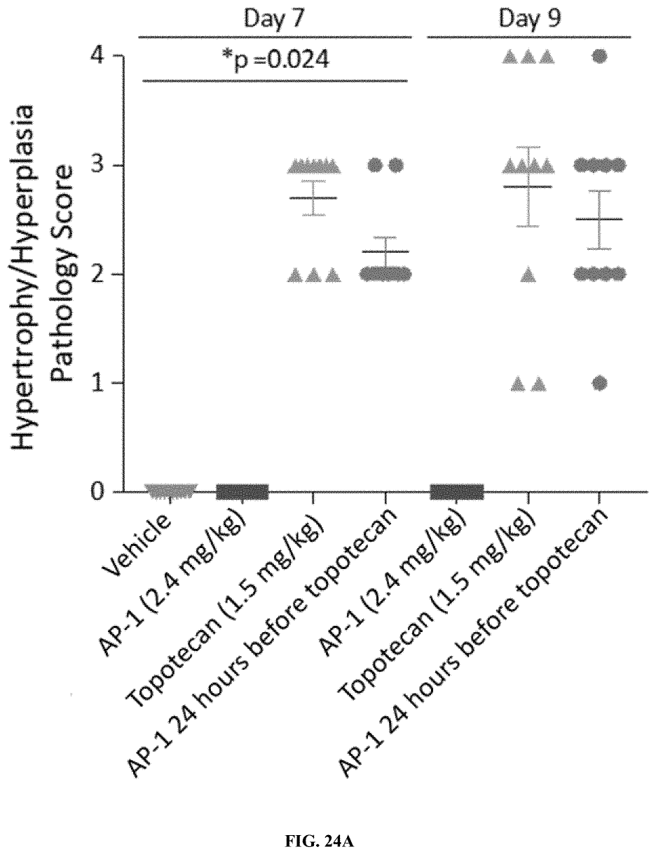

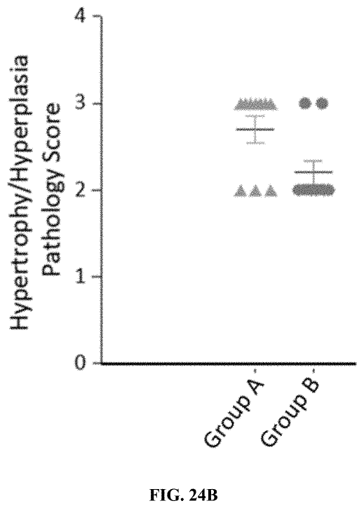

The effect of a peptidomimetic macrocycle of the disclosure on topotecan-induced mucositis is assessed in a controlled, in vivo study. Four groups of mice (10 mice per group) are involved in the study, and administration of agents occurs over a 6-day treatment period. The first group of mice (Group 1) is treated with a vehicle control on days 2, 3, 4, 5, and 6 of the treatment period. The second group of mice (Group 2) is treated intravenously with 2.4 mg/kg of a peptidomimetic macrocycle on days 1, 2, 3, 4, and 5 of a 6-day treatment period. The third group of mice (Group 3) is treated with 1.5 mg/kg of topotecan on days 2, 3, 4, 5, and 6 of the 6-day treatment period. The fourth group of mice (Group 4) is treated with 2.4 mg/kg of the peptidomimetic macrocycle on days 1, 2, 3, 4, and 5 of the 6-day treatment period and 1.5 mg/kg of topotecan on days 2, 3, 4, 5, and 6 of the 6-day treatment period. Gut samples are then taken from mice on days 7 and 9 post treatment. Histopathology analysis of gut samples is performed to assess hypertrophy/hyperplasia. In some embodiments, all gut samples from mice in Groups 1 and 2 receive a hypertrophy/hyperplasia score of 0. In some embodiments gut samples from about 70% (e.g. 7/10) mice from Group 3 receive a hyperplasia/hypertrophy score of 3, and gut samples from about 30% (e.g. 3/10) mice receive a hypertrophy/hyperplasia score of 2. In some embodiments, gut samples from about 80% (e.g. 8/10) mice from Group 4 receive a hyperplasia/hypertrophy score of 2, and gut samples from about 20% (e.g. 2/10) mice receive a hypertrophy/hyperplasia score of 3. In some embodiments, a measure of hypertrophy/hyperplasia in digestive tract tissue in mice of Group 4 is improved compared to a measure of hypertrophy/hyperplasia in digestive tract tissue in mice Group 3 as illustrated in FIG. 24A.

t. Clinical Trials

To determine the suitability of combination treatment with a peptidomimetic macrocycle disclosed herein and an additional therapy (e.g. any additional therapeutic agent disclosed herein) in humans, clinical trials are performed. For example, patients diagnosed with cancer and in need of treatment can be selected and separated into combination treatment and one or more control groups, wherein the combination treatment group is administered a peptidomimetic macrocycle in combination with an additional therapeutic agent, while the control groups receive a placebo or the additional therapeutic agent alone. The treatment safety and efficacy of the combination treatment can thus be evaluated by performing comparisons of the patient groups with respect to factors such as the presence and severity of side effects, survival, and quality-of-life. In this example, the patient group treated with a combination of a peptidomimetic macrocycle and an additional therapeutic agent can show improved long-term survival and/or decreased side effects compared to patient control groups treated with a placebo or the additional therapeutic agent alone.

Overall health of a subject can also be assessed before or after treatment with a peptidomimetic macrocycle via Eastern Cooperative Oncology Group (ECOG) performance status (PS). The ECOG performance status assigns a 0-5 score to a subject based on the criteria shown in the table below:

| GRADE | ECOG PERFORMANCE STATUS |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all selfcare but unable to carry |
| | out any work activities; up and about more than 50% of |
| | waking hours |
| 3 | Capable of only limited selfcare; confined to bed or chair |
| | more than 50% of waking hours |
| 4 | Completely disabled; cannot carry on any selfcare; totally |
| | confined to bed or chair |
| 5 | Dead |

### EXAMPLES

### Example 1: Synthesis of 6-Chlorotryptophan Fmoc Amino Acids

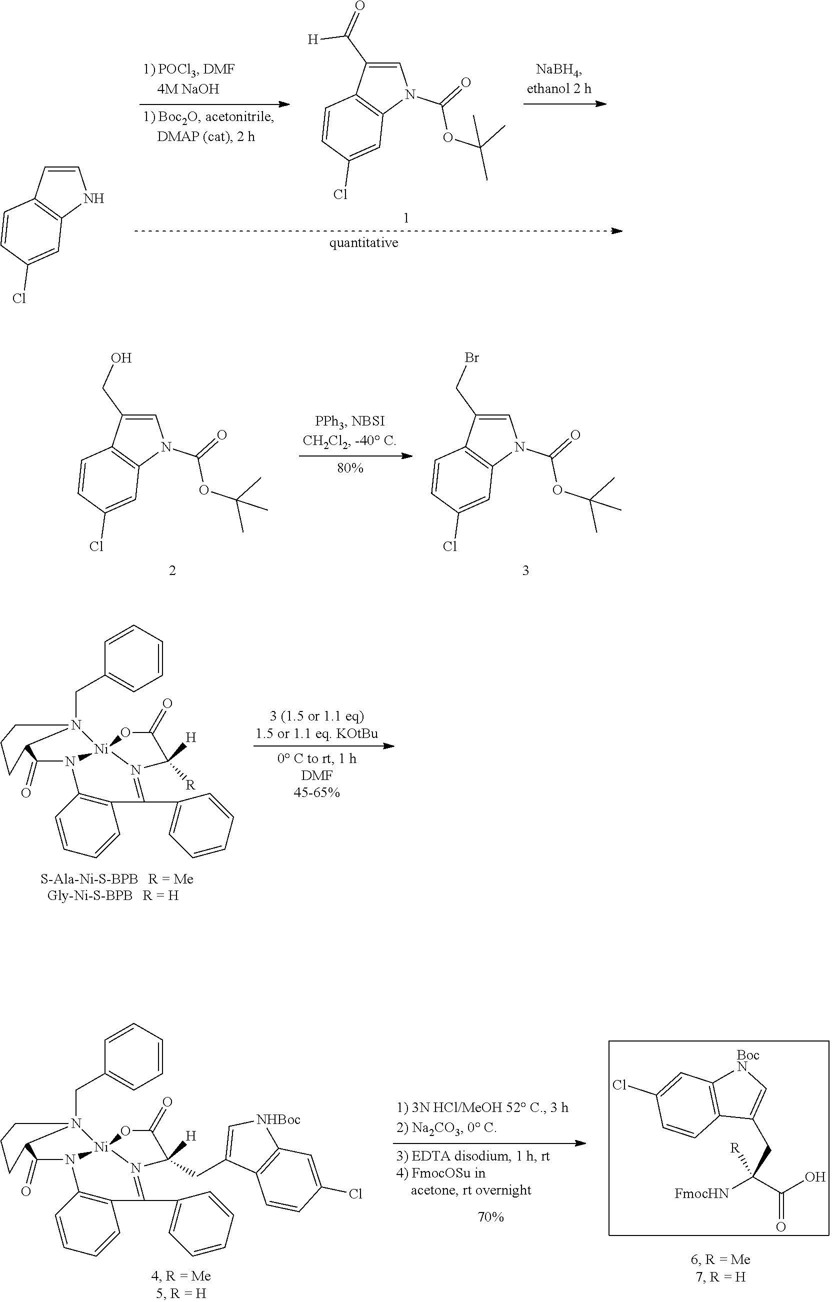

### Tert-butyl 6-chloro-3-formyl-1H-indole-1-carboxylate, 1

To a stirred solution of dry DMF (12 mL) was added dropwise POCl₃ (3.92 mL, 43 mmol, 1.3 equiv) at 0° C. under argon. The solution was stirred at 0° C. for 20 min before a solution of 6-chloroindole (5.0 g, 33 mmol, 1 eq.) in dry DMF (30 mL) was added dropwise. The resulting mixture was warmed to room temperature and stirred for an additional 2.5 h. Water (50 mL) was added to the reaction mixture, and the solution was neutralized with 4M aqueous NaOH (pH ˜8). The resulting solid was filtered off, washed with water, and dried under vacuum. This material was used in the next step without additional purification.

To a stirred solution of the crude formyl indole (33 mmol, 1 eq.) in THF (150 mL) was added successively Boc₂O (7.91 g, 36.3 mmol, 1.1 equiv) and DMAP (0.4 g, 3.3 mmol, 0.1 equiv) at room temperature under N₂. The resulting mixture was stirred at room temperature for 1.5 h, and the solvent was evaporated under reduced pressure. The residue was taken up in EtOAc and washed with 1N HCl, dried, and concentrated to afford formyl indole 1 (9 g, 98% over 2 steps) as a white solid. ¹H NMR (CDCl₃) δ: 1.70 (s, Boc, 9H); 7.35 (dd, 1H); 8.21 (m, 3H); 10.07 (s, 1H).

### Tert-butyl 6-chloro-3-(hydroxymethyl)-1H-indole-1-carboxylate, 2

To a solution of compound 1 (8.86 g, 32 mmol, 1 eq.) in ethanol (150 mL) was added NaBH₄ (2.4 g, 63 mmol, 2 eq.). The reaction was stirred for 3 h at room temperature. The reaction mixture was concentrated, and the residue was poured into diethyl ether and water. The organic layer was separated, dried over magnesium sulfate, and concentrated to give a white solid (8.7 g, 98%). This material was directly used in the next step without additional purification. ¹H NMR (CDCl₃) δ: 1.65 (s, Boc, 9H); 4.80 (s, 2H, CH₂); 7.21 (dd, 1H); 7.53 (m, 2H); 8.16 (bs, 1H).

### αMe-6Cl-Trp(Boc)-Ni—S-BPB, 4

To S-Ala-Ni—S-BPB (2.66 g, 5.2 mmol, 1 eq.) and KO-tBu (0.87 g, 7.8 mmol, 1.5 eq.) was added 50 mL of DMF under argon. The bromide derivative compound 3 (2.68 g, 7.8 mmol, 1.5 eq.) was dissolved in DMF (5.0 mL) and added to the reaction mixture using a syringe. The reaction mixture was stirred at ambient temperature for 1 h. The solution was then quenched with 5% aqueous acetic acid and diluted with water. The desired product was extracted in dichloromethane, dried, and concentrated. The oily product 4 was purified by flash chromatography (solid loading) on normal phase using EtOAc and hexanes as eluents to give a red solid (1.78 g, 45% yield). M+H calc. 775.21, M+H obs. 775.26; ¹H NMR (CDCl₃) δ: 1.23 (s, 3H, cMe); 1.56 (m, 11H, Boc+CH₂); 1.82-2.20 (m, 4H, 2CH₂); 3.03 (m, 1H, CH_{α}); 3.24 (m, 2H, CH₂); 3.57 and 4.29 (AB system, 2H, CH₂ (benzyl), J=12.8 Hz); 6.62 (d, 2H); 6.98 (d, 1H); 7.14 (m, 2H); 7.23 (m, 1H); 7.32-7.36 (m, 5H); 7.50 (m, 2H); 7.67 (bs, 1H); 7.98 (d, 2H); 8.27 (m, 2H).

### 6Cl-Trp(Boc)-Ni—S-BPB, 5

To Gly-Ni—S-BPB (4.6 g, 9.2 mmol, 1 eq.) and KO-tBu (1.14 g, 10.1 mmol, 1.1 eq.) was added 95 mL of DMF under argon. The bromide derivative compound 3 (3.5 g, 4.6 mmol, 1.1 eq.) was dissolved in DMF (10 mL) and added to the reaction mixture using a syringe. The reaction mixture was stirred at ambient temperature for 1 h. The solution was then quenched with 5% aqueous acetic acid and diluted with water. The desired product was extracted in dichloromethane, dried and concentrated. The oily product 5 was purified by flash chromatography (solid loading) on normal phase using EtOAc and hexanes as eluents to give a red solid (5 g, 71% yield). M+H calc. 761.20, M+H obs. 761.34; ¹H NMR (CDCl₃) δ: 1.58 (m, 11H, Boc+CH₂); 1.84 (m, 1H); 1.96 (m, 1H); 2.24 (m, 2H, CH₂); 3.00 (m, 1H, CHa); 3.22 (m, 2H, CH₂); 3.45 and 4.25 (AB system, 2H, CH₂ (benzyl), J=12.8 Hz); 4.27 (m, 1H, CHa); 6.65 (d, 2H); 6.88 (d, 1H); 7.07 (m, 2H); 7.14 (m, 2H); 7.28 (m, 3H); 7.35-7.39 (m, 2H); 7.52 (m, 2H); 7.96 (d, 2H); 8.28 (m, 2H).

Fmoc-αMe-6Cl-Trp(Boc)-OH, 6.

To a solution of 3N HCl/MeOH (⅓, 15 mL) at 50° C. was added a solution of compound 4 (1.75 g, 2.3 mmol, 1 eq.) in MeOH (5 ml) dropwise. The starting material disappeared within 3-4 h. The acidic solution was then cooled to 0° C. with an ice bath and quenched with an aqueous solution of Na₂CO₃ (1.21 g, 11.5 mmol, 5 eq.). Methanol was removed and 8 eq. of Na₂CO₃ (1.95 g, 18.4 mmol) were added to the suspension. EDTA disodium salt dihydrate (1.68 g, 4.5 mmol, 2 eq.) was then added, and the resulting suspension was stirred for 2 h. A solution of Fmoc-OSu (0.84 g, 2.5 mmol, 1.1 eq.) in acetone (50 mL) was added, and the reaction was stirred overnight. The reaction was diluted with diethyl ether and 1N HCl. The organic layer was then dried over magnesium sulfate and concentrated in vacuo. The desired product 6 was purified on normal phase using acetone and dichloromethane as eluents to give a white foam (0.9 g, 70% yield). M+H calc. 575.19, M+H obs. 575.37; ¹H NMR (CDCl₃) 1.59 (s, 9H, Boc); 1.68 (s, 3H, Me); 3.48 (bs, 2H, CH₂); 4.22 (m, 1H, CH); 4.39 (bs, 2H, CH₂); 5.47 (s, 1H, NH); 7.10 (m, 1H); 7.18 (m, 2H); 7.27 (m, 2H); 7.39 (m, 2H); 7.50 (m, 2H); 7.75 (d, 2H); 8.12 (bs, 1H).

Fmoc-6Cl-Trp(Boc)-OH, 7.

To a solution of 3N HCl/MeOH (⅓, 44 mL) at 50° C. was added a solution of compound 5 (5 g, 6.6 mmol, 1 eq.) in MeOH (10 ml) dropwise. The starting material disappeared within 3-4 h. The acidic solution was then cooled to 0° C. with an ice bath and quenched with an aqueous solution of Na₂CO₃ (3.48 g, 33 mmol, 5 eq.). Methanol was removed and 8 eq. of Na₂CO₃ (5.57 g, 52 mmol) were added to the suspension. EDTA disodium salt dihydrate (4.89 g, 13.1 mmol, 2 eq.) was added to the suspension, and the resulting suspension was stirred for 2 h. A solution of Fmoc-OSu (2.21 g, 6.55 mmol, 1.1 eq.) in acetone (100 mL) was added, and the reaction was stirred overnight. The reaction was diluted with diethyl ether and 1N HCl. The organic layer was then dried over magnesium sulfate and concentrated in vacuo. The desired product 7 was purified on normal phase using acetone and dichloromethane as eluents to give a white foam (2.6 g, 69% yield). M+H calc. 561.17, M+H obs. 561.37; ¹H NMR (CDCl₃) 1.63 (s, 9H, Boc); 3.26 (m, 2H, CH₂); 4.19 (m, 1H, CH); 4.39 (m, 2H, CH₂); 4.76 (m, 1H); 5.35 (d, 1H, NH); 7.18 (m, 2H); 7.28 (m, 2H); 7.39 (m, 3H); 7.50 (m, 2H); 7.75 (d, 2H); 8.14 (bs, 1H).

### Example 2: Peptidomimetic Macrocycles

Peptidomimetic macrocycles were designed by replacing two or more naturally-occurring amino acids with the corresponding synthetic amino acids. Substitutions were made at i and i+4, and i and i+7 positions. Peptide synthesis was performed manually or using an automated peptide synthesizer under solid phase conditions using rink amide AM resin and Fmoc main-chain protecting group chemistry. For the coupling of natural Fmoc-protected amino acids, 10 eq. of amino acid and a 1:1:2 molar ratio of coupling reagents HBTU/HOBt/DIEA were employed. Non-natural amino acids (4 eq.) were coupled with a 1:1:2 molar ratio of HATU/HOBt/DIEA. The N-termini of the synthetic peptides were acetylated, and the C-termini were amidated.

Purification of crosslinked compounds was achieved by HPLC on a reverse phase C18 column to yield the pure compounds. The chemical compositions of the pure products were confirmed by LC/MS mass spectrometry and amino acid analysis.

Synthesis of Dialkyne-Crosslinked Peptidomimetic Macrocycles, Including SP662, SP663 and SP664.

Fully protected resin-bound peptides were synthesized on a PEG-PS resin (loading 0.45 mmol/g) on a 0.2 mmol scale. Deprotection of the temporary Fmoc group was achieved by 3×10 min treatments of the resin bound peptide with 20% (v/v) piperidine in DMF. After washing with NMP (3×), dichloromethane (3×) and NMP (3×), coupling of each successive amino acid was achieved with 1×60 min incubation with the appropriate pre-activated Fmoc-amino acid derivative. All protected amino acids (0.4 mmol) were dissolved in NMP and activated with HCTU (0.4 mmol) and DIEA (0.8 mmol) prior to transfer of the coupling solution to the de-protected resin-bound peptide. After coupling was completed, the resin was washed in preparation for the next deprotection/coupling cycle.

Acetylation of the amino terminus was carried out in the presence of acetic anhydride/DIEA in NMP. The LC-MS analysis of a cleaved and de-protected sample obtained from an aliquot of the fully assembled resin-bound peptide was accomplished in order to verifying the completion of each coupling. In a typical example, tetrahydrofuran (4 ml) and triethylamine (2 ml) were added to the peptide resin (0.2 mmol) in a 40 ml glass vial and shaken for 10 minutes. Pd(PPh₃)₂Cl₂ (0.014 g, 0.02 mmol) and copper iodide (0.008 g, 0.04 mmol) were then added and the resulting reaction mixture was mechanically shaken 16 hours while open to atmosphere. The diyne-cyclized resin-bound peptides were de-protected and cleaved from the solid support by treatment with TFA/H₂O/TIS (95/5/5 v/v) for 2.5 h at room temperature. After filtration of the resin the TFA solution was precipitated in cold diethyl ether and centrifuged to yield the desired product as a solid. The crude product was purified by preparative HPLC.

Synthesis of Single Alkyne-Crosslinked Peptidomimetic Macrocycles, Including SP665.

Fully protected resin-bound peptides were synthesized on a Rink amide MBHA resin (loading 0.62 mmol/g) on a 0.1 mmol scale. Deprotection of the temporary Fmoc group was achieved by 2×20 min treatments of the resin bound peptide with 25% (v/v) piperidine in NMP. After extensive flow washing with NMP and dichloromethane, coupling of each successive amino acid was achieved with 1×60 min incubation with the appropriate pre-activated Fmoc-amino acid derivative. All protected amino acids (1 mmol) were dissolved in NMP and activated with HCTU (1 mmol) and DIEA (1 mmol) prior to transfer of the coupling solution to the de-protected resin-bound peptide. After coupling was completed, the resin was extensively flow washed in preparation for the next deprotection/coupling cycle.

Acetylation of the amino terminus was carried out in the presence of acetic anhydride/DIEA in NMP/NMM. The LC-MS analysis of a cleaved and de-protected sample obtained from an aliquot of the fully assembled resin-bound peptide was accomplished to verify the completion of each coupling reaction. In a typical example, the peptide resin (0.1 mmol) was washed with DCM. Resin was loaded into a microwave vial. The vessel was evacuated and purged with nitrogen. Molybdenum hexacarbonyl (0.01 eq.) was added. Anhydrous chlorobenzene was added to the reaction vessel. Then 2-fluorophenol (1 eq.) was added. The reaction was then loaded into the microwave and held at 130° C. for 10 minutes. The reaction pushed for a longer period time when needed to complete the reaction. The alkyne-metathesized resin-bound peptides were de-protected and cleaved from the solid support by treating the solid support with TFA/H₂O/TIS (94/3/3 v/v) for 3 h at room temperature. After filtration of the resin, the TFA solution was precipitated in cold diethyl ether and centrifuged to yield the desired product as a solid. The crude product was purified by preparative HPLC.

TABLE 1 shows a list of peptidomimetic macrocycles prepared.

TABLE 1

| | | SEQ | | Exact | Found | Calc | Calc | Calc |
|---|---|---|---|---|---|---|---|---|
| SP | Sequence | ID NO: | Isomer | Mass | Mass | (M + 1)/1 | (M + 2)/2 | (M + 3)/3 |
| 1 | Ac-F$r8AYWEAc3cL$AAA-NH₂ | 1 | | 1456.78 | 729.44 | 1457.79 | 729.4 | 486.6 |
| 2 | Ac-F$r8AYWEAc3cL$AAibA-NH₂ | 2 | | 1470.79 | 736.4 | 1471.8 | 736.4 | 491.27 |
| 3 | Ac-LTF$r8AYWAQLSSANle-NH₂ | 3 | | 1715.97 | 859.02 | 1716.98 | 858.99 | 573 |
| 4 | Ac-LTF$r8AYWAQLSSAL-NH₂ | 4 | | 1715.97 | 859.02 | 1716.98 | 858.99 | 573 |
| 5 | Ac-LTF$r8AYWAQLSSAM-NH₂ | 5 | | 1733.92 | 868.48 | 1734.93 | 867.97 | 578.98 |
| 6 | Ac-LTF$r8AYWAQLSSAhL-NH₂ | 6 | | 1729.98 | 865.98 | 1730.99 | 866 | 577.67 |
| 7 | Ac-LTF$r8AYWAQLSSAF-NH₂ | 7 | | 1749.95 | 876.36 | 1750.96 | 875.98 | 584.32 |
| 8 | Ac-LTF$r8AYWAQL$SAI-NH₂ | 8 | | 1715.97 | 859.02 | 1716.98 | 858.99 | 573 |
| 9 | Ac-LTF$r8AYWAQL$SAChg-NH₂ | 9 | | 1741.98 | 871.98 | 1742.99 | 872 | 581.67 |
| 10 | Ac-LTF$r8AYWAQL$SAAib-NH₂ | 10 | | 1687.93 | 845.36 | 1688.94 | 844.97 | 563.65 |
| 11 | Ac-LTF$r8AYWAQL$SAA-NH₂ | 11 | | 1673.92 | 838.01 | 1674.93 | 837.97 | 558.98 |
| 12 | Ac-LTF$r8AYWA$L$S$Nle-NH₂ | 12 | | 1767.04 | 884.77 | 1768.05 | 884.53 | 590.02 |
| 13 | Ac-LTF$r8AYWA$L$S$A-NH₂ | 13 | | 1724.99 | 864.23 | 1726 | 863.5 | 576 |
| 14 | Ac-F$r8AYWEAc3cL$AANle-NH₂ | 14 | | 1498.82 | 750.46 | 1499.83 | 750.42 | 500.61 |
| 15 | Ac-F$r8AYWEAc3cL$AAL-NH₂ | 15 | | 1498.82 | 750.46 | 1499.83 | 750.42 | 500.61 |
| 16 | Ac-F$r8AYWEAc3cL$AAM-NH₂ | 16 | | 1516.78 | 759.41 | 1517.79 | 759.4 | 506.6 |
| 17 | Ac-F$r8AYWEAc3cL$AAhL-NH₂ | 17 | | 1512.84 | 757.49 | 1513.85 | 757.43 | 505.29 |
| 18 | Ac-F$r8AYWEAc3cL$AAF-NH₂ | 18 | | 1532.81 | 767.48 | 1533.82 | 767.41 | 511.94 |
| 19 | Ac-F$r8AYWEAc3cL$AAI-NH₂ | 19 | | 1498.82 | 750.39 | 1499.83 | 750.42 | 500.61 |
| 20 | Ac-F$r8AYWEAc3cL$AAChg-NH₂ | 20 | | 1524.84 | 763.48 | 1525.85 | 763.43 | 509.29 |
| 21 | Ac-F$r8AYWEAc3cL$AACha-NH₂ | 21 | | 1538.85 | 770.44 | 1539.86 | 770.43 | 513.96 |
| 22 | Ac-F$r8AYWEAc3cL$AAAib-NH₂ | 22 | | 1470.79 | 736.84 | 1471.8 | 736.4 | 491.27 |
| 23 | Ac-LTF$r8AYWAQL$AAAibV-NH₂ | 23 | | 1771.01 | 885.81 | 1772.02 | 886.51 | 591.34 |
| 24 | Ac-LTF$r8AYWAQL$AAAibV-NH₂ | 24 | iso2 | 1771.01 | 886.26 | 1772.02 | 886.51 | 591.34 |
| 25 | Ac-LTF$r8AYWAQL$SAibAA-NH₂ | 25 | | 1758.97 | 879.89 | 1759.98 | 880.49 | 587.33 |
| 26 | Ac-LTF$r8AYWAQL$SAibAA-NH₂ | 26 | iso2 | 1758.97 | 880.34 | 1759.98 | 880.49 | 587.33 |
| 27 | Ac-HLTF$r8HHWHQL$AANleNle-NH₂ | 27 | | 2056.15 | 1028.86 | 2057.16 | 1029.08 | 686.39 |
| 28 | Ac-DLTF$r8HHWHQL$RRLV-NH₂ | 28 | | 2190.23 | 731.15 | 2191.24 | 1096.12 | 731.08 |
| 29 | Ac-HHTF$r8HHWHQL$AAML-NH₂ | 29 | | 2098.08 | 700.43 | 2099.09 | 1050.05 | 700.37 |
| 30 | Ac-F$r8HHWHQL$RRDCha-NH₂ | 30 | | 1917.06 | 959.96 | 1918.07 | 959.54 | 640.03 |
| 31 | Ac-F$r8HHWHQL$HRFV-NH₂ | 31 | | 1876.02 | 938.65 | 1877.03 | 939.02 | 626.35 |
| 32 | Ac-HLTF$r8HHWHQL$AAhLA-NH₂ | 32 | | 2028.12 | 677.2 | 2029.13 | 1015.07 | 677.05 |
| 33 | Ac-DLTF$r8HHWHQL$RRChgl-NH₂ | 33 | | 2230.26 | 1115.89 | 2231.27 | 1116.14 | 744.43 |
| 34 | Ac-DLTF$r8HHWHQL$RRChgl-NH₂ | 34 | iso2 | 2230.26 | 1115.96 | 2231.27 | 1116.14 | 744.43 |
| 35 | Ac-HHTF$r8HHWHQL$AAChav-NH₂ | 35 | | 2106.14 | 1053.95 | 2107.15 | 1054.08 | 703.05 |
| 36 | Ac-F$r8HHWHQL$RRDa-NH₂ | 36 | | 1834.99 | 918.3 | 1836 | 918.5 | 612.67 |
| 37 | Ac-F$r8HHWHQL$HRAibG-NH₂ | 37 | | 1771.95 | 886.77 | 1772.96 | 886.98 | 591.66 |
| 38 | Ac-F$r8AYWAQL$HHNleL-NH₂ | 38 | | 1730.97 | 866.57 | 1731.98 | 866.49 | 578 |
| 39 | Ac-F$r8AYWSAL$HQANle-NH₂ | 39 | | 1638.89 | 820.54 | 1639.9 | 820.45 | 547.3 |
| 40 | Ac-F$r8AYWVQL$QHChgl-NH₂ | 40 | | 1776.01 | 889.44 | 1777.02 | 889.01 | 593.01 |
| 41 | Ac-F$r8AYWTAL$QQNlev-NH₂ | 41 | | 1671.94 | 836.97 | 1672.95 | 836.98 | 558.32 |
| 42 | Ac-F$r8AYWYQL$HAibAa-NH₂ | 42 | | 1686.89 | 844.52 | 1687.9 | 844.45 | 563.3 |
| 43 | Ac-LTF$r8AYWAQL$HHLa-NH₂ | 43 | | 1903.05 | 952.27 | 1904.06 | 952.53 | 635.36 |
| 44 | Ac-LTF$r8AYWAQL$HHLa-NH₂ | 44 | iso2 | 1903.05 | 952.27 | 1904.06 | 952.53 | 635.36 |
| 45 | Ac-LTF$r8AYWAQL$HQNlev-NH₂ | 45 | | 1922.08 | 962.48 | 1923.09 | 962.05 | 641.7 |
| 46 | Ac-LTF$r8AYWAQL$HQNlev-NH₂ | 46 | iso2 | 1922.08 | 962.4 | 1923.09 | 962.05 | 641.7 |
| 47 | Ac-LTF$r8AYWAQL$QQMl-NH₂ | 47 | | 1945.05 | 973.95 | 1946.06 | 973.53 | 649.36 |
| 48 | Ac-LTF$r8AYWAQL$QQMl-NH₂ | 48 | iso2 | 1945.05 | 973.88 | 1946.06 | 973.53 | 649.36 |
| 49 | Ac-LTF$r8AYWAQL$HAibhLV-NH₂ | 49 | | 1893.09 | 948.31 | 1894.1 | 947.55 | 632.04 |
| 50 | Ac-LTF$r8AYWAQL$AHFA-NH₂ | 50 | | 1871.01 | 937.4 | 1872.02 | 936.51 | 624.68 |
| 51 | Ac-HLTF$r8HHWHQL$AANlel-NH₂ | 51 | | 2056.15 | 1028.79 | 2057.16 | 1029.08 | 686.39 |
| 52 | Ac-DLTF$r8HHWHQL$RRLa-NH₂ | 52 | | 2162.2 | 721.82 | 2163.21 | 1082.11 | 721.74 |
| 53 | Ac-HHTF$r8HHWHQL$AAMv-NH₂ | 53 | | 2084.07 | 1042.92 | 2085.08 | 1043.04 | 695.7 |
| 54 | Ac-F$r8HHWHQL$RRDA-NH₂ | 54 | | 1834.99 | 612.74 | 1836 | 918.5 | 612.67 |
| 55 | Ac-F$r8HHWHQL$HRFCha-NH₂ | 55 | | 1930.06 | 966.47 | 1931.07 | 966.04 | 644.36 |
| 56 | Ac-F$r8AYWEAL$AA-NHAm | 56 | | 1443.82 | 1445.71 | 1444.83 | 722.92 | 482.28 |
| 57 | Ac-F$r8AYWEAL$AA-NHiAm | 57 | | 1443.82 | 723.13 | 1444.83 | 722.92 | 482.28 |
| 58 | Ac-F$r8AYWEAL$AA-NHnPr3Ph | 58 | | 1491.82 | 747.3 | 1492.83 | 746.92 | 498.28 |
| 59 | Ac-F$r8AYWEAL$AA-NHnBu33Me | 59 | | 1457.83 | 1458.94 | 1458.84 | 729.92 | 486.95 |
| 60 | Ac-F$r8AYWEAL$AA-NHnPr | 60 | | 1415.79 | 709.28 | 1416.8 | 708.9 | 472.94 |
| 61 | Ac-F$r8AYWEAL$AA-NHnEt2Ch | 61 | | 1483.85 | 1485.77 | 1484.86 | 742.93 | 495.62 |
| 62 | Ac-F$r8AYWEAL$AA-NHnEt2Cp | 62 | | 1469.83 | 1470.78 | 1470.84 | 735.92 | 490.95 |
| 63 | Ac-F$r8AYWEAL$AA-NHHex | 63 | | 1457.83 | 730.19 | 1458.84 | 729.92 | 486.95 |
| 64 | Ac-LTF$r8AYWAQL$AAIA-NH₂ | 64 | | 1771.01 | 885.81 | 1772.02 | 886.51 | 591.34 |
| 65 | Ac-LTF$r8AYWAQL$AAIA-NH₂ | 65 | iso2 | 1771.01 | 866.8 | 1772.02 | 886.51 | 591.34 |
| 66 | Ac-LTF$r8AYWAAL$AAMA-NH₂ | 66 | | 1731.94 | 867.08 | 1732.95 | 866.98 | 578.32 |
| 67 | Ac-LTF$r8AYWAAL$AAMA-NH₂ | 67 | iso2 | 1731.94 | 867.28 | 1732.95 | 866.98 | 578.32 |
| 68 | Ac-LTF$r8AYWAQL$AANleA-NH₂ | 68 | | 1771.01 | 867.1 | 1772.02 | 886.51 | 591.34 |
| 69 | Ac-LTF$r8AYWAQL$AANleA-NH₂ | 69 | iso2 | 1771.01 | 886.89 | 1772.02 | 886.51 | 591.34 |
| 70 | Ac-LTF$r8AYWAQL$AAIa-NH₂ | 70 | | 1771.01 | 886.8 | 1772.02 | 886.51 | 591.34 |
| 71 | Ac-LTF$r8AYWAQL$AAIa-NH₂ | 71 | iso2 | 1771.01 | 887.09 | 1772.02 | 886.51 | 591.34 |
| 72 | Ac-LTF$r8AYWAAL$AAMa-NH₂ | 72 | | 1731.94 | 867.17 | 1732.95 | 866.98 | 578.32 |
| 73 | Ac-LTF$r8AYWAAL$AAMa-NH₂ | 73 | iso2 | 1731.94 | 867.37 | 1732.95 | 866.98 | 578.32 |
| 74 | Ac-LTF$r8AYWAQL$AANlea-NH₂ | 74 | | 1771.01 | 887.08 | 1772.02 | 886.51 | 591.34 |
| 75 | Ac-LTF$r8AYWAQL$AANlea-NH₂ | 75 | iso2 | 1771.01 | 887.08 | 1772.02 | 886.51 | 591.34 |
| 76 | Ac-LTF$r8AYWAAL$AAIv-NH₂ | 76 | | 1742.02 | 872.37 | 1743.03 | 872.02 | 581.68 |
| 77 | Ac-LTF$r8AYWAAL$AAIv-NH₂ | 77 | iso2 | 1742.02 | 872.74 | 1743.03 | 872.02 | 581.68 |
| 78 | Ac-LTF$r8AYWAQL$AAMv-NH₂ | 78 | | 1817 | 910.02 | 1818.01 | 909.51 | 606.67 |
| 79 | Ac-LTF$r8AYWAAL$AANlev-NH₂ | 79 | | 1742.02 | 872.37 | 1743.03 | 872.02 | 581.68 |
| 80 | Ac-LTF$r8AYWAAL$AANlev-NH₂ | 80 | iso2 | 1742.02 | 872.28 | 1743.03 | 872.02 | 581.68 |
| 81 | Ac-LTF$r8AYWAQL$AAIl-NH₂ | 81 | | 1813.05 | 907.81 | 1814.06 | 907.53 | 605.36 |
| 82 | Ac-LTF$r8AYWAQL$AAIl-NH₂ | 82 | iso2 | 1813.05 | 907.81 | 1814.06 | 907.53 | 605.36 |
| 83 | Ac-LTF$r8AYWAAL$AAMl-NH₂ | 83 | | 1773.99 | 887.37 | 1775 | 888 | 592.34 |
| 84 | Ac-LTF$r8AYWAQL$AANlel-NH₂ | 84 | | 1813.05 | 907.61 | 1814.06 | 907.53 | 605.36 |
| 85 | Ac-LTF$r8AYWAQL$AANlel-NH₂ | 85 | iso2 | 1813.05 | 907.71 | 1814.06 | 907.53 | 605.36 |
| 86 | Ac-F$r8AYWEAL$AAMA-NH₂ | 86 | | 1575.82 | 789.02 | 1576.83 | 788.92 | 526.28 |
| 87 | Ac-F$r8AYWEAL$AANleA-NH₂ | 87 | | 1557.86 | 780.14 | 1558.87 | 779.94 | 520.29 |
| 88 | Ac-F$r8AYWEAL$AAIa-NH₂ | 88 | | 1557.86 | 780.33 | 1558.87 | 779.94 | 520.29 |
| 89 | Ac-F$r8AYWEAL$AAMa-NH₂ | 89 | | 1575.82 | 789.3 | 1576.83 | 788.92 | 526.28 |
| 90 | Ac-F$r8AYWEAL$AANlea-NH₂ | 90 | | 1557.86 | 779.4 | 1558.87 | 779.94 | 520.29 |
| 91 | Ac-F$r8AYWEAL$AAIv-NH₂ | 91 | | 1585.89 | 794.29 | 1586.9 | 793.95 | 529.64 |
| 92 | Ac-F$r8AYWEAL$AAMv-NH₂ | 92 | | 1603.85 | 803.08 | 1604.86 | 802.93 | 535.62 |
| 93 | Ac-F$r8AYWEAL$AANlev-NH₂ | 93 | | 1585.89 | 793.46 | 1586.9 | 793.95 | 529.64 |
| 94 | Ac-F$r8AYWEAL$AAIl-NH₂ | 94 | | 1599.91 | 800.49 | 1600.92 | 800.96 | 534.31 |
| 95 | Ac-F$r8AYWEAL$AAMl-NH₂ | 95 | | 1617.86 | 809.44 | 1618.87 | 809.94 | 540.29 |
| 96 | Ac-F$r8AYWEAL$AANlel-NH₂ | 96 | | 1599.91 | 801.7 | 1600.92 | 800.96 | 534.31 |
| 97 | Ac-F$r8AYWEAL$AANlel-NH₂ | 97 | iso2 | 1599.91 | 801.42 | 1600.92 | 800.96 | 534.31 |
| 98 | Ac-LTF$r8AY6clWAQL$SAA-NH₂ | 98 | | 1707.88 | 855.72 | 1708.89 | 854.95 | 570.3 |
| 99 | Ac-LTF$r8AY6clWAQL$SAA-NH₂ | 99 | iso2 | 1707.88 | 855.35 | 1708.89 | 854.95 | 570.3 |
| 100 | Ac-WTF$r8FYWSQL$AVAa-NH₂ | 100 | | 1922.01 | 962.21 | 1923.02 | 962.01 | 641.68 |
| 101 | Ac-WTF$r8FYWSQL$AVAa-NH₂ | 101 | iso2 | 1922.01 | 962.49 | 1923.02 | 962.01 | 641.68 |
| 102 | Ac-WTF$r8VYWSQL$AVA-NH₂ | 102 | | 1802.98 | 902.72 | 1803.99 | 902.5 | 602 |
| 103 | Ac-WTF$r8VYWSQL$AVA-NH₂ | 103 | iso2 | 1802.98 | 903 | 1803.99 | 902.5 | 602 |
| 104 | Ac-WTF$r8FYWSQL$SAAa-NH₂ | 104 | | 1909.98 | 956.47 | 1910.99 | 956 | 637.67 |
| 105 | Ac-WTF$r8FYWSQL$SAAa-NH₂ | 105 | iso2 | 1909.98 | 956.47 | 1910.99 | 956 | 637.67 |
| 106 | Ac-WTF$r8VYWSQL$AVAaa-NH₂ | 106 | | 1945.05 | 974.15 | 1946.06 | 973.53 | 649.36 |
| 107 | Ac-WTF$r8VYWSQL$AVAaa-NH₂ | 107 | iso2 | 1945.05 | 973.78 | 1946.06 | 973.53 | 649.36 |
| 108 | Ac-LTF$r8AYWAQL$AVG-NH₂ | 108 | | 1671.94 | 837.52 | 1672.95 | 836.98 | 558.32 |
| 109 | Ac-LTF$r8AYWAQL$AVG-NH₂ | 109 | iso2 | 1671.94 | 837.21 | 1672.95 | 836.98 | 558.32 |
| 110 | Ac-LTF$r8AYWAQL$AVQ-NH₂ | 110 | | 1742.98 | 872.74 | 1743.99 | 872.5 | 582 |
| 111 | Ac-LTF$r8AYWAQL$AVQ-NH₂ | 111 | iso2 | 1742.98 | 872.74 | 1743.99 | 872.5 | 582 |
| 112 | Ac-LTF$r8AYWAQLSSAa-NH₂ | 112 | | 1673.92 | 838.23 | 1674.93 | 837.97 | 558.98 |
| 113 | Ac-LTF$r8AYWAQLSSAa-NH₂ | 113 | iso2 | 1673.92 | 838.32 | 1674.93 | 837.97 | 558.98 |
| 114 | Ac-LTF$r8AYWAQhL$SAA-NH₂ | 114 | | 1687.93 | 844.37 | 1688.94 | 844.97 | 563.65 |
| 115 | Ac-LTF$r8AYWAQhL$SAA-NH₂ | 115 | iso2 | 1687.93 | 844.81 | 1688.94 | 844.97 | 563.65 |
| 116 | Ac-LTF$r8AYWEQLStSA$-NH₂ | 116 | | 1826 | 905.27 | 1827.01 | 914.01 | 609.67 |
| 117 | Ac-LTF$r8AYWAQLSSLA-NH₂ | 117 | | 1715.97 | 858.48 | 1716.98 | 858.99 | 573 |
| 118 | Ac-LTF$r8AYWAQLSSLA-NH₂ | 118 | iso2 | 1715.97 | 858.87 | 1716.98 | 858.99 | 573 |
| 119 | Ac-LTF$r8AYWAQLSSWA-NH₂ | 119 | | 1788.96 | 895.21 | 1789.97 | 895.49 | 597.33 |
| 120 | Ac-LTF$r8AYWAQLSSWA-NH₂ | 120 | iso2 | 1788.96 | 895.28 | 1789.97 | 895.49 | 597.33 |
| 121 | Ac-LTF$r8AYWAQL$SVS-NH₂ | 121 | | 1717.94 | 859.84 | 1718.95 | 859.98 | 573.65 |
| 122 | Ac-LTF$r8AYWAQLSSAS-NH₂ | 122 | | 1689.91 | 845.85 | 1690.92 | 845.96 | 564.31 |
| 123 | Ac-LTF$r8AYWAQLSSVG-NH₂ | 123 | | 1687.93 | 844.81 | 1688.94 | 844.97 | 563.65 |
| 124 | Ac-ETF$r8VYWAQL$SAa-NH₂ | 124 | | 1717.91 | 859.76 | 1718.92 | 859.96 | 573.64 |
| 125 | Ac-ETF$r8VYWAQL$SAA-NH₂ | 125 | | 1717.91 | 859.84 | 1718.92 | 859.96 | 573.64 |
| 126 | Ac-ETF$r8VYWAQL$SVA-NH₂ | 126 | | 1745.94 | 873.82 | 1746.95 | 873.98 | 582.99 |
| 127 | Ac-ETF$r8VYWAQL$SLA-NH₂ | 127 | | 1759.96 | 880.85 | 1760.97 | 880.99 | 587.66 |
| 128 | Ac-ETF$r8VYWAQL$SWA-NH₂ | 128 | | 1832.95 | 917.34 | 1833.96 | 917.48 | 611.99 |
| 129 | Ac-ETF$r8KYWAQL$SWA-NH₂ | 129 | | 1861.98 | 931.92 | 1862.99 | 932 | 621.67 |
| 130 | Ac-ETF$r8VYWAQL$SVS-NH₂ | 130 | | 1761.93 | 881.89 | 1762.94 | 881.97 | 588.32 |
| 131 | Ac-ETF$r8VYWAQL$SAS-NH₂ | 131 | | 1733.9 | 867.83 | 1734.91 | 867.96 | 578.97 |
| 132 | Ac-ETF$r8VYWAQL$SVG-NH₂ | 132 | | 1731.92 | 866.87 | 1732.93 | 866.97 | 578.31 |
| 133 | Ac-LTF$r8VYWAQL$SSa-NH₂ | 133 | | 1717.94 | 859.47 | 1718.95 | 859.98 | 573.65 |
| 134 | Ac-ETF$r8VYWAQL$SSa-NH₂ | 134 | | 1733.9 | 867.83 | 1734.91 | 867.96 | 578.97 |
| 135 | Ac-LTF$r8VYWAQL$SNa-NH₂ | 135 | | 1744.96 | 873.38 | 1745.97 | 873.49 | 582.66 |
| 136 | Ac-ETF$r8VYWAQL$SNa-NH₂ | 136 | | 1760.91 | 881.3 | 1761.92 | 881.46 | 587.98 |
| 137 | Ac-LTF$r8VYWAQL$SAa-NH₂ | 137 | | 1701.95 | 851.84 | 1702.96 | 851.98 | 568.32 |
| 138 | Ac-LTF$r8VYWAQL$SVA-NH₂ | 138 | | 1729.98 | 865.53 | 1730.99 | 866 | 577.67 |
| 139 | Ac-LTF$r8VYWAQL$SVA-NH₂ | 139 | iso2 | 1729.98 | 865.9 | 1730.99 | 866 | 577.67 |
| 140 | Ac-LTF$r8VYWAQL$SWA-NH₂ | 140 | | 1816.99 | 909.42 | 1818 | 909.5 | 606.67 |
| 141 | Ac-LTF$r8VYWAQL$SVS-NH₂ | 141 | | 1745.98 | 873.9 | 1746.99 | 874 | 583 |
| 142 | Ac-LTF$r8VYWAQL$SVS-NH₂ | 142 | iso2 | 1745.98 | 873.9 | 1746.99 | 874 | 583 |
| 143 | Ac-LTF$r8VYWAQL$SAS-NH₂ | 143 | | 1717.94 | 859.84 | 1718.95 | 859.98 | 573.65 |
| 144 | Ac-LTF$r8VYWAQL$SAS-NH₂ | 144 | iso2 | 1717.94 | 859.91 | 1718.95 | 859.98 | 573.65 |
| 145 | Ac-LTF$r8VYWAQL$SVG-NH₂ | 145 | | 1715.97 | 858.87 | 1716.98 | 858.99 | 573 |
| 146 | Ac-LTF$r8VYWAQL$SVG-NH₂ | 146 | iso2 | 1715.97 | 858.87 | 1716.98 | 858.99 | 573 |
| 147 | Ac-LTF$r8EYWAQCha$SAA-NH₂ | 147 | | 1771.96 | 886.85 | 1772.97 | 886.99 | 591.66 |
| 148 | Ac-LTF$r8EYWAQCha$SAA-NH₂ | 148 | iso2 | 1771.96 | 886.85 | 1772.97 | 886.99 | 591.66 |
| 149 | Ac-LTF$r8EYWAQCpg$SAA-NH₂ | 149 | | 1743.92 | 872.86 | 1744.93 | 872.97 | 582.31 |
| 150 | Ac-LTF$r8EYWAQCpg$SAA-NH₂ | 150 | iso2 | 1743.92 | 872.86 | 1744.93 | 872.97 | 582.31 |
| 151 | Ac-LTF$r8EYWAQF$SAA-NH₂ | 151 | | 1765.91 | 883.44 | 1766.92 | 883.96 | 589.64 |
| 152 | Ac-LTF$r8EYWAQF$SAA-NH₂ | 152 | iso2 | 1765.91 | 883.89 | 1766.92 | 883.96 | 589.64 |
| 153 | Ac-LTF$r8EYWAQCba$SAA-NH₂ | 153 | | 1743.92 | 872.42 | 1744.93 | 872.97 | 582.31 |
| 154 | Ac-LTF$r8EYWAQCba$SAA-NH₂ | 154 | iso2 | 1743.92 | 873.39 | 1744.93 | 872.97 | 582.31 |
| 155 | Ac-LTF3Cl$r8EYWAQL$SAA-NH₂ | 155 | | 1765.89 | 883.89 | 1766.9 | 883.95 | 589.64 |
| 156 | Ac-LTF3Cl$r8EYWAQL$SAA-NH₂ | 156 | iso2 | 1765.89 | 883.96 | 1766.9 | 883.95 | 589.64 |
| 157 | Ac-LTF34F2$r8EYWAQL$SAA-NH₂ | 157 | | 1767.91 | 884.48 | 1768.92 | 884.96 | 590.31 |
| 158 | Ac-LTF34F2$r8EYWAQL$SAA-NH₂ | 158 | iso2 | 1767.91 | 884.48 | 1768.92 | 884.96 | 590.31 |
| 159 | Ac-LTF34F2$r8EYWAQhL$SAA-NH₂ | 159 | | 1781.92 | 891.44 | 1782.93 | 891.97 | 594.98 |
| 160 | Ac-LTF34F2$r8EYWAQhL$SAA-NH₂ | 160 | iso2 | 1781.92 | 891.88 | 1782.93 | 891.97 | 594.98 |
| 161 | Ac-ETF$r8EYWAQL$SAA-NH₂ | 161 | | 1747.88 | 874.34 | 1748.89 | 874.95 | 583.63 |
| 162 | Ac-LTF$r8AYWVQL$SAA-NH₂ | 162 | | 1701.95 | 851.4 | 1702.96 | 851.98 | 568.32 |
| 163 | Ac-LTF$r8AHWAQL$SAA-NH₂ | 163 | | 1647.91 | 824.83 | 1648.92 | 824.96 | 550.31 |
| 164 | Ac-LTF$r8AEWAQL$SAA-NH₂ | 164 | | 1639.9 | 820.39 | 1640.91 | 820.96 | 547.64 |
| 165 | Ac-LTF$r8ASWAQL$SAA-NH₂ | 165 | | 1597.89 | 799.38 | 1598.9 | 799.95 | 533.64 |
| 166 | Ac-LTF$r8AEWAQL$SAA-NH₂ | 166 | iso2 | 1639.9 | 820.39 | 1640.91 | 820.96 | 547.64 |
| 167 | Ac-LTF$r8ASWAQL$SAA-NH₂ | 167 | iso2 | 1597.89 | 800.31 | 1598.9 | 799.95 | 533.64 |
| 168 | Ac-LTF$r8AF4coohWAQL$SAA-NH₂ | 168 | | 1701.91 | 851.4 | 1702.92 | 851.96 | 568.31 |
| 169 | Ac-LTF$r8AF4coohWAQL$SAA-NH₂ | 169 | iso2 | 1701.91 | 851.4 | 1702.92 | 851.96 | 568.31 |
| 170 | Ac-LTF$r8AHWAQL$AAIa-NH₂ | 170 | | 1745 | 874.13 | 1746.01 | 873.51 | 582.67 |
| 171 | Ac-ITF$r8FYWAQL$AAIa-NH₂ | 171 | | 1847.04 | 923.92 | 1848.05 | 924.53 | 616.69 |
| 172 | Ac-ITF$r8EHWAQL$AAIa-NH₂ | 172 | | 1803.01 | 903.17 | 1804.02 | 902.51 | 602.01 |
| 173 | Ac-ITF$r8EHWAQL$AAIa-NH₂ | 173 | iso2 | 1803.01 | 903.17 | 1804.02 | 902.51 | 602.01 |
| 174 | Ac-ETF$r8EHWAQL$AAIa-NH₂ | 174 | | 1818.97 | 910.76 | 1819.98 | 910.49 | 607.33 |
| 175 | Ac-ETF$r8EHWAQL$AAIa-NH₂ | 175 | iso2 | 1818.97 | 910.85 | 1819.98 | 910.49 | 607.33 |
| 176 | Ac-LTF$r8AHWVQL$AAIa-NH₂ | 176 | | 1773.03 | 888.09 | 1774.04 | 887.52 | 592.02 |
| 177 | Ac-ITF$r8FYWVQL$AAIa-NH₂ | 177 | | 1875.07 | 939.16 | 1876.08 | 938.54 | 626.03 |
| 178 | Ac-ITF$r8EYWVQL$AAIa-NH₂ | 178 | | 1857.04 | 929.83 | 1858.05 | 929.53 | 620.02 |
| 179 | Ac-ITF$r8EHWVQL$AAIa-NH₂ | 179 | | 1831.04 | 916.86 | 1832.05 | 916.53 | 611.35 |
| 180 | Ac-LTF$r8AEWAQL$AAIa-NH₂ | 180 | | 1736.99 | 869.87 | 1738 | 869.5 | 580 |
| 181 | Ac-LTF$r8AF4coohWAQL$AAIa-NH₂ | 181 | | 1799 | 900.17 | 1800.01 | 900.51 | 600.67 |
| 182 | Ac-LTF$r8AF4coohWAQL$AAIa-NH₂ | 182 | iso2 | 1799 | 900.24 | 1800.01 | 900.51 | 600.67 |
| 183 | Ac-LTF$r8AHWAQL$AHFA-NH₂ | 183 | | 1845.01 | 923.89 | 1846.02 | 923.51 | 616.01 |
| 184 | Ac-ITF$r8FYWAQL$AHFA-NH₂ | 184 | | 1947.05 | 975.05 | 1948.06 | 974.53 | 650.02 |
| 185 | Ac-ITF$r8FYWAQL$AHFA-NH₂ | 185 | iso2 | 1947.05 | 976.07 | 1948.06 | 974.53 | 650.02 |
| 186 | Ac-ITF$r8FHWAQL$AEFA-NH₂ | 186 | | 1913.02 | 958.12 | 1914.03 | 957.52 | 638.68 |
| 187 | Ac-ITF$r8FHWAQL$AEFA-NH₂ | 187 | iso2 | 1913.02 | 957.86 | 1914.03 | 957.52 | 638.68 |
| 188 | Ac-ITF$r8EHWAQL$AHFA-NH₂ | 188 | | 1903.01 | 952.94 | 1904.02 | 952.51 | 635.34 |
| 189 | Ac-ITF$r8EHWAQL$AHFA-NH₂ | 189 | iso2 | 1903.01 | 953.87 | 1904.02 | 952.51 | 635.34 |
| 190 | Ac-LTF$r8AHWVQL$AHFA-NH₂ | 190 | | 1873.04 | 937.86 | 1874.05 | 937.53 | 625.35 |
| 191 | Ac-ITF$r8FYWVQL$AHFA-NH₂ | 191 | | 1975.08 | 988.83 | 1976.09 | 988.55 | 659.37 |
| 192 | Ac-ITF$r8EYWVQL$AHFA-NH₂ | 192 | | 1957.05 | 979.35 | 1958.06 | 979.53 | 653.36 |
| 193 | Ac-ITF$r8EHWVQL$AHFA-NH₂ | 193 | | 1931.05 | 967 | 1932.06 | 966.53 | 644.69 |
| 194 | Ac-ITF$r8EHWVQL$AHFA-NH₂ | 194 | iso2 | 1931.05 | 967.93 | 1932.06 | 966.53 | 644.69 |
| 195 | Ac-ETF$r8EYWAAL$SAA-NH₂ | 195 | | 1690.86 | 845.85 | 1691.87 | 846.44 | 564.63 |
| 196 | Ac-LTF$r8AYWVAL$SAA-NH₂ | 196 | | 1644.93 | 824.08 | 1645.94 | 823.47 | 549.32 |
| 197 | Ac-LTF$r8AHWAAL$SAA-NH₂ | 197 | | 1590.89 | 796.88 | 1591.9 | 796.45 | 531.3 |
| 198 | Ac-LTF$r8AEWAAL$SAA-NH₂ | 198 | | 1582.88 | 791.9 | 1583.89 | 792.45 | 528.63 |
| 199 | Ac-LTF$r8AEWAAL$SAA-NH₂ | 199 | iso2 | 1582.88 | 791.9 | 1583.89 | 792.45 | 528.63 |
| 200 | Ac-LTF$r8ASWAAL$SAA-NH₂ | 200 | | 1540.87 | 770.74 | 1541.88 | 771.44 | 514.63 |
| 201 | Ac-LTF$r8ASWAAL$SAA-NH₂ | 201 | iso2 | 1540.87 | 770.88 | 1541.88 | 771.44 | 514.63 |
| 202 | Ac-LTF$r8AYWAAL$AAIa-NH₂ | 202 | | 1713.99 | 857.39 | 1715 | 858 | 572.34 |
| 203 | Ac-LTF$r8AYWAAL$AAIa-NH₂ | 203 | iso2 | 1713.99 | 857.84 | 1715 | 858 | 572.34 |
| 204 | Ac-LTF$r8AYWAAL$AHFA-NH₂ | 204 | | 1813.99 | 907.86 | 1815 | 908 | 605.67 |
| 205 | Ac-LTF$r8EHWAQL$AHIa-NH₂ | 205 | | 1869.03 | 936.1 | 1870.04 | 935.52 | 624.02 |
| 206 | Ac-LTF$r8EHWAQL$AHIa-NH₂ | 206 | iso2 | 1869.03 | 937.03 | 1870.04 | 935.52 | 624.02 |
| 207 | Ac-LTF$r8AHWAQL$AHIa-NH₂ | 207 | | 1811.03 | 906.87 | 1812.04 | 906.52 | 604.68 |
| 208 | Ac-LTF$r8EYWAQL$AHIa-NH₂ | 208 | | 1895.04 | 949.15 | 1896.05 | 948.53 | 632.69 |
| 209 | Ac-LTF$r8AYWAQL$AAFa-NH₂ | 209 | | 1804.99 | 903.2 | 1806 | 903.5 | 602.67 |
| 210 | Ac-LTF$r8AYWAQL$AAFa-NH₂ | 210 | iso2 | 1804.99 | 903.28 | 1806 | 903.5 | 602.67 |
| 211 | Ac-LTF$r8AYWAQL$AAWa-NH₂ | 211 | | 1844 | 922.81 | 1845.01 | 923.01 | 615.67 |
| 212 | Ac-LTF$r8AYWAQL$AAVa-NH₂ | 212 | | 1756.99 | 878.86 | 1758 | 879.5 | 586.67 |
| 213 | Ac-LTF$r8AYWAQL$AAVa-NH₂ | 213 | iso2 | 1756.99 | 879.3 | 1758 | 879.5 | 586.67 |
| 214 | Ac-LTF$r8AYWAQL$AALa-NH₂ | 214 | | 1771.01 | 886.26 | 1772.02 | 886.51 | 591.34 |
| 215 | Ac-LTF$r8AYWAQL$AALa-NH₂ | 215 | iso2 | 1771.01 | 886.33 | 1772.02 | 886.51 | 591.34 |
| 216 | Ac-LTF$r8EYWAQL$AAIa-NH₂ | 216 | | 1829.01 | 914.89 | 1830.02 | 915.51 | 610.68 |
| 217 | Ac-LTF$r8EYWAQL$AAIa-NH₂ | 217 | iso2 | 1829.01 | 915.34 | 1830.02 | 915.51 | 610.68 |
| 218 | Ac-LTF$r8EYWAQL$AAFa-NH₂ | 218 | | 1863 | 932.87 | 1864.01 | 932.51 | 622.01 |
| 219 | Ac-LTF$r8EYWAQL$AAFa-NH₂ | 219 | iso2 | 1863 | 932.87 | 1864.01 | 932.51 | 622.01 |
| 220 | Ac-LTF$r8EYWAQL$AAVa-NH₂ | 220 | | 1815 | 908.23 | 1816.01 | 908.51 | 606.01 |
| 221 | Ac-LTF$r8EYWAQL$AAVa-NH₂ | 221 | iso2 | 1815 | 908.31 | 1816.01 | 908.51 | 606.01 |
| 222 | Ac-LTF$r8EHWAQL$AAIa-NH₂ | 222 | | 1803.01 | 903.17 | 1804.02 | 902.51 | 602.01 |
| 223 | Ac-LTF$r8EHWAQL$AAIa-NH₂ | 223 | iso2 | 1803.01 | 902.8 | 1804.02 | 902.51 | 602.01 |
| 224 | Ac-LTF$r8EHWAQL$AAWa-NH₂ | 224 | | 1876 | 939.34 | 1877.01 | 939.01 | 626.34 |
| 225 | Ac-LTF$r8EHWAQL$AAWa-NH₂ | 225 | iso2 | 1876 | 939.62 | 1877.01 | 939.01 | 626.34 |
| 226 | Ac-LTF$r8EHWAQL$AALa-NH₂ | 226 | | 1803.01 | 902.8 | 1804.02 | 902.51 | 602.01 |
| 227 | Ac-LTF$r8EHWAQL$AALa-NH₂ | 227 | iso2 | 1803.01 | 902.9 | 1804.02 | 902.51 | 602.01 |
| 228 | Ac-ETF$r8EHWVQL$AALa-NH₂ | 228 | | 1847 | 924.82 | 1848.01 | 924.51 | 616.67 |
| 229 | Ac-LTF$r8AYWAQL$AAAa-NH₂ | 229 | | 1728.96 | 865.89 | 1729.97 | 865.49 | 577.33 |
| 230 | Ac-LTF$r8AYWAQL$AAAa-NH₂ | 230 | iso2 | 1728.96 | 865.89 | 1729.97 | 865.49 | 577.33 |
| 231 | Ac-LTF$r8AYWAQL$AAAibA-NH₂ | 231 | | 1742.98 | 872.83 | 1743.99 | 872.5 | 582 |
| 232 | Ac-LTF$r8AYWAQL$AAAibA-NH₂ | 232 | iso2 | 1742.98 | 872.92 | 1743.99 | 872.5 | 582 |
| 233 | Ac-LTF$r8AYWAQL$AAAAa-NH₂ | 233 | | 1800 | 901.42 | 1801.01 | 901.01 | 601.01 |
| 234 | Ac-LTF$r5AYWAQL$s8AAIa-NH₂ | 234 | | 1771.01 | 887.17 | 1772.02 | 886.51 | 591.34 |
| 235 | Ac-LTF$r5AYWAQL$s8SAA-NH₂ | 235 | | 1673.92 | 838.33 | 1674.93 | 837.97 | 558.98 |
| 236 | Ac-LTF$r8AYWAQCba$AANleA-NH₂ | 236 | | 1783.01 | 892.64 | 1784.02 | 892.51 | 595.34 |
| 237 | Ac-ETF$r8AYWAQCba$AANleA-NH₂ | 237 | | 1798.97 | 900.59 | 1799.98 | 900.49 | 600.66 |
| 238 | Ac-LTF$r8EYWAQCba$AANleA-NH₂ | 238 | | 1841.01 | 922.05 | 1842.02 | 921.51 | 614.68 |
| 239 | Ac-LTF$r8AYWAQCba$AWNleA-NH₂ | 239 | | 1898.05 | 950.46 | 1899.06 | 950.03 | 633.69 |
| 240 | Ac-ETF$r8AYWAQCba$AWNleA-NH₂ | 240 | | 1914.01 | 958.11 | 1915.02 | 958.01 | 639.01 |
| 241 | Ac-LTF$r8EYWAQCba$AWNleA-NH₂ | 241 | | 1956.06 | 950.62 | 1957.07 | 979.04 | 653.03 |
| 242 | Ac-LTF$r8EYWAQCba$SAFA-NH₂ | 242 | | 1890.99 | 946.55 | 1892 | 946.5 | 631.34 |
| 243 | Ac-LTF34F2$r8EYWAQCba$SANleA-NH₂ | 243 | | 1892.99 | 947.57 | 1894 | 947.5 | 632 |
| 244 | Ac-LTF$r8EF4coohWAQCba$SANleA-NH₂ | 244 | | 1885 | 943.59 | 1886.01 | 943.51 | 629.34 |
| 245 | Ac-LTF$r8EYWSQCba$SANleA-NH₂ | 245 | | 1873 | 937.58 | 1874.01 | 937.51 | 625.34 |
| 246 | Ac-LTF$r8EYWWQCba$SANleA-NH₂ | 246 | | 1972.05 | 987.61 | 1973.06 | 987.03 | 658.36 |
| 247 | Ac-LTF$r8EYWAQCba$AAIa-NH₂ | 247 | | 1841.01 | 922.05 | 1842.02 | 921.51 | 614.68 |
| 248 | Ac-LTF34F2$r8EYWAQCba$AAIa-NH₂ | 248 | | 1876.99 | 939.99 | 1878 | 939.5 | 626.67 |
| 249 | Ac-LTF$r8EF4coohWAQCba$AAIa-NH₂ | 249 | | 1869.01 | 935.64 | 1870.02 | 935.51 | 624.01 |
| 250 | Pam-ETF$r8EYWAQCba$SAA-NH₂ | 250 | | 1956.1 | 979.57 | 1957.11 | 979.06 | 653.04 |
| 251 | Ac-LThF$r8EFWAQCba$SAA-NH₂ | 251 | | 1741.94 | 872.11 | 1742.95 | 871.98 | 581.65 |
| 252 | Ac-LTA$r8EYWAQCba$SAA-NH₂ | 252 | | 1667.89 | 835.4 | 1668.9 | 834.95 | 556.97 |
| 253 | Ac-LTF$r8EYAAQCba$SAA-NH₂ | 253 | | 1628.88 | 815.61 | 1629.89 | 815.45 | 543.97 |
| 254 | Ac-LTF$r8EY2NalAQCba$SAA-NH₂ | 254 | | 1754.93 | 879.04 | 1755.94 | 878.47 | 585.98 |
| 255 | Ac-LTF$r8AYWAQCba$SAA-NH₂ | 255 | | 1685.92 | 844.71 | 1686.93 | 843.97 | 562.98 |
| 256 | Ac-LTF$r8EYWAQCba$SAF-NH₂ | 256 | | 1819.96 | 911.41 | 1820.97 | 910.99 | 607.66 |
| 257 | Ac-LTF$r8EYWAQCba$SAFa-NH₂ | 257 | | 1890.99 | 947.41 | 1892 | 946.5 | 631.34 |
| 258 | Ac-LTF$r8AYWAQCba$SAF-NH₂ | 258 | | 1761.95 | 882.73 | 1762.96 | 881.98 | 588.32 |
| 259 | Ac-LTF34F2$r8AYWAQCba$SAF-NH₂ | 259 | | 1797.93 | 900.87 | 1798.94 | 899.97 | 600.32 |
| 260 | Ac-LTF$r8AF4coohWAQCba$SAF-NH₂ | 260 | | 1789.94 | 896.43 | 1790.95 | 895.98 | 597.65 |
| 261 | Ac-LTF$r8EY6clWAQCba$SAF-NH₂ | 261 | | 1853.92 | 929.27 | 1854.93 | 927.97 | 618.98 |
| 262 | Ac-LTF$r8AYWSQCba$SAF-NH₂ | 262 | | 1777.94 | 890.87 | 1778.95 | 889.98 | 593.65 |
| 263 | Ac-LTF$r8AYWWQCba$SAF-NH₂ | 263 | | 1876.99 | 939.91 | 1878 | 939.5 | 626.67 |
| 264 | Ac-LTF$r8AYWAQCba$AAIa-NH₂ | 264 | | 1783.01 | 893.19 | 1784.02 | 892.51 | 595.34 |
| 265 | Ac-LTF34F2$r8AYWAQCba$AAIa-NH₂ | 265 | | 1818.99 | 911.23 | 1820 | 910.5 | 607.34 |
| 266 | Ac-LTF$r8AY6clWAQCba$AAIa-NH₂ | 266 | | 1816.97 | 909.84 | 1817.98 | 909.49 | 606.66 |
| 267 | Ac-LTF$r8AF4coohWAQCba$AAIa-NH₂ | 267 | | 1811 | 906.88 | 1812.01 | 906.51 | 604.67 |
| 268 | Ac-LTF$r8EYWAQCba$AAFa-NH₂ | 268 | | 1875 | 938.6 | 1876.01 | 938.51 | 626.01 |
| 269 | Ac-LTF$r8EYWAQCba$AAFa-NH₂ | 269 | iso2 | 1875 | 938.6 | 1876.01 | 938.51 | 626.01 |
| 270 | Ac-ETF$r8AYWAQCba$AWNlea-NH₂ | 270 | | 1914.01 | 958.42 | 1915.02 | 958.01 | 639.01 |
| 271 | Ac-LTF$r8EYWAQCba$AWNlea-NH₂ | 271 | | 1956.06 | 979.42 | 1957.07 | 979.04 | 653.03 |
| 272 | Ac-ETF$r8EYWAQCba$AWNlea-NH₂ | 272 | | 1972.01 | 987.06 | 1973.02 | 987.01 | 658.34 |
| 273 | Ac-ETF$r8EYWAQCba$AWNlea-NH₂ | 273 | iso2 | 1972.01 | 987.06 | 1973.02 | 987.01 | 658.34 |
| 274 | Ac-LTF$r8AYWAQCba$SAFa-NH₂ | 274 | | 1832.99 | 917.89 | 1834 | 917.5 | 612 |
| 275 | Ac-LTF$r8AYWAQCba$SAFa-NH₂ | 275 | iso2 | 1832.99 | 918.07 | 1834 | 917.5 | 612 |
| 276 | Ac-ETF$r8AYWAQL$AWNlea-NH₂ | 276 | | 1902.01 | 952.22 | 1903.02 | 952.01 | 635.01 |
| 277 | Ac-LTF$r8EYWAQL$AWNlea-NH₂ | 277 | | 1944.06 | 973.5 | 1945.07 | 973.04 | 649.03 |
| 278 | Ac-ETF$r8EYWAQL$AWNlea-NH₂ | 278 | | 1960.01 | 981.46 | 1961.02 | 981.01 | 654.34 |
| 279 | Dmaac-LTF$r8EYWAQhL$SAA-NH₂ | 279 | | 1788.98 | 896.06 | 1789.99 | 895.5 | 597.33 |
| 280 | Hexac-LTF$r8EYWAQhL$SAA-NH₂ | 280 | | 1802 | 902.9 | 1803.01 | 902.01 | 601.67 |
| 281 | Napac-LTF$r8EYWAQhL$SAA-NH₂ | 281 | | 1871.99 | 937.58 | 1873 | 937 | 625 |
| 282 | Decac-LTF$r8EYWAQhL$SAA-NH₂ | 282 | | 1858.06 | 930.55 | 1859.07 | 930.04 | 620.36 |
| 283 | Admac-LTF$r8EYWAQhL$SAA-NH₂ | 283 | | 1866.03 | 934.07 | 1867.04 | 934.02 | 623.02 |
| 284 | Tmac-LTF$r8EYWAQhL$SAA-NH₂ | 284 | | 1787.99 | 895.41 | 1789 | 895 | 597 |
| 285 | Pam-LTF$r8EYWAQhL$SAA-NH₂ | 285 | | 1942.16 | 972.08 | 1943.17 | 972.09 | 648.39 |
| 286 | Ac-LTF$r8AYWAQCba$AANleA-NH₂ | 286 | iso2 | 1783.01 | 892.64 | 1784.02 | 892.51 | 595.34 |
| 287 | Ac-LTF34F2$r8EYWAQCba$AAIa-NH₂ | 287 | iso2 | 1876.99 | 939.62 | 1878 | 939.5 | 626.67 |
| 288 | Ac-LTF34F2$r8EYWAQCba$SAA-NH₂ | 288 | | 1779.91 | 892.07 | 1780.92 | 890.96 | 594.31 |
| 289 | Ac-LTF34F2$r8EYWAQCba$SAA-NH₂ | 289 | iso2 | 1779.91 | 891.61 | 1780.92 | 890.96 | 594.31 |
| 290 | Ac-LTF$r8EF4coohWAQCba$SAA-NH₂ | 290 | | 1771.92 | 887.54 | 1772.93 | 886.97 | 591.65 |
| 291 | Ac-LTF$r8EF4coohWAQCba$SAA-NH₂ | 291 | iso2 | 1771.92 | 887.63 | 1772.93 | 886.97 | 591.65 |
| 292 | Ac-LTF$r8EYWSQCba$SAA-NH₂ | 292 | | 1759.92 | 881.9 | 1760.93 | 880.97 | 587.65 |
| 293 | Ac-LTF$r8EYWSQCba$SAA-NH₂ | 293 | iso2 | 1759.92 | 881.9 | 1760.93 | 880.97 | 587.65 |
| 294 | Ac-LTF$r8EYWAQhL$SAA-NH₂ | 294 | | 1745.94 | 875.05 | 1746.95 | 873.98 | 582.99 |
| 295 | Ac-LTF$r8AYWAQhL$SAF-NH₂ | 295 | | 1763.97 | 884.02 | 1764.98 | 882.99 | 589 |
| 296 | Ac-LTF$r8AYWAQhL$SAF-NH₂ | 296 | iso2 | 1763.97 | 883.56 | 1764.98 | 882.99 | 589 |
| 297 | Ac-LTF34F2$r8AYWAQhL$SAA-NH₂ | 297 | | 1723.92 | 863.67 | 1724.93 | 862.97 | 575.65 |
| 298 | Ac-LTF34F2$r8AYWAQhL$SAA-NH₂ | 298 | iso2 | 1723.92 | 864.04 | 1724.93 | 862.97 | 575.65 |
| 299 | Ac-LTF$r8AF4coohWAQhL$SAA-NH₂ | 299 | | 1715.93 | 859.44 | 1716.94 | 858.97 | 572.98 |
| 300 | Ac-LTF$r8AF4coohWAQhL$SAA-NH₂ | 300 | iso2 | 1715.93 | 859.6 | 1716.94 | 858.97 | 572.98 |
| 301 | Ac-LTF$r8AYWSQhL$SAA-NH₂ | 301 | | 1703.93 | 853.96 | 1704.94 | 852.97 | 568.98 |
| 302 | Ac-LTF$r8AYWSQhL$SAA-NH₂ | 302 | iso2 | 1703.93 | 853.59 | 1704.94 | 852.97 | 568.98 |
| 303 | Ac-LTF$r8EYWAQL$AANleA-NH₂ | 303 | | 1829.01 | 915.45 | 1830.02 | 915.51 | 610.68 |
| 304 | Ac-LTF34F2$r8AYWAQL$AANleA-NH₂ | 304 | | 1806.99 | 904.58 | 1808 | 904.5 | 603.34 |
| 305 | Ac-LTF$r8AF4coohWAQL$AANleA-NH₂ | 305 | | 1799 | 901.6 | 1800.01 | 900.51 | 600.67 |
| 306 | Ac-LTF$r8AYWSQL$AANleA-NH₂ | 306 | | 1787 | 894.75 | 1788.01 | 894.51 | 596.67 |
| 307 | Ac-LTF34F2$r8AYWAQhL$AANleA-NH₂ | 307 | | 1821 | 911.79 | 1822.01 | 911.51 | 608.01 |
| 308 | Ac-LTF34F2$r8AYWAQhL$AANleA-NH₂ | 308 | iso2 | 1821 | 912.61 | 1822.01 | 911.51 | 608.01 |
| 309 | Ac-LTF$r8AF4coohWAQhL$AANleA-NH₂ | 309 | | 1813.02 | 907.95 | 1814.03 | 907.52 | 605.35 |
| 310 | Ac-LTF$r8AF4coohWAQhL$AANleA-NH₂ | 310 | iso2 | 1813.02 | 908.54 | 1814.03 | 907.52 | 605.35 |
| 311 | Ac-LTF$r8AYWSQhL$AANleA-NH₂ | 311 | | 1801.02 | 901.84 | 1802.03 | 901.52 | 601.35 |
| 312 | Ac-LTF$r8AYWSQhL$AANleA-NH₂ | 312 | iso2 | 1801.02 | 902.62 | 1802.03 | 901.52 | 601.35 |
| 313 | Ac-LTF$r8AYWAQhL$AAAAa-NH₂ | 313 | | 1814.01 | 908.63 | 1815.02 | 908.01 | 605.68 |
| 314 | Ac-LTF$r8AYWAQhL$AAAAa-NH₂ | 314 | iso2 | 1814.01 | 908.34 | 1815.02 | 908.01 | 605.68 |
| 315 | Ac-LTF$r8AYWAQL$AAAAAa-NH₂ | 315 | | 1871.04 | 936.94 | 1872.05 | 936.53 | 624.69 |
| 316 | Ac-LTF$r8AYWAQL$AAAAAAa-NH₂ | 316 | iso2 | 1942.07 | 972.5 | 1943.08 | 972.04 | 648.37 |
| 317 | Ac-LTF$r8AYWAQL$AAAAAAa-NH₂ | 317 | iso1 | 1942.07 | 972.5 | 1943.08 | 972.04 | 648.37 |
| 318 | Ac-LTF$r8EYWAQhL$AANleA-NH₂ | 318 | | 1843.03 | 922.54 | 1844.04 | 922.52 | 615.35 |
| 319 | Ac-AATF$r8AYWAQL$AANleA-NH₂ | 319 | | 1800 | 901.39 | 1801.01 | 901.01 | 601.01 |
| 320 | Ac-LTF$r8AYWAQL$AANleAA-NH₂ | 320 | | 1842.04 | 922.45 | 1843.05 | 922.03 | 615.02 |
| 321 | Ac-ALTF$r8AYWAQL$AANleAA-NH₂ | 321 | | 1913.08 | 957.94 | 1914.09 | 957.55 | 638.7 |
| 322 | Ac-LTF$r8AYWAQCba$AANleAA-NH₂ | 322 | | 1854.04 | 928.43 | 1855.05 | 928.03 | 619.02 |
| 323 | Ac-LTF$r8AYWAQhL$AANleAA-NH₂ | 323 | | 1856.06 | 929.4 | 1857.07 | 929.04 | 619.69 |
| 324 | Ac-LTF$r8EYWAQCba$SAAA-NH₂ | 324 | | 1814.96 | 909.37 | 1815.97 | 908.49 | 605.99 |
| 325 | Ac-LTF$r8EYWAQCba$SAAA-NH₂ | 325 | iso2 | 1814.96 | 909.37 | 1815.97 | 908.49 | 605.99 |
| 326 | Ac-LTF$r8EYWAQCba$SAAAA-NH₂ | 326 | | 1886 | 944.61 | 1887.01 | 944.01 | 629.67 |
| 327 | Ac-LTF$r8EYWAQCba$SAAAA-NH₂ | 327 | iso2 | 1886 | 944.61 | 1887.01 | 944.01 | 629.67 |
| 328 | Ac-ALTF$r8EYWAQCba$SAA-NH₂ | 328 | | 1814.96 | 909.09 | 1815.97 | 908.49 | 605.99 |
| 329 | Ac-ALTF$r8EYWAQCba$SAAA-NH₂ | 329 | | 1886 | 944.61 | 1887.01 | 944.01 | 629.67 |
| 330 | Ac-ALTF$r8EYWAQCba$SAA-NH₂ | 330 | iso2 | 1814.96 | 909.09 | 1815.97 | 908.49 | 605.99 |
| 331 | Ac-LTF$r8EYWAQL$AAAAAa-NH₂ | 331 | iso2 | 1929.04 | 966.08 | 1930.05 | 965.53 | 644.02 |
| 332 | Ac-LTF$r8EY6clWAQCba$SAA-NH₂ | 332 | | 1777.89 | 890.78 | 1778.9 | 889.95 | 593.64 |
| 333 | Ac-LTF$r8EF4cooh6clWAQCba$SANleA-NH₂ | 333 | | 1918.96 | 961.27 | 1919.97 | 960.49 | 640.66 |
| 334 | Ac-LTF$r8EF4cooh6clWAQCba$SANleA-NH₂ | 334 | iso2 | 1918.96 | 961.27 | 1919.97 | 960.49 | 640.66 |
| 335 | Ac-LTF$r8EF4cooh6clWAQCba$AAIa-NH₂ | 335 | | 1902.97 | 953.03 | 1903.98 | 952.49 | 635.33 |
| 336 | Ac-LTF$r8EF4cooh6clWAQCba$AAIa-NH₂ | 336 | iso2 | 1902.97 | 953.13 | 1903.98 | 952.49 | 635.33 |
| 337 | Ac-LTF$r8AY6clWAQL$AAAAAa-NH₂ | 337 | | 1905 | 954.61 | 1906.01 | 953.51 | 636.01 |
| 338 | Ac-LTF$r8AY6clWAQL$AAAAAa-NH₂ | 338 | iso2 | 1905 | 954.9 | 1906.01 | 953.51 | 636.01 |
| 339 | Ac-F$r8AY6clWEAL$AAAAAAa-NH₂ | 339 | | 1762.89 | 883.01 | 1763.9 | 882.45 | 588.64 |
| 340 | Ac-ETF$r8EYWAQL$AAAAAa-NH₂ | 340 | | 1945 | 974.31 | 1946.01 | 973.51 | 649.34 |
| 341 | Ac-ETF$r8EYWAQL$AAAAAa-NH₂ | 341 | iso2 | 1945 | 974.49 | 1946.01 | 973.51 | 649.34 |
| 342 | Ac-LTF$r8EYWAQL$AAAAAAa-NH₂ | 342 | | 2000.08 | 1001.6 | 2001.09 | 1001.05 | 667.7 |
| 343 | Ac-LTF$r8EYWAQL$AAAAAAa-NH₂ | 343 | iso2 | 2000.08 | 1001.6 | 2001.09 | 1001.05 | 667.7 |
| 344 | Ac-LTF$r8AYWAQL$AANleAAa-NH₂ | 344 | | 1913.08 | 958.58 | 1914.09 | 957.55 | 638.7 |
| 345 | Ac-LTF$r8AYWAQL$AANleAAa-NH₂ | 345 | iso2 | 1913.08 | 958.58 | 1914.09 | 957.55 | 638.7 |
| 346 | Ac-LTF$r8EYWAQCba$AAAAAa-NH₂ | 346 | | 1941.04 | 972.55 | 1942.05 | 971.53 | 648.02 |
| 347 | Ac-LTF$r8EYWAQCba$AAAAAa-NH₂ | 347 | iso2 | 1941.04 | 972.55 | 1942.05 | 971.53 | 648.02 |
| 348 | Ac-LTF$r8EF4coohWAQCba$AAAAAa-NH₂ | 348 | | 1969.04 | 986.33 | 1970.05 | 985.53 | 657.35 |
| 349 | Ac-LTF$r8EF4coohWAQCba$AAAAAa-NH₂ | 349 | iso2 | 1969.04 | 986.06 | 1970.05 | 985.53 | 657.35 |
| 350 | Ac-LTF$r8EYWSQCba$AAAAAa-NH₂ | 350 | | 1957.04 | 980.04 | 1958.05 | 979.53 | 653.35 |
| 351 | Ac-LTF$r8EYWSQCba$AAAAAa-NH₂ | 351 | iso2 | 1957.04 | 980.04 | 1958.05 | 979.53 | 653.35 |
| 352 | Ac-LTF$r8EYWAQCba$SAAa-NH₂ | 352 | | 1814.96 | 909 | 1815.97 | 908.49 | 605.99 |
| 353 | Ac-LTF$r8EYWAQCba$SAAa-NH₂ | 353 | iso2 | 1814.96 | 909 | 1815.97 | 908.49 | 605.99 |
| 354 | Ac-ALTF$r8EYWAQCba$SAAa-NH₂ | 354 | | 1886 | 944.52 | 1887.01 | 944.01 | 629.67 |
| 355 | Ac-ALTF$r8EYWAQCba$SAAa-NH₂ | 355 | iso2 | 1886 | 944.98 | 1887.01 | 944.01 | 629.67 |
| 356 | Ac-ALTF$r8EYWAQCba$SAAAa-NH₂ | 356 | | 1957.04 | 980.04 | 1958.05 | 979.53 | 653.35 |
| 357 | Ac-ALTF$r8EYWAQCba$SAAAa-NH₂ | 357 | iso2 | 1957.04 | 980.04 | 1958.05 | 979.53 | 653.35 |
| 358 | Ac-AALTF$r8EYWAQCba$SAAAa-NH₂ | 358 | | 2028.07 | 1016.1 | 2029.08 | 1015.04 | 677.03 |
| 359 | Ac-AALTF$r8EYWAQCba$SAAAa-NH₂ | 359 | iso2 | 2028.07 | 1015.57 | 2029.08 | 1015.04 | 677.03 |
| 360 | Ac-RTF$r8EYWAQCba$SAA-NH₂ | 360 | | 1786.94 | 895.03 | 1787.95 | 894.48 | 596.65 |
| 361 | Ac-LRF$r8EYWAQCba$SAA-NH₂ | 361 | | 1798.98 | 901.51 | 1799.99 | 900.5 | 600.67 |
| 362 | Ac-LTF$r8EYWRQCba$SAA-NH₂ | 362 | | 1828.99 | 916.4 | 1830 | 915.5 | 610.67 |
| 363 | Ac-LTF$r8EYWARCba$SAA-NH₂ | 363 | | 1771.97 | 887.63 | 1772.98 | 886.99 | 591.66 |
| 364 | Ac-LTF$r8EYWAQCba$RAA-NH₂ | 364 | | 1812.99 | 908.08 | 1814 | 907.5 | 605.34 |
| 365 | Ac-LTF$r8EYWAQCba$SRA-NH₂ | 365 | | 1828.99 | 916.12 | 1830 | 915.5 | 610.67 |
| 366 | Ac-LTF$r8EYWAQCba$SAR-NH₂ | 366 | | 1828.99 | 916.12 | 1830 | 915.5 | 610.67 |
| 367 | 5-FAM-BaLTF$r8EYWAQCba$SAA-NH₂ | 367 | | 2131 | 1067.09 | 2132.01 | 1066.51 | 711.34 |
| 368 | 5-FAM-BaLTF$r8AYWAQL$AANleA-NH₂ | 368 | | 2158.08 | 1080.6 | 2159.09 | 1080.05 | 720.37 |
| 369 | Ac-LAF$r8EYWAQL$AANleA-NH₂ | 369 | | 1799 | 901.05 | 1800.01 | 900.51 | 600.67 |
| 370 | Ac-ATF$r8EYWAQL$AANleA-NH₂ | 370 | | 1786.97 | 895.03 | 1787.98 | 894.49 | 596.66 |
| 371 | Ac-AAF$r8EYWAQL$AANleA-NH₂ | 371 | | 1756.96 | 880.05 | 1757.97 | 879.49 | 586.66 |
| 372 | Ac-AAAF$r8EYWAQL$AANleA-NH₂ | 372 | | 1827.99 | 915.57 | 1829 | 915 | 610.34 |
| 373 | Ac-AAAAF$r8EYWAQL$AANleA-NH₂ | 373 | | 1899.03 | 951.09 | 1900.04 | 950.52 | 634.02 |
| 374 | Ac-AATF$r8EYWAQL$AANleA-NH₂ | 374 | | 1858 | 930.92 | 1859.01 | 930.01 | 620.34 |
| 375 | Ac-AALTF$r8EYWAQL$AANleA-NH₂ | 375 | | 1971.09 | 987.17 | 1972.1 | 986.55 | 658.04 |
| 376 | Ac-AAALTF$r8EYWAQL$AANleA-NH₂ | 376 | | 2042.12 | 1023.15 | 2043.13 | 1022.07 | 681.71 |
| 377 | Ac-LTF$r8EYWAQL$AANleAA-NH₂ | 377 | | 1900.05 | 952.02 | 1901.06 | 951.03 | 634.36 |
| 378 | Ac-ALTF$r8EYWAQL$AANleAA-NH₂ | 378 | | 1971.09 | 987.63 | 1972.1 | 986.55 | 658.04 |
| 379 | Ac-AALTF$r8EYWAQL$AANleAA-NH₂ | 379 | | 2042.12 | 1022.69 | 2043.13 | 1022.07 | 681.71 |
| 380 | Ac-LTF$r8EYWAQCba$AANleAA-NH₂ | 380 | | 1912.05 | 958.03 | 1913.06 | 957.03 | 638.36 |
| 381 | Ac-LTF$r8EYWAQhL$AANleAA-NH₂ | 381 | | 1914.07 | 958.68 | 1915.08 | 958.04 | 639.03 |
| 382 | Ac-ALTF$r8EYWAQhL$AANleAA-NH₂ | 382 | | 1985.1 | 994.1 | 1986.11 | 993.56 | 662.71 |
| 383 | Ac-LTF$r8ANmYWAQL$AANleA-NH₂ | 383 | | 1785.02 | 894.11 | 1786.03 | 893.52 | 596.01 |
| 384 | Ac-LTF$r8ANmYWAQL$AANleA-NH₂ | 384 | iso2 | 1785.02 | 894.11 | 1786.03 | 893.52 | 596.01 |
| 385 | Ac-LTF$r8AYNmWAQL$AANleA-NH₂ | 385 | | 1785.02 | 894.11 | 1786.03 | 893.52 | 596.01 |
| 386 | Ac-LTF$r8AYNmWAQL$AANleA-NH₂ | 386 | iso2 | 1785.02 | 894.11 | 1786.03 | 893.52 | 596.01 |
| 387 | Ac-LTF$r8AYAmwAQL$AANleA-NH₂ | 387 | | 1785.02 | 894.01 | 1786.03 | 893.52 | 596.01 |
| 388 | Ac-LTF$r8AYAmwAQL$AANleA-NH₂ | 388 | iso2 | 1785.02 | 894.01 | 1786.03 | 893.52 | 596.01 |
| 389 | Ac-LTF$r8AYWAibQL$AANleA-NH₂ | 389 | | 1785.02 | 894.01 | 1786.03 | 893.52 | 596.01 |
| 390 | Ac-LTF$r8AYWAibQL$AANleA-NH₂ | 390 | iso2 | 1785.02 | 894.01 | 1786.03 | 893.52 | 596.01 |
| 391 | Ac-LTF$r8AYWAQL$AAibNleA-NH₂ | 391 | | 1785.02 | 894.38 | 1786.03 | 893.52 | 596.01 |
| 392 | Ac-LTF$r8AYWAQL$AAibNleA-NH₂ | 392 | iso2 | 1785.02 | 894.38 | 1786.03 | 893.52 | 596.01 |
| 393 | Ac-LTF$r8AYWAQL$AaNleA-NH₂ | 393 | | 1771.01 | 887.54 | 1772.02 | 886.51 | 591.34 |
| 394 | Ac-LTF$r8AYWAQL$AaNleA-NH₂ | 394 | iso2 | 1771.01 | 887.54 | 1772.02 | 886.51 | 591.34 |
| 395 | Ac-LTF$r8AYWAQL$ASarNleA-NH₂ | 395 | | 1771.01 | 887.35 | 1772.02 | 886.51 | 591.34 |
| 396 | Ac-LTF$r8AYWAQL$ASarNleA-NH₂ | 396 | iso2 | 1771.01 | 887.35 | 1772.02 | 886.51 | 591.34 |
| 397 | Ac-LTF$r8AYWAQL$AANleAib-NH₂ | 397 | | 1785.02 | 894.75 | 1786.03 | 893.52 | 596.01 |
| 398 | Ac-LTF$r8AYWAQL$AANleAib-NH₂ | 398 | iso2 | 1785.02 | 894.75 | 1786.03 | 893.52 | 596.01 |
| 399 | Ac-LTF$r8AYWAQL$AANleNmA-NH₂ | 399 | | 1785.02 | 894.6 | 1786.03 | 893.52 | 596.01 |
| 400 | Ac-LTF$r8AYWAQL$AANleNmA-NH₂ | 400 | iso2 | 1785.02 | 894.6 | 1786.03 | 893.52 | 596.01 |
| 401 | Ac-LTF$r8AYWAQL$AANleSar-NH₂ | 401 | | 1771.01 | 886.98 | 1772.02 | 886.51 | 591.34 |
| 402 | Ac-LTF$r8AYWAQL$AANleSar-NH₂ | 402 | iso2 | 1771.01 | 886.98 | 1772.02 | 886.51 | 591.34 |
| 403 | Ac-LTF$r8AYWAQL$AANleAAib-NH₂ | 403 | | 1856.06 | | 1857.07 | 929.04 | 619.69 |
| 404 | Ac-LTF$r8AYWAQL$AANleAAib-NH₂ | 404 | iso2 | 1856.06 | | 1857.07 | 929.04 | 619.69 |
| 405 | Ac-LTF$r8AYWAQL$AANleANmA-NH₂ | 405 | | 1856.06 | 930.37 | 1857.07 | 929.04 | 619.69 |
| 406 | Ac-LTF$r8AYWAQL$AANleANmA-NH₂ | 406 | iso2 | 1856.06 | 930.37 | 1857.07 | 929.04 | 619.69 |
| 407 | Ac-LTF$r8AYWAQL$AANleAa-NH₂ | 407 | | 1842.04 | 922.69 | 1843.05 | 922.03 | 615.02 |
| 408 | Ac-LTF$r8AYWAQL$AANleAa-NH₂ | 408 | iso2 | 1842.04 | 922.69 | 1843.05 | 922.03 | 615.02 |
| 409 | Ac-LTF$r8AYWAQL$AANleASar-NH₂ | 409 | | 1842.04 | 922.6 | 1843.05 | 922.03 | 615.02 |
| 410 | Ac-LTF$r8AYWAQL$AANleASar-NH₂ | 410 | iso2 | 1842.04 | 922.6 | 1843.05 | 922.03 | 615.02 |
| 411 | Ac-LTF$/r8AYWAQL$/AANleA-NH₂ | 411 | | 1799.04 | 901.14 | 1800.05 | 900.53 | 600.69 |
| 412 | Ac-LTFAibAYWAQLAibAANleA-NH₂ | 412 | | 1648.9 | 826.02 | 1649.91 | 825.46 | 550.64 |
| 413 | Ac-LTF$r8Cou4YWAQL$AANleA-NH₂ | 413 | | 1975.05 | 989.11 | 1976.06 | 988.53 | 659.36 |
| 414 | Ac-LTF$r8Cou4YWAQL$AANleA-NH₂ | 414 | iso2 | 1975.05 | 989.11 | 1976.06 | 988.53 | 659.36 |
| 415 | Ac-LTF$r8AYWCou4QL$AANleA-NH₂ | 415 | | 1975.05 | 989.11 | 1976.06 | 988.53 | 659.36 |
| 416 | Ac-LTF$r8AYWAQL$Cou4ANleA-NH₂ | 416 | | 1975.05 | 989.57 | 1976.06 | 988.53 | 659.36 |
| 417 | Ac-LTF$r8AYWAQL$Cou4ANleA-NH₂ | 417 | iso2 | 1975.05 | 989.57 | 1976.06 | 988.53 | 659.36 |
| 418 | Ac-LTF$r8AYWAQL$ACou4NleA-NH₂ | 418 | | 1975.05 | 989.57 | 1976.06 | 988.53 | 659.36 |
| 419 | Ac-LTF$r8AYWAQL$ACou4NleA-NH₂ | 419 | iso2 | 1975.05 | 989.57 | 1976.06 | 988.53 | 659.36 |
| 420 | Ac-LTF$r8AYWAQL$AANleA-OH | 420 | | 1771.99 | 887.63 | 1773 | 887 | 591.67 |
| 421 | Ac-LTF$r8AYWAQL$AANleA-OH | 421 | iso2 | 1771.99 | 887.63 | 1773 | 887 | 591.67 |
| 422 | Ac-LTF$r8AYWAQL$AANleA-NHnPr | 422 | | 1813.05 | 908.08 | 1814.06 | 907.53 | 605.36 |
| 423 | Ac-LTF$r8AYWAQL$AANleA-NHnPr | 423 | iso2 | 1813.05 | 908.08 | 1814.06 | 907.53 | 605.36 |
| 424 | Ac-LTF$r8AYWAQL$AANleA-NHnBu33Me | 424 | | 1855.1 | 929.17 | 1856.11 | 928.56 | 619.37 |
| 425 | Ac-LTF$r8AYWAQL$AANleA-NHnBu33Me | 425 | iso2 | 1855.1 | 929.17 | 1856.11 | 928.56 | 619.37 |
| 426 | Ac-LTF$r8AYWAQL$AANleA-NHHex | 426 | | 1855.1 | 929.17 | 1856.11 | 928.56 | 619.37 |
| 427 | Ac-LTF$r8AYWAQL$AANleA-NHHex | 427 | iso2 | 1855.1 | 929.17 | 1856.11 | 928.56 | 619.37 |
| 428 | Ac-LTA$r8AYWAQL$AANleA-NH₂ | 428 | | 1694.98 | 849.33 | 1695.99 | 848.5 | 566 |
| 429 | Ac-LThL$r8AYWAQL$AANleA-NH₂ | 429 | | 1751.04 | 877.09 | 1752.05 | 876.53 | 584.69 |
| 430 | Ac-LTF$r8AYAAQL$AANleA-NH₂ | 430 | | 1655.97 | 829.54 | 1656.98 | 828.99 | 553 |
| 431 | Ac-LTF$r8AY2NalAQL$AANleA-NH₂ | 431 | | 1782.01 | 892.63 | 1783.02 | 892.01 | 595.01 |
| 432 | Ac-LTF$r8EYWCou4QCba$SAA-NH₂ | 432 | | 1947.97 | 975.8 | 1948.98 | 974.99 | 650.33 |
| 433 | Ac-LTF$r8EYWCou7QCba$SAA-NH₂ | 433 | | 16.03 | 974.9 | 17.04 | 9.02 | 6.35 |
| 434 | Ac-LTF % r8EYWAQCba % SAA-NH₂ | 434 | | 1745.94 | 874.8 | 1746.95 | 873.98 | 582.99 |
| 435 | Dmaac-LTF$r8EYWAQCba$SAA-NH₂ | 435 | | 1786.97 | 894.8 | 1787.98 | 894.49 | 596.66 |
| 436 | Dmaac-LTF$r8AYWAQL$AAAAAa-NH₂ | 436 | | 1914.08 | 958.2 | 1915.09 | 958.05 | 639.03 |
| 437 | Dmaac-LTF$r8AYWAQL$AAAAAa-NH₂ | 437 | iso2 | 1914.08 | 958.2 | 1915.09 | 958.05 | 639.03 |
| 438 | Dmaac-LTF$r8EYWAQL$AAAAAa-NH₂ | 438 | | 1972.08 | 987.3 | 1973.09 | 987.05 | 658.37 |
| 439 | Dmaac-LTF$r8EYWAQL$AAAAAa-NH₂ | 439 | iso2 | 1972.08 | 987.3 | 1973.09 | 987.05 | 658.37 |
| 440 | Dmaac-LTF$r8EF4coohWAQCba$AAIa-NH₂ | 440 | | 1912.05 | 957.4 | 1913.06 | 957.03 | 638.36 |
| 441 | Dmaac-LTF$r8EF4coohWAQCba$AAIa-NH₂ | 441 | iso2 | 1912.05 | 957.4 | 1913.06 | 957.03 | 638.36 |
| 442 | Dmaac-LTF$r8AYWAQL$AANleA-NH₂ | 442 | | 1814.05 | 908.3 | 1815.06 | 908.03 | 605.69 |
| 443 | Dmaac-LTF$r8AYWAQL$AANleA-NH₂ | 443 | iso2 | 1814.05 | 908.3 | 1815.06 | 908.03 | 605.69 |
| 444 | Ac-LTF % r8AYWAQL % AANleA-NH₂ | 444 | | 1773.02 | 888.37 | 1774.03 | 887.52 | 592.01 |
| 445 | Ac-LTF % r8EYWAQL % AAAAAa-NH₂ | 445 | | 1931.06 | 966.4 | 1932.07 | 966.54 | 644.69 |
| 446 | Cou6BaLTF$r8EYWAQhL$SAA-NH₂ | 446 | | 2018.05 | 1009.9 | 2019.06 | 1010.03 | 673.69 |
| 447 | Cou8BaLTF$r8EYWAQhL$SAA-NH₂ | 447 | | 1962.96 | 982.34 | 1963.97 | 982.49 | 655.32 |
| 448 | Ac-LTF4I$r8EYWAQL$AAAAAa-NH₂ | 448 | | 2054.93 | 1028.68 | 2055.94 | 1028.47 | 685.98 |
| 449 | Ac-LTF$r8EYWAQL$AAAAAa-NH₂ | 449 | | 1929.04 | 966.17 | 1930.05 | 965.53 | 644.02 |
| 550 | Ac-LTF$r8EYWAQL$AAAAAa-OH | 450 | | 1930.02 | 966.54 | 1931.03 | 966.02 | 644.35 |
| 551 | Ac-LTF$r8EYWAQL$AAAAAa-OH | 451 | iso2 | 1930.02 | 965.89 | 1931.03 | 966.02 | 644.35 |
| 552 | Ac-LTF$r8EYWAEL$AAAAAa-NH₂ | 452 | | 1930.02 | 966.82 | 1931.03 | 966.02 | 644.35 |
| 553 | Ac-LTF$r8EYWAEL$AAAAAa-NH₂ | 453 | iso2 | 1930.02 | 966.91 | 1931.03 | 966.02 | 644.35 |
| 554 | Ac-LTF$r8EYWAEL$AAAAAa-OH | 454 | | 1931.01 | 967.28 | 1932.02 | 966.51 | 644.68 |
| 555 | Ac-LTF$r8EY6clWAQL$AAAAAa-NH₂ | 455 | | 1963 | 983.28 | 1964.01 | 982.51 | 655.34 |
| 556 | Ac-LTF$r8EF4bOH2WAQL$AAAAAa-NH₂ | 456 | | 1957.05 | 980.04 | 1958.06 | 979.53 | 653.36 |
| 557 | Ac-AAALTF$r8EYWAQL$AAAAAa-NH₂ | 457 | | 2142.15 | 1072.83 | 2143.16 | 1072.08 | 715.06 |
| 558 | Ac-LTF34F2$r8EYWAQL$AAAAAa-NH₂ | 458 | | 1965.02 | 984.3 | 1966.03 | 983.52 | 656.01 |
| 559 | Ac-RTF$r8EYWAQL$AAAAAa-NH₂ | 459 | | 1972.06 | 987.81 | 1973.07 | 987.04 | 658.36 |
| 560 | Ac-LTA$r8EYWAQL$AAAAAa-NH₂ | 460 | | 1853.01 | 928.33 | 1854.02 | 927.51 | 618.68 |
| 561 | Ac-LTF$r8EYWAibQL$AAAAAa-NH₂ | 461 | | 1943.06 | 973.48 | 1944.07 | 972.54 | 648.69 |
| 562 | Ac-LTF$r8EYWAQL$AAibAAAa-NH₂ | 462 | | 1943.06 | 973.11 | 1944.07 | 972.54 | 648.69 |
| 563 | Ac-LTF$r8EYWAQL$AAAibAAa-NH₂ | 463 | | 1943.06 | 973.48 | 1944.07 | 972.54 | 648.69 |
| 564 | Ac-LTF$r8EYWAQL$AAAAibAa-NH₂ | 464 | | 1943.06 | 973.48 | 1944.07 | 972.54 | 648.69 |
| 565 | Ac-LTF$r8EYWAQL$AAAAAiba-NH₂ | 465 | | 1943.06 | 973.38 | 1944.07 | 972.54 | 648.69 |
| 566 | Ac-LTF$r8EYWAQL$AAAAAiba-NH₂ | 466 | iso2 | 1943.06 | 973.38 | 1944.07 | 972.54 | 648.69 |
| 567 | Ac-LTF$r8EYWAQL$AAAAAAib-NH₂ | 467 | | 1943.06 | 973.01 | 1944.07 | 972.54 | 648.69 |
| 568 | Ac-LTF$r8EYWAQL$AaAAAa-NH₂ | 468 | | 1929.04 | 966.54 | 1930.05 | 965.53 | 644.02 |
| 569 | Ac-LTF$r8EYWAQL$AAaAAa-NH₂ | 469 | | 1929.04 | 966.35 | 1930.05 | 965.53 | 644.02 |
| 570 | Ac-LTF$r8EYWAQL$AAAaAa-NH₂ | 470 | | 1929.04 | 966.54 | 1930.05 | 965.53 | 644.02 |
| 571 | Ac-LTF$r8EYWAQL$AAAaAa-NH₂ | 471 | iso2 | 1929.04 | 966.35 | 1930.05 | 965.53 | 644.02 |
| 572 | Ac-LTF$r8EYWAQL$AAAAaa-NH₂ | 472 | | 1929.04 | 966.35 | 1930.05 | 965.53 | 644.02 |
| 573 | Ac-LTF$r8EYWAQL$AAAAAA-NH₂ | 473 | | 1929.04 | 966.35 | 1930.05 | 965.53 | 644.02 |
| 574 | Ac-LTF$r8EYWAQL$ASarAAAa-NH₂ | 474 | | 1929.04 | 966.54 | 1930.05 | 965.53 | 644.02 |
| 575 | Ac-LTF$r8EYWAQL$AASarAAa-NH₂ | 475 | | 1929.04 | 966.35 | 1930.05 | 965.53 | 644.02 |
| 576 | Ac-LTF$r8EYWAQL$AAASarAa-NH₂ | 476 | | 1929.04 | 966.35 | 1930.05 | 965.53 | 644.02 |
| 577 | Ac-LTF$r8EYWAQL$AAAASara-NH₂ | 477 | | 1929.04 | 966.35 | 1930.05 | 965.53 | 644.02 |
| 578 | Ac-LTF$r8EYWAQL$AAAAASar-NH₂ | 478 | | 1929.04 | 966.08 | 1930.05 | 965.53 | 644.02 |
| 579 | Ac-7LTF$r8EYWAQL$AAAAAa-NH₂ | 479 | | 1918.07 | 951.99 | 1919.08 | 960.04 | 640.37 |
| 581 | Ac-TF$r8EYWAQL$AAAAAa-NH₂ | 480 | | 1815.96 | 929.85 | 1816.97 | 908.99 | 606.33 |
| 582 | Ac-F$r8EYWAQL$AAAAAa-NH₂ | 481 | | 1714.91 | 930.92 | 1715.92 | 858.46 | 572.64 |
| 583 | Ac-LVF$r8EYWAQL$AAAAAa-NH₂ | 482 | | 1927.06 | 895.12 | 1928.07 | 964.54 | 643.36 |
| 584 | Ac-AAF$r8EYWAQL$AAAAAa-NH₂ | 483 | | 1856.98 | 859.51 | 1857.99 | 929.5 | 620 |
| 585 | Ac-LTF$r8EYWAQL$AAAAa-NH₂ | 484 | | 1858 | 824.08 | 1859.01 | 930.01 | 620.34 |
| 586 | Ac-LTF$r8EYWAQL$AAAa-NH₂ | 485 | | 1786.97 | 788.56 | 1787.98 | 894.49 | 596.66 |
| 587 | Ac-LTF$r8EYWAQL$AAa-NH₂ | 486 | | 1715.93 | 1138.57 | 1716.94 | 858.97 | 572.98 |
| 588 | Ac-LTF$r8EYWAQL$Aa-NH₂ | 487 | | 1644.89 | 1144.98 | 1645.9 | 823.45 | 549.3 |
| 589 | Ac-LTF$r8EYWAQL$a-NH₂ | 488 | | 1573.85 | 1113.71 | 1574.86 | 787.93 | 525.62 |
| 590 | Ac-LTF$r8EYWAQL$AAA-OH | 489 | | 1716.91 | 859.55 | 1717.92 | 859.46 | 573.31 |
| 591 | Ac-LTF$r8EYWAQL$A-OH | 490 | | 1574.84 | 975.14 | 1575.85 | 788.43 | 525.95 |
| 592 | Ac-LTF$r8EYWAQL$AAA-NH₂ | 491 | | 1715.93 | 904.75 | 1716.94 | 858.97 | 572.98 |
| 593 | Ac-LTF$r8EYWAQCba$SAA-OH | 492 | | 1744.91 | 802.49 | 1745.92 | 873.46 | 582.64 |
| 594 | Ac-LTF$r8EYWAQCba$S-OH | 493 | | 1602.83 | 913.53 | 1603.84 | 802.42 | 535.28 |
| 595 | Ac-LTF$r8EYWAQCba$S-NH₂ | 494 | | 1601.85 | 979.58 | 1602.86 | 801.93 | 534.96 |
| 596 | 4-FBzl-LTF$r8EYWAQL$AAAAAa-NH₂ | 495 | | 2009.05 | 970.52 | 2010.06 | 1005.53 | 670.69 |
| 597 | 4-FBzl-LTF$r8EYWAQCba$SAA-NH₂ | 496 | | 1823.93 | 965.8 | 1824.94 | 912.97 | 608.98 |
| 598 | Ac-LTF$r8RYWAQL$AAAAAa-NH₂ | 497 | | 1956.1 | 988.28 | 1957.11 | 979.06 | 653.04 |
| 599 | Ac-LTF$r8HYWAQL$AAAAAa-NH₂ | 498 | | 1937.06 | 1003.54 | 1938.07 | 969.54 | 646.69 |
| 600 | Ac-LTF$r8QYWAQL$AAAAAa-NH₂ | 499 | | 1928.06 | 993.92 | 1929.07 | 965.04 | 643.69 |
| 601 | Ac-LTF$r8CitYWAQL$AAAAAa-NH₂ | 500 | | 1957.08 | 987 | 1958.09 | 979.55 | 653.37 |
| 602 | Ac-LTF$r8GlaYWAQL$AAAAAa-NH₂ | 501 | | 1973.03 | 983 | 1974.04 | 987.52 | 658.68 |
| 603 | Ac-LTF$r8F4gYWAQL$AAAAAa-NH₂ | 502 | | 2004.1 | 937.86 | 2005.11 | 1003.06 | 669.04 |
| 604 | Ac-LTF$r82mRYWAQL$AAAAAa-NH₂ | 503 | | 1984.13 | 958.58 | 1985.14 | 993.07 | 662.38 |
| 605 | Ac-LTF$r8ipKYWAQL$AAAAAa-NH₂ | 504 | | 1970.14 | 944.52 | 1971.15 | 986.08 | 657.72 |
| 606 | Ac-LTF$r8F4NH₂YWAQL$AAAAAa-NH₂ | 505 | | 1962.08 | 946 | 1963.09 | 982.05 | 655.03 |
| 607 | Ac-LTF$r8EYWAAL$AAAAAa-NH₂ | 506 | | 1872.02 | 959.32 | 1873.03 | 937.02 | 625.01 |
| 608 | Ac-LTF$r8EYWALL$AAAAAa-NH₂ | 507 | | 1914.07 | 980.88 | 1915.08 | 958.04 | 639.03 |
| 609 | Ac-LTF$r8EYWAAibL$AAAAAa-NH₂ | 508 | | 1886.03 | 970.61 | 1887.04 | 944.02 | 629.68 |
| 610 | Ac-LTF$r8EYWASL$AAAAAa-NH₂ | 509 | | 1888.01 | 980.51 | 1889.02 | 945.01 | 630.34 |
| 611 | Ac-LTF$r8EYWANL$AAAAAa-NH₂ | 510 | | 1915.02 | 1006.41 | 1916.03 | 958.52 | 639.35 |
| 612 | Ac-LTF$r8EYWACitL$AAAAAa-NH₂ | 511 | | 1958.07 | | 1959.08 | 980.04 | 653.7 |
| 613 | Ac-LTF$r8EYWAHL$AAAAAa-NH₂ | 512 | | 1938.04 | 966.24 | 1939.05 | 970.03 | 647.02 |
| 614 | Ac-LTF$r8EYWARL$AAAAAa-NH₂ | 513 | | 1957.08 | | 1958.09 | 979.55 | 653.37 |
| 615 | Ac-LTF$r8EpYWAQL$AAAAAa-NH₂ | 514 | | 2009.01 | | 2010.02 | 1005.51 | 670.68 |
| 616 | Cbm-LTF$r8EYWAQCba$SAA-NH₂ | 515 | | 1590.85 | | 1591.86 | 796.43 | 531.29 |
| 617 | Cbm-LTF$r8EYWAQL$AAAAAa-NH₂ | 516 | | 1930.04 | | 1931.05 | 966.03 | 644.35 |
| 618 | Ac-LTF$r8EYWAQL$SAAAAa-NH₂ | 517 | | 1945.04 | 1005.11 | 1946.05 | 973.53 | 649.35 |
| 619 | Ac-LTF$r8EYWAQL$AAAASa-NH₂ | 518 | | 1945.04 | 986.52 | 1946.05 | 973.53 | 649.35 |
| 620 | Ac-LTF$r8EYWAQL$SAAASa-NH₂ | 519 | | 1961.03 | 993.27 | 1962.04 | 981.52 | 654.68 |
| 621 | Ac-LTF$r8EYWAQTba$AAAAAa-NH₂ | 520 | | 1943.06 | 983.1 | 1944.07 | 972.54 | 648.69 |
| 622 | Ac-LTF$r8EYWAQAdm$AAAAAa-NH₂ | 521 | | 2007.09 | 990.31 | 2008.1 | 1004.55 | 670.04 |
| 623 | Ac-LTF$r8EYWAQCha$AAAAAa-NH₂ | 522 | | 1969.07 | 987.17 | 1970.08 | 985.54 | 657.36 |
| 624 | Ac-LTF$r8EYWAQhCha$AAAAAa-NH₂ | 523 | | 1983.09 | 1026.11 | 1984.1 | 992.55 | 662.04 |
| 625 | Ac-LTF$r8EYWAQF$AAAAAa-NH₂ | 524 | | 1963.02 | 957.01 | 1964.03 | 982.52 | 655.35 |
| 626 | Ac-LTF$r8EYWAQhF$AAAAAa-NH₂ | 525 | | 1977.04 | 1087.81 | 1978.05 | 989.53 | 660.02 |
| 627 | Ac-LTF$r8EYWAQL$AANleAAa-NH₂ | 526 | | 1971.09 | 933.45 | 1972.1 | 986.55 | 658.04 |
| 628 | Ac-LTF$r8EYWAQAdm$AANleAAa-NH₂ | 527 | | 2049.13 | 1017.97 | 2050.14 | 1025.57 | 684.05 |
| 629 | 4-FBz-BaLTF$r8EYWAQL$AAAAAa-NH₂ | 528 | | 2080.08 | | 2081.09 | 1041.05 | 694.37 |
| 630 | 4-FBz-BaLTF$r8EYWAQCba$SAA-NH₂ | 529 | | 1894.97 | | 1895.98 | 948.49 | 632.66 |
| 631 | Ac-LTF$r5EYWAQL$s8AAAAAa-NH₂ | 530 | | 1929.04 | 1072.68 | 1930.05 | 965.53 | 644.02 |
| 632 | Ac-LTF$r5EYWAQCba$s8SAA-NH₂ | 531 | | 1743.92 | 1107.79 | 1744.93 | 872.97 | 582.31 |
| 633 | Ac-LTF$r8EYWAQL$AAhhLAAa-NH₂ | 532 | | 1999.12 | | 2000.13 | 1000.57 | 667.38 |
| 634 | Ac-LTF$r8EYWAQL$AAAAAAAa-NH₂ | 533 | | 2071.11 | | 2072.12 | 1036.56 | 691.38 |
| 635 | Ac-LTF$r8EYWAQL$AAAAAAAAa-NH₂ | 534 | | 2142.15 | 778.1 | 2143.16 | 1072.08 | 715.06 |
| 636 | Ac-LTF$r8EYWAQL$AAAAAAAAAa-NH₂ | 535 | | 2213.19 | 870.53 | 2214.2 | 1107.6 | 738.74 |
| 637 | Ac-LTA$r8EYAAQCba$SAA-NH₂ | 536 | | 1552.85 | | 1553.86 | 777.43 | 518.62 |
| 638 | Ac-LTA$r8EYAAQL$AAAAAa-NH₂ | 537 | | 1737.97 | 779.45 | 1738.98 | 869.99 | 580.33 |
| 639 | Ac-LTF$r8EPmpWAQL$AAAAAa-NH₂ | 538 | | 2007.03 | 779.54 | 2008.04 | 1004.52 | 670.02 |
| 640 | Ac-LTF$r8EPmpWAQCba$SAA-NH₂ | 539 | | 1821.91 | 838.04 | 1822.92 | 911.96 | 608.31 |
| 641 | Ac-ATF$r8HYWAQL$S-NH₂ | 540 | | 1555.82 | 867.83 | 1556.83 | 778.92 | 519.61 |
| 642 | Ac-LTF$r8HAWAQL$S-NH₂ | 541 | | 1505.84 | 877.91 | 1506.85 | 753.93 | 502.95 |
| 643 | Ac-LTF$r8HYWAQA$S-NH₂ | 542 | | 1555.82 | 852.52 | 1556.83 | 778.92 | 519.61 |
| 644 | Ac-LTF$r8EYWAQCba$SA-NH₂ | 543 | | 1672.89 | 887.18 | 1673.9 | 837.45 | 558.64 |
| 645 | Ac-LTF$r8EYWAQL$SAA-NH₂ | 544 | | 1731.92 | 873.32 | 1732.93 | 866.97 | 578.31 |
| 646 | Ac-LTF$r8HYWAQCba$SAA-NH₂ | 545 | | 1751.94 | 873.05 | 1752.95 | 876.98 | 584.99 |
| 647 | Ac-LTF$r8SYWAQCba$SAA-NH₂ | 546 | | 1701.91 | 844.88 | 1702.92 | 851.96 | 568.31 |
| 648 | Ac-LTF$r8RYWAQCba$SAA-NH₂ | 547 | | 1770.98 | 865.58 | 1771.99 | 886.5 | 591.33 |
| 649 | Ac-LTF$r8KYWAQCba$SAA-NH₂ | 548 | | 1742.98 | 936.57 | 1743.99 | 872.5 | 582 |
| 650 | Ac-LTF$r8QYWAQCba$SAA-NH₂ | 549 | | 1742.94 | 930.93 | 1743.95 | 872.48 | 581.99 |
| 651 | Ac-LTF$r8EYWAACba$SAA-NH₂ | 550 | | 1686.9 | 1032.45 | 1687.91 | 844.46 | 563.31 |
| 652 | Ac-LTF$r8EYWAQCba$AAA-NH₂ | 551 | | 1727.93 | 895.46 | 1728.94 | 864.97 | 576.98 |
| 653 | Ac-LTF$r8EYWAQL$AAAAA-OH | 552 | | 1858.99 | 824.54 | 1860 | 930.5 | 620.67 |
| 654 | Ac-LTF$r8EYWAQL$AAAA-OH | 553 | | 1787.95 | 894.48 | 1788.96 | 894.98 | 596.99 |
| 655 | Ac-LTF$r8EYWAQL$AA-OH | 554 | | 1645.88 | 856 | 1646.89 | 823.95 | 549.63 |
| 656 | Ac-LTF$r8AF4bOH2WAQL$AAAAAa-NH₂ | 555 | | | | | | |
| 657 | Ac-LTF$r8AF4bOH2WAAL$AAAAAa-NH₂ | 556 | | | | | | |
| 658 | Ac-LTF$r8EF4bOH2WAQCba$SAA-NH₂ | 557 | | | | | | |
| 659 | Ac-LTF$r8ApYWAQL$AAAAAa-NH₂ | 558 | | | | | | |
| 660 | Ac-LTF$r8ApYWAAL$AAAAAa-NH₂ | 559 | | | | | | |
| 661 | Ac-LTF$r8EpYWAQCba$SAA-NH₂ | 560 | | | | | | |
| 662 | Ac-LTF$rda6AYWAQL$da5AAAAAa-NH₂ | 561 | | 1974.06 | 934.44 | | | |
| 663 | Ac-LTF$rda6EYWAQCba$da5SAA-NH₂ | 562 | | 1846.95 | 870.52 | | 869.94 | |
| 664 | Ac-LTF$rda6EYWAQL$da5AAAAAa-NH₂ | 563 | | | | | | |
| 665 | Ac-LTF$ra9EYWAQL$a6AAAAAa-NH₂ | 564 | | | 936.57 | | 935.51 | |
| 666 | Ac-LTF$ra9EYWAQL$a6AAAAAa-NH₂ | 565 | | | | | | |
| 667 | Ac-LTF$ra9EYWAQCba$a6SAA-NH₂ | 566 | | | | | | |
| 668 | Ac-LTA$ra9EYWAQCba$a6SAA-NH₂ | 567 | | | | | | |
| 669 | 5-FAM-BaLTF$ra9EYWAQCba$a6SAA-NH₂ | 568 | | | | | | |
| 670 | 5-FAM-BaLTF$r8EYWAQL$AAAAAa-NH₂ | 569 | | 2316.11 | | | | |
| 671 | 5-FAM-BaLTF$/r8EYWAQL$/AAAAAa-NH₂ | 570 | | 2344.15 | | | | |
| 672 | 5-FAM-BaLTA$r8EYWAQL$AAAAAa-NH₂ | 571 | | 2240.08 | | | | |
| 673 | 5-FAM-BaLTF$r8AYWAQL$AAAAAa-NH₂ | 572 | | 2258.11 | | | | |
| 674 | 5-FAM-BaATF$r8EYWAQL$AAAAAa-NH₂ | 573 | | 2274.07 | | | | |
| 675 | 5-FAM-BaLAF$r8EYWAQL$AAAAAa-NH₂ | 574 | | 2286.1 | | | | |
| 676 | 5-FAM-BaLTF$r8EAWAQL$AAAAAa-NH₂ | 575 | | 2224.09 | | | | |
| 677 | 5-FAM-BaLTF$r8EYAAQL$AAAAAa-NH₂ | 576 | | 2201.07 | | | | |
| 678 | 5-FAM-BaLTA$r8EYAAQL$AAAAAa-NH₂ | 577 | | 2125.04 | | | | |
| 679 | 5-FAM-BaLTF$r8EYWAAL$AAAAAa-NH₂ | 578 | | 2259.09 | | | | |
| 680 | 5-FAM-BaLTF$r8EYWAQA$AAAAAa-NH₂ | 579 | | 2274.07 | | | | |
| 681 | 5-FAM-BaLTF$/r8EYWAQCba$/SAA-NH₂ | 580 | | 2159.03 | | | | |
| 682 | 5-FAM-BaLTA$r8EYWAQCba$SAA-NH₂ | 581 | | 2054.97 | | | | |
| 683 | 5-FAM-BaLTF$r8EYAAQCba$SAA-NH₂ | 582 | | 2015.96 | | | | |
| 684 | 5-FAM-BaLTA$r8EYAAQCba$SAA-NH₂ | 583 | | 1939.92 | | | | |
| 685 | 5-FAM-BaQSQQTF$r8NLWRLL$QN-NH₂ | 584 | | 2495.23 | | | | |
| 686 | 5-TAMRA-BaLTF$r8EYWAQCba$SAA-NH₂ | 585 | | 2186.1 | | | | |
| 687 | 5-TAMRA-BaLTA$r8EYWAQCba$SAA-NH₂ | 586 | | 2110.07 | | | | |
| 688 | 5-TAMRA-BaLTF$r8EYAAQCba$SAA-NH₂ | 587 | | 2071.06 | | | | |
| 689 | 5-TAMRA-BaLTA$r8EYAAQCba$SAA-NH₂ | 588 | | 1995.03 | | | | |
| 690 | 5-TAMRA-BaLTF$/r8EYWAQCba$/SAA-NH₂ | 589 | | 2214.13 | | | | |
| 691 | 5-TAMRA-BaLTF$r8EYWAQL$AAAAAa-NH₂ | 590 | | 2371.22 | | | | |
| 692 | 5-TAMRA-BaLTA$r8EYWAQL$AAAAAa-NH₂ | 591 | | 2295.19 | | | | |
| 693 | 5-TAMRA-BaLTF$/r8EYWAQL$/AAAAAa-NH₂ | 592 | | 2399.25 | | | | |
| 694 | Ac-LTF$r8EYWCou7QCba$SAA-OH | 593 | | 1947.93 | | | | |
| 695 | Ac-LTF$r8EYWCou7QCba$S-OH | 594 | | 1805.86 | | | | |
| 696 | Ac-LTA$r8EYWCou7QCba$SAA-NH₂ | 595 | | 1870.91 | | | | |
| 697 | Ac-LTF$r8EYACou7QCba$SAA-NH₂ | 596 | | 1831.9 | | | | |
| 698 | Ac-LTA$r8EYACou7QCba$SAA-NH₂ | 597 | | 1755.87 | | | | |
| 699 | Ac-LTF$/r8EYWCou7QCba$/SAA-NH₂ | 598 | | 1974.98 | | | | |
| 700 | Ac-LTF$r8EYWCou7QL$AAAAAa-NH₂ | 599 | | 2132.06 | | | | |
| 701 | Ac-LTF$/r8EYWCou7QL$/AAAAAa-NH₂ | 600 | | 2160.09 | | | | |
| 702 | Ac-LTF$r8EYWCou7QL$AAAAA-OH | 601 | | 2062.01 | | | | |
| 703 | Ac-LTF$r8EYWCou7QL$AAAA-OH | 602 | | 1990.97 | | | | |
| 704 | Ac-LTF$r8EYWCou7QL$AAA-OH | 603 | | 1919.94 | | | | |
| 705 | Ac-LTF$r8EYWCou7QL$AA-OH | 604 | | 1848.9 | | | | |
| 706 | Ac-LTF$r8EYWCou7QL$A-OH | 605 | | 1777.86 | | | | |
| 707 | Ac-LTF$r8EYWAQL$AAAASa-NH₂ | 606 | iso2 | | 974.4 | | 973.53 | |
| 708 | Ac-LTF$r8AYWAAL$AAAAAa-NH₂ | 607 | iso2 | 1814.01 | 908.82 | 1815.02 | 908.01 | 605.68 |
| 709 | Biotin-BaLTF$r8EYWAQL$AAAAAa-NH₂ | 608 | | 2184.14 | 1093.64 | 2185.15 | 1093.08 | 729.05 |
| 710 | Ac-LTF$r8HAWAQL$S-NH₂ | 609 | iso2 | 1505.84 | 754.43 | 1506.85 | 753.93 | 502.95 |
| 711 | Ac-LTF$r8EYWAQCba$SA-NH₂ | 610 | iso2 | 1672.89 | 838.05 | 1673.9 | 837.45 | 558.64 |
| 712 | Ac-LTF$r8HYWAQCba$SAA-NH₂ | 611 | iso2 | 1751.94 | 877.55 | 1752.95 | 876.98 | 584.99 |
| 713 | Ac-LTF$r8SYWAQCba$SAA-NH₂ | 612 | iso2 | 1701.91 | 852.48 | 1702.92 | 851.96 | 568.31 |
| 714 | Ac-LTF$r8RYWAQCba$SAA-NH₂ | 613 | iso2 | 1770.98 | 887.45 | 1771.99 | 886.5 | 591.33 |
| 715 | Ac-LTF$r8KYWAQCba$SAA-NH₂ | 614 | iso2 | 1742.98 | 872.92 | 1743.99 | 872.5 | 582 |
| 716 | Ac-LTF$r8EYWAQCba$AAA-NH₂ | 615 | iso2 | 1727.93 | 865.71 | 1728.94 | 864.97 | 576.98 |
| 717 | Ac-LTF$r8EYWAQL$AAAAAaBaC-NH₂ | 616 | | 2103.09 | 1053.12 | 2104.1 | 1052.55 | 702.04 |
| 718 | Ac-LTF$r8EYWAQL$AAAAAadPeg4C-NH₂ | 617 | | 2279.19 | 1141.46 | 2280.2 | 1140.6 | 760.74 |
| 719 | Ac-LTA$r8AYWAAL$AAAAAa-NH₂ | 618 | | 1737.98 | 870.43 | 1738.99 | 870 | 580.33 |
| 720 | Ac-LTF$r8AYAAAL$AAAAAa-NH₂ | 619 | | 1698.97 | 851 | 1699.98 | 850.49 | 567.33 |
| 721 | 5-FAM-BaLTF$r8AYWAAL$AAAAAa-NH₂ | 620 | | 2201.09 | 1101.87 | 2202.1 | 1101.55 | 734.7 |
| 722 | Ac-LTA$r8AYWAQL$AAAAAa-NH₂ | 621 | | 1795 | 898.92 | 1796.01 | 898.51 | 599.34 |
| 723 | Ac-LTF$r8AYAAQL$AAAAAa-NH₂ | 622 | | 1755.99 | 879.49 | 1757 | 879 | 586.34 |
| 724 | Ac-LTF$rda6AYWAAL$da5AAAAAa-NH₂ | 623 | | 1807.97 | | 1808.98 | 904.99 | 603.66 |
| 725 | FITC-BaLTF$r8EYWAQL$AAAAAa-NH₂ | 624 | | 2347.1 | 1174.49 | 2348.11 | 1174.56 | 783.37 |
| 726 | FITC-BaLTF$r8EYWAQCba$SAA-NH₂ | 625 | | 2161.99 | 1082.35 | 2163 | 1082 | 721.67 |
| 733 | Ac-LTF$r8EYWAQL$EAAAAa-NH₂ | 626 | | 1987.05 | 995.03 | 1988.06 | 994.53 | 663.36 |
| 734 | Ac-LTF$r8AYWAQL$EAAAAa-NH₂ | 627 | | 1929.04 | 966.35 | 1930.05 | 965.53 | 644.02 |
| 735 | Ac-LTF$r8EYWAQL$AAAAAaBaKbio-NH₂ | 628 | | 2354.25 | 1178.47 | 2355.26 | 1178.13 | 785.76 |
| 736 | Ac-LTF$r8AYWAAL$AAAAAa-NH₂ | 629 | | 1814.01 | 908.45 | 1815.02 | 908.01 | 605.68 |
| 737 | Ac-LTF$r8AYAAAL$AAAAAa-NH₂ | 630 | iso2 | 1698.97 | 850.91 | 1699.98 | 850.49 | 567.33 |
| 738 | Ac-LTF$r8AYAAQL$AAAAAa-NH₂ | 631 | iso2 | 1755.99 | 879.4 | 1757 | 879 | 586.34 |
| 739 | Ac-LTF$r8EYWAQL$EAAAAa-NH₂ | 632 | iso2 | 1987.05 | 995.21 | 1988.06 | 994.53 | 663.36 |
| 740 | Ac-LTF$r8AYWAQL$EAAAAa-NH₂ | 633 | iso2 | 1929.04 | 966.08 | 1930.05 | 965.53 | 644.02 |
| 741 | Ac-LTF$r8EYWAQCba$SAAAAa-NH₂ | 634 | | 1957.04 | 980.04 | 1958.05 | 979.53 | 653.35 |
| 742 | Ac-LTF$r8EYWAQLStAAA$r5AA-NH₂ | 635 | | 2023.12 | 1012.83 | 2024.13 | 1012.57 | 675.38 |
| 743 | Ac-LTF$r8EYWAQL$A$AAA$A-NH₂ | 636 | | 2108.17 | 1055.44 | 2109.18 | 1055.09 | 703.73 |
| 744 | Ac-LTF$r8EYWAQL$AA$AAA$A-NH₂ | 637 | | 2179.21 | 1090.77 | 2180.22 | 1090.61 | 727.41 |
| 745 | Ac-LTF$r8EYWAQL$AAA$AAA$A-NH₂ | 638 | | 2250.25 | 1126.69 | 2251.26 | 1126.13 | 751.09 |
| 746 | Ac-AAALTF$r8EYWAQL$AAA-OH | 639 | | 1930.02 | | 1931.03 | 966.02 | 644.35 |
| 747 | Ac-AAALTF$r8EYWAQL$AAA-NH₂ | 640 | | 1929.04 | 965.85 | 1930.05 | 965.53 | 644.02 |
| 748 | Ac-AAAALTF$r8EYWAQL$AAA-NH₂ | 641 | | 2000.08 | 1001.4 | 2001.09 | 1001.05 | 667.7 |
| 749 | Ac-AAAAALTF$r8EYWAQL$AAA-NH₂ | 642 | | 2071.11 | 1037.13 | 2072.12 | 1036.56 | 691.38 |
| 750 | Ac-AAAAAALTF$r8EYWAQL$AAA-NH₂ | 643 | | 2142.15 | | 2143.16 | 1072.08 | 715.06 |
| 751 | Ac-LTF$rda6EYWAQCba$da6SAA-NH₂ | 644 | iso2 | 1751.89 | 877.36 | 1752.9 | 876.95 | 584.97 |
| 752 | Ac-t$r5wya$r5f4CF3ekllr-NH₂ | 645 | | | 844.25 | | | |
| 753 | Ac-tawy$r5nf4CF3e$r5llr-NH₂ | 646 | | | 837.03 | | | |
| 754 | Ac-tawya$r5f4CF3ek$r51r-NH₂ | 647 | | | 822.97 | | | |
| 755 | Ac-tawyanf4CF3e$r511r$r5a-NH₂ | 648 | | | 908.35 | | | |
| 756 | Ac-t$s8wyanf4CF3e$r511r-NH₂ | 649 | | | 858.03 | | | |
| 757 | Ac-tawy$s8nf4CF3ekll$r5a-NH₂ | 650 | | | 879.86 | | | |
| 758 | Ac-tawya$s8f4CF3ekllr$r5a-NH₂ | 651 | | | 936.38 | | | |
| 759 | Ac-tawy$s8naekll$r5a-NH₂ | 652 | | | 844.25 | | | |
| 760 | 5-FAM-Batawy$s8nf4CF3ekll$r5a-NH₂ | 653 | | | | | | |
| 761 | 5-FAM-Batawy$s8naekll$r5a-NH₂ | 654 | | | | | | |
| 762 | Ac-tawy$s8nf4CF3eall$r5a-NH₂ | 655 | | | | | | |
| 763 | Ac-tawy$s8nf4CF3ekll$r5aaaaa-NH₂ | 656 | | | | | | |
| 764 | Ac-tawy$s8nf4CF3eall$r5aaaaa-NH₂ | 657 | | | | | | |

TABLE 1a shows a selection of peptidomimetic macrocycles.

TABLE 1a

| | | SEQ | | Exact | Found | Calc | Calc | Calc |
|---|---|---|---|---|---|---|---|---|
| SP | Sequence | ID NO: | Isomer | Mass | Mass | (M + 1)/1 | (M + 2)/2 | (M + 3)/3 |
| 244 | Ac-LTF$r8EF4coohWAQCba$SANleA-NH₂ | 658 | | 1885 | 943.59 | 1886.01 | 943.51 | 629.34 |
| 331 | Ac-LTF$r8EYWAQL$AAAAAa-NH₂ | 659 | iso2 | 1929.04 | 966.08 | 1930.05 | 965.53 | 644.02 |
| 555 | Ac-LTF$r8EY6clWAQL$AAAAAa-NH₂ | 660 | | 1963 | 983.28 | 1964.01 | 982.51 | 655.34 |
| 557 | Ac-AAALTF$r8EYWAQL$AAAAAa-NH₂ | 661 | | 2142.15 | 1072.83 | 2143.16 | 1072.08 | 715.06 |
| 558 | Ac-LTF34F2$r8EYWAQL$AAAAAa-NH₂ | 662 | | 1965.02 | 984.3 | 1966.03 | 983.52 | 656.01 |
| 562 | Ac-LTF$r8EYWAQL$AAibAAAa-NH₂ | 663 | | 1943.06 | 973.11 | 1944.07 | 972.54 | 648.69 |
| 564 | Ac-LTF$r8EYWAQL$AAAAibAa-NH₂ | 664 | | 1943.06 | 973.48 | 1944.07 | 972.54 | 648.69 |
| 566 | Ac-LTF$r8EYWAQL$AAAAAiba-NH₂ | 665 | iso2 | 1943.06 | 973.38 | 1944.07 | 972.54 | 648.69 |
| 567 | Ac-LTF$r8EYWAQL$AAAAAAib-NH₂ | 666 | | 1943.06 | 973.01 | 1944.07 | 972.54 | 648.69 |
| 572 | Ac-LTF$r8EYWAQL$AAAAaa-NH₂ | 667 | | 1929.04 | 966.35 | 1930.05 | 965.53 | 644.02 |
| 573 | Ac-LTF$r8EYWAQL$AAAAAA-NH₂ | 668 | | 1929.04 | 966.35 | 1930.05 | 965.53 | 644.02 |
| 578 | Ac-LTF$r8EYWAQL$AAAAASar-NH₂ | 669 | | 1929.04 | 966.08 | 1930.05 | 965.53 | 644.02 |
| 551 | Ac-LTF$r8EYWAQL$AAAAAa-OH | 670 | iso2 | 1930.02 | 965.89 | 1931.03 | 966.02 | 644.35 |
| 662 | Ac-LTF$rda6AYWAQL$da5AAAAAa-NH₂ | 671 | | 1974.06 | 934.44 | | 933.49 | |
| 367 | 5-FAM-BaLTF$r8EYWAQCba$SAA-NH₂ | 672 | | 2131 | 1067.09 | 2132.01 | 1066.51 | 711.34 |
| 349 | Ac-LTF$r8EF4coohWAQCba$AAAAAa-NH₂ | 673 | iso2 | 1969.04 | 986.06 | 1970.05 | 985.53 | 657.35 |
| 347 | Ac-LTF$r8EYWAQCba$AAAAAa-NH₂ | 674 | iso2 | 1941.04 | 972.55 | 1942.05 | 971.53 | 648.02 |

TABLE 1b shows a further selection of peptidomimetic macrocycles.

TABLE 1b

| | | SEQ | | Exact | Found | Calc | Calc | Calc |
|---|---|---|---|---|---|---|---|---|
| SP | Sequence | ID NO: | Isomer | Mass | Mass | (M + 1)/1 | (M + 2)/2 | (M+ 3)/3 |
| 581 | Ac-TF$r8EYWAQL$AAAAAa-NH₂ | 675 | z | 1815.96 | 929.85 | 1816.97 | 908.99 | 606.33 |
| 582 | Ac-F$r8EYWAQL$AAAAAa-NH₂ | 676 | | 1714.91 | 930.92 | 1715.92 | 858.46 | 572.64 |
| 583 | Ac-LVF$r8EYWAQL$AAAAAa-NH₂ | 677 | | 1927.06 | 895.12 | 1928.07 | 964.54 | 643.36 |
| 584 | Ac-AAF$r8EYWAQL$AAAAAa-NH₂ | 678 | | 1856.98 | 859.51 | 1857.99 | 929.5 | 620 |
| 585 | Ac-LTF$r8EYWAQL$AAAAa-NH₂ | 679 | | 1858 | 824.08 | 1859.01 | 930.01 | 620.34 |
| 586 | Ac-LTF$r8EYWAQL$AAAa-NH₂ | 680 | | 1786.97 | 788.56 | 1787.98 | 894.49 | 596.66 |
| 587 | Ac-LTF$r8EYWAQL$AAa-NH₂ | 681 | | 1715.93 | 1138.57 | 1716.94 | 858.97 | 572.98 |
| 588 | Ac-LTF$r8EYWAQL$Aa-NH₂ | 682 | | 1644.89 | 1144.98 | 1645.9 | 823.45 | 549.3 |
| 589 | Ac-LTF$r8EYWAQL$a-NH₂ | 683 | | 1573.85 | 1113.71 | 1574.86 | 787.93 | 525.62 |

In the sequences shown above and elsewhere, the following abbreviations are used: “Nle” represents norleucine, “Aib” represents 2-aminoisobutyric acid, “Ac” represents acetyl, and “Pr” represents propionyl. Amino acids represented as “$” are alpha-Me S5-pentenyl-alanine olefin amino acids connected by an all-carbon crosslinker comprising one double bond. Amino acids represented as “$r5” are alpha-Me R₅-pentenyl-alanine olefin amino acids connected by an all-carbon comprising one double bond. Amino acids represented as “$s8” are alpha-Me S8-octenyl-alanine olefin amino acids connected by an all-carbon crosslinker comprising one double bond. Amino acids represented as “$r8” are alpha-Me R8-octenyl-alanine olefin amino acids connected by an all-carbon crosslinker comprising one double bond. “Ahx” represents an aminocyclohexyl linker.

The crosslinkers are linear all-carbon crosslinker comprising eight or eleven carbon atoms between the alpha carbons of each amino acid. Amino acids represented as “$/” are alpha-Me S5-pentenyl-alanine olefin amino acids that are not connected by any crosslinker. Amino acids represented as “$/r5” are alpha-Me R5-pentenyl-alanine olefin amino acids that are not connected by any crosslinker. Amino acids represented as “$/s8” are alpha-Me S8-octenyl-alanine olefin amino acids that are not connected by any crosslinker. Amino acids represented as “$/r8” are alpha-Me R8-octenyl-alanine olefin amino acids that are not connected by any crosslinker.

Amino acids represented as “Amw” are alpha-Me tryptophan amino acids. Amino acids represented as “Aml” are alpha-Me leucine amino acids. Amino acids represented as “Amf” are alpha-Me phenylalanine amino acids. Amino acids represented as “2ff” are 2-fluoro-phenylalanine amino acids. Amino acids represented as “3ff” are 3-fluoro-phenylalanine amino acids. Amino acids represented as “St” are amino acids comprising two pentenyl-alanine olefin side chains, each of which is crosslinked to another amino acid as indicated. Amino acids represented as “St//” are amino acids comprising two pentenyl-alanine olefin side chains that are not crosslinked. Amino acids represented as “% St” are amino acids comprising two pentenyl-alanine olefin side chains, each of which is crosslinked to another amino acid as indicated via fully saturated hydrocarbon crosslinks. Amino acids represented as “Ba” are beta-alanine. The lower-case character “e” or “z” within the designation of a crosslinked amino acid (e.g. “$er8” or “$zr8”) represents the configuration of the double bond (E or Z, respectively). In other contexts, lower-case letters such as “a” or “f” represent D amino acids (e.g. D-alanine, or D-phenylalanine, respectively).

Amino acids designated as “NmW” represent N-methyltryptophan. Amino acids designated as “NmY” represent N-methyltyrosine. Amino acids designated as “NmA” represent N-methylalanine. “Kbio” represents a biotin group attached to the side chain amino group of a lysine residue. Amino acids designated as “Sar” represent sarcosine. Amino acids designated as “Cha” represent cyclohexyl alanine. Amino acids designated as “Cpg” represent cyclopentyl glycine. Amino acids designated as “Chg” represent cyclohexyl glycine. Amino acids designated as “Cba” represent cyclobutyl alanine. Amino acids designated as “F41” represent 4-iodo phenylalanine. “7L” represents N15 isotopic leucine. Amino acids designated as “F3Cl” represent 3-chloro phenylalanine. Amino acids designated as “F4cooh” represent 4-carboxy phenylalanine. Amino acids designated as “F34F2” represent 3,4-difluoro phenylalanine. Amino acids designated as “6clW” represent 6-chloro tryptophan. Amino acids designated as “$rda6” represent alpha-Me R6-hexynyl-alanine alkynyl amino acids, crosslinked via a dialkyne bond to a second alkynyl amino acid.

Amino acids designated as “$da5” represent alpha-Me S5-pentynyl-alanine alkynyl amino acids, wherein the alkyne forms one half of a dialkyne bond with a second alkynyl amino acid. Amino acids designated as “$ra9” represent alpha-Me R9-nonynyl-alanine alkynyl amino acids, crosslinked via an alkyne metathesis reaction with a second alkynyl amino acid. Amino acids designated as “$a6” represent alpha-Me S6-hexynyl-alanine alkynyl amino acids, crosslinked via an alkyne metathesis reaction with a second alkynyl amino acid. The designation “iso1” or “iso2” indicates that the peptidomimetic macrocycle is a single isomer.

Amino acids designated as “Cit” represent citrulline. Amino acids designated as “Cou4”, “Cou6”, “Cou7” and “Cou8”, respectively, represent the following structures:

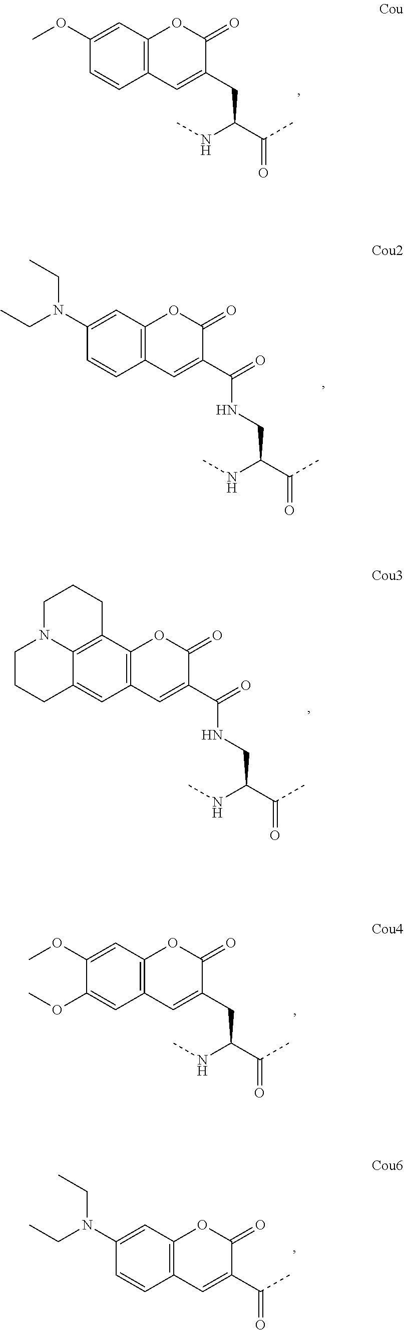

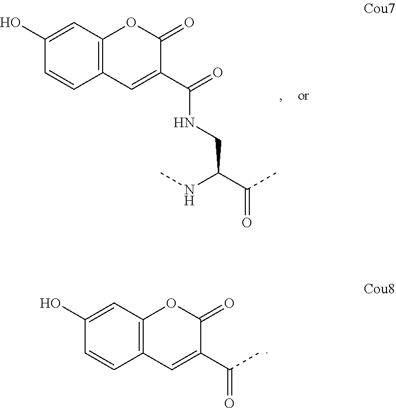

In some embodiments, a peptidomimetic macrocycle is obtained in more than one isomer, for example due to the configuration of a double bond within the structure of the crosslinker (E vs Z). Such isomers can or cannot be separable by conventional chromatographic methods. In some embodiments, one isomer has improved biological properties relative to the other isomer. In one embodiment, an E crosslinker olefin isomer of a peptidomimetic macrocycle has better solubility, better target affinity, better in vivo or in vitro efficacy, higher helicity, or improved cell permeability relative to its Z counterpart. In another embodiment, a Z crosslinker olefin isomer of a peptidomimetic macrocycle has better solubility, better target affinity, better in vivo or in vitro efficacy, higher helicity, or improved cell permeability relative to its E counterpart.

TABLE 1c shows non-limiting examples of peptidomimetic macrocycles.

TABLE 1c

| SP | SEQ ID | |
|---|---|---|
| # | NO: | Structure |
| 154 | 154 | 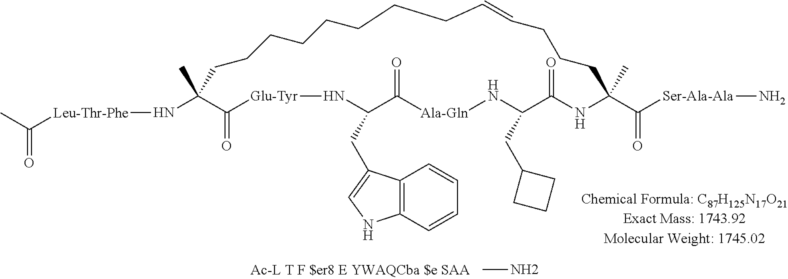 |
| 115 | 115 | 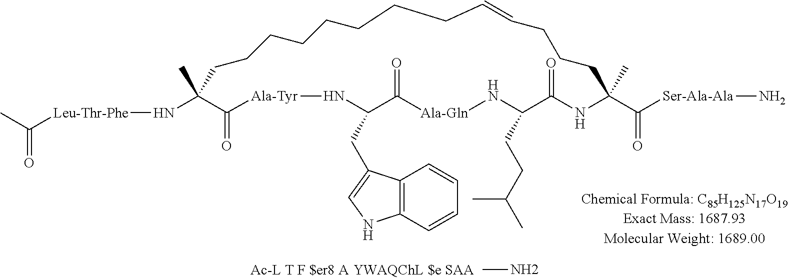 |
| 114 | 114 | 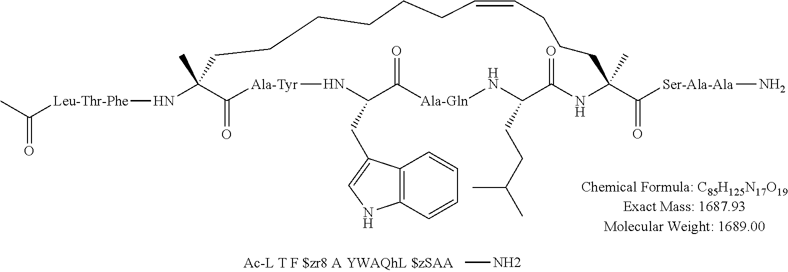 |
| 99 | 99 | 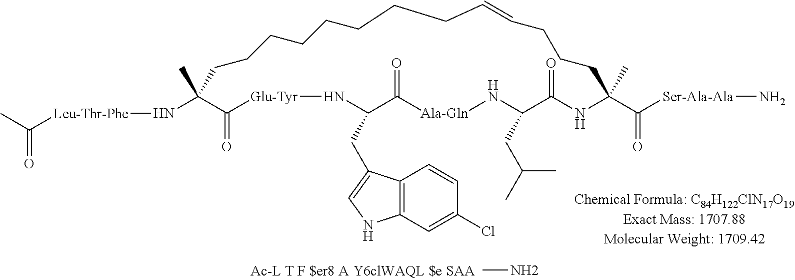 |
| 388 | 388 | 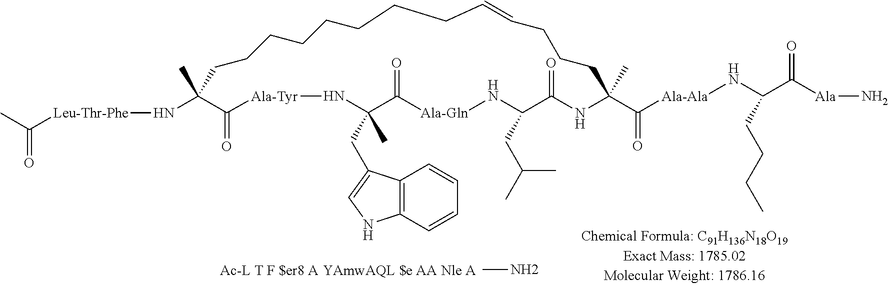 |
| 331 | 331 | 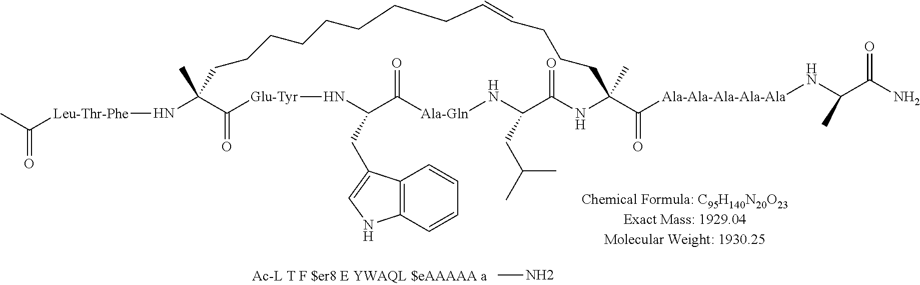 |
| 445 | 445 | 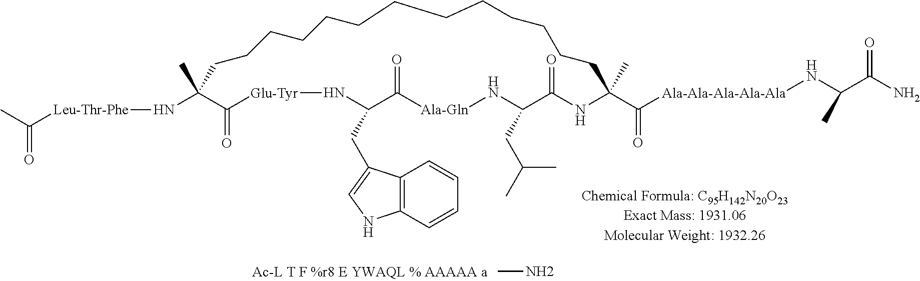 |
| 351 | 351 | 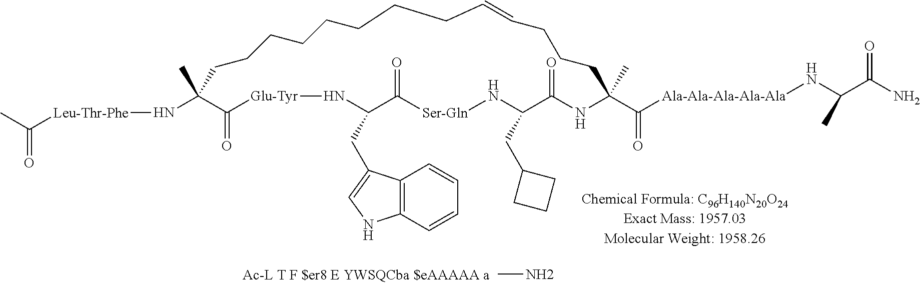 |
| 71 | 71 | 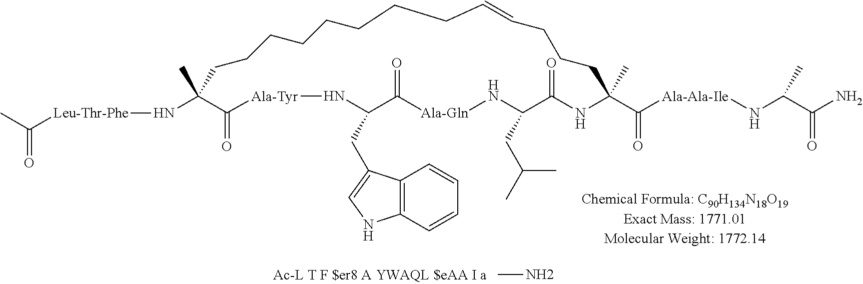 |
| 69 | 69 | 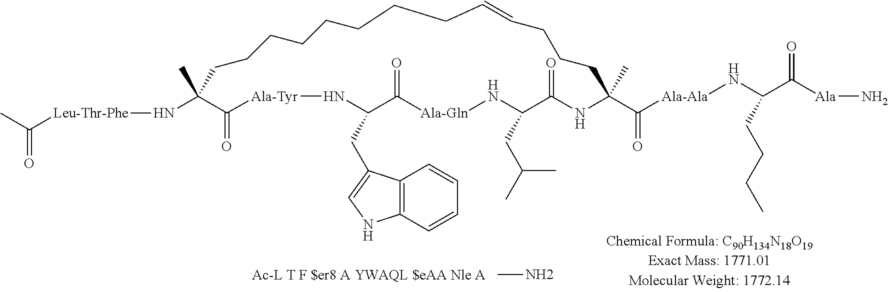 |
| 7 | 7 | 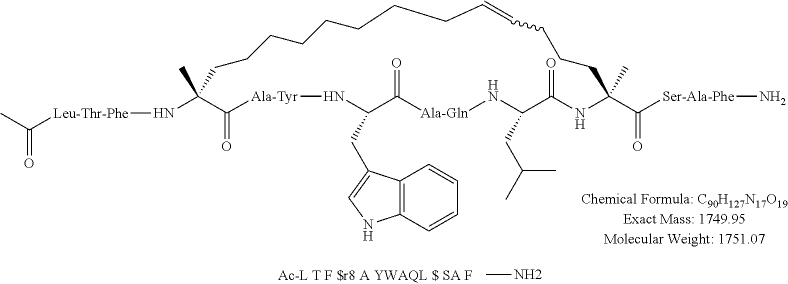 |
| 160 | 160 | 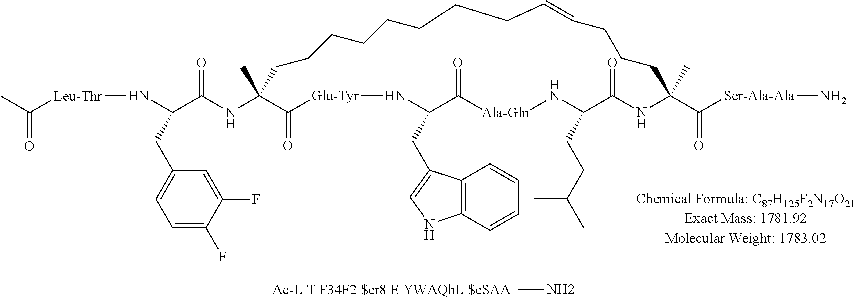 |
| 315 | 315 | 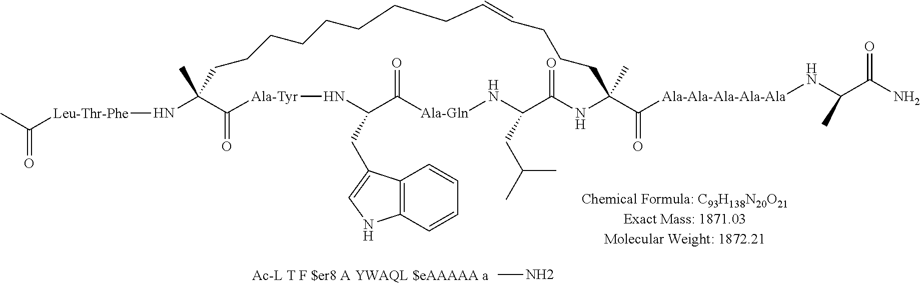 |
| 249 | 249 | 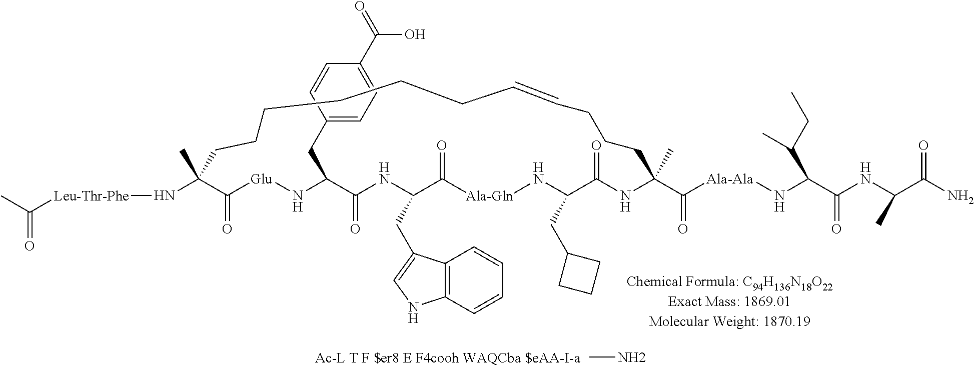 |
| 437 | 437 | 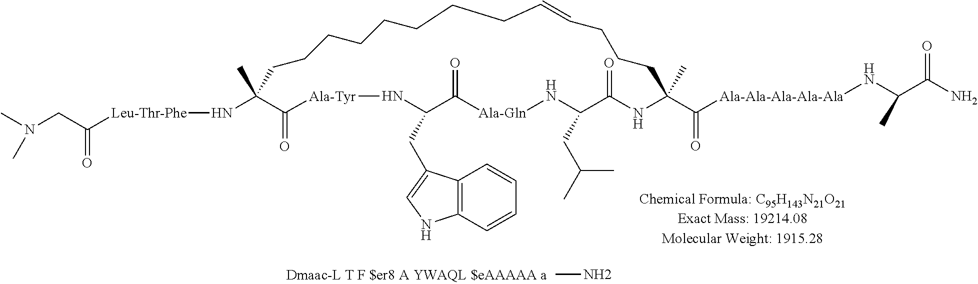 |
| 349 | 349 | 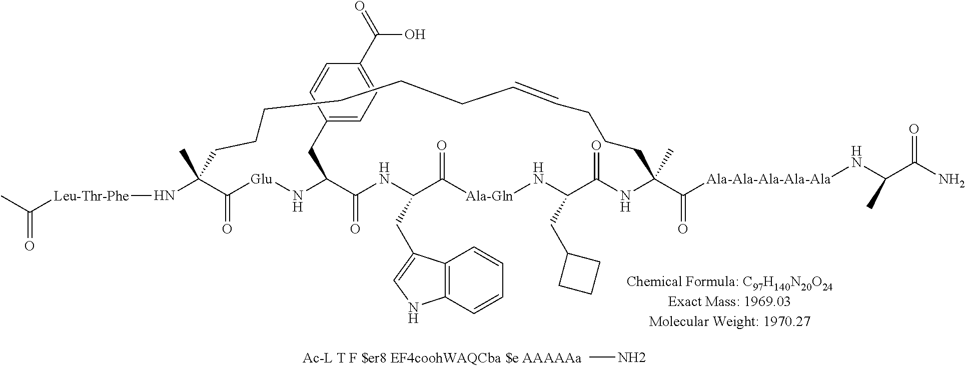 |
| 555 | 455 | 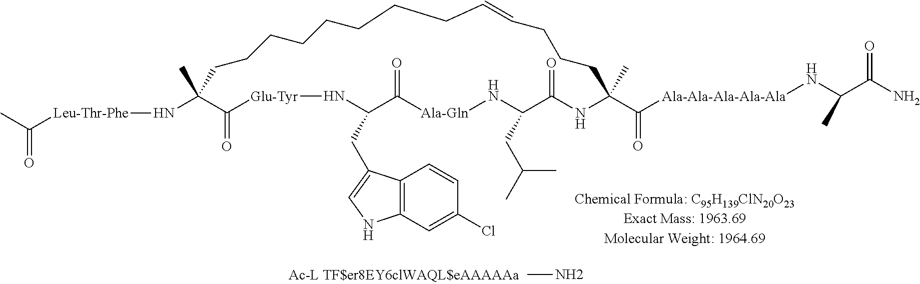 |
| 557 | 457 | 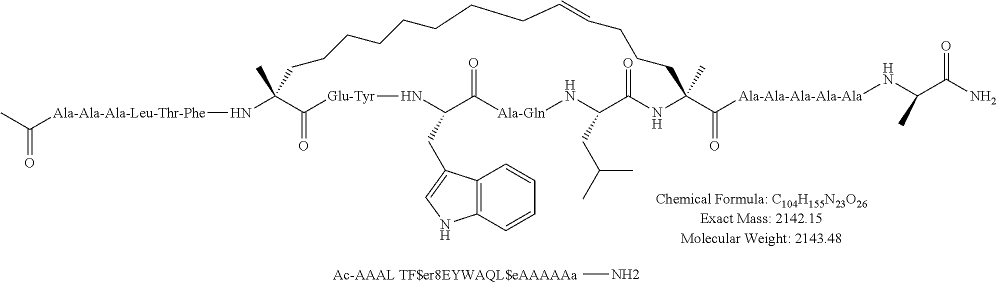 |
| 558 | 458 | 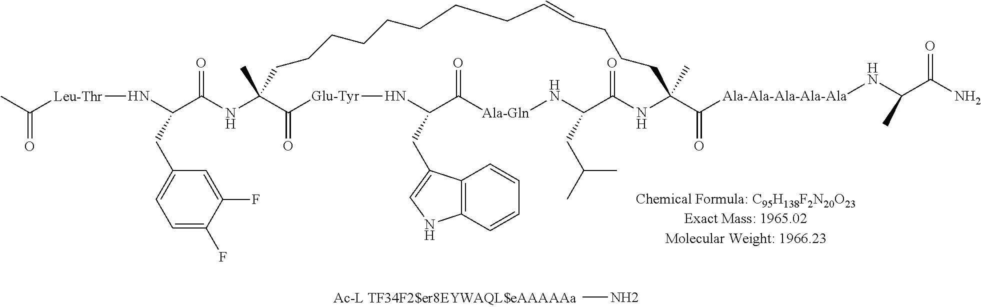 |
| 367 | 367 | 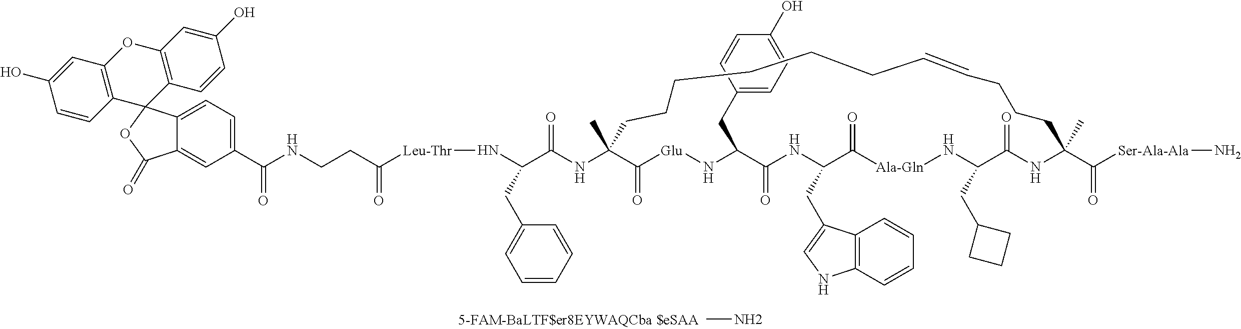 |
| 562 | 462 | 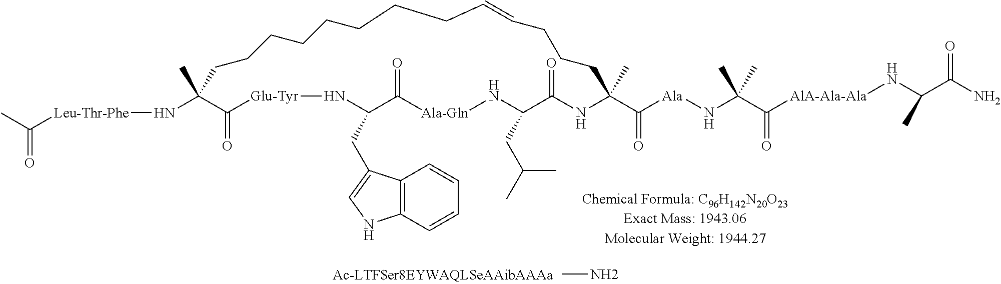 |
| 564 | 464 | 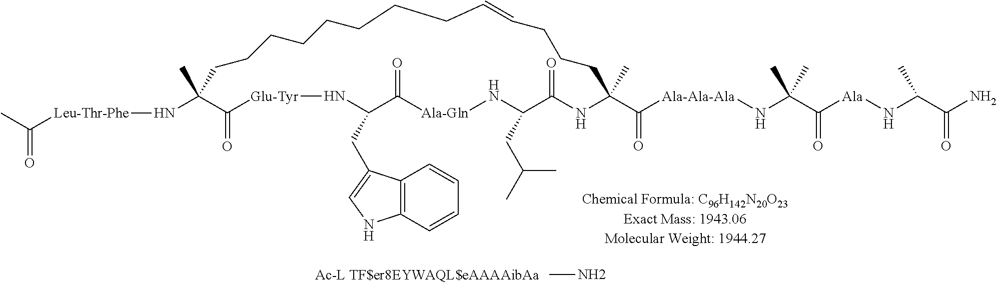 |
| 566 | 466 | 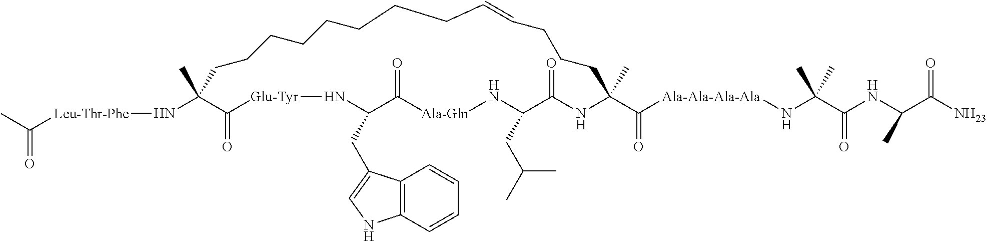 |
| 567 | 467 | 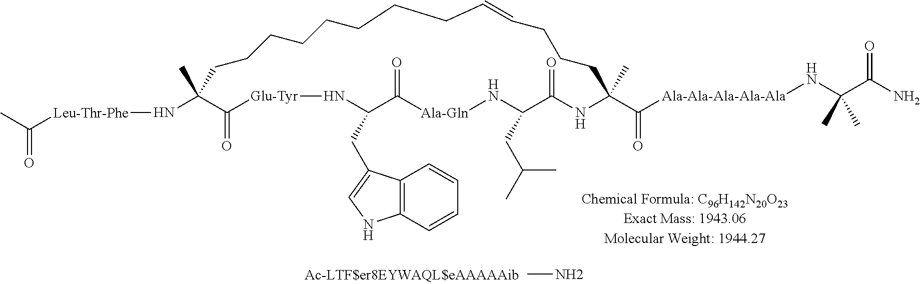 |
| 572 | 472 | 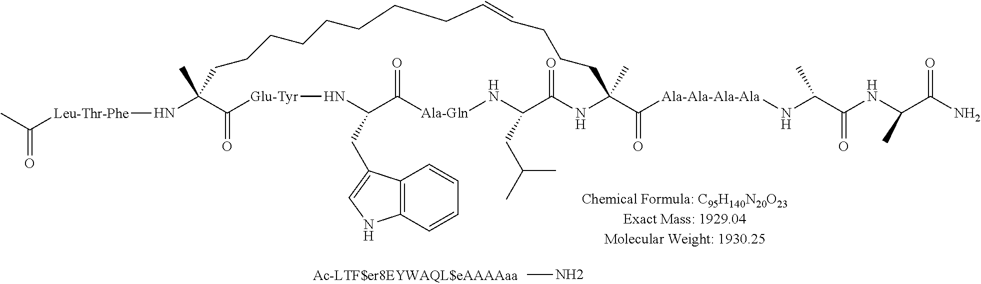 |
| 573 | 473 | 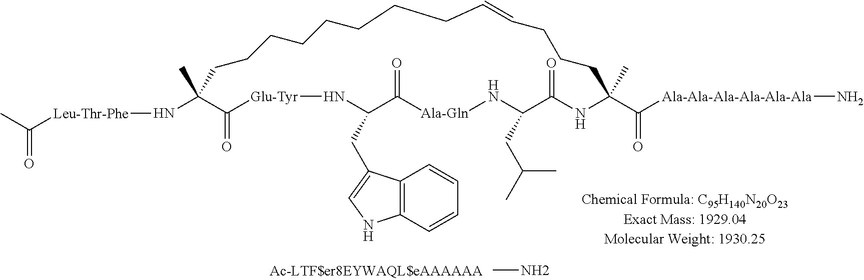 |
| 578 | 478 | 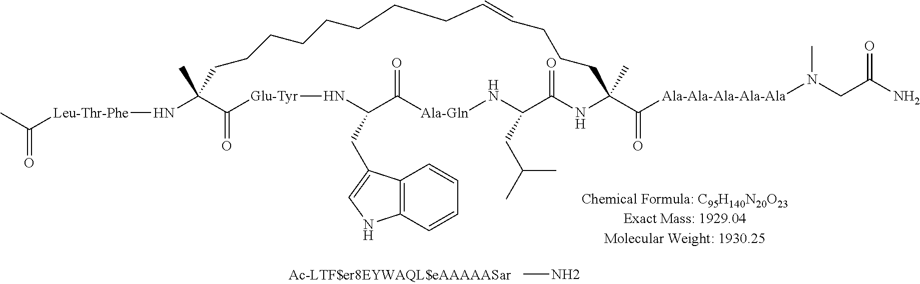 |
| 664 | 563 | 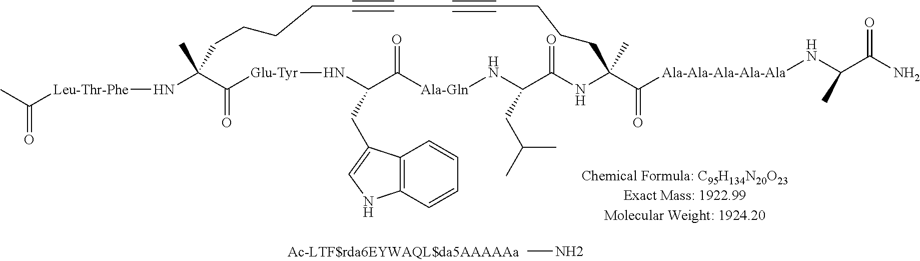 |
| 662 | 563 | 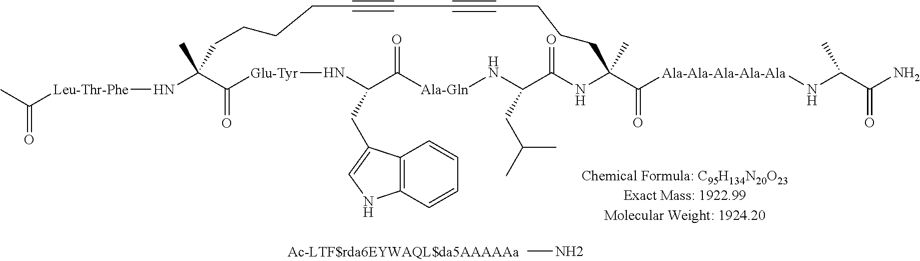 |
| | 684 | 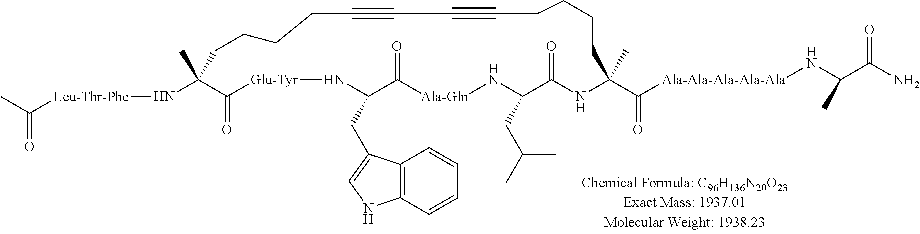 |

In some embodiments, peptidomimetic macrocycles include peptidomimetic macrocycles shown in TABLE 2a:

TABLE 2a

| Sequence | SEQ ID NO: |
|---|---|
| L$r5QETFSD$s8WKLLPEN | 685 |
| LSQ$r5TFSDLW$s8LLPEN | 686 |
| LSQE$r5FSDLWK$s8LPEN | 687 |
| LSQET$r5SDLWKL$s8PEN | 688 |
| LSQETF$r5DLWKLL$s8EN | 689 |
| LXQETFS$r5LWKLLP$s8N | 690 |
| LSQETFSD$r5WKLLPE$s8 | 691 |
| LSQQTF$r5DLWKLL$s8EN | 692 |
| LSQETF$r5DLWKLL$s8QN | 693 |
| LSQQTF$r5DLWKLL$s8QN | 694 |
| LSQETF$r5NLWKLL$s8QN | 695 |
| LSQQTF$r5NLWKLL$s8QN | 696 |
| LSQQTF$r5NLWRLL$s8QN | 697 |
| QSQQTF$r5NLWKLL$s8QN | 698 |
| QSQQTF$r5NLWRLL$s8QN | 699 |
| QSQQTA$r5NLWRLL$s8QN | 700 |
| L$r8QETFSD$WKLLPEN | 701 |
| LSQ$r8TFSDLW$LLPEN | 702 |
| LSQE$r8FSDLWK$LPEN | 703 |
| LSQET$r8SDLWKL$PEN | 704 |
| LSQETF$r8DLWKLL$EN | 705 |
| LXQETFS$r8LWKLLP$N | 706 |
| LSQETFSD$r8WKLLPE$ | 707 |
| LSQQTF$r8DLWKLL$EN | 708 |
| LSQETF$r8DLWKLL$QN | 709 |
| LSQQTF$r8DLWKLL$QN | 710 |
| LSQETF$r8NLWKLL$QN | 711 |
| LSQQTF$r8NLWKLL$QN | 712 |
| LSQQTF$r8NLWRLL$QN | 713 |
| QSQQTF$r8NLWKLL$QN | 714 |
| QSQQTF$r8NLWRLL$QN | 715 |
| QSQQTA$r8NLWRLL$QN | 716 |
| QSQQTF$r8NLWRKK$QN | 717 |
| QQTF$r8DLWRLL$EN | 718 |
| QQTF$r8DLWRLL$ | 719 |
| LSQQTF$DLW$LL | 720 |
| QQTF$DLW$LL | 721 |
| QQTA$r8DLWRLL$EN | 722 |
| QSQQTF$r5NLWRLL$s8QN (dihydroxylated olefin) | 723 |
| QSQQTA$r5NLWRLL$s8QN (dihydroxylated olefin) | 724 |
| QSQQTF$r8DLWRLL$QN | 725 |
| QTF$r8NLWRLL$ | 726 |
| QSQQTF$NLW$LLPQN | 727 |
| QS$QTF$NLWRLLPQN | 728 |
| $TFS$LWKLL | 729 |
| ETF$DLW$LL | 730 |
| QTF$NLW$LL | 731 |
| $SQE$FSNLWKLL | 732 |

In TABLE 2a, the peptides can comprise an N-terminal capping group such as acetyl or an additional linker such as beta-alanine between the capping group and the start of the peptide sequence.

In some embodiments, peptidomimetic macrocycles include those shown in TABLE 2b.

TABLE 2b

| | | | | | Observed mass |
|---|---|---|---|---|---|
| SP | Sequence | SEQ ID NO: | Exact Mass | M + 2 | (m/e) |
| 1 | Ac-LSQETF$r8DLWKLL$EN-NH₂ | 733 | 2068.13 | 1035.07 | 1035.36 |
| 2 | Ac-LSQETF$r8NLWKLL$QN-NH₂ | 734 | 2066.16 | 1034.08 | 1034.31 |
| 3 | Ac-LSQQTF$r8NLWRLL$QN-NH₂ | 735 | 2093.18 | 1047.59 | 1047.73 |
| 4 | Ac-QSQQTF$r8NLWKLL$QN-NH₂ | 736 | 2080.15 | 1041.08 | 1041.31 |
| 5 | Ac-QSQQTF$r8NLWRLL$QN-NH₂ | 737 | 2108.15 | 1055.08 | 1055.32 |
| 6 | Ac-QSQQTA$r8NLWRLL$QN-NH₂ | 738 | 2032.12 | 1017.06 | 1017.24 |
| 7 | Ac-QAibQQTF$r8NLWRLL$QN-NH₂ | 739 | 2106.17 | 1054.09 | 1054.34 |
| 8 | Ac-QSQQTFSNLWRLLPQN-NH₂ | 740 | 2000.02 | 1001.01 | 1001.26 |
| 9 | Ac-QSQQTF$/r8NLWRLL$/QN-NH₂ | 741 | 2136.18 | 1069.09 | 1069.37 |
| 10 | Ac-QSQAibTF$r8NLWRLL$QN-NH₂ | 742 | 2065.15 | 1033.58 | 1033.71 |
| 11 | Ac-QSQQTF$r8NLWRLL$AN-NH₂ | 743 | 2051.13 | 1026.57 | 1026.70 |
| 12 | Ac-ASQQTF$r8NLWRLL$QN-NH₂ | 744 | 2051.13 | 1026.57 | 1026.90 |
| 13 | Ac-QSQQTF$r8ALWRLL$QN-NH₂ | 745 | 2065.15 | 1033.58 | 1033.41 |
| 14 | Ac-QSQETF$r8NLWRLL$QN-NH₂ | 746 | 2109.14 | 1055.57 | 1055.70 |
| 15 | Ac-RSQQTF$r8NLWRLL$QN-NH₂ | 747 | 2136.20 | 1069.10 | 1069.17 |
| 16 | Ac-RSQQTF$r8NLWRLL$EN-NH₂ | 748 | 2137.18 | 1069.59 | 1069.75 |
| 17 | Ac-LSQETFSDLWKLLPEN-NH₂ | 749 | 1959.99 | 981.00 | 981.24 |
| 18 | Ac-QSQ$TFS$LWRLLPQN-NH₂ | 750 | 2008.09 | 1005.05 | 1004.97 |
| 19 | Ac-QSQQ$FSN$WRLLPQN-NH₂ | 751 | 2036.06 | 1019.03 | 1018.86 |
| 20 | Ac-QSQQT$SNL$RLLPQN-NH₂ | 752 | 1917.04 | 959.52 | 959.32 |
| 21 | Ac-QSQQTF$NLW$LLPQN-NH₂ | 753 | 2007.06 | 1004.53 | 1004.97 |
| 22 | Ac-RTQATF$r8NQWAibANle$TNAibTR-NH₂ | 754 | 2310.26 | 1156.13 | 1156.52 |
| 23 | Ac-QSQQTF$r8NLWRLL$RN-NH₂ | 755 | 2136.20 | 1069.10 | 1068.94 |
| 24 | Ac-QSQRTF$r8NLWRLL$QN-NH₂ | 756 | 2136.20 | 1069.10 | 1068.94 |
| 25 | Ac-QSQQTF$r8NNleWRLL$QN-NH₂ | 757 | 2108.15 | 1055.08 | 1055.44 |
| 26 | Ac-QSQQTF$r8NLWRNleL$QN-NH₂ | 758 | 2108.15 | 1055.08 | 1055.84 |
| 27 | Ac-QSQQTF$r8NLWRLNle$QN-NH₂ | 759 | 2108.15 | 1055.08 | 1055.12 |
| 28 | Ac-QSQQTY$r8NLWRLL$QN-NH₂ | 760 | 2124.15 | 1063.08 | 1062.92 |
| 29 | Ac-RAibQQTF$r8NLWRLL$QN-NH₂ | 761 | 2134.22 | 1068.11 | 1068.65 |
| 30 | Ac-MPRFMDYWEGLN-NH₂ | 762 | 1598.70 | 800.35 | 800.45 |
| 31 | Ac-RSQQRF$r8NLWRLL$QN-NH₂ | 763 | 2191.25 | 1096.63 | 1096.83 |
| 32 | Ac-QSQQRF$r8NLWRLL$QN-NH₂ | 764 | 2163.21 | 1082.61 | 1082.87 |
| 33 | Ac-RAibQQRF$r8NLWRLL$QN-NH₂ | 765 | 2189.27 | 1095.64 | 1096.37 |
| 34 | Ac-RSQQRF$r8NFWRLL$QN-NH₂ | 766 | 2225.23 | 1113.62 | 1114.37 |
| 35 | Ac-RSQQRF$r8NYWRLL$QN-NH₂ | 767 | 2241.23 | 1121.62 | 1122.37 |
| 36 | Ac-RSQQTF$r8NLWQLL$QN-NH₂ | 768 | 2108.15 | 1055.08 | 1055.29 |
| 37 | Ac-QSQQTF$r8NLWQAmlL$QN-NH₂ | 769 | 2094.13 | 1048.07 | 1048.32 |
| 38 | Ac-QSQQTF$r8NAmlWRLL$QN-NH₂ | 770 | 2122.17 | 1062.09 | 1062.35 |
| 39 | Ac-NlePRF$r8DYWEGL$QN-NH₂ | 771 | 1869.98 | 935.99 | 936.20 |
| 40 | Ac-NlePRF$r8NYWRLL$QN-NH₂ | 772 | 1952.12 | 977.06 | 977.35 |
| 41 | Ac-RF$r8NLWRLL$Q-NH₂ | 773 | 1577.96 | 789.98 | 790.18 |
| 42 | Ac-QSQQTF$r8N2ffWRLL$QN-NH₂ | 774 | 2160.13 | 1081.07 | 1081.40 |
| 43 | Ac-QSQQTF$r8N3ffWRLL$QN-NH₂ | 775 | 2160.13 | 1081.07 | 1081.34 |
| 44 | Ac-QSQQTF#r8NLWRLL#QN-NH₂ | 776 | 2080.12 | 1041.06 | 1041.34 |
| 45 | Ac-RSQQTA$r8NLWRLL$QN-NH₂ | 777 | 2060.16 | 1031.08 | 1031.38 |
| 46 | Ac-QSQQTF % r8NLWRLL % QN-NH₂ | 778 | 2110.17 | 1056.09 | 1056.55 |
| 47 | HepQSQ$TFSNLWRLLPQN-NH₂ | 779 | 2051.10 | 1026.55 | 1026.82 |
| 48 | HepQSQ$TF$r8NLWRLL$QN-NH₂ | 780 | 2159.23 | 1080.62 | 1080.89 |
| 49 | Ac-QSQQTF$r8NL6clWRLL$QN-NH₂ | 781 | 2142.11 | 1072.06 | 1072.35 |
| 50 | Ac-QSQQTF$r8NLMe6clwRLL$QN-NH₂ | 782 | 2156.13 | 1079.07 | 1079.27 |
| 51 | Ac-LTFEHYWAQLTS-NH₂ | 783 | 1535.74 | 768.87 | 768.91 |
| 52 | Ac-LTF$HYW$QLTS-NH₂ | 784 | 1585.83 | 793.92 | 794.17 |
| 53 | Ac-LTFE$YWA$LTS-NH₂ | 785 | 1520.79 | 761.40 | 761.67 |
| 54 | Ac-LTF$zr8HYWAQL$zS-NH₂ | 786 | 1597.87 | 799.94 | 800.06 |
| 55 | Ac-LTF$r8HYWRQL$S-NH₂ | 787 | 1682.93 | 842.47 | 842.72 |
| 56 | Ac-QS$QTFStNLWRLL$s8QN-NH₂ | 788 | 2145.21 | 1073.61 | 1073.90 |
| 57 | Ac-QSQQTASNLWRLLPQN-NH₂ | 789 | 1923.99 | 963.00 | 963.26 |
| 58 | Ac-QSQQTA$/r8NLWRLL$/QN-NH₂ | 790 | 2060.15 | 1031.08 | 1031.24 |
| 59 | Ac-ASQQTF$/r8NLWRLL$/QN-NH₂ | 791 | 2079.16 | 1040.58 | 1040.89 |
| 60 | Ac-$SQQ$FSNLWRLLAibQN-NH₂ | 792 | 2009.09 | 1005.55 | 1005.86 |
| 61 | Ac-QS$QTF$NLWRLLAibQN-NH₂ | 793 | 2023.10 | 1012.55 | 1012.79 |
| 62 | Ac-QSQQ$FSN$WRLLAibQN-NH₂ | 794 | 2024.06 | 1013.03 | 1013.31 |
| 63 | Ac-QSQQTF$NLW$LLAibQN-NH₂ | 795 | 1995.06 | 998.53 | 998.87 |
| 64 | Ac-QSQQTFS$LWR$LAibQN-NH₂ | 796 | 2011.06 | 1006.53 | 1006.83 |
| 65 | Ac-QSQQTFSNLW$LLA$N-NH₂ | 797 | 1940.02 | 971.01 | 971.29 |
| 66 | Ac-$/SQQ$/FSNLWRLLAibQN-NH₂ | 798 | 2037.12 | 1019.56 | 1019.78 |
| 67 | Ac-QS$/QTF$/NLWRLLAibQN-NH₂ | 799 | 2051.13 | 1026.57 | 1026.90 |
| 68 | Ac-QSQQ$/FSN$/WRLLAibQN-NH₂ | 800 | 2052.09 | 1027.05 | 1027.36 |
| 69 | Ac-QSQQTF$/NLW$/LLAibQN-NH₂ | 801 | 2023.09 | 1012.55 | 1013.82 |
| 70 | Ac-QSQ$TFS$LWRLLAibQN-NH₂ | 802 | 1996.09 | 999.05 | 999.39 |
| 71 | Ac-QSQ$/TFS$/LWRLLAibQN-NH₂ | 803 | 2024.12 | 1013.06 | 1013.37 |
| 72 | Ac-QS$/QTFSt//NLWRLL$/s8QN-NH₂ | 804 | 2201.27 | 1101.64 | 1102.00 |
| 73 | Ac-$r8SQQTFS$LWRLLAibQN-NH₂ | 805 | 2038.14 | 1020.07 | 1020.23 |
| 74 | Ac-QSQ$r8TFSNLW$LLAibQN-NH₂ | 806 | 1996.08 | 999.04 | 999.32 |
| 75 | Ac-QSQQTFS$r8LWRLLA$N-NH₂ | 807 | 2024.12 | 1013.06 | 1013.37 |
| 76 | Ac-QS$r5QTFStNLW$LLAibQN-NH₂ | 808 | 2032.12 | 1017.06 | 1017.39 |
| 77 | Ac-$/r8SQQTFS$/LWRLLAibQN-NH₂ | 809 | 2066.17 | 1034.09 | 1034.80 |
| 78 | Ac-QSQ$/r8TFSNLW$/LLAibQN-NH₂ | 810 | 2024.11 | 1013.06 | 1014.34 |
| 79 | Ac-QSQQTFS$/r8LWRLLA$/N-NH₂ | 811 | 2052.15 | 1027.08 | 1027.16 |
| 80 | Ac-QS$/r5QTFSt//NLW$/LLAibQN-NH₂ | 812 | 2088.18 | 1045.09 | 1047.10 |
| 81 | Ac-QSQQTFSNLWRLLAibQN-NH₂ | 813 | 1988.02 | 995.01 | 995.31 |
| 82 | Hep/QSQ$/TF$/r8NLWRLL$/QN-NH₂ | 814 | 2215.29 | 1108.65 | 1108.93 |
| 83 | Ac-ASQQTF$r8NLRWLL$QN-NH₂ | 815 | 2051.13 | 1026.57 | 1026.90 |
| 84 | Ac-QSQQTF$/r8NLWRLL$/Q-NH₂ | 816 | 2022.14 | 1012.07 | 1012.66 |
| 85 | Ac-QSQQTF$r8NLWRLL$Q-NH₂ | 817 | 1994.11 | 998.06 | 998.42 |
| 86 | Ac-AAARAA$r8AAARAA$AA-NH₂ | 818 | 1515.90 | 758.95 | 759.21 |
| 87 | Ac-LTFEHYWAQLTSA-NH₂ | 819 | 1606.78 | 804.39 | 804.59 |
| 88 | Ac-LTF$r8HYWAQL$SA-NH₂ | 820 | 1668.90 | 835.45 | 835.67 |
| 89 | Ac-ASQQTFSNLWRLLPQN-NH₂ | 821 | 1943.00 | 972.50 | 973.27 |
| 90 | Ac-QS$QTFStNLW$r5LLAibQN-NH₂ | 822 | 2032.12 | 1017.06 | 1017.30 |
| 91 | Ac-QSQQTFAibNLWRLLAibQN-NH₂ | 823 | 1986.04 | 994.02 | 994.19 |
| 92 | Ac-QSQQTFNleNLWRLLNleQN-NH₂ | 824 | 2042.11 | 1022.06 | 1022.23 |
| 93 | Ac-QSQQTF$/r8NLWRLLAibQN-NH₂ | 825 | 2082.14 | 1042.07 | 1042.23 |
| 94 | Ac-QSQQTF$/r8NLWRLLNleQN-NH₂ | 826 | 2110.17 | 1056.09 | 1056.29 |
| 95 | Ac-QSQQTFAibNLWRLL$/QN-NH₂ | 827 | 2040.09 | 1021.05 | 1021.25 |
| 96 | Ac-QSQQTFNleNLWRLL$/QN-NH₂ | 828 | 2068.12 | 1035.06 | 1035.31 |
| 97 | Ac-QSQQTF % r8NL6clWRNleL % QN-NH₂ | 829 | 2144.13 | 1073.07 | 1073.32 |
| 98 | Ac-QSQQTF % r8NLMe6clWRLL % QN-NH₂ | 830 | 2158.15 | 1080.08 | 1080.31 |
| 101 | Ac-FNle$YWE$L-NH₂ | 831 | 1160.63 | — | 1161.70 |
| 102 | Ac-F$r8AYWELL$A-NH₂ | 832 | 1344.75 | — | 1345.90 |
| 103 | Ac-F$r8AYWQLL$A-NH₂ | 833 | 1343.76 | — | 1344.83 |
| 104 | Ac-NlePRF$r8NYWELL$QN-NH₂ | 834 | 1925.06 | 963.53 | 963.69 |
| 105 | Ac-NlePRF$r8DYWRLL$QN-NH₂ | 835 | 1953.10 | 977.55 | 977.68 |
| 106 | Ac-NlePRF$r8NYWRLL$Q-NH₂ | 836 | 1838.07 | 920.04 | 920.18 |
| 107 | Ac-NlePRF$r8NYWRLL$-NH₂ | 837 | 1710.01 | 856.01 | 856.13 |
| 108 | Ac-QSQQTF$r8DLWRLL$QN-NH₂ | 838 | 2109.14 | 1055.57 | 1055.64 |
| 109 | Ac-QSQQTF$r8NLWRLL$EN-NH₂ | 839 | 2109.14 | 1055.57 | 1055.70 |
| 110 | Ac-QSQQTF$r8NLWRLL$QD-NH₂ | 840 | 2109.14 | 1055.57 | 1055.64 |
| 111 | Ac-QSQQTF$r8NLWRLL$S-NH₂ | 841 | 1953.08 | 977.54 | 977.60 |
| 112 | Ac-ESQQTF$r8NLWRLL$QN-NH₂ | 842 | 2109.14 | 1055.57 | 1055.70 |
| 113 | Ac-LTF$r8NLWRNleL$Q-NH₂ | 843 | 1635.99 | 819.00 | 819.10 |
| 114 | Ac-LRF$r8NLWRNleL$Q-NH₂ | 844 | 1691.04 | 846.52 | 846.68 |
| 115 | Ac-QSQQTF$r8NWWRNleL$QN-NH₂ | 845 | 2181.15 | 1091.58 | 1091.64 |
| 116 | Ac-QSQQTF$r8NLWRNleL$Q-NH₂ | 846 | 1994.11 | 998.06 | 998.07 |
| 117 | Ac-QTF$r8NLWRNleL$QN-NH₂ | 847 | 1765.00 | 883.50 | 883.59 |
| 118 | Ac-NlePRF$r8NWWRLL$QN-NH₂ | 848 | 1975.13 | 988.57 | 988.75 |
| 119 | Ac-NlePRF$r8NWWRLL$A-NH₂ | 849 | 1804.07 | 903.04 | 903.08 |
| 120 | Ac-TSFAEYWNLLNH₂ | 850 | 1467.70 | 734.85 | 734.90 |
| 121 | Ac-QTF$r8HWWSQL$S-NH₂ | 851 | 1651.85 | 826.93 | 827.12 |
| 122 | Ac-FM$YWE$L-NH₂ | 852 | 1178.58 | — | 1179.64 |
| 123 | Ac-QTFEHWWSQLLS-NH₂ | 853 | 1601.76 | 801.88 | 801.94 |
| 124 | Ac-QSQQTF$r8NLAmwRLNle$QN-NH₂ | 854 | 2122.17 | 1062.09 | 1062.24 |
| 125 | Ac-FMAibY6clWEAc3cL-NH₂ | 855 | 1130.47 | — | 1131.53 |
| 126 | Ac-FNle$Y6clWE$L-NH₂ | 856 | 1194.59 | — | 1195.64 |
| 127 | Ac-F$zr8AY6clWEAc3cL$z-NH₂ | 857 | 1277.63 | 639.82 | 1278.71 |
| 128 | Ac-F$r8AY6clWEAc3cL$A-NH₂ | 858 | 1348.66 | — | 1350.72 |
| 129 | Ac-NlePRF$r8NY6clWRLL$QN-NH₂ | 859 | 1986.08 | 994.04 | 994.64 |
| 130 | Ac-AF$r8AAWALA$A-NH₂ | 860 | 1223.71 | — | 1224.71 |
| 131 | Ac-TF$r8AAWRLA$Q-NH₂ | 861 | 1395.80 | 698.90 | 399.04 |
| 132 | Pr-TF$r8AAWRLA$Q-NH₂ | 862 | 1409.82 | 705.91 | 706.04 |
| 133 | Ac-QSQQTF % r8NLWRNleL % QN-NH₂ | 863 | 2110.17 | 1056.09 | 1056.22 |
| 134 | Ac-LTF % r8HYWAQL % SA-NH₂ | 864 | 1670.92 | 836.46 | 836.58 |
| 135 | Ac-NlePRF % r8NYWRLL % QN-NH₂ | 865 | 1954.13 | 978.07 | 978.19 |
| 136 | Ac-NlePRF % r8NY6clWRLL % QN-NH₂ | 866 | 1988.09 | 995.05 | 995.68 |
| 137 | Ac-LTF % r8HY6clWAQL % S-NH₂ | 867 | 1633.84 | 817.92 | 817.93 |
| 138 | Ac-QS % QTF % StNLWRLL % s8QN-NH₂ | 868 | 2149.24 | 1075.62 | 1075.65 |
| 139 | Ac-LTF % r8HY6clWRQL % S-NH₂ | 869 | 1718.91 | 860.46 | 860.54 |
| 140 | Ac-QSQQTF % r8NL6clWRLL % QN-NH₂ | 870 | 2144.13 | 1073.07 | 1073.64 |
| 141 | Ac- % r8SQQTFS % LWRLLAibQN-NH₂ | 871 | 2040.15 | 1021.08 | 1021.13 |
| 142 | Ac-LTF % r8HYWAQL % S-NH₂ | 872 | 1599.88 | 800.94 | 801.09 |
| 143 | Ac-TSF % r8QYWNLL % P-NH₂ | 873 | 1602.88 | 802.44 | 802.58 |
| 147 | Ac-LTFEHYWAQLTS-NH₂ | 874 | 1535.74 | 768.87 | 769.5 |
| 152 | Ac-F$er8AY6clWEAc3cL$e-NH₂ | 875 | 1277.63 | 639.82 | 1278.71 |
| 153 | Ac-AF$r8AAWALA$A-NH₂ | 876 | 1277.63 | 639.82 | 1277.84 |
| 154 | Ac-TF$r8AAWRLA$Q-NH₂ | 877 | 1395.80 | 698.90 | 699.04 |
| 155 | Pr-TF$r8AAWRLA$Q-NH₂ | 878 | 1409.82 | 705.91 | 706.04 |
| 156 | Ac-LTF$er8HYWAQL$eS-NH₂ | 879 | 1597.87 | 799.94 | 800.44 |
| 159 | Ac-CCPGCCBaQSQQTF$r8NLWRLL$QN-NH₂ | 880 | 2745.30 | 1373.65 | 1372.99 |
| 160 | Ac-CCPGCCBaQSQQTA$r8NLWRLL$QN-NH₂ | 881 | 2669.27 | 1335.64 | 1336.09 |
| 161 | Ac-CCPGCCBaNlePRF$r8NYWRLL$QN-NH₂ | 882 | 2589.26 | 1295.63 | 1296.2 |
| 162 | Ac-LTF$/r8HYWAQL$/S-NH₂ | 883 | 1625.90 | 813.95 | 814.18 |
| 163 | Ac-F % r8HY6clWRAc3cL % -NH₂ | 884 | 1372.72 | 687.36 | 687.59 |
| 164 | Ac-QTF % r8HWWSQL % S-NH₂ | 885 | 1653.87 | 827.94 | 827.94 |
| 165 | Ac-LTA$r8HYWRQL$S-NH₂ | 886 | 1606.90 | 804.45 | 804.66 |
| 166 | Ac-Q$r8QQTFSN$WRLLAibQN-NH₂ | 887 | 2080.12 | 1041.06 | 1041.61 |
| 167 | Ac-QSQQ$r8FSNLWR$LAibQN-NH₂ | 888 | 2066.11 | 1034.06 | 1034.58 |
| 168 | Ac-F$r8AYWEAc3cL$A-NH₂ | 889 | 1314.70 | 658.35 | 1315.88 |
| 169 | Ac-F$r8AYWEAc3cL$S-NH₂ | 890 | 1330.70 | 666.35 | 1331.87 |
| 170 | Ac-F$r8AYWEAc3cL$Q-NH₂ | 891 | 1371.72 | 686.86 | 1372.72 |
| 171 | Ac-F$r8AYWEAibL$S-NH₂ | 892 | 1332.71 | 667.36 | 1334.83 |
| 172 | Ac-F$r8AYWEAL$S-NH₂ | 893 | 1318.70 | 660.35 | 1319.73 |
| 173 | Ac-F$r8AYWEQL$S-NH₂ | 894 | 1375.72 | 688.86 | 1377.53 |
| 174 | Ac-F$r8HYWEQL$S-NH₂ | 895 | 1441.74 | 721.87 | 1443.48 |
| 175 | Ac-F$r8HYWAQL$S-NH₂ | 896 | 1383.73 | 692.87 | 1385.38 |
| 176 | Ac-F$r8HYWAAc3cL$S-NH₂ | 897 | 1338.71 | 670.36 | 1340.82 |
| 177 | Ac-F$r8HYWRAc3cL$S-NH₂ | 898 | 1423.78 | 712.89 | 713.04 |
| 178 | Ac-F$r8AYWEAc3cL#A-NH₂ | 899 | 1300.69 | 651.35 | 1302.78 |
| 179 | Ac-NlePTF % r8NYWRLL % QN-NH₂ | 900 | 1899.08 | 950.54 | 950.56 |
| 180 | Ac-TF$r8AAWRAL$Q-NH₂ | 901 | 1395.80 | 698.90 | 699.13 |
| 181 | Ac-TSF % r8HYWAQL % S-NH₂ | 902 | 1573.83 | 787.92 | 787.98 |
| 184 | Ac-F % r8AY6clWEAc3cL % A-NH₂ | 903 | 1350.68 | 676.34 | 676.91 |
| 185 | Ac-LTF$r8HYWAQI$S-NH₂ | 904 | 1597.87 | 799.94 | 800.07 |
| 186 | Ac-LTF$r8HYWAQNle$S-NH₂ | 905 | 1597.87 | 799.94 | 800.07 |
| 187 | Ac-LTF$r8HYWAQL$A-NH₂ | 906 | 1581.87 | 791.94 | 792.45 |
| 188 | Ac-LTF$r8HYWAQL$Abu-NH₂ | 907 | 1595.89 | 798.95 | 799.03 |
| 189 | Ac-LTF$r8HYWAbuQL$S-NH₂ | 908 | 1611.88 | 806.94 | 807.47 |
| 190 | Ac-LTF$er8AYWAQL$eS-NH₂ | 909 | 1531.84 | 766.92 | 766.96 |
| 191 | Ac-LAF$r8HYWAQL$S-NH₂ | 910 | 1567.86 | 784.93 | 785.49 |
| 192 | Ac-LAF$r8AYWAQL$S-NH₂ | 911 | 1501.83 | 751.92 | 752.01 |
| 193 | Ac-LTF$er8AYWAQL$eA-NH₂ | 912 | 1515.85 | 758.93 | 758.97 |
| 194 | Ac-LAF$r8AYWAQL$A-NH₂ | 913 | 1485.84 | 743.92 | 744.05 |
| 195 | Ac-LTF$r8NLWANleL$Q-NH₂ | 914 | 1550.92 | 776.46 | 776.61 |
| 196 | Ac-LTF$r8NLWANleL$A-NH₂ | 915 | 1493.90 | 747.95 | 1495.6 |
| 197 | Ac-LTF$r8ALWANleL$Q-NH₂ | 916 | 1507.92 | 754.96 | 755 |
| 198 | Ac-LAF$r8NLWANleL$Q-NH₂ | 917 | 1520.91 | 761.46 | 761.96 |
| 199 | Ac-LAF$r8ALWANleL$A-NH₂ | 918 | 1420.89 | 711.45 | 1421.74 |
| 200 | Ac-A$r8AYWEAc3cL$A-NH₂ | 919 | 1238.67 | 620.34 | 1239.65 |
| 201 | Ac-F$r8AYWEAc3cL$AA-NH₂ | 920 | 1385.74 | 693.87 | 1386.64 |
| 202 | Ac-F$r8AYWEAc3cL$Abu-NH₂ | 921 | 1328.72 | 665.36 | 1330.17 |
| 203 | Ac-F$r8AYWEAc3cL$Nle-NH₂ | 922 | 1356.75 | 679.38 | 1358.22 |
| 204 | Ac-F$r5AYWEAc3cL$s8A-NH₂ | 923 | 1314.70 | 658.35 | 1315.51 |
| 205 | Ac-F$AYWEAc3cL$r8A-NH₂ | 924 | 1314.70 | 658.35 | 1315.66 |
| 206 | Ac-F$r8AYWEAc3cI$A-NH₂ | 925 | 1314.70 | 658.35 | 1316.18 |
| 207 | Ac-F$r8AYWEAc3cNle$A-NH₂ | 926 | 1314.70 | 658.35 | 1315.66 |
| 208 | Ac-F$r8AYWEAmlL$A-NH₂ | 927 | 1358.76 | 680.38 | 1360.21 |
| 209 | Ac-F$r8AYWENleL$A-NH₂ | 928 | 1344.75 | 673.38 | 1345.71 |
| 210 | Ac-F$r8AYWQAc3cL$A-NH₂ | 929 | 1313.72 | 657.86 | 1314.7 |
| 211 | Ac-F$r8AYWAAc3cL$A-NH₂ | 930 | 1256.70 | 629.35 | 1257.56 |
| 212 | Ac-F$r8AYWAbuAc3cL$A-NH₂ | 931 | 1270.71 | 636.36 | 1272.14 |
| 213 | Ac-F$r8AYWNleAc3cL$A-NH₂ | 932 | 1298.74 | 650.37 | 1299.67 |
| 214 | Ac-F$r8AbuYWEAc3cL$A-NH₂ | 933 | 1328.72 | 665.36 | 1329.65 |
| 215 | Ac-F$r8NleYWEAc3cL$A-NH₂ | 934 | 1356.75 | 679.38 | 1358.66 |
| 216 | 5-FAM-BaLTFEHYWAQLTS-NH₂ | 935 | 1922.82 | 962.41 | 962.87 |
| 217 | 5-FAM-BaLTF % r8HYWAQL % S-NH₂ | 936 | 1986.96 | 994.48 | 994.97 |
| 218 | Ac-LTF$r8HYWAQhL$S-NH₂ | 937 | 1611.88 | 806.94 | 807 |
| 219 | Ac-LTF$r8HYWAQTle$S-NH₂ | 938 | 1597.87 | 799.94 | 799.97 |
| 220 | Ac-LTF$r8HYWAQAdm$S-NH₂ | 939 | 1675.91 | 838.96 | 839.09 |
| 221 | Ac-LTF$r8HYWAQhCha$S-NH₂ | 940 | 1651.91 | 826.96 | 826.98 |
| 222 | Ac-LTF$r8HYWAQCha$S-NH₂ | 941 | 1637.90 | 819.95 | 820.02 |
| 223 | Ac-LTF$r8HYWAc6cQL$S-NH₂ | 942 | 1651.91 | 826.96 | 826.98 |
| 224 | Ac-LTF$r8HYWAc5cQL$S-NH₂ | 943 | 1637.90 | 819.95 | 820.02 |
| 225 | Ac-LThF$r8HYWAQL$S-NH₂ | 944 | 1611.88 | 806.94 | 807 |
| 226 | Ac-LTIgl$r8HYWAQL$S-NH₂ | 945 | 1625.90 | 813.95 | 812.99 |
| 227 | Ac-LTF$r8HYWAQChg$S-NH₂ | 946 | 1623.88 | 812.94 | 812.99 |
| 228 | Ac-LTF$r8HYWAQF$S-NH₂ | 947 | 1631.85 | 816.93 | 816.99 |
| 229 | Ac-LTF$r8HYWAQIgl$S-NH₂ | 948 | 1659.88 | 830.94 | 829.94 |
| 230 | Ac-LTF$r8HYWAQCba$S-NH₂ | 949 | 1609.87 | 805.94 | 805.96 |
| 231 | Ac-LTF$r8HYWAQCpg$S-NH₂ | 950 | 1609.87 | 805.94 | 805.96 |
| 232 | Ac-LTF$r8HhYWAQL$S-NH₂ | 951 | 1611.88 | 806.94 | 807 |
| 233 | Ac-F$r8AYWEAc3chL$A-NH₂ | 952 | 1328.72 | 665.36 | 665.43 |
| 234 | Ac-F$r8AYWEAc3cTle$A-NH₂ | 953 | 1314.70 | 658.35 | 1315.62 |
| 235 | Ac-F$r8AYWEAc3cAdm$A-NH₂ | 954 | 1392.75 | 697.38 | 697.47 |
| 236 | Ac-F$r8AYWEAc3chCha$A-NH₂ | 955 | 1368.75 | 685.38 | 685.34 |
| 237 | Ac-F$r8AYWEAc3cCha$A-NH₂ | 956 | 1354.73 | 678.37 | 678.38 |
| 238 | Ac-F$r8AYWEAc6cL$A-NH₂ | 957 | 1356.75 | 679.38 | 679.42 |
| 239 | Ac-F$r8AYWEAc5cL$A-NH₂ | 958 | 1342.73 | 672.37 | 672.46 |
| 240 | Ac-hF$r8AYWEAc3cL$A-NH₂ | 959 | 1328.72 | 665.36 | 665.43 |
| 241 | Ac-Igl$r8AYWEAc3cL$A-NH₂ | 960 | 1342.73 | 672.37 | 671.5 |
| 243 | Ac-F$r8AYWEAc3cF$A-NH₂ | 961 | 1348.69 | 675.35 | 675.35 |
| 244 | Ac-F$r8AYWEAc3cIgl$A-NH₂ | 962 | 1376.72 | 689.36 | 688.37 |
| 245 | Ac-F$r8AYWEAc3cCba$A-NH₂ | 963 | 1326.70 | 664.35 | 664.47 |
| 246 | Ac-F$r8AYWEAc3cCpg$A-NH₂ | 964 | 1326.70 | 664.35 | 664.39 |
| 247 | Ac-F$r8AhYWEAc3cL$A-NH₂ | 965 | 1328.72 | 665.36 | 665.43 |
| 248 | Ac-F$r8AYWEAc3cL$Q-NH₂ | 966 | 1371.72 | 686.86 | 1372.87 |
| 249 | Ac-F$r8AYWEAibL$A-NH₂ | 967 | 1316.72 | 659.36 | 1318.18 |
| 250 | Ac-F$r8AYWEAL$A-NH₂ | 968 | 1302.70 | 652.35 | 1303.75 |
| 251 | Ac-LAF$r8AYWAAL$A-NH₂ | 969 | 1428.82 | 715.41 | 715.49 |
| 252 | Ac-LTF$r8HYWAAc3cL$S-NH₂ | 970 | 1552.84 | 777.42 | 777.5 |
| 253 | Ac-NleTF$r8HYWAQL$S-NH₂ | 971 | 1597.87 | 799.94 | 800.04 |
| 254 | Ac-VTF$r8HYWAQL$S-NH₂ | 972 | 1583.85 | 792.93 | 793.04 |
| 255 | Ac-FTF$r8HYWAQL$S-NH₂ | 973 | 1631.85 | 816.93 | 817.02 |
| 256 | Ac-WTF$r8HYWAQL$S-NH₂ | 974 | 1670.86 | 836.43 | 836.85 |
| 257 | Ac-RTF$r8HYWAQL$S-NH₂ | 975 | 1640.88 | 821.44 | 821.9 |
| 258 | Ac-KTF$r8HYWAQL$S-NH₂ | 976 | 1612.88 | 807.44 | 807.91 |
| 259 | Ac-LNleF$r8HYWAQL$S-NH₂ | 977 | 1609.90 | 805.95 | 806.43 |
| 260 | Ac-LVF$r8HYWAQL$S-NH₂ | 978 | 1595.89 | 798.95 | 798.93 |
| 261 | Ac-LFF$r8HYWAQL$S-NH₂ | 979 | 1643.89 | 822.95 | 823.38 |
| 262 | Ac-LWF$r8HYWAQL$S-NH₂ | 980 | 1682.90 | 842.45 | 842.55 |
| 263 | Ac-LRF$r8HYWAQL$S-NH₂ | 981 | 1652.92 | 827.46 | 827.52 |
| 264 | Ac-LKF$r8HYWAQL$S-NH₂ | 982 | 1624.91 | 813.46 | 813.51 |
| 265 | Ac-LTF$r8NleYWAQL$S-NH₂ | 983 | 1573.89 | 787.95 | 788.05 |
| 266 | Ac-LTF$r8VYWAQL$S-NH₂ | 984 | 1559.88 | 780.94 | 780.98 |
| 267 | Ac-LTF$r8FYWAQL$S-NH₂ | 985 | 1607.88 | 804.94 | 805.32 |
| 268 | Ac-LTF$r8WYWAQL$S-NH₂ | 986 | 1646.89 | 824.45 | 824.86 |
| 269 | Ac-LTF$r8RYWAQL$S-NH₂ | 987 | 1616.91 | 809.46 | 809.51 |
| 270 | Ac-LTF$r8KYWAQL$S-NH₂ | 988 | 1588.90 | 795.45 | 795.48 |
| 271 | Ac-LTF$r8HNleWAQL$S-NH₂ | 989 | 1547.89 | 774.95 | 774.98 |
| 272 | Ac-LTF$r8HVWAQL$S-NH₂ | 990 | 1533.87 | 767.94 | 767.95 |
| 273 | Ac-LTF$r8HFWAQL$S-NH₂ | 991 | 1581.87 | 791.94 | 792.3 |
| 274 | Ac-LTF$r8HWWAQL$S-NH₂ | 992 | 1620.88 | 811.44 | 811.54 |
| 275 | Ac-LTF$r8HRWAQL$S-NH₂ | 993 | 1590.90 | 796.45 | 796.52 |
| 276 | Ac-LTF$r8HKWAQL$S-NH₂ | 994 | 1562.90 | 782.45 | 782.53 |
| 277 | Ac-LTF$r8HYWNleQL$S-NH₂ | 995 | 1639.91 | 820.96 | 820.98 |
| 278 | Ac-LTF$r8HYWVQL$S-NH₂ | 996 | 1625.90 | 813.95 | 814.03 |
| 279 | Ac-LTF$r8HYWFQL$S-NH₂ | 997 | 1673.90 | 837.95 | 838.03 |
| 280 | Ac-LTF$r8HYWWQL$S-NH₂ | 998 | 1712.91 | 857.46 | 857.5 |
| 281 | Ac-LTF$r8HYWKQL$S-NH₂ | 999 | 1654.92 | 828.46 | 828.49 |
| 282 | Ac-LTF$r8HYWANleL$S-NH₂ | 1000 | 1582.89 | 792.45 | 792.52 |
| 283 | Ac-LTF$r8HYWAVL$S-NH₂ | 1001 | 1568.88 | 785.44 | 785.49 |
| 284 | Ac-LTF$r8HYWAFL$S-NH₂ | 1002 | 1616.88 | 809.44 | 809.47 |
| 285 | Ac-LTF$r8HYWAWL$S-NH₂ | 1003 | 1655.89 | 828.95 | 829 |
| 286 | Ac-LTF$r8HYWARL$S-NH₂ | 1004 | 1625.91 | 813.96 | 813.98 |
| 287 | Ac-LTF$r8HYWAQL$Nle-NH₂ | 1005 | 1623.92 | 812.96 | 813.39 |
| 288 | Ac-LTF$r8HYWAQL$V-NH₂ | 1006 | 1609.90 | 805.95 | 805.99 |
| 289 | Ac-LTF$r8HYWAQL$F-NH₂ | 1007 | 1657.90 | 829.95 | 830.26 |
| 290 | Ac-LTF$r8HYWAQL$W-NH₂ | 1008 | 1696.91 | 849.46 | 849.5 |
| 291 | Ac-LTF$r8HYWAQL$R-NH₂ | 1009 | 1666.94 | 834.47 | 834.56 |
| 292 | Ac-LTF$r8HYWAQL$K-NH₂ | 1010 | 1638.93 | 820.47 | 820.49 |
| 293 | Ac-Q$r8QQTFSN$WRLLAibQN-NH₂ | 1011 | 2080.12 | 1041.06 | 1041.54 |
| 294 | Ac-QSQQ$r8FSNLWR$LAibQN-NH₂ | 1012 | 2066.11 | 1034.06 | 1034.58 |
| 295 | Ac-LT2Pal$r8HYWAQL$S-NH₂ | 1013 | 1598.86 | 800.43 | 800.49 |
| 296 | Ac-LT3Pal$r8HYWAQL$S-NH₂ | 1014 | 1598.86 | 800.43 | 800.49 |
| 297 | Ac-LT4Pal$r8HYWAQL$S-NH₂ | 1015 | 1598.86 | 800.43 | 800.49 |
| 298 | Ac-LTF2CF3$r8HYWAQL$S-NH₂ | 1016 | 1665.85 | 833.93 | 834.01 |
| 299 | Ac-LTF2CN$r8HYWAQL$S-NH₂ | 1017 | 1622.86 | 812.43 | 812.47 |
| 300 | Ac-LTF2Me$r8HYWAQL$S-NH₂ | 1018 | 1611.88 | 806.94 | 807 |
| 301 | Ac-LTF3Cl$r8HYWAQL$S-NH₂ | 1019 | 1631.83 | 816.92 | 816.99 |
| 302 | Ac-LTF4CF3$r8HYWAQL$S-NH₂ | 1020 | 1665.85 | 833.93 | 833.94 |
| 303 | Ac-LTF4tBu$r8HYWAQL$S-NH₂ | 1021 | 1653.93 | 827.97 | 828.02 |
| 304 | Ac-LTF5F$r8HYWAQL$S-NH₂ | 1022 | 1687.82 | 844.91 | 844.96 |
| 305 | Ac-LTF$r8HY3BthAAQL$S-NH₂ | 1023 | 1614.83 | 808.42 | 808.48 |
| 306 | Ac-LTF2Br$r8HYWAQL$S-NH₂ | 1024 | 1675.78 | 838.89 | 838.97 |
| 307 | Ac-LTF4Br$r8HYWAQL$S-NH₂ | 1025 | 1675.78 | 838.89 | 839.86 |
| 308 | Ac-LTF2Cl$r8HYWAQL$S-NH₂ | 1026 | 1631.83 | 816.92 | 816.99 |
| 309 | Ac-LTF4Cl$r8HYWAQL$S-NH₂ | 1027 | 1631.83 | 816.92 | 817.36 |
| 310 | Ac-LTF3CN$r8HYWAQL$S-NH₂ | 1028 | 1622.86 | 812.43 | 812.47 |
| 311 | Ac-LTF4CN$r8HYWAQL$S-NH₂ | 1029 | 1622.86 | 812.43 | 812.47 |
| 312 | Ac-LTF34C12$r8HYWAQL$S-NH₂ | 1030 | 1665.79 | 833.90 | 833.94 |
| 313 | Ac-LTF34F2$r8HYWAQL$S-NH₂ | 1031 | 1633.85 | 817.93 | 817.95 |
| 314 | Ac-LTF35F2$r8HYWAQL$S-NH₂ | 1032 | 1633.85 | 817.93 | 817.95 |
| 315 | Ac-LTDip$r8HYWAQL$S-NH₂ | 1033 | 1673.90 | 837.95 | 838.01 |
| 316 | Ac-LTF2F$r8HYWAQL$S-NH₂ | 1034 | 1615.86 | 808.93 | 809 |
| 317 | Ac-LTF3F$r8HYWAQL$S-NH₂ | 1035 | 1615.86 | 808.93 | 809 |
| 318 | Ac-LTF4F$r8HYWAQL$S-NH₂ | 1036 | 1615.86 | 808.93 | 809 |
| 319 | Ac-LTF4I$r8HYWAQL$S-NH₂ | 1037 | 1723.76 | 862.88 | 862.94 |
| 320 | Ac-LTF3Me$r8HYWAQL$S-NH₂ | 1038 | 1611.88 | 806.94 | 807.07 |
| 321 | Ac-LTF4Me$r8HYWAQL$S-NH₂ | 1039 | 1611.88 | 806.94 | 807 |
| 322 | Ac-LT1Nal$r8HYWAQL$S-NH₂ | 1040 | 1647.88 | 824.94 | 824.98 |
| 323 | Ac-LT2Nal$r8HYWAQL$S-NH₂ | 1041 | 1647.88 | 824.94 | 825.06 |
| 324 | Ac-LTF3CF3$r8HYWAQL$S-NH₂ | 1042 | 1665.85 | 833.93 | 834.01 |
| 325 | Ac-LTF4NO2$r8HYWAQL$S-NH₂ | 1043 | 1642.85 | 822.43 | 822.46 |
| 326 | Ac-LTF3NO2$r8HYWAQL$S-NH₂ | 1044 | 1642.85 | 822.43 | 822.46 |
| 327 | Ac-LTF$r82ThiYWAQL$S-NH₂ | 1045 | 1613.83 | 807.92 | 807.96 |
| 328 | Ac-LTF$r8HBipWAQL$S-NH₂ | 1046 | 1657.90 | 829.95 | 830.01 |
| 329 | Ac-LTF$r8HF4tBuWAQL$S-NH₂ | 1047 | 1637.93 | 819.97 | 820.02 |
| 330 | Ac-LTF$r8HF4CF3WAQL$S-NH₂ | 1048 | 1649.86 | 825.93 | 826.02 |
| 331 | Ac-LTF$r8HF4ClWAQL$S-NH₂ | 1049 | 1615.83 | 808.92 | 809.37 |
| 332 | Ac-LTF$r8HF4MeWAQL$S-NH₂ | 1050 | 1595.89 | 798.95 | 799.01 |
| 333 | Ac-LTF$r8HF4BrWAQL$S-NH₂ | 1051 | 1659.78 | 830.89 | 830.98 |
| 334 | Ac-LTF$r8HF4CNWAQL$S-NH₂ | 1052 | 1606.87 | 804.44 | 804.56 |
| 335 | Ac-LTF$r8HF4NO2WAQL$S-NH₂ | 1053 | 1626.86 | 814.43 | 814.55 |
| 336 | Ac-LTF$r8HlNalWAQL$S-NH₂ | 1054 | 1631.89 | 816.95 | 817.06 |
| 337 | Ac-LTF$r8H2NalWAQL$S-NH₂ | 1055 | 1631.89 | 816.95 | 816.99 |
| 338 | Ac-LTF$r8HWAQL$S-NH₂ | 1056 | 1434.80 | 718.40 | 718.49 |
| 339 | Ac-LTF$r8HYlNalAQL$S-NH₂ | 1057 | 1608.87 | 805.44 | 805.52 |
| 340 | Ac-LTF$r8HY2NalAQL$S-NH₂ | 1058 | 1608.87 | 805.44 | 805.52 |
| 341 | Ac-LTF$r8HYWAQI$S-NH₂ | 1059 | 1597.87 | 799.94 | 800.07 |
| 342 | Ac-LTF$r8HYWAQNle$S-NH₂ | 1060 | 1597.87 | 799.94 | 800.44 |
| 343 | Ac-LTF$er8HYWAQL$eA-NH₂ | 1061 | 1581.87 | 791.94 | 791.98 |
| 344 | Ac-LTF$r8HYWAQL$Abu-NH₂ | 1062 | 1595.89 | 798.95 | 799.03 |
| 345 | Ac-LTF$r8HYWAbuQL$S-NH₂ | 1063 | 1611.88 | 806.94 | 804.47 |
| 346 | Ac-LAF$r8HYWAQL$S-NH₂ | 1064 | 1567.86 | 784.93 | 785.49 |
| 347 | Ac-LTF$r8NLWANleL$Q-NH₂ | 1065 | 1550.92 | 776.46 | 777.5 |
| 348 | Ac-LTF$r8ALWANleL$Q-NH₂ | 1066 | 1507.92 | 754.96 | 755.52 |
| 349 | Ac-LAF$r8NLWANleL$Q-NH₂ | 1067 | 1520.91 | 761.46 | 762.48 |
| 350 | Ac-F$r8AYWAAc3cL$A-NH₂ | 1068 | 1256.70 | 629.35 | 1257.56 |
| 351 | Ac-LTF$r8AYWAAL$S-NH₂ | 1069 | 1474.82 | 738.41 | 738.55 |
| 352 | Ac-LVF$r8AYWAQL$S-NH₂ | 1070 | 1529.87 | 765.94 | 766 |
| 353 | Ac-LTF$r8AYWAbuQL$S-NH₂ | 1071 | 1545.86 | 773.93 | 773.92 |
| 354 | Ac-LTF$r8AYWNleQL$S-NH₂ | 1072 | 1573.89 | 787.95 | 788.17 |
| 355 | Ac-LTF$r8AbuYWAQL$S-NH₂ | 1073 | 1545.86 | 773.93 | 773.99 |
| 356 | Ac-LTF$r8AYWHQL$S-NH₂ | 1074 | 1597.87 | 799.94 | 799.97 |
| 357 | Ac-LTF$r8AYWKQL$S-NH₂ | 1075 | 1588.90 | 795.45 | 795.53 |
| 358 | Ac-LTF$r8AYWOQL$S-NH₂ | 1076 | 1574.89 | 788.45 | 788.5 |
| 359 | Ac-LTF$r8AYWRQL$S-NH₂ | 1077 | 1616.91 | 809.46 | 809.51 |
| 360 | Ac-LTF$r8AYWSQL$S-NH₂ | 1078 | 1547.84 | 774.92 | 774.96 |
| 361 | Ac-LTF$r8AYWRAL$S-NH₂ | 1079 | 1559.89 | 780.95 | 780.95 |
| 362 | Ac-LTF$r8AYWRQL$A-NH₂ | 1080 | 1600.91 | 801.46 | 801.52 |
| 363 | Ac-LTF$r8AYWRAL$A-NH₂ | 1081 | 1543.89 | 772.95 | 773.03 |
| 364 | Ac-LTF$r5HYWAQL$s8S-NH₂ | 1082 | 1597.87 | 799.94 | 799.97 |
| 365 | Ac-LTF$HYWAQL$r8S-NH₂ | 1083 | 1597.87 | 799.94 | 799.97 |
| 366 | Ac-LTF$r8HYWAAL$S-NH₂ | 1084 | 1540.84 | 771.42 | 771.48 |
| 367 | Ac-LTF$r8HYWAAbuL$S-NH₂ | 1085 | 1554.86 | 778.43 | 778.51 |
| 368 | Ac-LTF$r8HYWALL$S-NH₂ | 1086 | 1582.89 | 792.45 | 792.49 |
| 369 | Ac-F$r8AYWHAL$A-NH₂ | 1087 | 1310.72 | 656.36 | 656.4 |
| 370 | Ac-F$r8AYWAAL$A-NH₂ | 1088 | 1244.70 | 623.35 | 1245.61 |
| 371 | Ac-F$r8AYWSAL$A-NH₂ | 1089 | 1260.69 | 631.35 | 1261.6 |
| 372 | Ac-F$r8AYWRAL$A-NH₂ | 1090 | 1329.76 | 665.88 | 1330.72 |
| 373 | Ac-F$r8AYWKAL$A-NH₂ | 1091 | 1301.75 | 651.88 | 1302.67 |
| 374 | Ac-F$r8AYWOAL$A-NH₂ | 1092 | 1287.74 | 644.87 | 1289.13 |
| 375 | Ac-F$r8VYWEAc3cL$A-NH₂ | 1093 | 1342.73 | 672.37 | 1343.67 |
| 376 | Ac-F$r8FYWEAc3cL$A-NH₂ | 1094 | 1390.73 | 696.37 | 1392.14 |
| 377 | Ac-F$r8WYWEAc3cL$A-NH₂ | 1095 | 1429.74 | 715.87 | 1431.44 |
| 378 | Ac-F$r8RYWEAc3cL$A-NH₂ | 1096 | 1399.77 | 700.89 | 700.95 |
| 379 | Ac-F$r8KYWEAc3cL$A-NH₂ | 1097 | 1371.76 | 686.88 | 686.97 |
| 380 | Ac-F$r8ANleWEAc3cL$A-NH₂ | 1098 | 1264.72 | 633.36 | 1265.59 |
| 381 | Ac-F$r8AVWEAc3cL$A-NH₂ | 1099 | 1250.71 | 626.36 | 1252.2 |
| 382 | Ac-F$r8AFWEAc3cL$A-NH₂ | 1100 | 1298.71 | 650.36 | 1299.64 |
| 383 | Ac-F$r8AWWEAc3cL$A-NH₂ | 1101 | 1337.72 | 669.86 | 1338.64 |
| 384 | Ac-F$r8ARWEAc3cL$A-NH₂ | 1102 | 1307.74 | 654.87 | 655 |
| 385 | Ac-F$r8AKWEAc3cL$A-NH₂ | 1103 | 1279.73 | 640.87 | 641.01 |
| 386 | Ac-F$r8AYWVAc3cL$A-NH₂ | 1104 | 1284.73 | 643.37 | 643.38 |
| 387 | Ac-F$r8AYWFAc3cL$A-NH₂ | 1105 | 1332.73 | 667.37 | 667.43 |
| 388 | Ac-F$r8AYWWAc3cL$A-NH₂ | 1106 | 1371.74 | 686.87 | 686.97 |
| 389 | Ac-F$r8AYWRAc3cL$A-NH₂ | 1107 | 1341.76 | 671.88 | 671.94 |
| 390 | Ac-F$r8AYWKAc3cL$A-NH₂ | 1108 | 1313.75 | 657.88 | 657.88 |
| 391 | Ac-F$r8AYWEVL$A-NH₂ | 1109 | 1330.73 | 666.37 | 666.47 |
| 392 | Ac-F$r8AYWEFL$A-NH₂ | 1110 | 1378.73 | 690.37 | 690.44 |
| 393 | Ac-F$r8AYWEWL$A-NH₂ | 1111 | 1417.74 | 709.87 | 709.91 |
| 394 | Ac-F$r8AYWERL$A-NH₂ | 1112 | 1387.77 | 694.89 | 1388.66 |
| 395 | Ac-F$r8AYWEKL$A-NH₂ | 1113 | 1359.76 | 680.88 | 1361.21 |
| 396 | Ac-F$r8AYWEAc3cL$V-NH₂ | 1114 | 1342.73 | 672.37 | 1343.59 |
| 397 | Ac-F$r8AYWEAc3cL$F-NH₂ | 1115 | 1390.73 | 696.37 | 1392.58 |
| 398 | Ac-F$r8AYWEAc3cL$W-NH₂ | 1116 | 1429.74 | 715.87 | 1431.29 |
| 399 | Ac-F$r8AYWEAc3cL$R-NH₂ | 1117 | 1399.77 | 700.89 | 700.95 |
| 400 | Ac-F$r8AYWEAc3cL$K-NH₂ | 1118 | 1371.76 | 686.88 | 686.97 |
| 401 | Ac-F$r8AYWEAc3cL$AV-NH₂ | 1119 | 1413.77 | 707.89 | 707.91 |
| 402 | Ac-F$r8AYWEAc3cL$AF-NH₂ | 1120 | 1461.77 | 731.89 | 731.96 |
| 403 | Ac-F$r8AYWEAc3cL$AW-NH₂ | 1121 | 1500.78 | 751.39 | 751.5 |
| 404 | Ac-F$r8AYWEAc3cL$AR-NH₂ | 1122 | 1470.80 | 736.40 | 736.47 |
| 405 | Ac-F$r8AYWEAc3cL$AK-NH₂ | 1123 | 1442.80 | 722.40 | 722.41 |
| 406 | Ac-F$r8AYWEAc3cL$AH-NH₂ | 1124 | 1451.76 | 726.88 | 726.93 |
| 407 | Ac-LTF2NO2$r8HYWAQL$S-NH₂ | 1125 | 1642.85 | 822.43 | 822.54 |
| 408 | Ac-LTA$r8HYAAQL$S-NH₂ | 1126 | 1406.79 | 704.40 | 704.5 |
| 409 | Ac-LTF$r8HYAAQL$S-NH₂ | 1127 | 1482.82 | 742.41 | 742.47 |
| 410 | Ac-QSQQTF$r8NLWALL$AN-NH₂ | 1128 | 1966.07 | 984.04 | 984.38 |
| 411 | Ac-QAibQQTF$r8NLWALL$AN-NH₂ | 1129 | 1964.09 | 983.05 | 983.42 |
| 412 | Ac-QAibQQTF$r8ALWALL$AN-NH₂ | 1130 | 1921.08 | 961.54 | 961.59 |
| 413 | Ac-AAAATF$r8AAWAAL$AA-NH₂ | 1131 | 1608.90 | 805.45 | 805.52 |
| 414 | Ac-F$r8AAWRAL$Q-NH₂ | 1132 | 1294.76 | 648.38 | 648.48 |
| 415 | Ac-TF$r8AAWAAL$Q-NH₂ | 1133 | 1310.74 | 656.37 | 1311.62 |
| 416 | Ac-TF$r8AAWRAL$A-NH₂ | 1134 | 1338.78 | 670.39 | 670.46 |
| 417 | Ac-VF$r8AAWRAL$Q-NH₂ | 1135 | 1393.82 | 697.91 | 697.99 |
| 418 | Ac-AF$r8AAWAAL$A-NH₂ | 1136 | 1223.71 | 612.86 | 1224.67 |
| 420 | Ac-TF$r8AAWKAL$Q-NH₂ | 1137 | 1367.80 | 684.90 | 684.97 |
| 421 | Ac-TF$r8AAWOAL$Q-NH₂ | 1138 | 1353.78 | 677.89 | 678.01 |
| 422 | Ac-TF$r8AAWSAL$Q-NH₂ | 1139 | 1326.73 | 664.37 | 664.47 |
| 423 | Ac-LTF$r8AAWRAL$Q-NH₂ | 1140 | 1508.89 | 755.45 | 755.49 |
| 424 | Ac-F$r8AYWAQL$A-NH₂ | 1141 | 1301.72 | 651.86 | 651.96 |
| 425 | Ac-F$r8AWWAAL$A-NH₂ | 1142 | 1267.71 | 634.86 | 634.87 |
| 426 | Ac-F$r8AWWAQL$A-NH₂ | 1143 | 1324.73 | 663.37 | 663.43 |
| 427 | Ac-F$r8AYWEAL$-NH₂ | 1144 | 1231.66 | 616.83 | 1232.93 |
| 428 | Ac-F$r8AYWAAL$-NH₂ | 1145 | 1173.66 | 587.83 | 1175.09 |
| 429 | Ac-F$r8AYWKAL$-NH₂ | 1146 | 1230.72 | 616.36 | 616.44 |
| 430 | Ac-F$r8AYWOAL$-NH₂ | 1147 | 1216.70 | 609.35 | 609.48 |
| 431 | Ac-F$r8AYWQAL$-NH₂ | 1148 | 1230.68 | 616.34 | 616.44 |
| 432 | Ac-F$r8AYWAQL$-NH₂ | 1149 | 1230.68 | 616.34 | 616.37 |
| 433 | Ac-F$r8HYWDQL$S-NH₂ | 1150 | 1427.72 | 714.86 | 714.86 |
| 434 | Ac-F$r8HFWEQL$S-NH₂ | 1151 | 1425.74 | 713.87 | 713.98 |
| 435 | Ac-F$r8AYWHQL$S-NH₂ | 1152 | 1383.73 | 692.87 | 692.96 |
| 436 | Ac-F$r8AYWKQL$S-NH₂ | 1153 | 1374.77 | 688.39 | 688.45 |
| 437 | Ac-F$r8AYWOQL$S-NH₂ | 1154 | 1360.75 | 681.38 | 681.49 |
| 438 | Ac-F$r8HYWSQL$S-NH₂ | 1155 | 1399.73 | 700.87 | 700.95 |
| 439 | Ac-F$r8HWWEQL$S-NH₂ | 1156 | 1464.76 | 733.38 | 733.44 |
| 440 | Ac-F$r8HWWAQL$S-NH₂ | 1157 | 1406.75 | 704.38 | 704.43 |
| 441 | Ac-F$r8AWWHQL$S-NH₂ | 1158 | 1406.75 | 704.38 | 704.43 |
| 442 | Ac-F$r8AWWKQL$S-NH₂ | 1159 | 1397.79 | 699.90 | 699.92 |
| 443 | Ac-F$r8AWWOQL$S-NH₂ | 1160 | 1383.77 | 692.89 | 692.96 |
| 444 | Ac-F$r8HWWSQL$S-NH₂ | 1161 | 1422.75 | 712.38 | 712.42 |
| 445 | Ac-LTF$r8NYWANleL$Q-NH₂ | 1162 | 1600.90 | 801.45 | 801.52 |
| 446 | Ac-LTF$r8NLWAQL$Q-NH₂ | 1163 | 1565.90 | 783.95 | 784.06 |
| 447 | Ac-LTF$r8NYWANleL$A-NH₂ | 1164 | 1543.88 | 772.94 | 773.03 |
| 448 | Ac-LTF$r8NLWAQL$A-NH₂ | 1165 | 1508.88 | 755.44 | 755.49 |
| 449 | Ac-LTF$r8AYWANleL$Q-NH₂ | 1166 | 1557.90 | 779.95 | 780.06 |
| 450 | Ac-LTF$r8ALWAQL$Q-NH₂ | 1167 | 1522.89 | 762.45 | 762.45 |
| 451 | Ac-LAF$r8NYWANleL$Q-NH₂ | 1168 | 1570.89 | 786.45 | 786.5 |
| 452 | Ac-LAF$r8NLWAQL$Q-NH₂ | 1169 | 1535.89 | 768.95 | 769.03 |
| 453 | Ac-LAF$r8AYWANleL$A-NH₂ | 1170 | 1470.86 | 736.43 | 736.47 |
| 454 | Ac-LAF$r8ALWAQL$A-NH₂ | 1171 | 1435.86 | 718.93 | 719.01 |
| 455 | Ac-LAF$r8AYWAAL$A-NH₂ | 1172 | 1428.82 | 715.41 | 715.41 |
| 456 | Ac-F$r8AYWEAc3cL$AAib-NH₂ | 1173 | 1399.75 | 700.88 | 700.95 |
| 457 | Ac-F$r8AYWAQL$AA-NH₂ | 1174 | 1372.75 | 687.38 | 687.78 |
| 458 | Ac-F$r8AYWAAc3cL$AA-NH₂ | 1175 | 1327.73 | 664.87 | 664.84 |
| 459 | Ac-F$r8AYWSAc3cL$AA-NH₂ | 1176 | 1343.73 | 672.87 | 672.9 |
| 460 | Ac-F$r8AYWEAc3cL$AS-NH₂ | 1177 | 1401.73 | 701.87 | 701.84 |
| 461 | Ac-F$r8AYWEAc3cL$AT-NH₂ | 1178 | 1415.75 | 708.88 | 708.87 |
| 462 | Ac-F$r8AYWEAc3cL$AL-NH₂ | 1179 | 1427.79 | 714.90 | 714.94 |
| 463 | Ac-F$r8AYWEAc3cL$AQ-NH₂ | 1180 | 1442.76 | 722.38 | 722.41 |
| 464 | Ac-F$r8AFWEAc3cL$AA-NH₂ | 1181 | 1369.74 | 685.87 | 685.93 |
| 465 | Ac-F$r8AWWEAc3cL$AA-NH₂ | 1182 | 1408.75 | 705.38 | 705.39 |
| 466 | Ac-F$r8AYWEAc3cL$SA-NH₂ | 1183 | 1401.73 | 701.87 | 701.99 |
| 467 | Ac-F$r8AYWEAL$AA-NH₂ | 1184 | 1373.74 | 687.87 | 687.93 |
| 468 | Ac-F$r8AYWENleL$AA-NH₂ | 1185 | 1415.79 | 708.90 | 708.94 |
| 469 | Ac-F$r8AYWEAc3cL$AbuA-NH₂ | 1186 | 1399.75 | 700.88 | 700.95 |
| 470 | Ac-F$r8AYWEAc3cL$NleA-NH₂ | 1187 | 1427.79 | 714.90 | 714.86 |
| 471 | Ac-F$r8AYWEAibL$NleA-NH₂ | 1188 | 1429.80 | 715.90 | 715.97 |
| 472 | Ac-F$r8AYWEAL$NleA-NH₂ | 1189 | 1415.79 | 708.90 | 708.94 |
| 473 | Ac-F$r8AYWENleL$NleA-NH₂ | 1190 | 1457.83 | 729.92 | 729.96 |
| 474 | Ac-F$r8AYWEAibL$Abu-NH₂ | 1191 | 1330.73 | 666.37 | 666.39 |
| 475 | Ac-F$r8AYWENleL$Abu-NH₂ | 1192 | 1358.76 | 680.38 | 680.39 |
| 476 | Ac-F$r8AYWEAL$Abu-NH₂ | 1193 | 1316.72 | 659.36 | 659.36 |
| 477 | Ac-LTF$r8AFWAQL$S-NH₂ | 1194 | 1515.85 | 758.93 | 759.12 |
| 478 | Ac-LTF$r8AWWAQL$S-NH₂ | 1195 | 1554.86 | 778.43 | 778.51 |
| 479 | Ac-LTF$r8AYWAQI$S-NH₂ | 1196 | 1531.84 | 766.92 | 766.96 |
| 480 | Ac-LTF$r8AYWAQNle$S-NH₂ | 1197 | 1531.84 | 766.92 | 766.96 |
| 481 | Ac-LTF$r8AYWAQL$SA-NH₂ | 1198 | 1602.88 | 802.44 | 802.48 |
| 482 | Ac-LTF$r8AWWAQL$A-NH₂ | 1199 | 1538.87 | 770.44 | 770.89 |
| 483 | Ac-LTF$r8AYWAQI$A-NH₂ | 1200 | 1515.85 | 758.93 | 759.42 |
| 484 | Ac-LTF$r8AYWAQNle$A-NH₂ | 1201 | 1515.85 | 758.93 | 759.42 |
| 485 | Ac-LTF$r8AYWAQL$AA-NH₂ | 1202 | 1586.89 | 794.45 | 794.94 |
| 486 | Ac-LTF$r8HWWAQL$S-NH₂ | 1203 | 1620.88 | 811.44 | 811.47 |
| 487 | Ac-LTF$r8HRWAQL$S-NH₂ | 1204 | 1590.90 | 796.45 | 796.52 |
| 488 | Ac-LTF$r8HKWAQL$S-NH₂ | 1205 | 1562.90 | 782.45 | 782.53 |
| 489 | Ac-LTF$r8HYWAQL$W-NH₂ | 1206 | 1696.91 | 849.46 | 849.5 |
| 491 | Ac-F$r8AYWAbuAL$A-NH₂ | 1207 | 1258.71 | 630.36 | 630.5 |
| 492 | Ac-F$r8AbuYWEAL$A-NH₂ | 1208 | 1316.72 | 659.36 | 659.51 |
| 493 | Ac-NlePRF % r8NYWRLL % QN-NH₂ | 1209 | 1954.13 | 978.07 | 978.54 |
| 494 | Ac-TSF % r8HYWAQL % S-NH₂ | 1210 | 1573.83 | 787.92 | 787.98 |
| 495 | Ac-LTF % r8AYWAQL % S-NH₂ | 1211 | 1533.86 | 767.93 | 768 |
| 496 | Ac-HTF$r8HYWAQL$S-NH₂ | 1212 | 1621.84 | 811.92 | 811.96 |
| 497 | Ac-LHF$r8HYWAQL$S-NH₂ | 1213 | 1633.88 | 817.94 | 818.02 |
| 498 | Ac-LTF$r8HHWAQL$S-NH₂ | 1214 | 1571.86 | 786.93 | 786.94 |
| 499 | Ac-LTF$r8HYWHQL$S-NH₂ | 1215 | 1663.89 | 832.95 | 832.38 |
| 500 | Ac-LTF$r8HYWAHL$S-NH₂ | 1216 | 1606.87 | 804.44 | 804.48 |
| 501 | Ac-LTF$r8HYWAQL$H-NH₂ | 1217 | 1647.89 | 824.95 | 824.98 |
| 502 | Ac-LTF$r8HYWAQL$S-NHPr | 1218 | 1639.91 | 820.96 | 820.98 |
| 503 | Ac-LTF$r8HYWAQL$S-NHsBu | 1219 | 1653.93 | 827.97 | 828.02 |
| 504 | Ac-LTF$r8HYWAQL$S-NHiBu | 1220 | 1653.93 | 827.97 | 828.02 |
| 505 | Ac-LTF$r8HYWAQL$S-NHBn | 1221 | 1687.91 | 844.96 | 844.44 |
| 506 | Ac-LTF$r8HYWAQL$S-NHPe | 1222 | 1700.92 | 851.46 | 851.99 |
| 507 | Ac-LTF$r8HYWAQL$S-NHChx | 1223 | 1679.94 | 840.97 | 841.04 |
| 508 | Ac-ETF$r8AYWAQL$S-NH₂ | 1224 | 1547.80 | 774.90 | 774.96 |
| 509 | Ac-STF$r8AYWAQL$S-NH₂ | 1225 | 1505.79 | 753.90 | 753.94 |
| 510 | Ac-LEF$r8AYWAQL$S-NH₂ | 1226 | 1559.84 | 780.92 | 781.25 |
| 511 | Ac-LSF$r8AYWAQL$S-NH₂ | 1227 | 1517.83 | 759.92 | 759.93 |
| 512 | Ac-LTF$r8EYWAQL$S-NH₂ | 1228 | 1589.85 | 795.93 | 795.97 |
| 513 | Ac-LTF$r8SYWAQL$S-NH₂ | 1229 | 1547.84 | 774.92 | 774.96 |
| 514 | Ac-LTF$r8AYWEQL$S-NH₂ | 1230 | 1589.85 | 795.93 | 795.9 |
| 515 | Ac-LTF$r8AYWAEL$S-NH₂ | 1231 | 1532.83 | 767.42 | 766.96 |
| 516 | Ac-LTF$r8AYWASL$S-NH₂ | 1232 | 1490.82 | 746.41 | 746.46 |
| 517 | Ac-LTF$r8AYWAQL$E-NH₂ | 1233 | 1573.85 | 787.93 | 787.98 |
| 518 | Ac-LTF2CN$r8HYWAQL$S-NH₂ | 1234 | 1622.86 | 812.43 | 812.47 |
| 519 | Ac-LTF3Cl$r8HYWAQL$S-NH₂ | 1235 | 1631.83 | 816.92 | 816.99 |
| 520 | Ac-LTDip$r8HYWAQL$S-NH₂ | 1236 | 1673.90 | 837.95 | 838.01 |
| 521 | Ac-LTF$r8HYWAQTle$S-NH₂ | 1237 | 1597.87 | 799.94 | 800.04 |
| 522 | Ac-F$r8AY6clWEAL$A-NH₂ | 1238 | 1336.66 | 669.33 | 1338.56 |
| 523 | Ac-F$r8AYdl6brWEAL$A-NH₂ | 1239 | 1380.61 | 691.31 | 692.2 |
| 524 | Ac-F$r8AYdl6fWEAL$A-NH₂ | 1240 | 1320.69 | 661.35 | 1321.61 |
| 525 | Ac-F$r8AYdl4mWEAL$A-NH₂ | 1241 | 1316.72 | 659.36 | 659.36 |
| 526 | Ac-F$r8AYdl5clWEAL$A-NH₂ | 1242 | 1336.66 | 669.33 | 669.35 |
| 527 | Ac-F$r8AYdl7mWEAL$A-NH₂ | 1243 | 1316.72 | 659.36 | 659.36 |
| 528 | Ac-LTF % r8HYWAQL % A-NH₂ | 1244 | 1583.89 | 792.95 | 793.01 |
| 529 | Ac-LTF$r8HCouWAQL$S-NH₂ | 1245 | 1679.87 | 840.94 | 841.38 |
| 530 | Ac-LTFEHCouWAQLTS-NH₂ | 1246 | 1617.75 | 809.88 | 809.96 |
| 531 | Ac-LTA$r8HCouWAQL$S-NH₂ | 1247 | 1603.84 | 802.92 | 803.36 |
| 532 | Ac-F$r8AYWEAL$AbuA-NH₂ | 1248 | 1387.75 | 694.88 | 694.88 |
| 533 | Ac-F$r8AYWEAI$AA-NH₂ | 1249 | 1373.74 | 687.87 | 687.93 |
| 534 | Ac-F$r8AYWEANle$AA-NH₂ | 1250 | 1373.74 | 687.87 | 687.93 |
| 535 | Ac-F$r8AYWEAmlL$AA-NH₂ | 1251 | 1429.80 | 715.90 | 715.97 |
| 536 | Ac-F$r8AYWQAL$AA-NH₂ | 1252 | 1372.75 | 687.38 | 687.48 |
| 537 | Ac-F$r8AYWAAL$AA-NH₂ | 1253 | 1315.73 | 658.87 | 658.92 |
| 538 | Ac-F$r8AYWAbuAL$AA-NH₂ | 1254 | 1329.75 | 665.88 | 665.95 |
| 539 | Ac-F$r8AYWNleAL$AA-NH₂ | 1255 | 1357.78 | 679.89 | 679.94 |
| 540 | Ac-F$r8AbuYWEAL$AA-NH₂ | 1256 | 1387.75 | 694.88 | 694.96 |
| 541 | Ac-F$r8NleYWEAL$AA-NH₂ | 1257 | 1415.79 | 708.90 | 708.94 |
| 542 | Ac-F$r8FYWEAL$AA-NH₂ | 1258 | 1449.77 | 725.89 | 725.97 |
| 543 | Ac-LTF$r8HYWAQhL$S-NH₂ | 1259 | 1611.88 | 806.94 | 807 |
| 544 | Ac-LTF$r8HYWAQAdm$S-NH₂ | 1260 | 1675.91 | 838.96 | 839.04 |
| 545 | Ac-LTF$r8HYWAQIgl$S-NH₂ | 1261 | 1659.88 | 830.94 | 829.94 |
| 546 | Ac-F$r8AYWAQL$AA-NH₂ | 1262 | 1372.75 | 687.38 | 687.48 |
| 547 | Ac-LTF$r8ALWAQL$Q-NH₂ | 1263 | 1522.89 | 762.45 | 762.52 |
| 548 | Ac-F$r8AYWEAL$AA-NH₂ | 1264 | 1373.74 | 687.87 | 687.93 |
| 549 | Ac-F$r8AYWENleL$AA-NH₂ | 1265 | 1415.79 | 708.90 | 708.94 |
| 550 | Ac-F$r8AYWEAibL$Abu-NH₂ | 1266 | 1330.73 | 666.37 | 666.39 |
| 551 | Ac-F$r8AYWENleL$Abu-NH₂ | 1267 | 1358.76 | 680.38 | 680.38 |
| 552 | Ac-F$r8AYWEAL$Abu-NH₂ | 1268 | 1316.72 | 659.36 | 659.36 |
| 553 | Ac-F$r8AYWEAc3cL$AbuA-NH₂ | 1269 | 1399.75 | 700.88 | 700.95 |
| 554 | Ac-F$r8AYWEAc3cL$NleA-NH₂ | 1270 | 1427.79 | 714.90 | 715.01 |
| 555 | H-LTF$r8AYWAQL$S-NH₂ | 1271 | 1489.83 | 745.92 | 745.95 |
| 556 | mdPEG3-LTF$r8AYWAQL$S-NH₂ | 1272 | 1679.92 | 840.96 | 840.97 |
| 557 | mdPEG7-LTF$r8AYWAQL$S-NH₂ | 1273 | 1856.02 | 929.01 | 929.03 |
| 558 | Ac-F$r8ApmpEt6clWEAL$A-NH₂ | 1274 | 1470.71 | 736.36 | 788.17 |
| 559 | Ac-LTF3Cl$r8AYWAQL$S-NH₂ | 1275 | 1565.81 | 783.91 | 809.18 |
| 560 | Ac-LTF3Cl$r8HYWAQL$A-NH₂ | 1276 | 1615.83 | 808.92 | 875.24 |
| 561 | Ac-LTF3Cl$r8HYWWQL$S-NH₂ | 1277 | 1746.87 | 874.44 | 841.65 |
| 562 | Ac-LTF3Cl$r8AYWWQL$S-NH₂ | 1278 | 1680.85 | 841.43 | 824.63 |
| 563 | Ac-LTF$r8AYWWQL$S-NH₂ | 1279 | 1646.89 | 824.45 | 849.98 |
| 564 | Ac-LTF$r8HYWWQL$A-NH₂ | 1280 | 1696.91 | 849.46 | 816.67 |
| 565 | Ac-LTF$r8AYWWQL$A-NH₂ | 1281 | 1630.89 | 816.45 | 776.15 |
| 566 | Ac-LTF4F$r8AYWAQL$S-NH₂ | 1282 | 1549.83 | 775.92 | 776.15 |
| 567 | Ac-LTF2F$r8AYWAQL$S-NH₂ | 1283 | 1549.83 | 775.92 | 776.15 |
| 568 | Ac-LTF3F$r8AYWAQL$S-NH₂ | 1284 | 1549.83 | 775.92 | 785.12 |
| 569 | Ac-LTF34F2$r8AYWAQL$S-NH₂ | 1285 | 1567.83 | 784.92 | 785.12 |
| 570 | Ac-LTF35F2$r8AYWAQL$S-NH₂ | 1286 | 1567.83 | 784.92 | 1338.74 |
| 571 | Ac-F3Cl$r8AYWEAL$A-NH₂ | 1287 | 1336.66 | 669.33 | 705.28 |
| 572 | Ac-F3Cl$r8AYWEAL$AA-NH₂ | 1288 | 1407.70 | 704.85 | 680.11 |
| 573 | Ac-F$r8AY6clWEAL$AA-NH₂ | 1289 | 1407.70 | 704.85 | 736.83 |
| 574 | Ac-F$r8AY6clWEAL$-NH₂ | 1290 | 1265.63 | 633.82 | 784.1 |
| 575 | Ac-LTF$r8HYWAQLSt/S-NH₂ | 1291 | 16.03 | 9.02 | 826.98 |
| 576 | Ac-LTF$r8HYWAQL$S-NHsBu | 1292 | 1653.93 | 827.97 | 828.02 |
| 577 | Ac-STF$r8AYWAQL$S-NH₂ | 1293 | 1505.79 | 753.90 | 753.94 |
| 578 | Ac-LTF$r8AYWAEL$S-NH₂ | 1294 | 1532.83 | 767.42 | 767.41 |
| 579 | Ac-LTF$r8AYWAQL$E-NH₂ | 1295 | 1573.85 | 787.93 | 787.98 |
| 580 | mdPEG3-LTF$r8AYWAQL$S-NH₂ | 1296 | 1679.92 | 840.96 | 840.97 |
| 581 | Ac-LTF$r8AYWAQhL$S-NH₂ | 1297 | 1545.86 | 773.93 | 774.31 |
| 583 | Ac-LTF$r8AYWAQCha$S-NH₂ | 1298 | 1571.88 | 786.94 | 787.3 |
| 584 | Ac-LTF$r8AYWAQChg$S-NH₂ | 1299 | 1557.86 | 779.93 | 780.4 |
| 585 | Ac-LTF$r8AYWAQCba$S-NH₂ | 1300 | 1543.84 | 772.92 | 780.13 |
| 586 | Ac-LTF$r8AYWAQF$S-NH₂ | 1301 | 1565.83 | 783.92 | 784.2 |
| 587 | Ac-LTF4F$r8HYWAQhL$S-NH₂ | 1302 | 1629.87 | 815.94 | 815.36 |
| 588 | Ac-LTF4F$r8HYWAQCha$S-NH₂ | 1303 | 1655.89 | 828.95 | 828.39 |
| 589 | Ac-LTF4F$r8HYWAQChg$S-NH₂ | 1304 | 1641.87 | 821.94 | 821.35 |
| 590 | Ac-LTF4F$r8HYWAQCba$S-NH₂ | 1305 | 1627.86 | 814.93 | 814.32 |
| 591 | Ac-LTF4F$r8AYWAQhL$S-NH₂ | 1306 | 1563.85 | 782.93 | 782.36 |
| 592 | Ac-LTF4F$r8AYWAQCha$S-NH₂ | 1307 | 1589.87 | 795.94 | 795.38 |
| 593 | Ac-LTF4F$r8AYWAQChg$S-NH₂ | 1308 | 1575.85 | 788.93 | 788.35 |
| 594 | Ac-LTF4F$r8AYWAQCba$S-NH₂ | 1309 | 1561.83 | 781.92 | 781.39 |
| 595 | Ac-LTF3Cl$r8AYWAQhL$S-NH₂ | 1310 | 1579.82 | 790.91 | 790.35 |
| 596 | Ac-LTF3Cl$r8AYWAQCha$S-NH₂ | 1311 | 1605.84 | 803.92 | 803.67 |
| 597 | Ac-LTF3Cl$r8AYWAQChg$S-NH₂ | 1312 | 1591.82 | 796.91 | 796.34 |
| 598 | Ac-LTF3Cl$r8AYWAQCba$S-NH₂ | 1313 | 1577.81 | 789.91 | 789.39 |
| 599 | Ac-LTF$r8AYWAQhF$S-NH₂ | 1314 | 1579.84 | 790.92 | 791.14 |
| 600 | Ac-LTF$r8AYWAQF3CF3$S-NH₂ | 1315 | 1633.82 | 817.91 | 818.15 |
| 601 | Ac-LTF$r8AYWAQF3Me$S-NH₂ | 1316 | 1581.86 | 791.93 | 791.32 |
| 602 | Ac-LTF$r8AYWAQ1Nal$S-NH₂ | 1317 | 1615.84 | 808.92 | 809.18 |
| 603 | Ac-LTF$r8AYWAQBip$S-NH₂ | 1318 | 1641.86 | 821.93 | 822.13 |
| 604 | Ac-LTF$r8FYWAQL$A-NH₂ | 1319 | 1591.88 | 796.94 | 797.33 |
| 605 | Ac-LTF$r8HYWAQL$S-NHAm | 1320 | 1667.94 | 834.97 | 835.92 |
| 606 | Ac-LTF$r8HYWAQL$S-NHiAm | 1321 | 1667.94 | 834.97 | 835.55 |
| 607 | Ac-LTF$r8HYWAQL$S-NHnPr3Ph | 1322 | 1715.94 | 858.97 | 859.79 |
| 608 | Ac-LTF$r8HYWAQL$S-NHnBu3,3Me | 1323 | 1681.96 | 841.98 | 842.49 |
| 610 | Ac-LTF$r8HYWAQL$S-NHnPr | 1324 | 1639.91 | 820.96 | 821.58 |
| 611 | Ac-LTF$r8HYWAQL$S-NHnEt2Ch | 1325 | 1707.98 | 854.99 | 855.35 |
| 612 | Ac-LTF$r8HYWAQL$S-NHHex | 1326 | 1681.96 | 841.98 | 842.4 |
| 613 | Ac-LTF$r8AYWAQL$S-NHmdPeg2 | 1327 | 1633.91 | 817.96 | 818.35 |
| 614 | Ac-LTF$r8AYWAQL$A-NHmdPeg2 | 1328 | 1617.92 | 809.96 | 810.3 |
| 615 | Ac-LTF$r8AYWAQL$A-NHmdPeg4 | 1329 | 1705.97 | 853.99 | 854.33 |
| 616 | Ac-F$r8AYdl4mWEAL$A-NH₂ | 1330 | 1316.72 | 659.36 | 659.44 |
| 617 | Ac-F$r8AYdl5clWEAL$A-NH₂ | 1331 | 1336.66 | 669.33 | 669.43 |
| 618 | Ac-LThF$r8AYWAQL$S-NH₂ | 1332 | 1545.86 | 773.93 | 774.11 |
| 619 | Ac-LT2Nal$r8AYWAQL$S-NH₂ | 1333 | 1581.86 | 791.93 | 792.43 |
| 620 | Ac-LTA$r8AYWAQL$S-NH₂ | 1334 | 1455.81 | 728.91 | 729.15 |
| 621 | Ac-LTF$r8AYWVQL$S-NH₂ | 1335 | 1559.88 | 780.94 | 781.24 |
| 622 | Ac-LTF$r8HYWAAL$A-NH₂ | 1336 | 1524.85 | 763.43 | 763.86 |
| 623 | Ac-LTF$r8VYWAQL$A-NH₂ | 1337 | 1543.88 | 772.94 | 773.37 |
| 624 | Ac-LTF$r8IYWAQL$S-NH₂ | 1338 | 1573.89 | 787.95 | 788.17 |
| 625 | Ac-FTF$r8VYWSQL$S-NH₂ | 1339 | 1609.85 | 805.93 | 806.22 |
| 626 | Ac-ITF$r8FYWAQL$S-NH₂ | 1340 | 1607.88 | 804.94 | 805.2 |
| 627 | Ac-2NalTF$r8VYWSQL$S-NH₂ | 1341 | 1659.87 | 830.94 | 831.2 |
| 628 | Ac-ITF$r8LYWSQL$S-NH₂ | 1342 | 1589.89 | 795.95 | 796.13 |
| 629 | Ac-FTF$r8FYWAQL$S-NH₂ | 1343 | 1641.86 | 821.93 | 822.13 |
| 630 | Ac-WTF$r8VYWAQL$S-NH₂ | 1344 | 1632.87 | 817.44 | 817.69 |
| 631 | Ac-WTF$r8WYWAQL$S-NH₂ | 1345 | 1719.88 | 860.94 | 861.36 |
| 632 | Ac-VTF$r8AYWSQL$S-NH₂ | 1346 | 1533.82 | 767.91 | 768.19 |
| 633 | Ac-WTF$r8FYWSQL$S-NH₂ | 1347 | 1696.87 | 849.44 | 849.7 |
| 634 | Ac-FTF$r8IYWAQL$S-NH₂ | 1348 | 1607.88 | 804.94 | 805.2 |
| 635 | Ac-WTF$r8VYWSQL$S-NH₂ | 1349 | 1648.87 | 825.44 | 824.8 |
| 636 | Ac-FTF$r8LYWSQL$S-NH₂ | 1350 | 1623.87 | 812.94 | 812.8 |
| 637 | Ac-YTF$r8FYWSQL$S-NH₂ | 1351 | 1673.85 | 837.93 | 837.8 |
| 638 | Ac-LTF$r8AY6clWEAL$A-NH₂ | 1352 | 1550.79 | 776.40 | 776.14 |
| 639 | Ac-LTF$r8AY6clWSQL$S-NH₂ | 1353 | 1581.80 | 791.90 | 791.68 |
| 640 | Ac-F$r8AY6clWSAL$A-NH₂ | 1354 | 1294.65 | 648.33 | 647.67 |
| 641 | Ac-F$r8AY6clWQAL$AA-NH₂ | 1355 | 1406.72 | 704.36 | 703.84 |
| 642 | Ac-LHF$r8AYWAQL$S-NH₂ | 1356 | 1567.86 | 784.93 | 785.21 |
| 643 | Ac-LTF$r8AYWAQL$S-NH₂ | 1357 | 1531.84 | 766.92 | 767.17 |
| 644 | Ac-LTF$r8AHWAQL$S-NH₂ | 1358 | 1505.84 | 753.92 | 754.13 |
| 645 | Ac-LTF$r8AYWAHL$S-NH₂ | 1359 | 1540.84 | 771.42 | 771.61 |
| 646 | Ac-LTF$r8AYWAQL$H-NH₂ | 1360 | 1581.87 | 791.94 | 792.15 |
| 647 | H-LTF$r8AYWAQL$A-NH₂ | 1361 | 1473.84 | 737.92 | 737.29 |
| 648 | Ac-HHF$r8AYWAQL$S-NH₂ | 1362 | 1591.83 | 796.92 | 797.35 |
| 649 | Ac-aAibWTF$r8VYWSQL$S-NH₂ | 1363 | 1804.96 | 903.48 | 903.64 |
| 650 | Ac-AibWTF$r8HYWAQL$S-NH₂ | 1364 | 1755.91 | 878.96 | 879.4 |
| 651 | Ac-AibAWTF$r8HYWAQL$S-NH₂ | 1365 | 1826.95 | 914.48 | 914.7 |
| 652 | Ac-fWTF$r8HYWAQL$S-NH₂ | 1366 | 1817.93 | 909.97 | 910.1 |
| 653 | Ac-AibWWTF$r8HYWAQL$S-NH₂ | 1367 | 1941.99 | 972.00 | 972.2 |
| 654 | Ac-WTF$r8LYWSQL$S-NH₂ | 1368 | 1662.88 | 832.44 | 832.8 |
| 655 | Ac-WTF$r8NleYWSQL$S-NH₂ | 1369 | 1662.88 | 832.44 | 832.6 |
| 656 | Ac-LTF$r8AYWSQL$a-NH₂ | 1370 | 1531.84 | 766.92 | 767.2 |
| 657 | Ac-LTF$r8EYWARL$A-NH₂ | 1371 | 1601.90 | 801.95 | 802.1 |
| 658 | Ac-LTF$r8EYWAHL$A-NH₂ | 1372 | 1582.86 | 792.43 | 792.6 |
| 659 | Ac-aTF$r8AYWAQL$S-NH₂ | 1373 | 1489.80 | 745.90 | 746.08 |
| 660 | Ac-AibTF$r8AYWAQL$S-NH₂ | 1374 | 1503.81 | 752.91 | 753.11 |
| 661 | Ac-AmfTF$r8AYWAQL$S-NH₂ | 1375 | 1579.84 | 790.92 | 791.14 |
| 662 | Ac-AmwTF$r8AYWAQL$S-NH₂ | 1376 | 1618.86 | 810.43 | 810.66 |
| 663 | Ac-NmLTF$r8AYWAQL$S-NH₂ | 1377 | 1545.86 | 773.93 | 774.11 |
| 664 | Ac-LNmTF$r8AYWAQL$S-NH₂ | 1378 | 1545.86 | 773.93 | 774.11 |
| 665 | Ac-LSarF$r8AYWAQL$S-NH₂ | 1379 | 1501.83 | 751.92 | 752.18 |
| 667 | Ac-LGF$r8AYWAQL$S-NH₂ | 1380 | 1487.82 | 744.91 | 745.15 |
| 668 | Ac-LTNmF$r8AYWAQL$S-NH₂ | 1381 | 1545.86 | 773.93 | 774.2 |
| 669 | Ac-TF$r8AYWAQL$S-NH₂ | 1382 | 1418.76 | 710.38 | 710.64 |
| 670 | Ac-ETF$r8AYWAQL$A-NH₂ | 1383 | 1531.81 | 766.91 | 767.2 |
| 671 | Ac-LTF$r8EYWAQL$A-NH₂ | 1384 | 1573.85 | 787.93 | 788.1 |
| 672 | Ac-LT2Nal$r8AYWSQL$S-NH₂ | 1385 | 1597.85 | 799.93 | 800.4 |
| 673 | Ac-LTF$r8AYWAAL$S-NH₂ | 1386 | 1474.82 | 738.41 | 738.68 |
| 674 | Ac-LTF$r8AYWAQhCha$S-NH₂ | 1387 | 1585.89 | 793.95 | 794.19 |
| 675 | Ac-LTF$r8AYWAQChg$S-NH₂ | 1388 | 1557.86 | 779.93 | 780.97 |
| 676 | Ac-LTF$r8AYWAQCba$S-NH₂ | 1389 | 1543.84 | 772.92 | 773.19 |
| 677 | Ac-LTF$r8AYWAQF3CF3$S-NH₂ | 1390 | 1633.82 | 817.91 | 818.15 |
| 678 | Ac-LTF$r8AYWAQ1Nal$S-NH₂ | 1391 | 1615.84 | 808.92 | 809.18 |
| 679 | Ac-LTF$r8AYWAQBip$S-NH₂ | 1392 | 1641.86 | 821.93 | 822.32 |
| 680 | Ac-LT2Nal$r8AYWAQL$S-NH₂ | 1393 | 1581.86 | 791.93 | 792.15 |
| 681 | Ac-LTF$r8AYWVQL$S-NH₂ | 1394 | 1559.88 | 780.94 | 781.62 |
| 682 | Ac-LTF$r8AWWAQL$S-NH₂ | 1395 | 1554.86 | 778.43 | 778.65 |
| 683 | Ac-FTF$r8VYWSQL$S-NH₂ | 1396 | 1609.85 | 805.93 | 806.12 |
| 684 | Ac-ITF$r8FYWAQL$S-NH₂ | 1397 | 1607.88 | 804.94 | 805.2 |
| 685 | Ac-ITF$r8LYWSQL$S-NH₂ | 1398 | 1589.89 | 795.95 | 796.22 |
| 686 | Ac-FTF$r8FYWAQL$S-NH₂ | 1399 | 1641.86 | 821.93 | 822.41 |
| 687 | Ac-VTF$r8AYWSQL$S-NH₂ | 1400 | 1533.82 | 767.91 | 768.19 |
| 688 | Ac-LTF$r8AHWAQL$S-NH₂ | 1401 | 1505.84 | 753.92 | 754.31 |
| 689 | Ac-LTF$r8AYWAQL$H-NH₂ | 1402 | 1581.87 | 791.94 | 791.94 |
| 690 | Ac-LTF$r8AYWAHL$S-NH₂ | 1403 | 1540.84 | 771.42 | 771.61 |
| 691 | Ac-aAibWTF$r8VYWSQL$S-NH₂ | 1404 | 1804.96 | 903.48 | 903.9 |
| 692 | Ac-AibWTF$r8HYWAQL$S-NH₂ | 1405 | 1755.91 | 878.96 | 879.5 |
| 693 | Ac-AibAWTF$r8HYWAQL$S-NH₂ | 1406 | 1826.95 | 914.48 | 914.7 |
| 694 | Ac-fWTF$r8HYWAQL$S-NH₂ | 1407 | 1817.93 | 909.97 | 910.2 |
| 695 | Ac-AibWWTF$r8HYWAQL$S-NH₂ | 1408 | 1941.99 | 972.00 | 972.7 |
| 696 | Ac-WTF$r8LYWSQL$S-NH₂ | 1409 | 1662.88 | 832.44 | 832.7 |
| 697 | Ac-WTF$r8NleYWSQL$S-NH₂ | 1410 | 1662.88 | 832.44 | 832.7 |
| 698 | Ac-LTF$r8AYWSQL$a-NH₂ | 1411 | 1531.84 | 766.92 | 767.2 |
| 699 | Ac-LTF$r8EYWARL$A-NH₂ | 1412 | 1601.90 | 801.95 | 802.2 |
| 700 | Ac-LTF$r8EYWAHL$A-NH₂ | 1413 | 1582.86 | 792.43 | 792.6 |
| 701 | Ac-aTF$r8AYWAQL$S-NH₂ | 1414 | 1489.80 | 745.90 | 746.1 |
| 702 | Ac-AibTF$r8AYWAQL$S-NH₂ | 1415 | 1503.81 | 752.91 | 753.2 |
| 703 | Ac-AmfTF$r8AYWAQL$S-NH₂ | 1416 | 1579.84 | 790.92 | 791.2 |
| 704 | Ac-AmwTF$r8AYWAQL$S-NH₂ | 1417 | 1618.86 | 810.43 | 810.7 |
| 705 | Ac-NmLTF$r8AYWAQL$S-NH₂ | 1418 | 1545.86 | 773.93 | 774.1 |
| 706 | Ac-LNmTF$r8AYWAQL$S-NH₂ | 1419 | 1545.86 | 773.93 | 774.4 |
| 707 | Ac-LSarF$r8AYWAQL$S-NH₂ | 1420 | 1501.83 | 751.92 | 752.1 |
| 708 | Ac-TF$r8AYWAQL$S-NH₂ | 1421 | 1418.76 | 710.38 | 710.8 |
| 709 | Ac-ETF$r8AYWAQL$A-NH₂ | 1422 | 1531.81 | 766.91 | 767.4 |
| 710 | Ac-LTF$r8EYWAQL$A-NH₂ | 1423 | 1573.85 | 787.93 | 788.2 |
| 711 | Ac-WTF$r8VYWSQL$S-NH₂ | 1424 | 1648.87 | 825.44 | 825.2 |
| 713 | Ac-YTF$r8FYWSQL$S-NH₂ | 1425 | 1673.85 | 837.93 | 837.3 |
| 714 | Ac-F$r8AY6clWSAL$A-NH₂ | 1426 | 1294.65 | 648.33 | 647.74 |
| 715 | Ac-ETF$r8EYWVQL$S-NH₂ | 1427 | 1633.84 | 817.92 | 817.36 |
| 716 | Ac-ETF$r8EHWAQL$A-NH₂ | 1428 | 1563.81 | 782.91 | 782.36 |
| 717 | Ac-ITF$r8EYWAQL$S-NH₂ | 1429 | 1589.85 | 795.93 | 795.38 |
| 718 | Ac-ITF$r8EHWVQL$A-NH₂ | 1430 | 1575.88 | 788.94 | 788.42 |
| 719 | Ac-ITF$r8EHWAQL$S-NH₂ | 1431 | 1563.85 | 782.93 | 782.43 |
| 720 | Ac-LTF4F$r8AYWAQCba$S-NH₂ | 1432 | 1561.83 | 781.92 | 781.32 |
| 721 | Ac-LTF3Cl$r8AYWAQhL$S-NH₂ | 1433 | 1579.82 | 790.91 | 790.64 |
| 722 | Ac-LTF3Cl$r8AYWAQCha$S-NH₂ | 1434 | 1605.84 | 803.92 | 803.37 |
| 723 | Ac-LTF3Cl$r8AYWAQChg$S-NH₂ | 1435 | 1591.82 | 796.91 | 796.27 |
| 724 | Ac-LTF3Cl$r8AYWAQCba$S-NH₂ | 1436 | 1577.81 | 789.91 | 789.83 |
| 725 | Ac-LTF$r8AY6clWSQL$S-NH₂ | 1437 | 1581.80 | 791.90 | 791.75 |
| 726 | Ac-LTF4F$r8HYWAQhL$S-NH₂ | 1438 | 1629.87 | 815.94 | 815.36 |
| 727 | Ac-LTF4F$r8HYWAQCba$S-NH₂ | 1439 | 1627.86 | 814.93 | 814.32 |
| 728 | Ac-LTF4F$r8AYWAQhL$S-NH₂ | 1440 | 1563.85 | 782.93 | 782.36 |
| 729 | Ac-LTF4F$r8AYWAQChg$S-NH₂ | 1441 | 1575.85 | 788.93 | 788.35 |
| 730 | Ac-ETF$r8EYWVAL$S-NH₂ | 1442 | 1576.82 | 789.41 | 788.79 |
| 731 | Ac-ETF$r8EHWAAL$A-NH₂ | 1443 | 1506.79 | 754.40 | 754.8 |
| 732 | Ac-ITF$r8EYWAAL$S-NH₂ | 1444 | 1532.83 | 767.42 | 767.75 |
| 733 | Ac-ITF$r8EHWVAL$A-NH₂ | 1445 | 1518.86 | 760.43 | 760.81 |
| 734 | Ac-ITF$r8EHWAAL$S-NH₂ | 1446 | 1506.82 | 754.41 | 754.8 |
| 735 | Pam-LTF$r8EYWAQL$S-NH₂ | 1447 | 1786.07 | 894.04 | 894.48 |
| 736 | Pam-ETF$r8EYWAQL$S-NH₂ | 1448 | 1802.03 | 902.02 | 902.34 |
| 737 | Ac-LTF$r8AYWLQL$S-NH₂ | 1449 | 1573.89 | 787.95 | 787.39 |
| 738 | Ac-LTF$r8EYWLQL$S-NH₂ | 1450 | 1631.90 | 816.95 | 817.33 |
| 739 | Ac-LTF$r8EHWLQL$S-NH₂ | 1451 | 1605.89 | 803.95 | 804.29 |
| 740 | Ac-LTF$r8VYWAQL$S-NH₂ | 1452 | 1559.88 | 780.94 | 781.34 |
| 741 | Ac-LTF$r8AYWSQL$S-NH₂ | 1453 | 1547.84 | 774.92 | 775.33 |
| 742 | Ac-ETF$r8AYWAQL$S-NH₂ | 1454 | 1547.80 | 774.90 | 775.7 |
| 743 | Ac-LTF$r8EYWAQL$S-NH₂ | 1455 | 1589.85 | 795.93 | 796.33 |
| 744 | Ac-LTF$r8HYWAQL$S-NHAm | 1456 | 1667.94 | 834.97 | 835.37 |
| 745 | Ac-LTF$r8HYWAQL$S-NHiAm | 1457 | 1667.94 | 834.97 | 835.27 |
| 746 | Ac-LTF$r8HYWAQL$S-NHnPr3Ph | 1458 | 1715.94 | 858.97 | 859.42 |
| 747 | Ac-LTF$r8HYWAQL$S-NHnBu3,3Me | 1459 | 1681.96 | 841.98 | 842.67 |
| 748 | Ac-LTF$r8HYWAQL$S-NHnBu | 1460 | 1653.93 | 827.97 | 828.24 |
| 749 | Ac-LTF$r8HYWAQL$S-NHnPr | 1461 | 1639.91 | 820.96 | 821.31 |
| 750 | Ac-LTF$r8HYWAQL$S-NHnEt2Ch | 1462 | 1707.98 | 854.99 | 855.35 |
| 751 | Ac-LTF$r8HYWAQL$S-NHHex | 1463 | 1681.96 | 841.98 | 842.4 |
| 752 | Ac-LTF$r8AYWAQL$S-NHmdPeg2 | 1464 | 1633.91 | 817.96 | 855.35 |
| 753 | Ac-LTF$r8AYWAQL$A-NHmdPeg2 | 1465 | 1617.92 | 809.96 | 810.58 |
| 754 | Ac-LTF$r5AYWAAL$s8S-NH₂ | 1466 | 1474.82 | 738.41 | 738.79 |
| 755 | Ac-LTF$r8AYWCouQL$S-NH₂ | 1467 | 1705.88 | 853.94 | 854.61 |
| 756 | Ac-LTF$r8CouYWAQL$S-NH₂ | 1468 | 1705.88 | 853.94 | 854.7 |
| 757 | Ac-CouTF$r8AYWAQL$S-NH₂ | 1469 | 1663.83 | 832.92 | 833.33 |
| 758 | H-LTF$r8AYWAQL$A-NH₂ | 1470 | 1473.84 | 737.92 | 737.29 |
| 759 | Ac-HHF$r8AYWAQL$S-NH₂ | 1471 | 1591.83 | 796.92 | 797.72 |
| 760 | Ac-LT2Nal$r8AYWSQL$S-NH₂ | 1472 | 1597.85 | 799.93 | 800.68 |
| 761 | Ac-LTF$r8HCouWAQL$S-NH₂ | 1473 | 1679.87 | 840.94 | 841.38 |
| 762 | Ac-LTF$r8AYWCou2QL$S-NH₂ | 1474 | 1789.94 | 895.97 | 896.51 |
| 763 | Ac-LTF$r8Cou2YWAQL$S-NH₂ | 1475 | 1789.94 | 895.97 | 896.5 |
| 764 | Ac-Cou2TF$r8AYWAQL$S-NH₂ | 1476 | 1747.90 | 874.95 | 875.42 |
| 765 | Ac-LTF$r8ACou2WAQL$S-NH₂ | 1477 | 1697.92 | 849.96 | 850.82 |
| 766 | Dmaac-LTF$r8AYWAQL$S-NH₂ | 1478 | 1574.89 | 788.45 | 788.82 |
| 767 | Hexac-LTF$r8AYWAQL$S-NH₂ | 1479 | 1587.91 | 794.96 | 795.11 |
| 768 | Napac-LTF$r8AYWAQL$S-NH₂ | 1480 | 1657.89 | 829.95 | 830.36 |
| 769 | Pam-LTF$r8AYWAQL$S-NH₂ | 1481 | 1728.06 | 865.03 | 865.45 |
| 770 | Ac-LT2Nal$r8HYAAQL$S-NH₂ | 1482 | 1532.84 | 767.42 | 767.61 |
| 771 | Ac-LT2Nal$/r8HYWAQL$/S-NH₂ | 1483 | 1675.91 | 838.96 | 839.1 |
| 772 | Ac-LT2Nal$r8HYFAQL$S-NH₂ | 1484 | 1608.87 | 805.44 | 805.9 |
| 773 | Ac-LT2Nal$r8HWAAQL$S-NH₂ | 1485 | 1555.86 | 778.93 | 779.08 |
| 774 | Ac-LT2Nal$r8HYAWQL$S-NH₂ | 1486 | 1647.88 | 824.94 | 825.04 |
| 775 | Ac-LT2Nal$r8HYAAQW$S-NH₂ | 1487 | 1605.83 | 803.92 | 804.05 |
| 776 | Ac-LTW$r8HYWAQL$S-NH₂ | 1488 | 1636.88 | 819.44 | 819.95 |
| 777 | Ac-LT1Nal$r8HYWAQL$S-NH₂ | 1489 | 1647.88 | 824.94 | 825.41 |

TABLE 2c shows examples of crosslinked and non-crosslinked polypeptides comprising D-amino acids.

TABLE 2c

| | | SEQ | | Exact | Found | Calc | Calc | Calc |
|---|---|---|---|---|---|---|---|---|
| SP | Sequence | ID NO: | Isomer | Mass | Mass | (M + 1)/1 | (M + 2)/2 | (M + 3)/3 |
| 765 | Ac-tawyanfekllr-NH2 | 1490 | z | z | 777.46 | z | z | z |
| 766 | Ac-tawyanf4CF3ekllr-NH2 | 1491 | | | 811.41 | | | |

### Example 3: Synthesis of Triazole-Crosslinked Peptidomimetic Macrocycles

In a typical example for the preparation of a peptidomimetic macrocycle comprising a 1,4-triazole group (e.g. SP153), 20% (v/v) 2,6-lutidine in DMF was added to the peptide resin (0.5 mmol) in a 40 ml glass vial and shaken for 10 minutes. Sodium ascorbate (0.25 g, 1.25 mmol) and diisopropylethylamine (0.22 ml, 1.25 mmol) were then added, followed by copper(I) iodide (0.24 g, 1.25 mmol) and the resulting reaction mixture was mechanically shaken 16 hours at ambient temperature.

In a typical example for the preparation of a peptidomimetic macrocycle comprising a 1,5-triazole group (SP932, SP933), a peptide resin (0.25 mmol) was washed with anhydrous DCM. Resin was loaded into a microwave vial. Vessel was evacuated and purged with nitrogen. Chloro(pentamethylcyclopentadienyl) bis(triphenylphosphine)ruthenium(II), 10% loading, (Strem 44-0117) was added. Anhydrous toluene was added to the reaction vessel. The reaction was then loaded into the microwave and held at 90° C. for 10 minutes. Reaction may need to be pushed a subsequent time for completion. In other cases, Chloro(1,5-cyclooctadiene)(pentamethylcyclopentadienyl)ruthenium (“Cp*RuCl(cod)”) may be used, for example at room temperature in a solvent comprising toluene.

In a typical example for the preparation of a peptidomimetic macrocycle comprising an iodo-substituted triazole group (e.g. SP457), THF (2 ml) was added to the peptide resin (0.05 mmol) in a 40 ml glass vial and shaken for 10 minutes. N-bromosuccimide (0.04 g, 0.25 mmol), copper(I) iodide (0.05 g, 0.25 mmol) and diisopropylethylamine (0.04 ml, 0.25 mmol) were then added and the resulting reaction mixture was mechanically shaken 16 hours at ambient temperature. Iodo-triazole crosslinkers may be further substituted by a coupling reaction, for example with boronic acids, to result in a peptidomimetic macrocycle such as SP465. In a typical example, DMF (3 ml) was added to the iodo-triazole peptide resin (0.1 mmol) in a 40 ml glass vial and shaken for 10 minutes. Phenyl boronic acid (0.04 g, 0.3 mmol), tetrakis(triphenylphosphine)palladium(0) (0.006 g, 0.005 mmol) and potassium carbonate (0.083 g, 0.6 mmol) were then added and the resulting reaction mixture was mechanically shaken 16 hours at 70° C. Iodo-triazole crosslinkers may also be further substituted by a coupling reaction, for example with a terminal alkyne (e.g. Sonogashira coupling), to result in a peptidomimetic macrocycle such as SP468. In a typical example, 2:1 THF:triethylamine (3 ml) was added to the iodo-triazole peptide resin (0.1 mmol) in a 40 ml glass vial and shaken for 10 minutes. N—BOC-4-pentyne-1-amine (0.04 g, 0.2 mmol) and bis(triphenylphosphine)palladiumchloride (0.014 g, 0.02 mmol) were added and shaken for 5 minutes. Copper(I) iodide (0.004 g, 0.02 mmol) was then added and the resulting reaction mixture was mechanically shaken 16 hours at 70° C.

The triazole-cyclized resin-bound peptides were deprotected and cleaved from the solid support by treatment with TFA/H₂O/TIS (95/5/5 v/v) for 2.5 h at room temperature. After filtration of the resin the TFA solution was precipitated in cold diethyl ether and centrifuged to yield the desired product as a solid. The crude product was purified by preparative HPLC. For example, purification of cross-linked compounds is achieved by high performance liquid chromatography (HPLC) (Varian ProStar) on a reverse phase C18 column (Varian) to yield the pure compounds. Chemical composition of the pure products is confirmed by LC/MS mass spectrometry (Micromass LCT interfaced with Agilent 1100 HPLC system) and amino acid analysis (Applied Biosystems, model 420A).

TABLE 3 and TABLE 3A show lists of peptidomimetic macrocycles of Formula I.

TABLE 3

| | SEQ | |
|---|---|---|
| SP- | ID NO: | Sequence |
| 778 | 1492 | Ac-F$4rn6AYWEAc3cL$4a5AAA-NH2 |
| 779 | 1493 | Ac-F$4rn6AYWEAc3cL$4a5AAibA-NH2 |
| 780 | 1494 | Ac-LTF$4rn6AYWAQL$4a5SANle-NH2 |
| 781 | 1495 | Ac-LTF$4rn6AYWAQL$4a5SAL-NH2 |
| 782 | 1496 | Ac-LTF$4rn6AYWAQL$4a5SAM-NH2 |
| 783 | 1497 | Ac-LTF$4rn6AYWAQL$4a5SAhL-NH2 |
| 784 | 1498 | Ac-LTF$4rn6AYWAQL$4a5SAF-NH2 |
| 785 | 1499 | Ac-LTF$4rn6AYWAQL$4a5SAI-NH2 |
| 786 | 1500 | Ac-LTF$4rn6AYWAQL$4a5SAChg-NH2 |
| 787 | 1501 | Ac-LTF$4rn6AYWAQL$4a5SAAib-NH2 |
| 788 | 1502 | Ac-LTF$4rn6AYWAQL$4a5SAA-NH2 |
| 789 | 1503 | Ac-LTF$4rn6AYWA$4a5L$S$Nle-NH2 |
| 790 | 1504 | Ac-LTF$4rn6AYWA$4a5L$S$A-NH2 |
| 791 | 1505 | Ac-F$4rn6AYWEAc3cL$4a5AANle-NH2 |
| 792 | 1506 | Ac-F$4rn6AYWEAc3cL$4a5AAL-NH2 |
| 793 | 1507 | Ac-F$4rn6AYWEAc3cL$4a5AAM-NH2 |
| 794 | 1508 | Ac-F$4rn6AYWEAc3cL$4a5AAhL-NH2 |
| 795 | 1509 | Ac-F$4rn6AYWEAc3cL$4a5AAF-NH2 |
| 796 | 1510 | Ac-F$4rn6AYWEAc3cL$4a5AAI-NH2 |
| 797 | 1511 | Ac-F$4rn6AYWEAc3cL$4a5AAChg-NH2 |
| 798 | 1512 | Ac-F$4rn6AYWEAc3cL$4a5AACha-NH2 |
| 799 | 1513 | Ac-F$4rn6AYWEAc3cL$4a5AAAib-NH2 |
| 800 | 1514 | Ac-LTF$4rn6AYWAQL$4a5AAAibV-NH2 |
| 801 | 1515 | Ac-LTF$4rn6AYWAQL$4a5AAAibV-NH2 |
| 802 | 1516 | Ac-LTF$4rn6AYWAQL$4a5SAibAA-NH2 |
| 803 | 1517 | Ac-LTF$4rn6AYWAQL$4a5SAibAA-NH2 |
| 804 | 1518 | Ac-HLTF$4rn6HHWHQL$4a5AANleNle-NH2 |
| 805 | 1519 | Ac-DLTF$4rn6HHWHQL$4a5RRLV-NH2 |
| 806 | 1520 | Ac-HHTF$4rn6HHWHQL$4a5AAML-NH2 |
| 807 | 1521 | Ac-F$4rn6HHWHQL$4a5RRDCha-NH2 |
| 808 | 1522 | Ac-F$4rn6HHWHQL$4a5HRFV-NH2 |
| 809 | 1523 | Ac-HLTF$4rn6HHWHQL$4a5AAhLA-NH2 |
| 810 | 1524 | Ac-DLTF$4rn6HHWHQL$4a5RRChgl-NH2 |
| 811 | 1525 | Ac-DLTF$4rn6HHWHQL$4a5RRChgl-NH2 |
| 812 | 1526 | Ac-HHTF$4rn6HHWHQL$4a5AAChav-NH2 |
| 813 | 1527 | Ac-F$4rn6HHWHQL$4a5RRDa-NH2 |
| 814 | 1528 | Ac-F$4rn6HHWHQL$4a5HRAibG-NH2 |
| 815 | 1529 | Ac-F$4rn6AYWAQL$4a5HHNleL-NH2 |
| 816 | 1530 | Ac-F$4rn6AYWSAL$4a5HQANle-NH2 |
| 817 | 1531 | Ac-F$4rn6AYWVQL$4a5QHChgl-NH2 |
| 818 | 1532 | Ac-F$4rn6AYWTAL$4a5QQNlev-NH2 |
| 819 | 1533 | Ac-F$4rn6AYWYQL$4a5HAibAa-NH2 |
| 820 | 1534 | Ac-LTF$4rn6AYWAQL$4a5HHLa-NH2 |
| 821 | 1535 | Ac-LTF$4rn6AYWAQL$4a5HHLa-NH2 |
| 822 | 1536 | Ac-LTF$4rn6AYWAQL$4a5HQNlev-NH2 |
| 823 | 1537 | Ac-LTF$4rn6AYWAQL$4a5HQNlev-NH2 |
| 824 | 1538 | Ac-LTF$4rn6AYWAQL$4a5QQMl-NH2 |
| 825 | 1539 | Ac-LTF$4rn6AYWAQL$4a5QQMl-NH2 |
| 826 | 1540 | Ac-LTF$4rn6AYWAQL$4a5HAibhLV-NH2 |
| 827 | 1541 | Ac-LTF$4rn6AYWAQL$4a5AHFA-NH2 |
| 828 | 1542 | Ac-HLTF$4rn6HHWHQL$4a5AANlel-NH2 |
| 829 | 1543 | Ac-DLTF$4rn6HHWHQL$4a5RRLa-NH2 |
| 830 | 1544 | Ac-HHTF$4rn6HHWHQL$4a5AAMv-NH2 |
| 831 | 1545 | Ac-F$4rn6HHWHQL$4a5RRDA-NH2 |
| 832 | 1546 | Ac-F$4rn6HHWHQL$4a5HRFCha-NH2 |
| 833 | 1547 | Ac-F$4rn6AYWEAL$4a5AA-NHAm |
| 834 | 1548 | Ac-F$4rn6AYWEAL$4a5AA-NHiAm |
| 835 | 1549 | Ac-F$4rn6AYWEAL$4a5AA-NHnPr3Ph |
| 836 | 1550 | Ac-F$4rn6AYWEAL$4a5AA-NHnBu33Me |
| 837 | 1551 | Ac-F$4rn6AYWEAL$4a5AA-NHnPr |
| 838 | 1552 | Ac-F$4rn6AYWEAL$4a5AA-NHnEt2Ch |
| 839 | 1553 | Ac-F$4rn6AYWEAL$4a5AA-NHnEt2Cp |
| 840 | 1554 | Ac-F$4rn6AYWEAL$4a5AA-NHHex |
| 841 | 1555 | Ac-LTF$4rn6AYWAQL$4a5AAIA-NH2 |
| 842 | 1556 | Ac-LTF$4rn6AYWAQL$4a5AAIA-NH2 |
| 843 | 1557 | Ac-LTF$4rn6AYWAAL$4a5AAMA-NH2 |
| 844 | 1558 | Ac-LTF$4rn6AYWAAL$4a5AAMA-NH2 |
| 845 | 1559 | Ac-LTF$4rn6AYWAQL$4a5AAIa-NH2 |
| 846 | 1560 | Ac-LTF$4rn6AYWAQL$4a5AAIa-NH2 |
| 847 | 1561 | Ac-LTF$4rn6AYWAAL$4a5AAMa-NH2 |
| 848 | 1562 | Ac-LTF$4rn6AYWAAL$4a5AAMa-NH2 |
| 849 | 1563 | Ac-LTF$4rn6AYWAAL$4a5AAIv-NH2 |
| 850 | 1564 | Ac-LTF$4rn6AYWAAL$4a5AAIv-NH2 |
| 851 | 1565 | Ac-LTF$4rn6AYWAQL$4a5AAMv-NH2 |
| 852 | 1566 | Ac-LTF$4rn6AYWAAL$4a5AANlev-NH2 |
| 853 | 1567 | Ac-LTF$4rn6AYWAAL$4a5AANlev-NH2 |
| 854 | 1568 | Ac-LTF$4rn6AYWAQL$4a5AAIl-NH2 |
| 855 | 1569 | Ac-LTF$4rn6AYWAQL$4a5AAIl-NH2 |
| 856 | 1570 | Ac-LTF$4rn6AYWAAL$4a5AAMl-NH2 |
| 857 | 1571 | Ac-LTF$4rn6AYWAQL$4a5AANlel-NH2 |
| 858 | 1572 | Ac-LTF$4rn6AYWAQL$4a5AANlel-NH2 |
| 859 | 1573 | Ac-F$4rn6AYWEAL$4a5AAMA-NH2 |
| 860 | 1574 | Ac-F$4rn6AYWEAL$4a5AANleA-NH2 |
| 861 | 1575 | Ac-F$4rn6AYWEAL$4a5AAIa-NH2 |
| 862 | 1576 | Ac-F$4rn6AYWEAL$4a5AAMa-NH2 |
| 863 | 1577 | Ac-F$4rn6AYWEAL$4a5AANlea-NH2 |
| 864 | 1578 | Ac-F$4rn6AYWEAL$4a5AAIv-NH2 |
| 865 | 1579 | Ac-F$4rn6AYWEAL$4a5AAMv-NH2 |
| 866 | 1580 | Ac-F$4rn6AYWEAL$4a5AANlev-NH2 |
| 867 | 1581 | Ac-F$4rn6AYWEAL$4a5AAIl-NH2 |
| 868 | 1582 | Ac-F$4rn6AYWEAL$4a5AAMl-NH2 |
| 869 | 1583 | Ac-F$4rn6AYWEAL$4a5AANlel-NH2 |
| 870 | 1584 | Ac-F$4rn6AYWEAL$4a5AANlel-NH2 |
| 871 | 1585 | Ac-LTF$4rn6AY6clWAQL$4a5SAA-NH2 |
| 872 | 1586 | Ac-LTF$4rn6AY6clWAQL$4a5SAA-NH2 |
| 873 | 1587 | Ac-WTF$4rn6FYWSQL$4a5AVAa-NH2 |
| 874 | 1588 | Ac-WTF$4rn6FYWSQL$4a5AVAa-NH2 |
| 875 | 1589 | Ac-WTF$4rn6VYWSQL$4a5AVA-NH2 |
| 876 | 1590 | Ac-WTF$4rn6VYWSQL$4a5AVA-NH2 |
| 877 | 1591 | Ac-WTF$4rn6FYWSQL$4a5SAAa-NH2 |
| 878 | 1592 | Ac-WTF$4rn6FYWSQL$4a5SAAa-NH2 |
| 879 | 1593 | Ac-WTF$4rn6VYWSQL$4a5AVAaa-NH2 |
| 880 | 1594 | Ac-WTF$4rn6VYWSQL$4a5AVAaa-NH2 |
| 881 | 1595 | Ac-LTF$4rn6AYWAQL$4a5AVG-NH2 |
| 882 | 1596 | Ac-LTF$4rn6AYWAQL$4a5AVG-NH2 |
| 883 | 1597 | Ac-LTF$4rn6AYWAQL$4a5AVQ-NH2 |
| 884 | 1598 | Ac-LTF$4rn6AYWAQL$4a5AVQ-NH2 |
| 885 | 1599 | Ac-LTF$4rn6AYWAQL$4a5SAa-NH2 |
| 886 | 1600 | Ac-LTF$4rn6AYWAQL$4a5SAa-NH2 |
| 887 | 1601 | Ac-LTF$4rn6AYWAQhL$4a5SAA-NH2 |
| 888 | 1602 | Ac-LTF$4rn6AYWAQhL$4a5SAA-NH2 |
| 889 | 1603 | Ac-LTF$4rn6AYWEQLStSA$4a5-NH2 |
| 890 | 1604 | Ac-LTF$4rn6AYWAQL$4a5SLA-NH2 |
| 891 | 1605 | Ac-LTF$4rn6AYWAQL$4a5SLA-NH2 |
| 892 | 1606 | Ac-LTF$4rn6AYWAQL$4a5SWA-NH2 |
| 893 | 1607 | Ac-LTF$4rn6AYWAQL$4a5SWA-NH2 |
| 894 | 1608 | Ac-LTF$4rn6AYWAQL$4a5SVS-NH2 |
| 895 | 1609 | Ac-LTF$4rn6AYWAQL$4a5SAS-NH2 |
| 896 | 1610 | Ac-LTF$4rn6AYWAQL$4a5SVG-NH2 |
| 897 | 1611 | Ac-ETF$4rn6VYWAQL$4a5SAa-NH2 |
| 898 | 1612 | Ac-ETF$4rn6VYWAQL$4a5SAA-NH2 |
| 899 | 1613 | Ac-ETF$4rn6VYWAQL$4a5SVA-NH2 |
| 900 | 1614 | Ac-ETF$4rn6VYWAQL$4a5SLA-NH2 |
| 901 | 1615 | Ac-ETF$4rn6VYWAQL$4a5SWA-NH2 |
| 902 | 1616 | Ac-ETF$4rn6KYWAQL$4a5SWA-NH2 |
| 903 | 1617 | Ac-ETF$4rn6VYWAQL$4a5SVS-NH2 |
| 904 | 1618 | Ac-ETF$4rn6VYWAQL$4a5SAS-NH2 |
| 905 | 1619 | Ac-ETF$4rn6VYWAQL$4a5SVG-NH2 |
| 906 | 1620 | Ac-LTF$4rn6VYWAQL$4a5SSa-NH2 |
| 907 | 1621 | Ac-ETF$4rn6VYWAQL$4a5SSa-NH2 |
| 908 | 1622 | Ac-LTF$4rn6VYWAQL$4a5SNa-NH2 |
| 909 | 1623 | Ac-ETF$4rn6VYWAQL$4a5SNa-NH2 |
| 910 | 1624 | Ac-LTF$4rn6VYWAQL$4a5SAa-NH2 |
| 911 | 1625 | Ac-LTF$4rn6VYWAQL$4a5SVA-NH2 |
| 912 | 1626 | Ac-LTF$4rn6VYWAQL$4a5SVA-NH2 |
| 913 | 1627 | Ac-LTF$4rn6VYWAQL$4a5SWA-NH2 |
| 914 | 1628 | Ac-LTF$4rn6VYWAQL$4a5SVS-NH2 |
| 915 | 1629 | Ac-LTF$4rn6VYWAQL$4a5SVS-NH2 |
| 916 | 1630 | Ac-LTF$4rn6VYWAQL$4a5SAS-NH2 |
| 917 | 1631 | Ac-LTF$4rn6VYWAQL$4a5SAS-NH2 |
| 918 | 1632 | Ac-LTF$4rn6VYWAQL$4a5SVG-NH2 |
| 919 | 1633 | Ac-LTF$4rn6VYWAQL$4a5SVG-NH2 |
| 920 | 1634 | Ac-LTF$4rn6EYWAQCha$4a5SAA-NH2 |
| 921 | 1635 | Ac-LTF$4rn6EYWAQCha$4a5SAA-NH2 |
| 922 | 1636 | Ac-LTF$4rn6EYWAQCpg$4a5SAA-NH2 |
| 923 | 1637 | Ac-LTF$4rn6EYWAQCpg$4a5SAA-NH2 |
| 924 | 1638 | Ac-LTF$4rn6EYWAQF$4a5SAA-NH2 |
| 925 | 1639 | Ac-LTF$4rn6EYWAQF$4a5SAA-NH2 |
| 926 | 1640 | Ac-LTF3Cl$4rn6EYWAQL$4a5SAA-NH2 |
| 927 | 1641 | Ac-LTF3Cl$4rn6EYWAQL$4a5SAA-NH2 |
| 928 | 1642 | Ac-LTF34F2$4rn6EYWAQL$4a5SAA-NH2 |
| 929 | 1643 | Ac-LTF34F2$4rn6EYWAQL$4a5SAA-NH2 |
| 930 | 1644 | Ac-LTF34F2$4rn6EYWAQhL$4a5SAA-NH2 |
| 931 | 1645 | Ac-LTF34F2$4rn6EYWAQhL$4a5SAA-NH2 |
| 932 | 1646 | Ac-ETF$4rn6EYWAQL$4a5SAA-NH2 |
| 933 | 1647 | Ac-LTF$4rn6AYWVQL$4a5SAA-NH2 |
| 934 | 1648 | Ac-LTF$4rn6AHWAQL$4a5SAA-NH2 |
| 935 | 1649 | Ac-LTF$4rn6AEWAQL$4a5SAA-NH2 |
| 936 | 1650 | Ac-LTF$4rn6ASWAQL$4a5SAA-NH2 |
| 937 | 1651 | Ac-LTF$4rn6AEWAQL$4a5SAA-NH2 |
| 938 | 1652 | Ac-LTF$4rn6ASWAQL$4a5SAA-NH2 |
| 939 | 1653 | Ac-LTF$4rn6AF4coohWAQL$4a5SAA-NH2 |
| 940 | 1654 | Ac-LTF$4rn6AF4coohWAQL$4a5SAA-NH2 |
| 941 | 1655 | Ac-LTF$4rn6AHWAQL$4a5AAIa-NH2 |
| 942 | 1656 | Ac-ITF$4rn6FYWAQL$4a5AAIa-NH2 |
| 943 | 1657 | Ac-ITF$4rn6EHWAQL$4a5AAIa-NH2 |
| 944 | 1658 | Ac-ITF$4rn6EHWAQL$4a5AAIa-NH2 |
| 945 | 1659 | Ac-ETF$4rn6EHWAQL$4a5AAIa-NH2 |
| 946 | 1660 | Ac-ETF$4rn6EHWAQL$4a5AAIa-NH2 |
| 947 | 1661 | Ac-LTF$4rn6AHWVQL$4a5AAIa-NH2 |
| 948 | 1662 | Ac-ITF$4rn6FYWVQL$4a5AAIa-NH2 |
| 949 | 1663 | Ac-ITF$4rn6EYWVQL$4a5AAIa-NH2 |
| 950 | 1664 | Ac-ITF$4rn6EHWVQL$4a5AAIa-NH2 |
| 951 | 1665 | Ac-LTF$4rn6AEWAQL$4a5AAIa-NH2 |
| 952 | 1666 | Ac-LTF$4rn6AF4coohWAQL$4a5AAIa-NH2 |
| 953 | 1667 | Ac-LTF$4rn6AF4coohWAQL$4a5AAIa-NH2 |
| 954 | 1668 | Ac-LTF$4rn6AHWAQL$4a5AHFA-NH2 |
| 955 | 1669 | Ac-ITF$4rn6FYWAQL$4a5AHFA-NH2 |
| 956 | 1670 | Ac-ITF$4rn6FYWAQL$4a5AHFA-NH2 |
| 957 | 1671 | Ac-ITF$4rn6FHWAQL$4a5AEFA-NH2 |
| 958 | 1672 | Ac-ITF$4rn6FHWAQL$4a5AEFA-NH2 |
| 959 | 1673 | Ac-ITF$4rn6EHWAQL$4a5AHFA-NH2 |
| 960 | 1674 | Ac-ITF$4rn6EHWAQL$4a5AHFA-NH2 |
| 961 | 1675 | Ac-LTF$4rn6AHWVQL$4a5AHFA-NH2 |
| 962 | 1676 | Ac-ITF$4rn6FYWVQL$4a5AHFA-NH2 |
| 963 | 1677 | Ac-ITF$4rn6EYWVQL$4a5AHFA-NH2 |
| 964 | 1678 | Ac-ITF$4rn6EHWVQL$4a5AHFA-NH2 |
| 965 | 1679 | Ac-ITF$4rn6EHWVQL$4a5AHFA-NH2 |
| 966 | 1680 | Ac-ETF$4rn6EYWAAL$4a5SAA-NH2 |
| 967 | 1681 | Ac-LTF$4rn6AYWVAL$4a5SAA-NH2 |
| 968 | 1682 | Ac-LTF$4rn6AHWAAL$4a5SAA-NH2 |
| 969 | 1683 | Ac-LTF$4rn6AEWAAL$4a5SAA-NH2 |
| 970 | 1684 | Ac-LTF$4rn6AEWAAL$4a5SAA-NH2 |
| 971 | 1685 | Ac-LTF$4rn6ASWAAL$4a5SAA-NH2 |
| 972 | 1686 | Ac-LTF$4rn6ASWAAL$4a5SAA-NH2 |
| 973 | 1687 | Ac-LTF$4rn6AYWAAL$4a5AAIa-NH2 |
| 974 | 1688 | Ac-LTF$4rn6AYWAAL$4a5AAIa-NH2 |
| 975 | 1689 | Ac-LTF$4rn6AYWAAL$4a5AHFA-NH2 |
| 976 | 1690 | Ac-LTF$4rn6EHWAQL$4a5AHIa-NH2 |
| 977 | 1691 | Ac-LTF$4rn6EHWAQL$4a5AHIa-NH2 |
| 978 | 1692 | Ac-LTF$4rn6AHWAQL$4a5AHIa-NH2 |
| 979 | 1693 | Ac-LTF$4rn6EYWAQL$4a5AHIa-NH2 |
| 980 | 1694 | Ac-LTF$4rn6AYWAQL$4a5AAFa-NH2 |
| 981 | 1695 | Ac-LTF$4rn6AYWAQL$4a5AAFa-NH2 |
| 982 | 1696 | Ac-LTF$4rn6AYWAQL$4a5AAWa-NH2 |
| 983 | 1697 | Ac-LTF$4rn6AYWAQL$4a5AAVa-NH2 |
| 984 | 1698 | Ac-LTF$4rn6AYWAQL$4a5AAVa-NH2 |
| 985 | 1699 | Ac-LTF$4rn6AYWAQL$4a5AALa-NH2 |
| 986 | 1700 | Ac-LTF$4rn6AYWAQL$4a5AALa-NH2 |
| 987 | 1701 | Ac-LTF$4rn6EYWAQL$4a5AAIa-NH2 |
| 988 | 1702 | Ac-LTF$4rn6EYWAQL$4a5AAIa-NH2 |
| 989 | 1703 | Ac-LTF$4rn6EYWAQL$4a5AAFa-NH2 |
| 990 | 1704 | Ac-LTF$4rn6EYWAQL$4a5AAFa-NH2 |
| 991 | 1705 | Ac-LTF$4rn6EYWAQL$4a5AAVa-NH2 |
| 992 | 1706 | Ac-LTF$4rn6EYWAQL$4a5AAVa-NH2 |
| 993 | 1707 | Ac-LTF$4rn6EHWAQL$4a5AAIa-NH2 |
| 994 | 1708 | Ac-LTF$4rn6EHWAQL$4a5AAIa-NH2 |
| 995 | 1709 | Ac-LTF$4rn6EHWAQL$4a5AAWa-NH2 |
| 996 | 1710 | Ac-LTF$4rn6EHWAQL$4a5AAWa-NH2 |
| 997 | 1711 | Ac-LTF$4rn6EHWAQL$4a5AALa-NH2 |
| 998 | 1712 | Ac-LTF$4rn6EHWAQL$4a5AALa-NH2 |
| 999 | 1713 | Ac-ETF$4rn6EHWVQL$4a5AALa-NH2 |
| 1000 | 1714 | Ac-LTF$4rn6AYWAQL$4a5AAAa-NH2 |
| 1001 | 1715 | Ac-LTF$4rn6AYWAQL$4a5AAAa-NH2 |
| 1002 | 1716 | Ac-LTF$4rn6AYWAQL$4a5AAAibA-NH2 |
| 1003 | 1717 | Ac-LTF$4rn6AYWAQL$4a5AAAibA-NH2 |
| 1004 | 1718 | Ac-LTF$4rn6AYWAQL$4a5AAAAa-NH2 |
| 1005 | 1719 | Ac-LTF$r5AYWAQL$4a5s8AAIa-NH2 |
| 1006 | 1720 | Ac-LTF$r5AYWAQL$4a5s8SAA-NH2 |
| 1007 | 1721 | Ac-LTF$4rn6AYWAQCba$4a5AANleA-NH2 |
| 1008 | 1722 | Ac-ETF$4rn6AYWAQCba$4a5AANleA-NH2 |
| 1009 | 1723 | Ac-LTF$4rn6EYWAQCba$4a5AANleA-NH2 |
| 1010 | 1724 | Ac-LTF$4rn6AYWAQCba$4a5AWNleA-NH2 |
| 1011 | 1725 | Ac-ETF$4rn6AYWAQCba$4a5AWNleA-NH2 |
| 1012 | 1726 | Ac-LTF$4rn6EYWAQCba$4a5AWNleA-NH2 |
| 1013 | 1727 | Ac-LTF$4rn6EYWAQCba$4a5SAFA-NH2 |
| 1014 | 1728 | Ac-LTF34F2$4rn6EYWAQCba$4a5SANleA-NH2 |
| 1015 | 1729 | Ac-LTF$4rn6EF4coohWAQCba$4a5SANleA-NH2 |
| 1016 | 1730 | Ac-LTF$4rn6EYWSQCba$4a5SANleA-NH2 |
| 1017 | 1731 | Ac-LTF$4rn6EYWWQCba$4a5SANleA-NH2 |
| 1018 | 1732 | Ac-LTF$4rn6EYWAQCba$4a5AAIa-NH2 |
| 1019 | 1733 | Ac-LTF34F2$4rn6EYWAQCba$4a5AAIa-NH2 |
| 1020 | 1734 | Ac-LTF$4rn6EF4coohWAQCba$4a5AAIa-NH2 |
| 1021 | 1735 | Pam-ETF$4rn6EYWAQCba$4a5SAA-NH2 |
| 1022 | 1736 | Ac-LThF$4rn6EFWAQCba$4a5SAA-NH2 |
| 1023 | 1737 | Ac-LTF$4rn6EYAAQCba$4a5SAA-NH2 |
| 1024 | 1738 | Ac-LTF$4rn6EY2NalAQCba$4a5SAA-NH2 |
| 1025 | 1739 | Ac-LTF$4rn6AYWAQCba$4a5SAA-NH2 |
| 1026 | 1740 | Ac-LTF$4rn6EYWAQCba$4a5SAF-NH2 |
| 1027 | 1741 | Ac-LTF$4rn6EYWAQCba$4a5SAFa-NH2 |
| 1028 | 1742 | Ac-LTF$4rn6AYWAQCba$4a5SAF-NH2 |
| 1029 | 1743 | Ac-LTF34F2$4rn6AYWAQCba$4a5SAF-NH2 |
| 1030 | 1744 | Ac-LTF$4rn6AF4coohWAQCba$4a5SAF-NH2 |
| 1031 | 1745 | Ac-LTF$4rn6EY6clWAQCba$4a5SAF-NH2 |
| 1032 | 1746 | Ac-LTF$4rn6AYWSQCba$4a5SAF-NH2 |
| 1033 | 1747 | Ac-LTF$4rn6AYWWQCba$4a5SAF-NH2 |
| 1034 | 1748 | Ac-LTF$4rn6AYWAQCba$4a5AAIa-NH2 |
| 1035 | 1749 | Ac-LTF34F2$4rn6AYWAQCba$4a5AAIa-NH2 |
| 1036 | 1750 | Ac-LTF$4rn6AY6clWAQCba$4a5AAIa-NH2 |
| 1037 | 1751 | Ac-LTF$4rn6AF4coohWAQCba$4a5AAIa-NH2 |
| 1038 | 1752 | Ac-LTF$4rn6EYWAQCba$4a5AAFa-NH2 |
| 1039 | 1753 | Ac-LTF$4rn6EYWAQCba$4a5AAFa-NH2 |
| 1040 | 1754 | Ac-ETF$4rn6AYWAQCba$4a5AWNlea-NH2 |
| 1041 | 1755 | Ac-LTF$4rn6EYWAQCba$4a5AWNlea-NH2 |
| 1042 | 1756 | Ac-ETF$4rn6EYWAQCba$4a5AWNlea-NH2 |
| 1043 | 1757 | Ac-ETF$4rn6EYWAQCba$4a5AWNlea-NH2 |
| 1044 | 1758 | Ac-LTF$4rn6AYWAQCba$4a5SAFa-NH2 |
| 1045 | 1759 | Ac-LTF$4rn6AYWAQCba$4a5SAFa-NH2 |
| 1046 | 1760 | Ac-ETF$4rn6AYWAQL$4a5AWNlea-NH2 |
| 1047 | 1761 | Ac-LTF$4rn6EYWAQL$4a5AWNlea-NH2 |
| 1048 | 1762 | Ac-ETF$4rn6EYWAQL$4a5AWNlea-NH2 |
| 1049 | 1763 | Dmaac-LTF$4rn6EYWAQhL$4a5SAA-NH2 |
| 1050 | 1764 | Hexac-LTF$4rn6EYWAQhL$4a5SAA-NH2 |
| 1051 | 1765 | Napac-LTF$4rn6EYWAQhL$4a5SAA-NH2 |
| 1052 | 1766 | Decac-LTF$4rn6EYWAQhL$4a5SAA-NH2 |
| 1053 | 1767 | Admac-LTF$4rn6EYWAQhL$4a5SAA-NH2 |
| 1054 | 1768 | Tmac-LTF$4rn6EYWAQhL$4a5SAA-NH2 |
| 1055 | 1769 | Pam-LTF$4rn6EYW AQhL$4a5SAA-NH2 |
| 1056 | 1770 | Ac-LTF$4rn6AYWAQCba$4a5AANleA-NH2 |
| 1057 | 1771 | Ac-LTF34F2$4rn6EYWAQCba$4a5AAIa-NH2 |
| 1058 | 1772 | Ac-LTF34F2$4rn6EYWAQCba$4a5SAA-NH2 |
| 1059 | 1773 | Ac-LTF34F2$4rn6EYWAQCba$4a5SAA-NH2 |
| 1060 | 1774 | Ac-LTF$4rn6EF4coohWAQCba$4a5SAA-NH2 |
| 1061 | 1775 | Ac-LTF$4rn6EF4coohWAQCba$4a5SAA-NH2 |
| 1062 | 1776 | Ac-LTF$4rn6EYWSQCba$4a5SAA-NH2 |
| 1063 | 1777 | Ac-LTF$4rn6EYWSQCba$4a5SAA-NH2 |
| 1064 | 1778 | Ac-LTF$4rn6EYWAQhL$4a5SAA-NH2 |
| 1065 | 1779 | Ac-LTF$4rn6AYWAQhL$4a5SAF-NH2 |
| 1066 | 1780 | Ac-LTF$4rn6AYWAQhL$4a5SAF-NH2 |
| 1067 | 1781 | Ac-LTF34F2$4rn6AYWAQhL$4a5SAA-NH2 |
| 1068 | 1782 | Ac-LTF34F2$4rn6AYWAQhL$4a5SAA-NH2 |
| 1069 | 1783 | Ac-LTF$4rn6AF4coohWAQhL$4a5SAA-NH2 |
| 1070 | 1784 | Ac-LTF$4rn6AF4coohWAQhL$4a5SAA-NH2 |
| 1071 | 1785 | Ac-LTF$4rn6AYWSQhL$4a5SAA-NH2 |
| 1072 | 1786 | Ac-LTF$4rn6AYWSQhL$4a5SAA-NH2 |
| 1073 | 1787 | Ac-LTF$4rn6EYWAQL$4a5AANleA-NH2 |
| 1074 | 1788 | Ac-LTF34F2$4rn6AYWAQL$4a5AANleA-NH2 |
| 1075 | 1789 | Ac-LTF$4rn6AF4coohWAQL$4a5AANleA-NH2 |
| 1076 | 1790 | Ac-LTF$4rn6AYWSQL$4a5AANleA-NH2 |
| 1077 | 1791 | Ac-LTF34F2$4rn6AYWAQhL$4a5AANleA-NH2 |
| 1078 | 1792 | Ac-LTF34F2$4rn6AYWAQhL$4a5AANleA-NH2 |
| 1079 | 1793 | Ac-LTF$4rn6AF4coohWAQhL$4a5AANleA-NH2 |
| 1080 | 1794 | Ac-LTF$4rn6AF4coohWAQhL$4a5AANleA-NH2 |
| 1081 | 1795 | Ac-LTF$4rn6AYWSQhL$4a5AANleA-NH2 |
| 1082 | 1796 | Ac-LTF$4rn6AYWSQhL$4a5AANleA-NH2 |
| 1083 | 1797 | Ac-LTF$4rn6AYWAQhL$4a5AAAAa-NH2 |
| 1084 | 1798 | Ac-LTF$4rn6AYWAQhL$4a5AAAAa-NH2 |
| 1085 | 1799 | Ac-LTF$4rn6AYWAQL$4a5AAAAAAa-NH2 |
| 1086 | 1800 | Ac-LTF$4rn6AYWAQL$4a5AAAAAAa-NH2 |
| 1087 | 1801 | Ac-LTF$4rn6EYWAQhL$4a5AANleA-NH2 |
| 1088 | 1802 | Ac-AATF$4rn6AYWAQL$4a5AANleA-NH2 |
| 1089 | 1803 | Ac-LTF$4rn6AYWAQL$4a5AANleAA-NH2 |
| 1090 | 1804 | Ac-ALTF$4rn6AYWAQL$4a5AANleAA-NH2 |
| 1091 | 1805 | Ac-LTF$4rn6AYWAQCba$4a5AANleAA-NH2 |
| 1092 | 1806 | Ac-LTF$4rn6AYWAQhL$4a5AANleAA-NH2 |
| 1093 | 1807 | Ac-LTF$4rn6EYWAQCba$4a5SAAA-NH2 |
| 1094 | 1808 | Ac-LTF$4rn6EYWAQCba$4a5SAAA-NH2 |
| 1095 | 1809 | Ac-LTF$4rn6EYWAQCba$4a5SAAAA-NH2 |
| 1096 | 1810 | Ac-LTF$4rn6EYWAQCba$4a5SAAAA-NH2 |
| 1097 | 1811 | Ac-ALTF$4rn6EYWAQCba$4a5SAA-NH2 |
| 1098 | 1812 | Ac-ALTF$4rn6EYWAQCba$4a5SAAA-NH2 |
| 1099 | 1813 | Ac-ALTF$4rn6EYWAQCba$4a5SAA-NH2 |
| 1100 | 1814 | Ac-LTF$4rn6EY6clWAQCba$4a5SAA-NH2 |
| 1101 | 1815 | Ac-LTF$4rn6EF4cooh6clWAQCba$4a5SANleA-NH2 |
| 1102 | 1816 | Ac-LTF$4rn6EF4cooh6clWAQCba$4a5SANleA-NH2 |
| 1103 | 1817 | Ac-LTF$4rn6EF4cooh6clWAQCba$4a5AAIa-NH2 |
| 1104 | 1818 | Ac-LTF$4rn6EF4cooh6clWAQCba$4a5AAIa-NH2 |
| 1105 | 1819 | Ac-LTF$4rn6AY6clWAQL$4a5AAAAAa-NH2 |
| 1106 | 1820 | Ac-LTF$4rn6AY6clWAQL$4a5AAAAAa-NH2 |
| 1107 | 1821 | Ac-F$4rn6AY6clWEAL$4a5AAAAAAa-NH2 |
| 1108 | 1822 | Ac-ETF$4rn6EYWAQL$4a5AAAAAa-NH2 |
| 1109 | 1823 | Ac-ETF$4rn6EYWAQL$4a5AAAAAa-NH2 |
| 1110 | 1824 | Ac-LTF$4rn6EYWAQL$4a5AAAAAAa-NH2 |
| 1111 | 1825 | Ac-LTF$4rn6EYWAQL$4a5AAAAAAa-NH2 |
| 1112 | 1826 | Ac-LTF$4rn6AYWAQL$4a5AANleAAa-NH2 |
| 1113 | 1827 | Ac-LTF$4rn6AYWAQL$4a5AANleAAa-NH2 |
| 1114 | 1828 | Ac-LTF$4rn6EYWAQCba$4a5AAAAAa-NH2 |
| 1115 | 1829 | Ac-LTF$4rn6EYWAQCba$4a5AAAAAa-NH2 |
| 1116 | 1830 | Ac-LTF$4rn6EF4coohWAQCba$4a5AAAAAa-NH2 |
| 1117 | 1831 | Ac-LTF$4rn6EF4coohWAQCba$4a5AAAAAa-NH2 |
| 1118 | 1832 | Ac-LTF$4rn6EYWSQCba$4a5AAAAAa-NH2 |
| 1119 | 1833 | Ac-LTF$4rn6EYWSQCba$4a5AAAAAa-NH2 |
| 1120 | 1834 | Ac-LTF$4rn6EYWAQCba$4a5SAAa-NH2 |
| 1121 | 1835 | Ac-LTF$4rn6EYWAQCba$4a5SAAa-NH2 |
| 1122 | 1836 | Ac-ALTF$4rn6EYWAQCba$4a5SAAa-NH2 |
| 1123 | 1837 | Ac-ALTF$4rn6EYWAQCba$4a5SAAa-NH2 |
| 1124 | 1838 | Ac-ALTF$4rn6EYWAQCba$4a5SAAAa-NH2 |
| 1125 | 1839 | Ac-ALTF$4rn6EYWAQCba$4a5SAAAa-NH2 |
| 1126 | 1840 | Ac-AALTF$4rn6EYWAQCba$4a5SAAAa-NH2 |
| 1127 | 1841 | Ac-AALTF$4rn6EYWAQCba$4a5SAAAa-NH2 |
| 1128 | 1842 | Ac-RTF$4rn6EYWAQCba$4a5SAA-NH2 |
| 1129 | 1843 | Ac-LRF$4rn6EYWAQCba$4a5SAA-NH2 |
| 1130 | 1844 | Ac-LTF$4rn6EYWRQCba$4a5SAA-NH2 |
| 1131 | 1845 | Ac-LTF$4rn6EYWARCba$4a5SAA-NH2 |
| 1132 | 1846 | Ac-LTF$4rn6EYWAQCba$4a5RAA-NH2 |
| 1133 | 1847 | Ac-LTF$4rn6EYWAQCba$4a5SRA-NH2 |
| 1134 | 1848 | Ac-LTF$4rn6EYWAQCba$4a5SAR-NH2 |
| 1135 | 1849 | 5-FAM-BaLTF$4rn6AYWAQL$4a5AANleA-NH2 |
| 1136 | 1850 | Ac-LAF$4rn6EYWAQL$4a5AANleA-NH2 |
| 1137 | 1851 | Ac-ATF$4rn6EYWAQL$4a5AANleA-NH2 |
| 1138 | 1852 | Ac-AAF$4rn6EYWAQL$4a5AANleA-NH2 |
| 1139 | 1853 | Ac-AAAF$4rn6EYWAQL$4a5AANleA-NH2 |
| 1140 | 1854 | Ac-AAAAF$4rn6EYWAQL$4a5AANleA-NH2 |
| 1141 | 1855 | Ac-AATF$4rn6EYWAQL$4a5AANleA-NH2 |
| 1142 | 1856 | Ac-AALTF$4rn6EYWAQL$4a5AANleA-NH2 |
| 1143 | 1857 | Ac-AAALTF$4rn6EYWAQL$4a5AANleA-NH2 |
| 1144 | 1858 | Ac-LTF$4rn6EYWAQL$4a5AANleAA-NH2 |
| 1145 | 1859 | Ac-ALTF$4rn6EYWAQL$4a5AANleAA-NH2 |
| 1146 | 1860 | Ac-AALTF$4rn6EYWAQL$4a5AANleAA-NH2 |
| 1147 | 1861 | Ac-LTF$4rn6EYWAQCba$4a5AANleAA-NH2 |
| 1148 | 1862 | Ac-LTF$4rn6EYWAQhL$4a5AANleAA-NH2 |
| 1149 | 1863 | Ac-ALTF$4rn6EYWAQhL$4a5AANleAA-NH2 |
| 1150 | 1864 | Ac-LTF$4rn6ANmYWAQL$4a5AANleA-NH2 |
| 1151 | 1865 | Ac-LTF$4rn6ANmYWAQL$4a5AANleA-NH2 |
| 1152 | 1866 | Ac-LTF$4rn6AYNmWAQL$4a5AANleA-NH2 |
| 1153 | 1867 | Ac-LTF$4rn6AYNmWAQL$4a5AANleA-NH2 |
| 1154 | 1868 | Ac-LTF$4rn6AYAmwAQL$4a5AANleA-NH2 |
| 1155 | 1869 | Ac-LTF$4rn6AYAmwAQL$4a5AANleA-NH2 |
| 1156 | 1870 | Ac-LTF$4rn6AYWAibQL$4a5AANleA-NH2 |
| 1157 | 1871 | Ac-LTF$4rn6AYWAibQL$4a5AANleA-NH2 |
| 1158 | 1872 | Ac-LTF$4rn6AYWAQL$4a5AAibNleA-NH2 |
| 1159 | 1873 | Ac-LTF$4rn6AYWAQL$4a5AAibNleA-NH2 |
| 1160 | 1874 | Ac-LTF$4rn6AYWAQL$4a5ASarNleA-NH2 |
| 1161 | 1875 | Ac-LTF$4rn6AYWAQL$4a5ASarNleA-NH2 |
| 1162 | 1876 | Ac-LTF$4rn6AYWAQL$4a5AANleAib-NH2 |
| 1163 | 1877 | Ac-LTF$4rn6AYWAQL$4a5AANleAib-NH2 |
| 1164 | 1878 | Ac-LTF$4rn6AYWAQL$4a5AANleNmA-NH2 |
| 1165 | 1879 | Ac-LTF$4rn6AYWAQL$4a5AANleNmA-NH2 |
| 1166 | 1880 | Ac-LTF$4rn6AYWAQL$4a5AANleSar-NH2 |
| 1167 | 1881 | Ac-LTF$4rn6AYWAQL$4a5AANleSar-NH2 |
| 1168 | 1882 | Ac-LTF$4rn6AYWAQL$4a5AANleAAib-NH2 |
| 1169 | 1883 | Ac-LTF$4rn6AYWAQL$4a5AANleAAib-NH2 |
| 1170 | 1884 | Ac-LTF$4rn6AYWAQL$4a5AANleANmA-NH2 |
| 1171 | 1885 | Ac-LTF$4rn6AYWAQL$4a5AANleANmA-NH2 |
| 1172 | 1886 | Ac-LTF$4rn6AYWAQL$4a5AANleAa-NH2 |
| 1173 | 1887 | Ac-LTF$4rn6AYWAQL$4a5AANleAa-NH2 |
| 1174 | 1888 | Ac-LTF$4rn6AYWAQL$4a5AANleASar-NH2 |
| 1175 | 1889 | Ac-LTF$4rn6AYWAQL$4a5AANleASar-NH2 |
| 1176 | 1890 | Ac-LTF$4rn6Cou4YWAQL$4a5AANleA-NH2 |
| 1177 | 1891 | Ac-LTF$4rn6Cou4YWAQL$4a5AANleA-NH2 |
| 1178 | 1892 | Ac-LTF$4rn6AYWCou4QL$4a5AANleA-NH2 |
| 1179 | 1893 | Ac-LTF$4rn6AYWAQL$4a5Cou4ANleA-NH2 |
| 1180 | 1894 | Ac-LTF$4rn6AYWAQL$4a5Cou4ANleA-NH2 |
| 1181 | 1895 | Ac-LTF$4rn6AYWAQL$4a5ACou4NleA-NH2 |
| 1182 | 1896 | Ac-LTF$4rn6AYWAQL$4a5ACou4NleA-NH2 |
| 1183 | 1897 | Ac-LTF$4rn6AYWAQL$4a5AANleA-OH |
| 1184 | 1898 | Ac-LTF$4rn6AYWAQL$4a5AANleA-OH |
| 1185 | 1899 | Ac-LTF$4rn6AYWAQL$4a5AANleA-NHnPr |
| 1186 | 1900 | Ac-LTF$4rn6AYWAQL$4a5AANleA-NHnPr |
| 1187 | 1901 | Ac-LTF$4rn6AYWAQL$4a5AANleA-NHnBu33Me |
| 1188 | 1902 | Ac-LTF$4rn6AYWAQL$4a5AANleA-NHnBu33Me |
| 1189 | 1903 | Ac-LTF$4rn6AYWAQL$4a5AANleA-NHHex |
| 1190 | 1904 | Ac-LTF$4rn6AYWAQL$4a5AANleA-NHHex |
| 1191 | 1905 | Ac-LTA$4rn6AYWAQL$4a5AANleA-NH2 |
| 1192 | 1906 | Ac-LThL$4rn6AYWAQL$4a5AANleA-NH2 |
| 1193 | 1907 | Ac-LTF$4rn6AYAAQL$4a5AANleA-NH2 |
| 1194 | 1908 | Ac-LTF$4rn6AY2NalAQL$4a5AANleA-NH2 |
| 1195 | 1909 | Ac-LTF$4rn6EYWCou4QCba$4a5SAA-NH2 |
| 1196 | 1910 | Ac-LTF$4rn6EYWCou7QCba$4a5SAA-NH2 |
| 1197 | 1911 | Dmaac-LTF$4rn6EYWAQCba$4a5SAA-NH2 |
| 1198 | 1912 | Dmaac-LTF$4rn6AYWAQL$4a5AAAAAa-NH2 |
| 1199 | 1913 | Dmaac-LTF$4rn6EYWAQL$4a5AAAAAa-NH2 |
| 1200 | 1914 | Dmaac-LTF$4rn6EF4coohWAQCba$4a5AAIa-NH2 |
| 1201 | 1915 | Dmaac-LTF$4rn6EF4coohWAQCba$4a5AAIa-NH2 |
| 1202 | 1916 | Dmaac-LTF$4rn6AYWAQL$4a5AANleA-NH2 |
| 1203 | 1917 | Cou6BaLTF$4rn6EYWAQhL$4a5SAA-NH2 |
| 1204 | 1918 | Cou8BaLTF$4rn6EYWAQhL$4a5SAA-NH2 |
| 1205 | 1919 | Ac-LTF4I$4rn6EYWAQL$4a5AAAAAa-NH2 |

TABLE 3A

| | SEQ | | Exact | Found | Calc | Calc | Calc |
|---|---|---|---|---|---|---|---|
| SP | ID NO: | Sequence | Mass | Mass | (M + 1)/1 | (M + 2)/2 | (M + 3)/3 |
| 1206 | 1920 | Ac-LTF$4rn6AYWAQL$4a5AANleA-NH2 | 1812.01 | 907.89 | 1813.02 | 907.01 | 605.01 |
| 1207 | 1921 | Ac-LTF$4rn6AYWAQL$4a5AAAAAa-NH2 | 1912.04 | 957.75 | 1913.05 | 957.03 | 638.35 |
| 1208 | 1922 | Ac-LTF$4rn6EYWAQL$4a5AAAAAa-NH2 | 1970.04 | 986.43 | 1971.05 | 986.03 | 657.69 |
| 1209 | 1923 | Ac-LTF$5rn6AYWAQL$5a5AAAAAa-NH2 | 1912.04 | 957.38 | 1913.05 | 957.03 | 638.35 |
| 1210 | 1924 | Ac-LTF$4rn6EYWAQCba$4a5SAA-NH2 | 1784.93 | 894.38 | 1785.94 | 893.47 | 595.98 |
| 1211 | 1925 | Ac-LTF$4rn4EYWAQCba$4a5SAA-NH2 | 1756.89 | 880.05 | 1757.9 | 879.45 | 586.64 |
| 1212 | 1926 | Ac-LTF$4rn5EYWAQCba$4a5SAA-NH2 | 1770.91 | 887.08 | 1771.92 | 886.46 | 591.31 |
| 1213 | 1927 | Ac-LTF$5rn6EYWAQCba$5a5SAA-NH2 | 1784.92 | 894.11 | 1785.93 | 893.47 | 595.98 |
| 1214 | 1928 | Ac-LTF$4rn6EYWAQCba5I-$4a5SAA-NH2 | 1910.82 | 957.01 | 1911.83 | 956.42 | 637.95 |
| 1215 | 1929 | Ac-LTA$5rn6EYWAQCba$5a5SAA-NH2 | 1708.89 | 856 | 1709.9 | 855.45 | 570.64 |
| 1216 | 1930 | Ac-LTA$4rn6EYWAQCba$4a5SAA-NH2 | 1708.89 | 856 | 1709.9 | 855.45 | 570.64 |
| 1217 | 1931 | 5-FAM-BaLTF$4rn6EYWAQCba$4a5SAA-NH2 | 2172 | 1087.81 | 2173.01 | 1087.01 | 725.01 |
| 1218 | 1932 | 5-FAM-BaLTA$4rn6EYWAQCba$4a5SAA-NH2 | 2095.97 | 1049.79 | 2096.98 | 1048.99 | 699.66 |
| 1219 | 1933 | 5-FAM-BaLTF$5rn6EYWAQCba$5a5SAA-NH2 | 2172 | 1087.53 | 2173.01 | 1087.01 | 725.01 |
| 1220 | 1934 | 5-FAM-BaLTA$5rn6EYWAQCba$5a5SAA-NH2 | 2095.97 | 1049.98 | 2096.98 | 1048.99 | 699.66 |
| 1221 | 1935 | Ac-LTF$4rn6EYWAQCba5Ph-$4a5SAA-NH2 | 1675.87 | 932.31 | 1676.88 | 931.48 | 559.63 |
| 1222 | 1936 | Ac-LTF$4rn6EYWAQCba5Prp-$4a5SAA-NH2 | 1675.87 | 914.46 | 1676.88 | 913.48 | 559.63 |
| 1223 | 1937 | Ac-LTF$4rn6AYWAAL$4a5AAAAAa-NH2 | 1855.01 | | 1856.02 | 928.51 | 619.34 |
| 1224 | 1938 | Ac-LTF$4rn6EYWAQCba5penNH2-$4a5SAA-NH2 | 1675.87 | | 1676.88 | 838.94 | 559.63 |
| 1225 | 1939 | Ac-LTF$4rn6EYWAQCba5BnzNH2-$4a5SAA-NH2 | 1675.87 | | 1676.88 | 838.94 | 559.63 |
| 1226 | 1940 | Ac-LTF$4rn6EYWAQCba5prpOMe-$4a5SAA-NH2 | | 929.17 | | 928.48 | |
| 1227 | 1941 | Ac-LTF$5rn6EYWAQL4Me$5a5AAAAAa-NH2 | 1926.05 | | 1927.06 | 964.03 | 643.02 |
| 1228 | 1942 | Ac-LTF$5rn6EYWAQL4Ph$5a5AAAAAa-NH2 | 1988.07 | | 1989.07 | 995.04 | 663.70 |
| 1229 | 1943 | Ac-LTF$5rn6EYWAQCba4Me$5a5SAANH2 | 1740.93 | | 1741.94 | 871.48 | 581.32 |
| 1230 | 1944 | Ac-LTF$5rn6EYWAQCba4Ph$5a5SAANH2 | 1802.95 | | 1803.96 | 902.48 | 601.99 |

### Example 4: Preparation of Peptidomimetic Macrocycles Using a Boc-Protected Amino Acid

Peptidomimetic macrocycle precursors comprising an R8 amino acid at position “i” and an S5 amino acid at position “i+7” were prepared. The amino acid at position “i+3” was a Boc-protected tryptophan, which was incorporated during solid-phase synthesis. Specifically, the Boc-protected tryptophan amino acid shown below was used during solid phase synthesis:

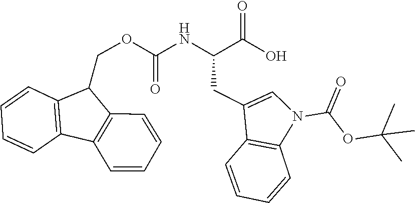 [SMILES: CC(C)(C)OC(=O)n1cc(C[C@H](NC(=O)OCC2c3ccccc3-c3ccccc32)C(=O)O)c2ccccc21]

Metathesis was performed using a ruthenium catalyst prior to the cleavage and deprotection steps. The composition obtained following cyclization was determined by HPLC analysis and was found to contain primarily peptidomimetic macrocycles having a crosslinker comprising a trans olefin (“iso2”, comprising the double bond in an E configuration). Unexpectedly, a ratio of 90:10 was observed for the trans and cis products, respectively.

### Example 5: Peptidomimetic Macrocycle Solubility

Peptidomimetic macrocycles were first dissolved in neat N, N-dimethylacetamide (DMA) to make 20× stock solutions over a concentration range of 20-140 mg/mL. The DMA stock solutions were diluted 20-fold in an aqueous vehicle containing 2% Solutol-HS-15, 25 mM histidine, and 45 mg/mL mannitol to obtain final concentrations of 1-7 mg/ml of the peptidomimetic macrocycles in 5% DMA, 2% Solutol-HS-15, 25 mM histidine, and 45 mg/mL mannitol. The final solutions were mixed gently by repeat pipetting or light vortexing. The final solutions were sonicated for 10 min at room temperature in an ultrasonic water bath. Careful visual observations were performed under a hood light using a 7× visual amplifier to determine if precipitates existed on the bottom of the flasks or as a suspension. Additional concentration ranges were tested as needed to determine the maximum solubility limit for each peptidomimetic macrocycle.

### Example 6: X-Ray Co-Crystallography of Peptidomimetic Macrocycles in Complex with MDMX

For co-crystallization with peptide 46 (TABLE 2b), a stoichiometric amount of compound from a 100 mM stock solution in DMSO was added to a zebrafish MDMX protein solution. The solution was allowed to sit overnight at 4° C. before setting up crystallization experiments. Protein (residues 15-129, L46V/V95L) was obtained from an E. coli BL21 (DE3) expression system using the pET15b vector. Cells were grown at 37° C. and induced with 1 mM IPTG at an OD₆₀₀ of 0.7. Cells were allowed to grow an additional 18 hrs at 23° C. The protein was purified using Ni-NT Agarose followed by Superdex 75 buffered with 50 mM NaPO₄, pH 8.0, 150 mM NaCl, and 2 mM TCEP, and concentrating to 24 mg/ml. The buffer was exchanged to 20 mM Tris, pH 8.0, 50 mM NaCl, and 2 mM DTT for crystallization experiments. Initial crystals were obtained with the Nextal AMS screen #94, and the final optimized reservoir was 2.6 M AMS, 75 mM Hepes, pH 7.5. Crystals grew routinely as thin plates at 4° C. and were cryo-protected by pulling the crystals through a solution containing concentrated (3.4 M) malonate followed by flash cooling, storage, and shipment in liquid nitrogen.

Data collection was performed at the APS at beamline 31-ID (SGX-CAT) at 100° K and wavelength 0.97929 Å. The beamline was equipped with a Rayonix 225-HE detector. For data collection, crystals were rotated through 180° in 1° increments using 0.8 second exposure times. Data were processed and reduced using Mosflm/scala (CCP4) in space group C2 (unit cell: a=109.2786, b=81.0836, c=30.9058 Å, α=90, β=89.8577, γ=90°). Molecular replacement with program Molrep (CCP4) was performed with the MDMX component of the structure, and two molecules were identified in the asymmetric unit. Initial refinement of just the two molecules of the zebrafish MDMX with program Refmac (CCP4) resulted in an R-factor of 0.3424 (R_{free}=0.3712) and rmsd values for bonds (0.018 Å) and angles (1.698°). The electron densities of the stapled peptide components, starting with Gln¹⁹ and including the entire aliphatic staple, were very clear. Further refinement with CNX using data to 2.3 Å resolution resulted in a model (comprised of 1448 atoms from MDMX, 272 atoms from the stapled peptides and 46 water molecules) that was well refined (Rf=0.2601, R_{free}=0.3162, rmsd bonds=0.007 Å and rmsd angles=0.916°).

### Example 7: Circular Dichroism (CD) Analysis of Alpha-Helicity

Peptide solutions were analyzed by CD spectroscopy using a spectropolarimeter. A temperature controller was used to maintain temperature control of the optical cell. Results are expressed as mean molar ellipticity [θ] (deg cm² dmol⁻¹) as calculated from the equation [θ]=θobs·MRW/10*1*c where θobs is the observed ellipticity in millidegrees, MRW is the mean residue weight of the peptide (peptide molecular weight/number of residues), 1 is the optical path length of the cell in centimeters, and c is the peptide concentration in mg/ml. Peptide concentrations were determined by amino acid analysis. Stock solutions of peptides were prepared in benign CD buffer (20 mM phosphoric acid, pH 2). The stock solutions were used to prepare peptide solutions of 0.05 mg/ml in either benign CD buffer or CD buffer with 50% trifluoroethanol (TFE) for analyses in a 10 mm path length cell. Variable wavelength measurements of peptide solutions were scanned at 4° C. from 195 to 250 nm, in 0.2 nm increments, and a scan rate 50 nm per minute. The average of six scans is reported.

TABLE 4 shows CD data for selected peptidomimetic macrocycles:

TABLE 4

| | | | Molar | % Helix | % Helix |
|---|---|---|---|---|---|
| | Molar | Molar | Ellipticity | 50% TFE | benign |
| | Ellipticity | Ellipticity | TFE − Molar | compared | compared |
| | Benign (222 | 50% TFE (222 | Ellipticity | to 50% TFE | to 50% TFE |
| SP# | in 0% TFE) | in 50% TFE) | Benign | parent (CD) | parent (CD) |
| 7 | 124 | −19921.4 | −20045.4 | 137.3 | −0.9 |
| 11 | −398.2 | −16623.4 | 16225.2 | 106.1 | 2.5 |
| 41 | −909 | −21319.4 | 20410.4 | 136 | 5.8 |
| 43 | −15334.5 | −18247.4 | 2912.9 | 116.4 | 97.8 |
| 69 | −102.6 | −21509.7 | −21407.1 | 148.2 | 0.7 |
| 71 | −121.2 | −17957 | −17835.9 | 123.7 | 0.8 |
| 154 | −916.2 | −30965.1 | −30048.9 | 213.4 | 6.3 |
| 230 | −213.2 | −17974 | −17760.8 | 123.9 | 1.5 |
| 233 | −477.9 | −19032.6 | −18554.7 | 131.2 | 3.3 |

### Example 8: Direct Binding Assay MDM2 with Fluorescence Polarization (FP)

The assay was performed according to the following general protocol:

1. Dilute MDM2 (In-house, 41 kD) into FP buffer (High salt buffer-200 mM NaCl, 5 mM CHAPS, pH 7.5) to make 10 μM working stock solution.
2. Add 30 μl of 10 μM of protein stock solution into A1 and B1 well of 96-well black HE microplate (Molecular Devices).
3. Fill in 30 μl of FP buffer into column A2 to A12, B2 to B12, C1 to C12, and D1 to D12.
4. 2- or 3-fold series dilution of protein stock from A1, B1 into A2, B2; A2, B2 to A3, B3; . . . to reach the single digit nM concentration at the last dilution point.
5. Dilute 1 mM (in 100% DMSO) of FAM labeled linear peptide with DMSO to 100 μM (dilution 1:10). Then, dilute from 100 μM to 10 μM with water (dilution 1:10) and then dilute with FP buffer from 10 μM to 40 nM (dilution 1:250). This is the working solution which is a 10 nM concentration in well (dilution 1:4). Keep the diluted FAM labeled peptide in the dark until use.
6. Add 10 μl of 10 nM of FAM labeled peptide into each well and incubate and read at different time points. K_{D} with 5-FAM-BaLTFEHYWAQLTS-NH₂ (SEQ ID NO: 1947) is ˜13.38 nM.

### Example 9: Competitive Fluorescence Polarization Assay for MDM2

MDM2 (41 kD) was diluted into FP buffer (high-salt buffer-200 mM NaCl, 5 mM CHAPS, pH 7.5) to make a 84 nM (2×) working stock solution. 20p1 of the 84 nM (2×) protein stock solution was added into each well of a 96-well black microplate. 1 mM of FAM-labeled linear peptide (in 100% DMSO) was diluted to 100 μM with DMSO (dilution 1:10). Then, diluted solution was further diluted from 100 μM to 10 μM with water (dilution 1:10), and diluted again with FP buffer from 10 μM to 40 nM (dilution 1:250). The resulting working solution resulted in a 10 nM concentration in each well (dilution 1:4). The diluted FAM-labeled peptides were kept in the dark until use.

Unlabeled peptide dose plates were prepared with FP buffer starting with 1 μM (final) of the peptide. 5-fold serial dilutions were made for 6 points using the following dilution scheme. 10 mM of the solution (in 100% DMSO) with DMSO to 5 mM (dilution 1:2); dilution from 5 mM to 500 μM with H₂O (dilution 1:10); and dilution with FP buffer from 500 μM to 20 μM (dilution 1:25). 5-fold serial dilutions from 4 μM (4×) were made for 6 points. 10 μl of the serial diluted unlabeled peptides were transferred to each well, which was filled with 20 μl of 84 nM of protein. 10 μl of 10 nM (4×) of FAM-labeled peptide was added into each well, and the wells were incubated for 3 h before being read.

### Example 10: Direct Binding Assay MDMX with Fluorescence Polarization (FP)

MDMX (40 kD) was diluted into FP buffer (high-salt buffer-200 mM NaCl, 5 mM CHAPS, pH 7.5) to make a 10 μM working stock solution. 30 μl of the 10 μM of protein stock solution was added into the A1 and B1 wells of a 96-well black microplate. 30 μl of FP buffer was added to columns A2 to A12, B2 to B12, C1 to C12, and D1 to D12. 2-fold or 3-fold series dilutions of protein stocks were created from A1, B1 into A2, B2; A2, B2 to A3, B3; . . . to reach the single digit nM concentration at the last dilution point. 1 mM (in 100% DMSO) of a FAM-labeled linear peptide was diluted with DMSO to 100 μM (dilution 1:10). The resulting solution was diluted from 100 μM to 10 μM with water (dilution 1:10), and diluted again with FP buffer from 10 μM to 40 nM (dilution 1:250). The working solution resulted in 10 nM concentration in each well (dilution 1:4). The FAM-labeled peptides were kept in the dark until use. 10 μl of the 10 nM FAM-labeled peptide was added into each well, and the plate was incubated and read at different time points. The K_{D} with 5-FAM-BaLTFEHYWAQLTS-NH₂ (SEQ ID NO: 1947) was ˜51 nM.

### Example 11: Competitive Fluorescence Polarization Assay for MDMX

MDMX (40 kD) was diluted into FP buffer (high-salt buffer 200 mM NaCl, 5 mM CHAPS, pH 7.5) to make a 300 nM (2×) working stock solution. 20 μl of the 300 nM (2×) of protein stock solution was added into each well of 96-well black microplate. 1 mM (in 100% DMSO) of a FAM-labeled linear peptide was diluted with DMSO to a concentration of 100 μM (dilution 1:10). The solution was diluted from 100 μM to 10 μM with water (dilution 1:10), and diluted further with FP buffer from 10 μM to 40 nM (dilution 1:250). The final working solution resulted in a concentration of 10 nM per well (dilution 1:4). The diluted FAM-labeled peptide was kept in the dark until use. An unlabeled peptide dose plate was prepared with FP buffer starting with a concentration of 5 μM (final) of a peptide. 5-fold serial dilutions were prepared for 6 points using the following dilution scheme. 10 mM (in 100% DMSO) of the solution was diluted with DMSO to prepare a 5 mM (dilution 1:2) solution. The solution was diluted from 5 mM to 500 μM with H₂O (dilution 1:10), and diluted further with FP buffer from 500 μM to 20 μM (dilution 1:25). 5-fold serial dilutions from 20 μM (4×) were prepared for 6 points. 10 μl of the serially diluted unlabeled peptides were added to each well, which was filled with 20 μl of the 300 nM protein solution. 10 μl of the 10 nM (4×) FAM-labeled peptide solution was added into each well, and the wells were incubated for 3 h before reading.

Results from EXAMPLE 8-EXAMPLE 11 are shown in TABLE 5. The following scale is used: “+” represents a value greater than 1000 nM, “++” represents a value greater than 100 and less than or equal to 1000 nM, “+++” represents a value greater than 10 nM and less than or equal to 100 nM, and “++++” represents a value of less than or equal to 10 nM.

TABLE 5

| SP# | IC₅₀ (MDM2) | IC₅₀ (MDMX) | Ki (MDM2) | Ki (MDMX) |
|---|---|---|---|---|
| 3 | ++ | ++ | +++ | +++ |
| 4 | +++ | ++ | ++++ | +++ |
| 5 | +++ | ++ | ++++ | +++ |
| 6 | ++ | ++ | +++ | +++ |
| 7 | +++ | +++ | ++++ | +++ |
| 8 | ++ | ++ | +++ | +++ |
| 9 | ++ | ++ | +++ | +++ |
| 10 | ++ | ++ | +++ | +++ |
| 11 | +++ | ++ | ++++ | +++ |
| 12 | + | + | +++ | ++ |
| 13 | ++ | ++ | +++ | ++ |
| 14 | +++ | +++ | ++++ | ++++ |
| 15 | +++ | ++ | +++ | +++ |
| 16 | +++ | +++ | ++++ | +++ |
| 17 | +++ | +++ | ++++ | +++ |
| 18 | +++ | +++ | ++++ | ++++ |
| 19 | ++ | +++ | +++ | +++ |
| 20 | ++ | ++ | +++ | +++ |
| 21 | ++ | +++ | +++ | +++ |
| 22 | +++ | +++ | ++++ | +++ |
| 23 | ++ | ++ | +++ | +++ |
| 24 | +++ | ++ | ++++ | +++ |
| 26 | +++ | ++ | ++++ | +++ |
| 28 | +++ | +++ | ++++ | +++ |
| 30 | ++ | ++ | +++ | +++ |
| 32 | +++ | ++ | ++++ | +++ |
| 38 | + | ++ | ++ | +++ |
| 39 | + | ++ | ++ | ++ |
| 40 | ++ | ++ | ++ | +++ |
| 41 | ++ | +++ | +++ | +++ |
| 42 | ++ | ++ | +++ | ++ |
| 43 | +++ | +++ | ++++ | +++ |
| 45 | +++ | +++ | ++++ | ++++ |
| 46 | +++ | +++ | ++++ | +++ |
| 47 | ++ | ++ | +++ | +++ |
| 48 | ++ | ++ | +++ | +++ |
| 49 | ++ | ++ | +++ | +++ |
| 50 | +++ | ++ | ++++ | +++ |
| 52 | +++ | +++ | ++++ | ++++ |
| 54 | ++ | ++ | +++ | +++ |
| 55 | + | + | ++ | ++ |
| 65 | +++ | ++ | ++++ | +++ |
| 68 | ++ | ++ | +++ | +++ |
| 69 | +++ | ++ | ++++ | +++ |
| 70 | ++ | ++ | ++++ | +++ |
| 71 | +++ | ++ | ++++ | +++ |
| 75 | +++ | ++ | ++++ | +++ |
| 77 | +++ | ++ | ++++ | +++ |
| 80 | +++ | ++ | ++++ | +++ |
| 81 | ++ | ++ | +++ | +++ |
| 82 | ++ | ++ | +++ | +++ |
| 85 | +++ | ++ | ++++ | +++ |
| 99 | ++++ | ++ | ++++ | +++ |
| 100 | ++ | ++ | ++++ | +++ |
| 101 | +++ | ++ | ++++ | +++ |
| 102 | ++ | ++ | ++++ | +++ |
| 103 | ++ | ++ | ++++ | +++ |
| 104 | +++ | ++ | ++++ | +++ |
| 105 | +++ | ++ | ++++ | +++ |
| 106 | ++ | ++ | +++ | +++ |
| 107 | ++ | ++ | +++ | +++ |
| 108 | +++ | ++ | ++++ | +++ |
| 109 | +++ | ++ | ++++ | +++ |
| 110 | ++ | ++ | ++++ | +++ |
| 111 | ++ | ++ | ++++ | +++ |
| 112 | ++ | ++ | +++ | +++ |
| 113 | ++ | ++ | +++ | +++ |
| 114 | +++ | ++ | ++++ | +++ |
| 115 | ++++ | ++ | ++++ | +++ |
| 116 | + | + | ++ | ++ |
| 118 | ++++ | ++ | ++++ | +++ |
| 120 | +++ | ++ | ++++ | +++ |
| 121 | ++++ | ++ | ++++ | +++ |
| 122 | ++++ | ++ | ++++ | +++ |
| 123 | ++++ | ++ | ++++ | +++ |
| 124 | ++++ | ++ | ++++ | +++ |
| 125 | ++++ | ++ | ++++ | +++ |
| 126 | ++++ | ++ | ++++ | +++ |
| 127 | ++++ | ++ | ++++ | +++ |
| 128 | ++++ | ++ | ++++ | +++ |
| 129 | ++++ | ++ | ++++ | +++ |
| 130 | ++++ | ++ | ++++ | +++ |
| 133 | ++++ | ++ | ++++ | +++ |
| 134 | ++++ | ++ | ++++ | +++ |
| 135 | ++++ | ++ | ++++ | +++ |
| 136 | ++++ | ++ | ++++ | +++ |
| 137 | ++++ | ++ | ++++ | +++ |
| 139 | ++++ | ++ | ++++ | +++ |
| 142 | ++++ | +++ | ++++ | +++ |
| 144 | ++++ | ++ | ++++ | +++ |
| 146 | ++++ | ++ | ++++ | +++ |
| 148 | ++++ | ++ | ++++ | +++ |
| 150 | ++++ | ++ | ++++ | +++ |
| 153 | ++++ | +++ | ++++ | +++ |
| 154 | ++++ | +++ | ++++ | ++++ |
| 156 | ++++ | ++ | ++++ | +++ |
| 158 | ++++ | ++ | ++++ | +++ |
| 160 | ++++ | ++ | ++++ | +++ |
| 161 | ++++ | ++ | ++++ | +++ |
| 166 | ++++ | ++ | ++++ | +++ |
| 167 | +++ | ++ | ++++ | ++ |
| 169 | ++++ | +++ | ++++ | +++ |
| 170 | ++++ | ++ | ++++ | +++ |
| 173 | ++++ | ++ | ++++ | +++ |
| 175 | ++++ | ++ | ++++ | +++ |
| 177 | +++ | ++ | ++++ | +++ |
| 180 | +++ | ++ | ++++ | +++ |
| 182 | ++++ | ++ | ++++ | +++ |
| 185 | +++ | + | ++++ | ++ |
| 186 | +++ | ++ | ++++ | +++ |
| 189 | +++ | ++ | ++++ | +++ |
| 192 | +++ | ++ | ++++ | +++ |
| 194 | +++ | ++ | ++++ | ++ |
| 196 | +++ | ++ | ++++ | +++ |
| 197 | ++++ | ++ | ++++ | +++ |
| 199 | +++ | ++ | ++++ | ++ |
| 201 | +++ | ++ | ++++ | ++ |
| 203 | +++ | ++ | ++++ | +++ |
| 204 | +++ | ++ | ++++ | +++ |
| 206 | +++ | ++ | ++++ | +++ |
| 207 | ++++ | ++ | ++++ | +++ |
| 210 | ++++ | ++ | ++++ | +++ |
| 211 | ++++ | ++ | ++++ | +++ |
| 213 | ++++ | ++ | ++++ | +++ |
| 215 | +++ | ++ | ++++ | +++ |
| 217 | ++++ | ++ | ++++ | +++ |
| 218 | ++++ | ++ | ++++ | +++ |
| 221 | ++++ | +++ | ++++ | +++ |
| 227 | ++++ | ++ | ++++ | +++ |
| 230 | ++++ | +++ | ++++ | ++++ |
| 232 | ++++ | ++ | ++++ | +++ |
| 233 | ++++ | +++ | ++++ | +++ |
| 236 | +++ | ++ | ++++ | +++ |
| 237 | +++ | ++ | ++++ | +++ |
| 238 | +++ | +++ | ++++ | +++ |
| 239 | +++ | ++ | +++ | +++ |
| 240 | +++ | ++ | ++++ | +++ |
| 241 | +++ | ++ | ++++ | +++ |
| 242 | +++ | ++ | ++++ | +++ |
| 243 | +++ | +++ | ++++ | +++ |
| 244 | +++ | +++ | ++++ | ++++ |
| 245 | +++ | +++ | ++++ | +++ |
| 246 | +++ | ++ | ++++ | +++ |
| 247 | +++ | +++ | ++++ | +++ |
| 248 | +++ | +++ | ++++ | +++ |
| 249 | +++ | +++ | ++++ | ++++ |
| 250 | ++ | + | ++ | + |
| 252 | ++ | + | ++ | + |
| 254 | +++ | ++ | ++++ | +++ |
| 255 | +++ | +++ | ++++ | +++ |
| 256 | +++ | +++ | ++++ | +++ |
| 257 | +++ | +++ | ++++ | +++ |
| 258 | +++ | ++ | ++++ | +++ |
| 259 | +++ | +++ | ++++ | +++ |
| 260 | +++ | +++ | ++++ | +++ |
| 261 | +++ | ++ | ++++ | +++ |
| 262 | +++ | ++ | ++++ | +++ |
| 263 | +++ | ++ | ++++ | +++ |
| 264 | +++ | +++ | ++++ | +++ |
| 266 | +++ | ++ | ++++ | +++ |
| 267 | +++ | +++ | ++++ | ++++ |
| 270 | ++++ | +++ | ++++ | +++ |
| 271 | ++++ | +++ | ++++ | ++++ |
| 272 | ++++ | +++ | ++++ | ++++ |
| 276 | +++ | +++ | ++++ | ++++ |
| 277 | +++ | +++ | ++++ | ++++ |
| 278 | +++ | +++ | ++++ | ++++ |
| 279 | ++++ | +++ | ++++ | +++ |
| 280 | +++ | ++ | ++++ | +++ |
| 281 | +++ | + | +++ | ++ |
| 282 | ++ | + | +++ | + |
| 283 | +++ | ++ | +++ | ++ |
| 284 | +++ | ++ | ++++ | +++ |
| 289 | +++ | +++ | ++++ | +++ |
| 291 | +++ | +++ | ++++ | ++++ |
| 293 | ++++ | +++ | ++++ | +++ |
| 306 | ++++ | ++ | ++++ | +++ |
| 308 | ++ | ++ | +++ | +++ |
| 310 | +++ | +++ | ++++ | +++ |
| 312 | +++ | ++ | +++ | +++ |
| 313 | ++++ | ++ | ++++ | +++ |
| 314 | ++++ | +++ | ++++ | ++++ |
| 315 | +++ | +++ | ++++ | +++ |
| 316 | ++++ | ++ | ++++ | +++ |
| 317 | +++ | ++ | +++ | +++ |
| 318 | +++ | ++ | +++ | +++ |
| 319 | +++ | ++ | +++ | ++ |
| 320 | +++ | ++ | +++ | ++ |
| 321 | +++ | ++ | ++++ | +++ |
| 322 | +++ | ++ | +++ | ++ |
| 323 | +++ | + | +++ | ++ |
| 328 | +++ | +++ | ++++ | +++ |
| 329 | +++ | +++ | ++++ | +++ |
| 331 | ++++ | +++ | ++++ | ++++ |
| 332 | ++++ | +++ | ++++ | ++++ |
| 334 | ++++ | +++ | ++++ | ++++ |
| 336 | ++++ | +++ | ++++ | ++++ |
| 339 | ++++ | ++ | ++++ | +++ |
| 341 | +++ | +++ | ++++ | ++++ |
| 343 | +++ | +++ | ++++ | ++++ |
| 347 | +++ | +++ | ++++ | +++ |
| 349 | ++++ | +++ | ++++ | ++++ |
| 351 | ++++ | +++ | ++++ | ++++ |
| 353 | ++++ | +++ | ++++ | ++++ |
| 355 | ++++ | +++ | ++++ | ++++ |
| 357 | ++++ | +++ | ++++ | ++++ |
| 359 | ++++ | +++ | ++++ | +++ |
| 360 | ++++ | ++++ | ++++ | ++++ |
| 363 | +++ | +++ | ++++ | ++++ |
| 364 | +++ | +++ | ++++ | ++++ |
| 365 | +++ | +++ | ++++ | ++++ |
| 366 | +++ | +++ | ++++ | +++ |
| 369 | ++ | ++ | +++ | +++ |
| 370 | +++ | +++ | ++++ | +++ |
| 371 | ++ | ++ | +++ | +++ |
| 372 | ++ | ++ | +++ | +++ |
| 373 | +++ | +++ | +++ | +++ |
| 374 | +++ | +++ | ++++ | ++++ |
| 375 | +++ | +++ | ++++ | ++++ |
| 376 | +++ | +++ | ++++ | ++++ |
| 377 | +++ | +++ | ++++ | +++ |
| 378 | +++ | +++ | ++++ | +++ |
| 379 | +++ | +++ | ++++ | +++ |
| 380 | +++ | +++ | ++++ | +++ |
| 381 | +++ | +++ | ++++ | +++ |
| 382 | +++ | +++ | ++++ | ++++ |
| 384 | ++ | + | ++ | + |
| 386 | ++ | + | ++ | + |
| 388 | ++ | +++ | +++ | ++++ |
| 390 | +++ | +++ | ++++ | +++ |
| 392 | +++ | +++ | ++++ | ++++ |
| 394 | ++++ | +++ | ++++ | ++++ |
| 396 | ++++ | ++++ | ++++ | ++++ |
| 398 | +++ | +++ | ++++ | +++ |
| 402 | ++++ | ++++ | ++++ | ++++ |
| 404 | +++ | +++ | ++++ | ++++ |
| 408 | +++ | +++ | ++++ | +++ |
| 410 | ++++ | ++++ | ++++ | ++++ |
| 411 | ++ | + | ++ | + |
| 412 | ++++ | +++ | ++++ | ++++ |
| 415 | ++++ | ++++ | ++++ | ++++ |
| 416 | +++ | +++ | ++++ | +++ |
| 417 | +++ | +++ | ++++ | +++ |
| 418 | ++++ | +++ | ++++ | ++++ |
| 419 | +++ | +++ | +++ | ++++ |
| 421 | ++++ | ++++ | ++++ | ++++ |
| 423 | +++ | +++ | ++++ | +++ |
| 425 | +++ | +++ | +++ | +++ |
| 427 | ++ | ++ | +++ | +++ |
| 432 | ++++ | +++ | ++++ | ++++ |
| 434 | +++ | +++ | ++++ | +++ |
| 435 | ++++ | +++ | ++++ | ++++ |
| 437 | +++ | +++ | ++++ | +++ |
| 439 | ++++ | +++ | ++++ | ++++ |
| 441 | ++++ | ++++ | ++++ | ++++ |
| 443 | +++ | +++ | ++++ | +++ |
| 445 | +++ | ++ | ++++ | +++ |
| 446 | +++ | + | ++++ | + |
| 447 | ++ | + | ++ | + |
| 551 | N/A | N/A | ++++ | +++ |
| 555 | N/A | N/A | ++++ | +++ |
| 556 | N/A | N/A | ++++ | +++ |
| 557 | N/A | N/A | +++ | +++ |
| 558 | N/A | N/A | +++ | +++ |
| 559 | N/A | N/A | +++ | +++ |
| 560 | N/A | N/A | + | + |
| 561 | N/A | N/A | ++++ | +++ |
| 562 | N/A | N/A | +++ | +++ |
| 563 | N/A | N/A | +++ | +++ |
| 564 | N/A | N/A | ++++ | +++ |
| 565 | N/A | N/A | +++ | +++ |
| 566 | N/A | N/A | ++++ | +++ |
| 567 | N/A | N/A | ++++ | +++ |
| 568 | N/A | N/A | ++++ | ++++ |
| 569 | N/A | N/A | ++++ | +++ |
| 570 | N/A | N/A | ++++ | +++ |
| 571 | N/A | N/A | ++++ | +++ |
| 572 | N/A | N/A | +++ | +++ |
| 573 | N/A | N/A | +++ | +++ |
| 574 | N/A | N/A | ++++ | +++ |
| 575 | N/A | N/A | ++++ | +++ |
| 576 | N/A | N/A | ++++ | +++ |
| 577 | N/A | N/A | ++++ | +++ |
| 578 | N/A | N/A | ++++ | +++ |
| 585 | N/A | N/A | +++ | +++ |
| 586 | N/A | N/A | ++++ | +++ |
| 587 | N/A | N/A | ++++ | ++++ |
| 589 | N/A | N/A | ++++ | |
| 594 | N/A | N/A | ++++ | ++++ |
| 596 | N/A | N/A | ++++ | +++ |
| 597 | N/A | N/A | ++++ | +++ |
| 598 | N/A | N/A | ++++ | +++ |
| 600 | N/A | N/A | ++++ | ++++ |
| 602 | N/A | N/A | ++++ | ++++ |
| 603 | N/A | N/A | ++++ | ++++ |
| 604 | N/A | N/A | +++ | +++ |
| 608 | N/A | N/A | ++++ | +++ |
| 609 | N/A | N/A | ++++ | +++ |
| 610 | N/A | N/A | ++++ | +++ |
| 611 | N/A | N/A | ++++ | +++ |
| 612 | N/A | N/A | ++++ | +++ |
| 613 | N/A | N/A | ++++ | +++ |
| 615 | N/A | N/A | ++++ | ++++ |
| 433 | N/A | N/A | ++++ | +++ |
| 686 | N/A | N/A | ++++ | +++ |
| 687 | N/A | N/A | ++ | ++ |
| 595 | N/A | N/A | + | N/A |
| 665 | N/A | N/A | +++ | N/A |
| 708 | N/A | N/A | +++ | +++ |
| 710 | N/A | N/A | +++ | +++ |
| 711 | N/A | N/A | +++ | ++ |
| 712 | N/A | N/A | ++++ | ++++ |
| 713 | N/A | N/A | ++++ | ++++ |
| 716 | N/A | N/A | ++++ | ++++ |
| 765 | + | + | | |
| 766 | +++ | + | | |
| 752 | ++ | + | | |
| 753 | +++ | + | | |
| 754 | ++ | + | | |
| 755 | ++++ | + | | |
| 756 | +++ | + | | |
| 757 | ++++ | + | | |
| 758 | +++ | + | | |

### Example 12: Competition Binding ELISA Assay for MDM2 and MDMX

p53-His6 protein (“His6” disclosed as SEQ ID NO: 1948) (30 nM/well) was coated overnight at room temperature in the wells of 96-well plates. On the day of the experiment, the plates were washed with 1×PBS-Tween 20 (0.05%) using an automated ELISA plate washer and blocked with ELISA microwell blocking buffer for 30 minutes at room temperature. The excess blocking agent was washed off by washing the plates with 1×PBS-Tween 20 (0.05%). The peptides were diluted from 10 mM DMSO stock solutions to 500 μM working stock solutions using sterile water. Further dilutions were made in 0.5% DMSO to keep the concentration of DMSO constant across the samples. The peptide solutions were added to the wells at 2× the desired concentrations in 50 μL volumes, followed by addition of diluted GST-MDM2 or GST-HMDX protein (final concentration: 10 nM). The samples were incubated at room temperature for 2 h, and the plates were washed with PBS-Tween 20 (0.05%) prior to adding 100 μL of HRP-conjugated anti-GST antibody diluted to 0.5 μg/ml in HRP-stabilizing buffer. The plates were incubated with a detection antibody for 30 min, and the plates were washed and incubated with 100 μL per well of TMB-E substrate solution for up to 30 minutes. The reactions were stopped using 1M HCL, and absorbance was measured at 450 nm using a micro plate reader. The data were analyzed using Graph Pad PRISM software.

### Example 13: Cell Viability Assay

Cells were trypsinized, counted, and seeded at pre-determined densities in 96-well plates one day prior to conducting the cell viability assay. The following cell densities were used for each cell line: SJSA-1: 7500 cells/well; RKO: 5000 cells/well; RKO-E6: 5000 cells/well; HCT-116: 5000 cells/well; SW-480: 2000 cells/well; and MCF7: 5000 cells/well. On the day of cell viability assay, the media was replaced with fresh media containing 11% FBS (assay media) at room temperature. 180 μL of the assay media was added to each well. Control wells were prepared with no cells, and the control wells received 200 μL of media.

Peptide dilutions were made at room temperature, and the diluted peptide solutions were added to the cells at room temperature. 10 mM stock solutions of the peptides were prepared in DMSO. The stock solutions were serially diluted using a 1:3 dilution scheme to obtain 10 mM, 3.3 mM, 1.1 mM, 0.33 mM, 0.11 mM, 0.03 mM, and 0.01 mM solutions in DMSO. The serially DMSO-diluted peptides were diluted 33.3 times using sterile water, resulting in a range of 10× working stock solutions. A DMSO/sterile water (3% DMSO) solution was prepared for use in the control well. The working stock solution concentrations ranges were 300 μM, 100 μM, 30 μM, 10 μM, 3 μM, 1 μM, 0.3 μM, and 0 μM. The solutions were mixed well at each dilution step using a multichannel pipette.

Row H of the plate contained the controls. Wells H1-H3 received 20 μL of assay media. Rows H4-H9 received 20 μL of the 3% DMSO-water vehicle. Wells H10-H12 received media alone control with no cells. The MDM2 small molecule inhibitor Nutlin-3a (10 mM) was used as a positive control. Nutlin-3a was diluted using the same dilution scheme used for the peptides.

20 μL of a 10× concentration peptide stock solution was added to the appropriate well to achieve the final concentration in 200 μL in each well. For example, 20 μL of 300 μM peptide solution+180 μL of cells in media=3 μM final concentration in 200 μL volume in wells. The solution was mixed gently a few times using a pipette. The final concentration range was 30 μM, 10 μM, 3 μM, 1 μM, 0.3 μM, 0.1 μM, 0.03 μM, and 0 μM. Further dilutions were used for potent peptides. Controls included wells that received no peptides but contained the same concentration of DMSO as the wells containing peptides and wells containing no cells. The plates were incubated for 72 hours at 37° C. in a humidified 5% CO₂ atmosphere.

The viability of the cells was determined using MTT reagent. The viability of SJSA-1, RKO, RKO-E6, HCT-116 cells was determined on day 3. The viability of MCF7 cells was determined on day 5. The viability of SW-480 cells was determined on day 6. At the end of the designated incubation time, the plates were cooled to room temperature. 80 L of assay media was removed from each well. 15 μL of thawed MTT reagent was then added to each well. The plate was incubated for 2 h at 37° C. in a humidified 5% CO₂ atmosphere. 100 μL of the solubilization reagent was added to each well. The plates were incubated with agitation for 1 h at room temperature and read using a multiplate reader for absorbance at 570 nm. Cell viability was analyzed against the DMSO controls.

Results from cell viability assays are shown in TABLE 6 and TABLE 7. “+” represents a value greater than 30 μM, “++” represents a value greater than 1 μM and less than or equal to 30 μM, “+++” represents a value greater than 5 μM and less than or equal to 15 μM, and “++++” represents a value of less than or equal to 5 μM. “IC₅₀ ratio” represents the ratio of average IC₅₀ in p53+/+ cells relative to average IC₅₀ in p53−/− cells.

TABLE 6

| SP# | SJSA-1 EC50 (72 h) |
|---|---|
| 3 | +++ |
| 4 | +++ |
| 5 | ++++ |
| 6 | ++ |
| 7 | ++++ |
| 8 | +++ |
| 9 | +++ |
| 10 | +++ |
| 11 | ++++ |
| 12 | ++ |
| 13 | +++ |
| 14 | + |
| 15 | ++ |
| 16 | + |
| 17 | + |
| 18 | + |
| 19 | ++ |
| 20 | + |
| 21 | + |
| 22 | + |
| 24 | +++ |
| 26 | ++++ |
| 28 | + |
| 29 | + |
| 30 | + |
| 32 | ++ |
| 38 | + |
| 39 | + |
| 40 | + |
| 41 | + |
| 42 | + |
| 43 | ++ |
| 45 | + |
| 46 | + |
| 47 | + |
| 48 | + |
| 49 | +++ |
| 50 | ++++ |
| 52 | + |
| 54 | + |
| 55 | + |
| 65 | ++++ |
| 68 | ++++ |
| 69 | ++++ |
| 70 | ++++ |
| 71 | ++++ |
| 72 | ++++ |
| 74 | ++++ |
| 75 | ++++ |
| 77 | ++++ |
| 78 | ++ |
| 80 | ++++ |
| 81 | +++ |
| 82 | +++ |
| 83 | +++ |
| 84 | + |
| 85 | +++ |
| 99 | ++++ |
| 102 | +++ |
| 103 | +++ |
| 104 | +++ |
| 105 | +++ |
| 108 | +++ |
| 109 | +++ |
| 110 | +++ |
| 111 | ++ |
| 114 | ++++ |
| 115 | ++++ |
| 118 | ++++ |
| 120 | ++++ |
| 121 | ++++ |
| 122 | ++++ |
| 123 | ++++ |
| 124 | +++ |
| 125 | ++++ |
| 126 | ++++ |
| 127 | ++++ |
| 128 | +++ |
| 129 | ++ |
| 130 | ++++ |
| 131 | +++ |
| 132 | ++++ |
| 133 | +++ |
| 134 | +++ |
| 135 | +++ |
| 136 | ++ |
| 137 | +++ |
| 139 | ++++ |
| 142 | +++ |
| 144 | ++++ |
| 147 | ++++ |
| 148 | ++++ |
| 149 | ++++ |
| 150 | ++++ |
| 152 | +++ |
| 153 | ++++ |
| 154 | ++++ |
| 155 | ++ |
| 156 | +++ |
| 157 | +++ |
| 158 | +++ |
| 160 | ++++ |
| 161 | ++++ |
| 162 | +++ |
| 163 | +++ |
| 166 | ++ |
| 167 | +++ |
| 168 | ++ |
| 169 | ++++ |
| 170 | ++++ |
| 171 | ++ |
| 173 | +++ |
| 174 | ++++ |
| 175 | +++ |
| 176 | +++ |
| 177 | ++++ |
| 179 | +++ |
| 180 | +++ |
| 181 | +++ |
| 182 | ++++ |
| 183 | ++++ |
| 184 | +++ |
| 185 | +++ |
| 186 | ++ |
| 188 | ++ |
| 190 | ++++ |
| 192 | +++ |
| 193 | ++ |
| 194 | + |
| 195 | ++++ |
| 196 | ++++ |
| 197 | ++++ |
| 198 | ++ |
| 199 | +++ |
| 200 | +++ |
| 201 | ++++ |
| 202 | +++ |
| 203 | ++++ |
| 204 | ++++ |
| 205 | ++ |
| 206 | ++ |
| 207 | +++ |
| 208 | +++ |
| 209 | ++++ |
| 210 | +++ |
| 211 | ++++ |
| 213 | ++++ |
| 214 | ++++ |
| 215 | ++++ |
| 216 | ++++ |
| 217 | ++++ |
| 218 | ++++ |
| 219 | ++++ |
| 220 | +++ |
| 221 | ++++ |
| 222 | +++ |
| 223 | ++++ |
| 224 | ++ |
| 225 | +++ |
| 226 | ++ |
| 227 | +++ |
| 228 | ++++ |
| 229 | ++++ |
| 230 | ++++ |
| 231 | ++++ |
| 232 | ++++ |
| 233 | ++++ |
| 234 | ++++ |
| 235 | ++++ |
| 236 | ++++ |
| 237 | ++++ |
| 238 | ++++ |
| 239 | +++ |
| 240 | ++ |
| 241 | +++ |
| 242 | ++++ |
| 243 | ++++ |
| 244 | ++++ |
| 245 | ++++ |
| 246 | +++ |
| 247 | ++++ |
| 248 | ++++ |
| 249 | ++++ |
| 250 | ++ |
| 251 | + |
| 252 | + |
| 253 | + |
| 254 | +++ |
| 255 | +++ |
| 256 | ++ |
| 257 | +++ |
| 258 | +++ |
| 259 | ++ |
| 260 | ++ |
| 261 | ++ |
| 262 | +++ |
| 263 | ++ |
| 264 | ++++ |
| 266 | +++ |
| 267 | ++++ |
| 270 | ++ |
| 271 | ++ |
| 272 | ++ |
| 276 | ++ |
| 277 | ++ |
| 278 | ++ |
| 279 | ++++ |
| 280 | +++ |
| 281 | ++ |
| 282 | ++ |
| 283 | ++ |
| 284 | ++++ |
| 289 | ++++ |
| 290 | +++ |
| 291 | ++++ |
| 292 | ++++ |
| 293 | ++++ |
| 294 | ++++ |
| 295 | +++ |
| 296 | ++++ |
| 297 | +++ |
| 298 | ++++ |
| 300 | ++++ |
| 301 | ++++ |
| 302 | ++++ |
| 303 | ++++ |
| 304 | ++++ |
| 305 | ++++ |
| 306 | ++++ |
| 307 | +++ |
| 308 | ++++ |
| 309 | +++ |
| 310 | ++++ |
| 312 | ++++ |
| 313 | ++++ |
| 314 | ++++ |
| 315 | ++++ |
| 316 | ++++ |
| 317 | ++++ |
| 318 | ++++ |
| 319 | ++++ |
| 320 | ++++ |
| 321 | ++++ |
| 322 | ++++ |
| 323 | ++++ |
| 324 | ++++ |
| 326 | ++++ |
| 327 | ++++ |
| 328 | ++++ |
| 329 | ++++ |
| 330 | ++++ |
| 331 | ++++ |
| 332 | ++++ |
| 333 | ++ |
| 334 | +++ |
| 335 | ++++ |
| 336 | ++++ |
| 337 | ++++ |
| 338 | ++++ |
| 339 | ++++ |
| 340 | ++++ |
| 341 | ++++ |
| 342 | ++++ |
| 343 | ++++ |
| 344 | ++++ |
| 345 | ++++ |
| 346 | ++++ |
| 347 | ++++ |
| 348 | ++++ |
| 349 | ++++ |
| 350 | ++++ |
| 351 | ++++ |
| 352 | ++++ |
| 353 | ++++ |
| 355 | ++++ |
| 357 | ++++ |
| 358 | ++++ |
| 359 | ++++ |
| 360 | ++++ |
| 361 | +++ |
| 362 | ++++ |
| 363 | ++++ |
| 364 | ++++ |
| 365 | +++ |
| 366 | ++++ |
| 367 | ++++ |
| 368 | + |
| 369 | ++++ |
| 370 | ++++ |
| 371 | ++++ |
| 372 | +++ |
| 373 | +++ |
| 374 | ++++ |
| 375 | ++++ |
| 376 | ++++ |
| 377 | ++++ |
| 378 | ++++ |
| 379 | ++++ |
| 380 | ++++ |
| 381 | ++++ |
| 382 | ++++ |
| 386 | +++ |
| 388 | ++ |
| 390 | ++++ |
| 392 | +++ |
| 394 | +++ |
| 396 | +++ |
| 398 | +++ |
| 402 | +++ |
| 404 | +++ |
| 408 | ++++ |
| 410 | +++ |
| 411 | +++ |
| 412 | + |
| 421 | +++ |
| 423 | ++++ |
| 425 | ++++ |
| 427 | ++++ |
| 434 | +++ |
| 435 | ++++ |
| 436 | ++++ |
| 437 | ++++ |
| 438 | ++++ |
| 439 | ++++ |
| 440 | ++++ |
| 441 | ++++ |
| 442 | ++++ |
| 443 | ++++ |
| 444 | +++ |
| 445 | ++++ |
| 449 | ++++ |
| 551 | ++++ |
| 552 | ++++ |
| 554 | + |
| 555 | ++++ |
| 586 | ++++ |
| 587 | ++++ |
| 588 | ++++ |
| 589 | +++ |
| 432 | ++++ |
| 672 | + |
| 673 | ++ |
| 682 | + |
| 686 | + |
| 557 | ++++ |
| 558 | ++++ |
| 560 | + |
| 561 | ++++ |
| 562 | ++++ |
| 563 | ++++ |
| 564 | ++++ |
| 566 | ++++ |
| 567 | ++++ |
| 568 | +++ |
| 569 | ++++ |
| 571 | ++++ |
| 572 | ++++ |
| 573 | ++++ |
| 574 | ++++ |
| 575 | ++++ |
| 576 | ++++ |
| 577 | ++++ |
| 578 | ++++ |
| 585 | ++++ |
| 687 | + |
| 662 | ++++ |
| 663 | ++++ |
| 553 | +++ |
| 559 | ++++ |
| 579 | ++++ |
| 581 | ++++ |
| 582 | ++ |
| 582 | ++++ |
| 584 | +++ |
| 675 | ++++ |
| 676 | ++++ |
| 677 | + |
| 679 | ++++ |
| 700 | +++ |
| 704 | +++ |
| 591 | + |
| 706 | ++ |
| 695 | ++ |
| 595 | ++++ |
| 596 | ++++ |
| 597 | +++ |
| 598 | +++ |
| 599 | ++++ |
| 600 | ++++ |
| 601 | +++ |
| 602 | +++ |
| 603 | +++ |
| 604 | +++ |
| 606 | ++++ |
| 607 | ++++ |
| 608 | ++++ |
| 610 | ++++ |
| 611 | ++++ |
| 612 | ++++ |
| 613 | +++ |
| 614 | +++ |
| 615 | ++++ |
| 618 | ++++ |
| 619 | ++++ |
| 707 | ++++ |
| 620 | ++++ |
| 621 | ++++ |
| 622 | ++++ |
| 623 | ++++ |
| 624 | ++++ |
| 625 | ++++ |
| 626 | +++ |
| 631 | ++++ |
| 633 | ++++ |
| 634 | ++++ |
| 635 | +++ |
| 636 | +++ |
| 638 | + |
| 641 | +++ |
| 665 | ++++ |
| 708 | ++++ |
| 709 | +++ |
| 710 | + |
| 711 | ++++ |
| 712 | ++++ |
| 713 | ++++ |
| 714 | +++ |
| 715 | +++ |
| 716 | ++++ |
| 765 | + |
| 753 | + |
| 754 | + |
| 755 | + |
| 756 | + |
| 757 | ++++ |
| 758 | +++ |

TABLE 7

| | HCT-116 | RKO | RKO-E6 | SW480 | |
|---|---|---|---|---|---|
| | EC50 | EC₅₀ | EC₅₀ | EC50 | IC₅₀ |
| SP# | (72 h) | (72 h) | (72 h) | (6 days) | Ratio |
| 4 | ++++ | ++++ | +++ | ++++ | |
| 5 | ++++ | ++++ | +++ | ++++ | |
| 7 | ++++ | ++++ | +++ | ++++ | |
| 10 | ++++ | +++ | +++ | +++ | |
| 11 | ++++ | ++++ | ++ | +++ | |
| 50 | ++++ | ++++ | ++ | +++ | |
| 65 | +++ | +++ | +++ | +++ | |
| 69 | ++++ | ++++ | + | ++++ | |
| 70 | ++++ | ++++ | ++ | +++ | |
| 71 | ++++ | ++++ | +++ | +++ | |
| 81 | +++ | +++ | +++ | +++ | |
| 99 | ++++ | ++++ | +++ | ++++ | |
| 109 | ++++ | ++++ | ++ | +++ | |
| 114 | | +++ | + | +++ | |
| 115 | | +++ | + | +++ | 1-29 |
| 118 | +++ | ++++ | + | ++++ | |
| 120 | ++++ | ++++ | + | ++++ | |
| 121 | ++++ | ++++ | + | ++++ | |
| 122 | | +++ | + | +++ | 1-29 |
| 125 | +++ | +++ | + | + | |
| 126 | + | + | + | + | |
| 148 | | ++ | + | + | |
| 150 | | ++ | + | + | |
| 153 | +++ | | + | | |
| 154 | +++ | +++ | + | + | 30-49 |
| 158 | + | + | + | + | |
| 160 | +++ | + | + | + | 1-29 |
| 161 | +++ | + | + | + | |
| 175 | + | + | + | + | |
| 196 | ++++ | ++++ | +++ | ++++ | |
| 219 | ++++ | +++ | + | + | 1-29 |
| 233 | ++++ | | | | |
| 237 | ++++ | | + | + | |
| 238 | ++++ | | + | + | |
| 243 | ++++ | | + | + | |
| 244 | ++++ | | + | + | >50 |
| 245 | ++++ | | + | + | |
| 247 | ++++ | | + | + | |
| 249 | ++++ | ++++ | + | + | >50 |
| 255 | ++++ | | + | | |
| 291 | | | + | | |
| 293 | +++ | | + | | |
| 303 | +++ | | + | | 1-29 |
| 305 | | | + | | |
| 306 | ++++ | | + | | |
| 310 | ++++ | | + | | |
| 312 | ++++ | | | | |
| 313 | ++++ | | ++ | | |
| 314 | | | + | | |
| 315 | ++++ | ++++ | ++ | ++++ | >50 |
| 316 | ++++ | ++++ | + | +++ | >50 |
| 317 | +++ | | + | ++ | |
| 321 | ++++ | | + | | |
| 324 | +++ | | + | | |
| 325 | +++ | | | | |
| 326 | +++ | | + | | |
| 327 | +++ | | + | | |
| 328 | +++ | | ++ | | |
| 329 | ++++ | | + | | |
| 330 | | | + | | |
| 331 | ++++ | ++++ | + | + | >50 |
| 338 | ++++ | ++++ | ++ | +++ | |
| 341 | +++ | ++ | + | + | |
| 343 | +++ | | + | + | |
| 346 | ++++ | | + | + | |
| 347 | +++ | | + | + | |
| 349 | ++++ | +++ | + | + | 30-49 |
| 350 | ++++ | | + | + | |
| 351 | ++++ | +++ | + | + | 30-49 |
| 353 | ++ | ++ | + | + | |
| 355 | ++++ | ++ | + | + | 1-29 |
| 357 | ++++ | ++++ | + | + | |
| 358 | ++++ | ++ | + | + | |
| 359 | ++++ | ++ | + | + | |
| 367 | ++++ | | + | + | 30-49 |
| 386 | ++++ | ++++ | ++++ | ++++ | |
| 388 | ++ | ++ | + | +++ | 1-29 |
| 390 | ++++ | ++++ | +++ | ++++ | |
| 435 | +++ | ++ | + | | |
| 436 | ++++ | ++++ | ++ | | |
| 437 | ++++ | ++++ | ++ | ++++ | 30-49 |
| 440 | ++ | ++ | + | | |
| 442 | ++++ | ++++ | ++ | | |
| 444 | ++++ | ++++ | +++ | | |
| 445 | ++++ | +++ | + | + | >50 |
| 555 | | | | | >50 |
| 557 | | | | | >50 |
| 558 | | | | | 30-49 |
| 562 | | | | | 30-49 |
| 564 | | | | | 30-49 |
| 566 | | | | | 30-49 |
| 567 | | | | | >50 |
| 572 | | | | | >50 |
| 573 | | | | | 30-49 |
| 578 | | | | | 30-49 |
| 662 | | | | | >50 |
| 379 | | | | | 1-29 |
| 375 | | | | | 1-29 |
| 559 | | | | | >50 |
| 561 | | | | | 1-29 |
| 563 | | | | | 1-29 |
| 568 | | | | | 1-29 |
| 569 | | | | | 1-29 |
| 571 | | | | | 1-29 |
| 574 | | | | | 1-29 |
| 575 | | | | | 1-29 |
| 576 | | | | | 1-29 |
| 577 | | | | | 30-49 |
| 433 | | | | | 1-29 |
| 551 | | | | | 30-49 |
| 553 | | | | | 1-29 |
| 710 | | | | + | |
| 711 | | | | + | |
| 712 | | | | ++ | |
| 713 | | | | ++ | |
| 714 | | | | +++ | |
| 715 | | | | +++ | |
| 716 | | | | + | |

### Example 14: p²¹ ELISA Assay

SJSA-1 cells were trypsinized, counted, and seeded at a density of 7500 cells/100 μL/well in 96-well plates one day prior to running the assay. On the day of the assay, the media was replaced with fresh RPMI-11% FBS assay media. 90 μL of the assay media was added to each well. The control wells contained no cells and received 100 μL of the assay media.

10 mM stock solutions of the peptides were prepared in DMSO. The stock solutions were serially diluted using a 1:3 dilution scheme to obtain 10 mM, 3.3 mM, 1.1 mM, 0.33 mM, 0.11 mM, 0.03 mM, and 0.01 mM solutions in DMSO. The solutions were serially diluted 33.3 times using sterile water to provide a range of 10× working stock solutions. A DMSO/sterile water (3% DMSO) solution was prepared for use in the control wells. The working stock solution concentration range was 300 μM, 100 μM, 30 μM, 10 μM, 3 μM, 1 μM, 0.3 μM, and 0 μM. Each solution was mixed well at each dilution step using a multichannel pipette. Row H contained the control wells. Wells H1-H3 received 10 μL of the assay media. Wells H4-H9 received 10 μL of the 3% DMSO-water solution. Wells H10-H12 received media alone and contained no cells. The MDM2 small molecule inhibitor Nutlin-3a (10 mM) was used as a positive control. Nutlin-3a was diluted using the same dilution scheme used for the peptides.

10 μL of a 10× peptide solution was added to the appropriate well to achieve a final concentration in a volume of 100 μL. For example, 10 μL of 300 μM peptide+90 μL of cells in media=30 μM final concentration in 100 μL volume in wells. The final concentration range used was 30 μM, 10 μM, 3 μM, 1 μM, 0.3 μM, and 0 μM. Control wells included wells that did not receive peptides but contained the same concentration of DMSO as the wells containing the peptides and wells containing no cells.

20 h after incubation, the media was aspirated from the wells. The cells were washed with 1×PBS (without Cα++/Mg⁺) and lysed in 60 μL of 1× cell lysis buffer (10× buffer diluted to 1× and supplemented with protease inhibitors and phosphatase inhibitors) on ice for 30 min. The plates were centrifuged at 5000 rpm at 4° C. for 8 min. The clear supernatants were collected and frozen at −80° C. until further use. The total protein contents of the lysates were measured using a BCA protein detection kit and BSA standards. Each well provided about 6-7 μg of protein. 50 μL of the lysate was used per well to set up the p21 ELISA assay. For the human total p21 ELISA assay, 50 μL of lysate was used for each well, and each well was set up in triplicate.

### Example 15: Caspase 3 Detection Assay

SJSA-1 cells were trypsinized, counted, and seeded at a density of 7500 cells/100 μL/well in 96-well plates one day prior to conducting the assay. One the day of the assay, the media was replaced with fresh RPMI-11% FBS assay media. 180 μL of the assay media was added to each well. Control wells contained no cells and received 200 μL of the assay media.

10 mM stock solutions of the peptides were prepared in DMSO. The stock solutions were serially diluted using a 1:3 dilution scheme to obtain 10 mM, 3.3 mM, 1.1 mM, 0.33 mM, 0.11 mM, 0.03 mM, and 0.01 mM solutions in DMSO. The solutions were serially diluted 33.3 times using sterile water to provide a range of 10× working stock solutions. A DMSO/sterile water (3% DMSO) solution was prepared for use in the control wells. The working stock solution concentration range was 300 μM, 100 μM, 30 μM, 10 μM, 3 μM, 1 μM, 0.3 μM, and 0 μM. Each well was mixed well at each dilution step using a multichannel pipette. 20 μL of the 10× working stock solutions were added to the appropriate wells. Row H of the plates had control wells. Wells H1-H3 received 20 μL of the assay media. Wells H4-H9 received 20 μL of the 3% DMSO-water solutions. Wells H10-H12 received media and had no cells. The MDM2 small molecule inhibitor Nutlin-3a (10 mM) was used as a positive control. Nutlin-3a was diluted using the same dilution scheme as the peptides.

10 μL of the 10× stock solutions were added to the appropriate wells to achieve the final concentrations in a total volume of 100 μL. For example, 10 μL of 300 μM peptide+90 μL of cells in media=30 μM final concentration in 100 μL volume in wells. The final concentration range used was 30 μM, 10 μM, 3 μM, 1 μM, 0.3 μM, and 0 μM. Control wells contained no peptides but contained the same concentration of DMSO as the wells containing the peptides and well containing no cells. 48 h after incubation, 80 L of the media was aspirated from each well. 100 μL of Caspase 3/7Glo assay reagent was added to each well. The plates were incubated with gentle shaking for 1 h at room temperature and read using a multi-plate reader for luminescence. Data were analyzed as Caspase 3 activation over DMSO-treated cells. Results from EXAMPLE 14 and EXAMPLE 15 are shown in TABLE 8.

TABLE 8

| | caspase | caspase | caspase | caspase | caspase | p21 | p21 | p21 | p21 | p21 |
|---|---|---|---|---|---|---|---|---|---|---|
| SP# | 0.3 μM | 1 μM | 3 μM | 10 μM | 30 μM | 0.3 μM | 1 μM | 3 μM | 10 μM | 30 μM |
| 4 | | | 9 | 37 | 35 | | | 317 | 3049 | 3257 |
| 7 | 0.93 | 1.4 | 5.08 | 21.7 | 23.96 | | 18 | 368 | 1687 | 2306 |
| 8 | | | 1 | 19 | 25 | | | 34 | 972 | 2857 |
| 10 | 1 | | 1 | 17 | 32 | | 10 | 89 | 970 | 2250 |
| 11 | 1 | | 5 | 23 | 33.5 | | 140 | 350 | 2075.5 | 3154 |
| 26 | 1 | | 1 | 3 | 14 | | | | | |
| 50 | | | 8 | 29 | 29 | | 44 | 646 | 1923 | 1818 |
| 65 | 1 | | 6 | 28 | 34 | −69 | −24 | 122 | 843 | 1472 |
| 69 | 4.34 | 9.51 | 16.39 | 26.59 | 26.11 | 272 | 458.72 | 1281.39 | 2138.88 | 1447.22 |
| 70 | | | 1 | 9 | 26 | | −19 | 68 | 828 | 1871 |
| 71 | 0.95 | 1.02 | 3.68 | 14.72 | 23.52 | | 95 | 101 | 1204 | 2075 |
| 72 | 1 | | 1 | 4 | 10 | −19 | 57 | 282 | 772 | 1045 |
| 77 | 1 | | 2 | 19 | 23 | | | | | |
| 80 | 1 | | 2 | 13 | 20 | | | | | |
| 81 | 1 | | 1 | 6 | 21 | | 0 | 0 | 417 | 1649 |
| 99 | 1 | | 7 | 31 | 33 | −19 | 117 | 370 | 996 | 1398 |
| 109 | | | 4 | 16 | 25 | | 161 | 445 | 1221 | 1680 |
| 114 | 1 | | 6 | 28 | 34 | −21 | 11 | 116 | 742 | 910 |
| 115 | 1 | | 10 | 26 | 32 | −10 | 36 | 315 | 832 | 1020 |
| 118 | 1 | | 2 | 18 | 27 | −76 | −62 | −11 | 581 | 1270 |
| 120 | 2 | | 11 | 20 | 30 | −4 | 30 | 164 | 756 | 1349 |
| 121 | 1 | | 5 | 19 | 30 | 9 | 33 | 81 | 626 | 1251 |
| 122 | 1 | | 2 | 15 | 30 | −39 | −18 | 59 | 554 | 1289 |
| 123 | 1 | | 1 | 6 | 14 | | | | | |
| 125 | 1 | | 3 | 9 | 29 | 50 | 104 | 196 | 353 | 1222 |
| 126 | 1 | | 1 | 6 | 30 | −47 | −10 | 90 | 397 | 1443 |
| 127 | 1 | | 1 | 4 | 13 | | | | | |
| 130 | 1 | | 2 | 6 | 17 | | | | | |
| 139 | 1 | | 2 | 9 | 18 | | | | | |
| 142 | 1 | | 2 | 15 | 20 | | | | | |
| 144 | 1 | | 4 | 10 | 16 | | | | | |
| 148 | 1 | | 11 | 23 | 31 | −23 | 55 | 295 | 666 | 820 |
| 149 | 1 | | 2 | 4 | 10 | 35 | 331 | 601 | 1164 | 1540 |
| 150 | 2 | | 11 | 19 | 35 | −37 | 24 | 294 | 895 | 906 |
| 153 | 2 | | 10 | 15 | 20 | | | | | |
| 154 | 2.68 | 4 | 13.93 | 19.86 | 30.14 | 414.04 | 837.45 | 1622.42 | 2149.51 | 2156.98 |
| 158 | 1 | | 1.67 | 5 | 16.33 | −1.5 | 95 | 209.5 | 654 | 1665.5 |
| 160 | 2 | | 10 | 16 | 31 | −43 | 46 | 373 | 814 | 1334 |
| 161 | 2 | | 8 | 14 | 22 | 13 | 128 | 331 | 619 | 1078 |
| 170 | 1 | | 1 | 16 | 20 | | | | | |
| 175 | 1 | | 5 | 12 | 21 | −65 | 1 | 149 | 543 | 1107 |
| 177 | 1 | | 1 | 8 | 20 | | | | | |
| 183 | 1 | | 1 | 4 | 8 | −132 | −119 | −14 | 1002 | 818 |
| 196 | 1 | | 4 | 33 | 26 | −49 | −1 | 214 | 1715 | 687 |
| 197 | 1 | | 1 | 10 | 20 | | | | | |
| 203 | 1 | | 3 | 12 | 10 | 77 | 329 | 534 | 1805 | 380 |
| 204 | 1 | | 4 | 10 | 10 | 3 | 337 | 928 | 1435 | 269 |
| 218 | 1 | | 2 | 8 | 18 | | | | | |
| 219 | 1 | | 5 | 17 | 34 | 28 | 53 | 289 | 884 | 1435 |
| 221 | 1 | | 3 | 6 | 12 | 127 | 339 | 923 | 1694 | 1701 |
| 223 | 1 | | 1 | 5 | 18 | | | | | |
| 230 | 1 | | 2 | 3 | 11 | 245.5 | 392 | 882 | 1549 | 2086 |
| 233 | 6 | 8 | 17 | 22 | 23 | 2000 | 2489 | 3528 | 3689 | 2481 |
| 237 | 1 | | 5 | 9 | 15 | 0 | 0 | 2 | 284 | 421 |
| 238 | 1 | | 2 | 4 | 21 | 0 | 149 | 128 | 825 | 2066 |
| 242 | 1 | | 4 | 5 | 18 | 0 | 0 | 35 | 577 | 595 |
| 243 | 1 | | 2 | 5 | 23 | 0 | 0 | 0 | 456 | 615 |
| 244 | 1 | | 2 | 7 | 17 | 0 | 178 | 190 | 708 | 1112 |
| 245 | 1 | | 3 | 9 | 16 | 0 | 0 | 0 | 368 | 536 |
| 247 | 1 | | 3 | 11 | 24 | 0 | 0 | 49 | 492 | 699 |
| 248 | | | | | | 0 | 50 | 22 | 174 | 1919 |
| 249 | 2 | | 5 | 11 | 23 | 0 | 0 | 100 | 907 | 1076 |
| 251 | | | | | | 0 | 0 | 0 | 0 | 0 |
| 252 | | | | | | 0 | 0 | 0 | 0 | 0 |
| 253 | | | | | | 0 | 0 | 0 | 0 | 0 |
| 254 | 1 | 3 | 7 | 14 | 22 | 118 | 896 | 1774 | 3042 | 3035 |
| 286 | 1 | 4 | 11 | 20 | 22 | 481 | 1351 | 2882 | 3383 | 2479 |
| 287 | 1 | 1 | 3 | 11 | 23 | 97 | 398 | 986 | 2828 | 3410 |
| 315 | 11 | 14.5 | 25.5 | 32 | 34 | 2110 | 2209 | 2626 | 2965 | 2635 |
| 316 | 6.5 | 10.5 | 21 | 32 | 32.5 | 1319 | 1718 | 2848 | 2918 | 2540 |
| 317 | 3 | 4 | 9 | 26 | 35 | 551 | 624 | 776 | 1367 | 1076 |
| 331 | 4.5 | 8 | 11 | 14.5 | 30.5 | 1510 | 1649 | 2027 | 2319 | 2509 |
| 338 | 1 | 5 | 23 | 20 | 29 | 660.37 | 1625.38 | 3365.87 | 2897.62 | 2727 |
| 341 | 3 | 8 | 11 | 14 | 21 | 1325.62 | 1873 | 2039.75 | 2360.75 | 2574 |
| 343 | 1 | 1 | 2 | 5 | 29 | 262 | 281 | 450 | 570 | 1199 |
| 346 | | | | | | 235.86 | 339.82 | 620.36 | 829.32 | 1695.78 |
| 347 | 2 | 3 | 5 | 8 | 29 | 374 | 622 | 659 | 905 | 1567 |
| 349 | 1 | 8 | 11 | 16 | 24 | 1039.5 | 1598.88 | 1983.75 | 2191.25 | 2576.38 |
| 351 | 3 | 9 | 13 | 15 | 24 | 1350.67 | 1710.67 | 2030.92 | 2190.67 | 2668.54 |
| 353 | 1 | 2 | 5 | 7 | 30 | 390 | 490 | 709 | 931 | 1483 |
| 355 | 1 | 4 | 11 | 13 | 30 | 191 | 688 | 1122 | 1223 | 1519 |
| 357 | 2 | 7 | 11 | 15 | 23 | 539 | 777 | 1080 | 1362 | 1177 |
| 358 | 1 | 2 | 3 | 6 | 24 | 252 | 321 | 434 | 609 | 1192 |
| 359 | 3 | 9 | 11 | 13 | 23 | 1163.29 | 1508.79 | 1780.29 | 2067.67 | 2479.29 |
| 416 | | | | | | 33.74 | 39.82 | 56.57 | 86.78 | 1275.28 |
| 417 | | | | | | 0 | 0 | 101.13 | 639.04 | 2016.58 |
| 419 | | | | | | 58.28 | 97.36 | 221.65 | 1520.69 | 2187.94 |
| 432 | | | | | | 54.86 | 68.86 | 105.11 | 440.28 | 1594.4 |

### Example 16: Cell Lysis by Peptidomimetic Macrocycles

SJSA-1 cells were plated out one day in advance in clear, flat-bottom plates at a density of 7500 cells/well with 100 μL/well of growth media. Row H columns 10-12 were left empty to be treated with media alone. On the day of the assay, the media was exchanged with RPMI 1% FBS media to result in 90 μL of media per well. 10 mM stock solutions of the peptidomimetic macrocycles were prepared in 100% DMSO. The peptidomimetic macrocycles were diluted serially in 100% DMSO, and further diluted 20-fold in sterile water to prepare working stock solutions in 5% DMSO/water. The concentrations of the peptidomimetic macrocycles ranged from 500 μM to 62. μM.

10 μL of each compound solution was added to the 90 μL of SJSA-1 cells to yield final concentration of 50 μM to 6.25 μM in 0.5% DMSO-containing media. The negative control (non-lytic sample) was 0.5% of DMSO alone. The positive control (lytic) samples included 10 μM of Melittin and 1% Triton X-100. The cell plates were incubated for 1 h at 37° C. After incubation for 1 h, the morphology of the cells was examined by microscope. The plates were then centrifuged at 1200 rpm for 5 min at room temperature. 40 μL of the supernatant for each peptidomimetic macrocycle and control sample was transferred to clear assay plates. LDH release was measured using an LDH cytotoxicity assay kit. The results of the cell lysis assay are shown in TABLE 9:

TABLE 9

| | 6.25 μM % | 12.5 μM % | 25 μM % | 50 μM % |
|---|---|---|---|---|
| | Lysed cells | Lysed cells | Lysed cells | Lysed cells |
| SP# | (1 h LDH) | (1 h LDH) | (1 h LDH) | (1 h LDH) |
| 3 | 1 | 0 | 1 | 3 |
| 4 | −2 | 1 | 1 | 2 |
| 6 | 1 | 1 | 1 | 1 |
| 7 | 0 | 0 | 0 | 0 |
| 8 | −1 | 0 | 1 | 1 |
| 9 | −3 | 0 | 0 | 2 |
| 11 | −2 | 1 | 2 | 3 |
| 15 | 1 | 2 | 2 | 5 |
| 18 | 0 | 1 | 2 | 4 |
| 19 | 2 | 2 | 3 | 21 |
| 22 | 0 | −1 | 0 | 0 |
| 26 | 2 | 5 | −1 | 0 |
| 32 | 0 | 0 | 2 | 0 |
| 39 | 0 | −1 | 0 | 3 |
| 43 | 0 | 0 | −1 | −1 |
| 55 | 1 | 5 | 9 | 13 |
| 65 | 0 | 0 | 0 | 2 |
| 69 | 1 | 0.5 | −0.5 | 5 |
| 71 | 0 | 0 | 0 | 0 |
| 72 | 2 | 1 | 0 | 3 |
| 75 | −1 | 3 | 1 | 1 |
| 77 | −2 | −2 | 1 | −1 |
| 80 | 0 | 1 | 1 | 5 |
| 81 | 1 | 1 | 0 | 0 |
| 82 | 0 | 0 | 0 | 1 |
| 99 | 1.5 | 3 | 2 | 3.5 |
| 108 | 0 | 0 | 0 | 1 |
| 114 | 3 | −1 | 4 | 9 |
| 115 | 0 | 1 | −1 | 6 |
| 118 | 4 | 2 | 2 | 4 |
| 120 | 0 | −1 | 0 | 6 |
| 121 | 1 | 0 | 1 | 7 |
| 122 | 1 | 3 | 0 | 6 |
| 123 | −2 | 2 | 5 | 3 |
| 125 | 0 | 1 | 0 | 2 |
| 126 | 1 | 2 | 1 | 1 |
| 130 | 1 | 3 | 0 | −1 |
| 139 | −2 | −3 | −1 | −1 |
| 142 | 1 | 0 | 1 | 3 |
| 144 | 1 | 2 | −1 | 2 |
| 147 | 8 | 9 | 16 | 55 |
| 148 | 0 | 1 | −1 | 0 |
| 149 | 6 | 7 | 7 | 21 |
| 150 | −1 | −2 | 0 | 2 |
| 153 | 4 | 3 | 2 | 3 |
| 154 | −1 | −1.5 | −1 | −1 |
| 158 | 0 | −6 | −2 | |
| 160 | −1 | 0 | −1 | 1 |
| 161 | 1 | 1 | −1 | 0 |
| 169 | 2 | 3 | 3 | 7 |
| 170 | 2 | 2 | 1 | −1 |
| 174 | 5 | 3 | 2 | 5 |
| 175 | 3 | 2 | 1 | 0 |
| 177 | −1 | −1 | 0 | 1 |
| 182 | 0 | 2 | 3 | 6 |
| 183 | 2 | 1 | 0 | 3 |
| 190 | −1 | −1 | 0 | 1 |
| 196 | 0 | −2 | 0 | 3 |
| 197 | 1 | −4 | −1 | −2 |
| 203 | 0 | −1 | 2 | 2 |
| 204 | 4 | 3 | 2 | 0 |
| 211 | 5 | 4 | 3 | 1 |
| 217 | 2 | 1 | 1 | 2 |
| 218 | 0 | −3 | −4 | 1 |
| 219 | 0 | 0 | −1 | 2 |
| 221 | 3 | 3 | 3 | 11 |
| 223 | −2 | −2 | −4 | −1 |
| 230 | 0.5 | −0.5 | 0 | 3 |
| 232 | 6 | 6 | 5 | 5 |
| 233 | 2.5 | 4.5 | 3.5 | 6 |
| 237 | 0 | 3 | 7 | 55 |
| 243 | 4 | 23 | 39 | 64 |
| 244 | 0 | 1 | 0 | 4 |
| 245 | 1 | 14 | 11 | 56 |
| 247 | 0 | 0 | 0 | 4 |
| 249 | 0 | 0 | 0 | 0 |
| 254 | 11 | 34 | 60 | 75 |
| 279 | 6 | 4 | 5 | 6 |
| 280 | 5 | 4 | 6 | 18 |
| 284 | 5 | 4 | 5 | 6 |
| 286 | 0 | 0 | 0 | 0 |
| 287 | 0 | 6 | 11 | 56 |
| 316 | 0 | 1 | 0 | 1 |
| 317 | 0 | 1 | 0 | 0 |
| 331 | 0 | 0 | 0 | 0 |
| 335 | 0 | 0 | 0 | 1 |
| 336 | 0 | 0 | 0 | 0 |
| 338 | 0 | 0 | 0 | 1 |
| 340 | 0 | 2 | 0 | 0 |
| 341 | 0 | 0 | 0 | 0 |
| 343 | 0 | 1 | 0 | 0 |
| 347 | 0 | 0 | 0 | 0 |
| 349 | 0 | 0 | 0 | 0 |
| 351 | 0 | 0 | 0 | 0 |
| 353 | 0 | 0 | 0 | 0 |
| 355 | 0 | 0 | 0 | 0 |
| 357 | 0 | 0 | 0 | 0 |
| 359 | 0 | 0 | 0 | 0 |
| 413 | 5 | 3 | 3 | 3 |
| 414 | 3 | 3 | 2 | 2 |
| 415 | 4 | 4 | 2 | 2 |

### Example 17: Mechanistic Rationale for Peptidomimetic Macrocycles as Myelopreservation Agents

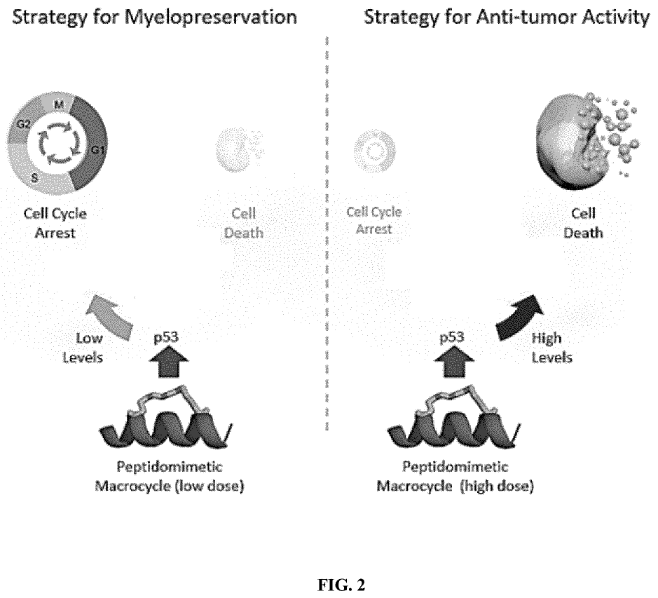

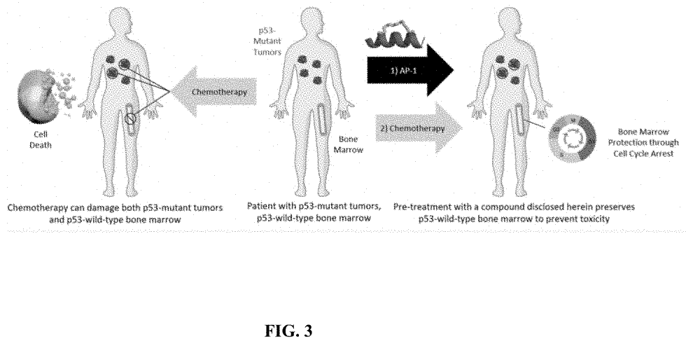

The cytotoxicity of some chemotherapeutic agents (e.g. topoisomerase inhibitors) is reduced in cells that are not actively dividing. In cells with wild-type p53, p53 activation via administration of a peptidomimetic macrocycle can induce transient, dose-dependent cell cycle arrest, as shown in FIG. 2. Induction of cell cycle arrest can reduce sensitivity to chemotherapy induced cytotoxicity. The cell cycle of p53 mutant cancer cells is unaffected by the peptidomimetic macrocycle leaving the cells vulnerable to the cytotoxic effects of the chemotherapy, as shown in FIG. 3.

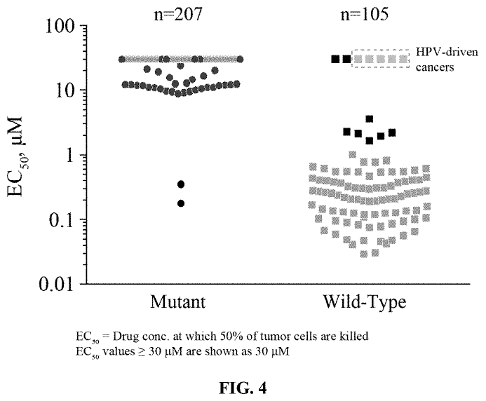

FIG. 4, presents data that shows the effect of AP-1 in p53 wild type cells vs. p53 mutant cells. Various cell lines were grown in RPMI1640, 10% FBS, 2 mM L-alanyl-L-Glutamine, 1 mM Na pyruvate or a special medium in a humidified atmosphere of 5% CO2 at 37° C. Cells were seeded in 384 well plates and AP-1 was added to cells 24 hours post seeding. AP-1 was serially diluted 3.16-fold and assayed over 10 concentrations. 16-bit TIFF images were collected with 4× objective. EC50 values were calculated using non-linear regression to fit data to a sigmoidal 4-point, 4 parameter one-site dose response model, where: y (fit)=A+[(B−A)/(1+((C/x)\hat{ }D))]. As can be seen in FIG. 4, the EC50 of AP-1 was increased in p53 mutant cell lines compared to cell lines with wild-type p53.

### Example 18: Effect of AP-1 on Cell Death and Cell Cycle Arrest in p53-WT MOLM13 Cells

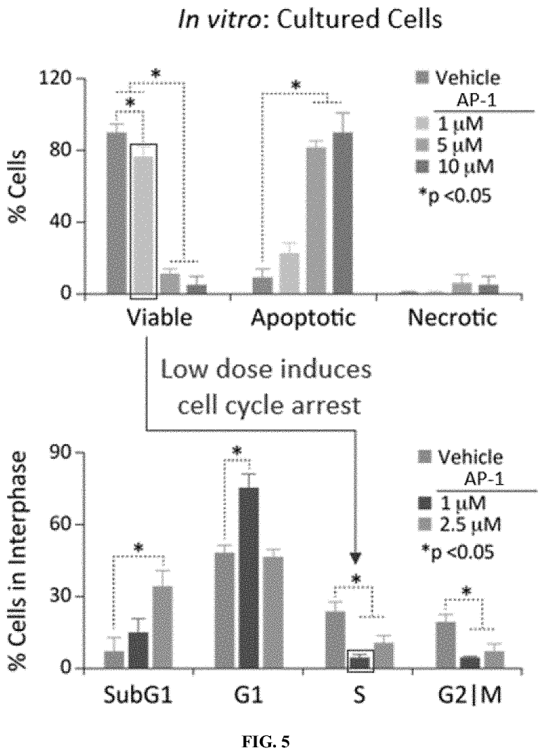

MOLM13 cells were cultured in vitro and treated with a vehicle control or varying concentrations of AP-1 for 24 hours. After the treatment period, the effects of AP-1 on cell cycle arrest and apoptosis were analyzed by propidium iodide and annexin V staining, respectively. As can be seen in FIG. 5, AP-1 triggered dose dependent apoptosis. When cells were treated with 1 μM AP-1, cell cycle arrest was induced in cells.

### Example 19: Induction of Cell Cycle Arrest by AP-1 in CD34⁺ Bone Marrow Cells

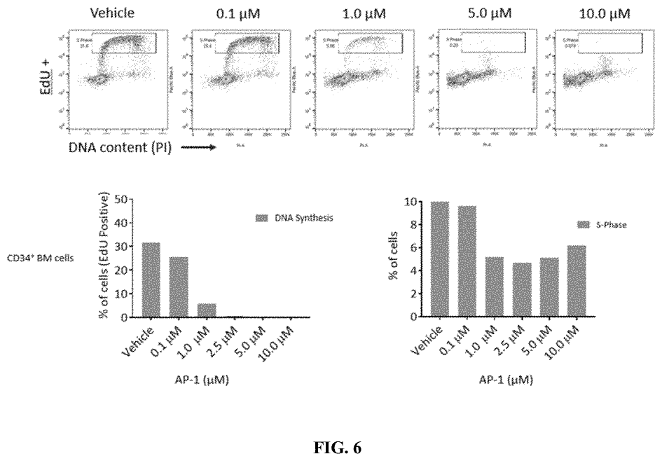

CD34⁺ bone marrow cells were cultured and treated with various concentrations of AP-1. DNA synthesis and the percentage of cells in S-phase was detected via EdU staining of cells as measured via flow cytometry. Results are shown in FIG. 6 and indicate that treatment with AP-1 decreased DNA synthesis and the percentage of cells in S-phase compared to vehicle controls.

### Example 20: Reversibility of AP-1 Induced Cell Cycle Arrest in Bone Marrow CD34+ Cells

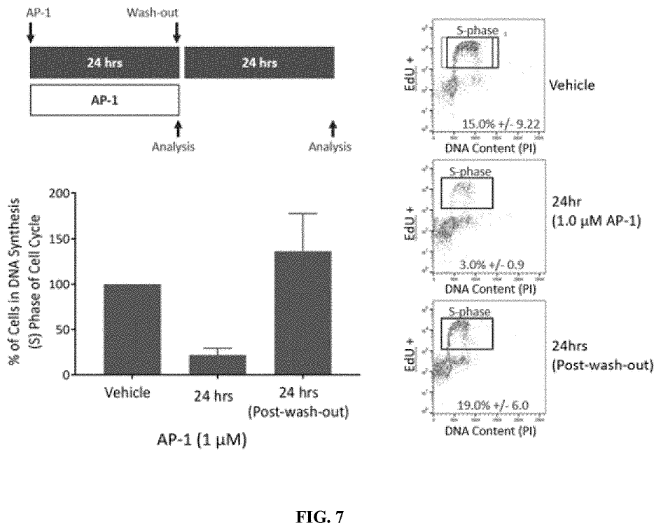

Two groups of bone marrow CD34+ cells were incubated with 1 μM AP-1 or vehicle control in vitro for 24 hours. Cell cycle arrest was assessed via EdU staining and flow cytometry immediately following AP-1 incubation for one group of bone marrow CD34+ cells (Group 1) and 24 hours after washing out AP-1 in a second group (Group 2). As can be seen in FIG. 7, a decrease in the percentage of cells in S-phase compared to vehicle control for cells in Group 1 was observed, but not for cells in Group 2 as measured via EdU staining and flow cytometry. These results indicate that AP-1 induces transient, reversible cell cycle arrest in human bone marrow cells.

### Example 21: Protection Against Topotecan-Induced DNA Damage

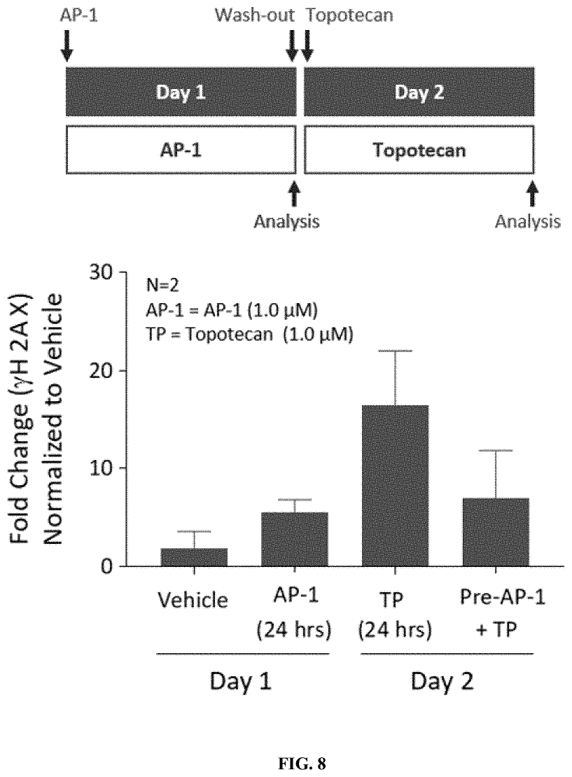

Bone marrow CD34+ cells were treated with 1 μM AP-1 or vehicle control in vitro for 24 hours. Following treatment with AP-1 or vehicle, cells were washed and treated with 1 μM topotecan for 24 hours. DNA damage in topotecan incubated cells was then assessed via measurement of γ-H2AX. As can be seen in FIG. 8, pre-treatment with AP-1 reduced topotecan induced DNA damaged compared to cells pre-treated with vehicle control as indicated by a decrease in γ-H2AX.

### Example 22: Induction p21 Expression In Vivo

mRNA was extracted from total bone marrow samples from mice treated with either 2.4 mg/kg or 10 mg/kg AP-1 at 0, 4, 8, 16, and 24 hours post drug administration. Murine p21 (a downstream mediator of p53 dependent cell cycle arrest), Noxa (an apoptosis marker), and p53 upregulated modulator of apoptosis (PUMA) mRNA expression was then assessed by real time PCR. As can be seen in FIG. 9, results showed an increase in p21, Noxa and PUMA mRNA expression following AP-1 treatment. At the 2.4 mg/kg dose, the increase in p21 expression was greater than the increase in PUMA and Noxa expression, while at the 10 mg/kg dose the increase in p21 expression was greater than the increase in PUMA expression, but not greater than the increase in Noxa expression.

### Example 23: Induction of Cell Cycle Arrest in Bone Marrow In Vivo

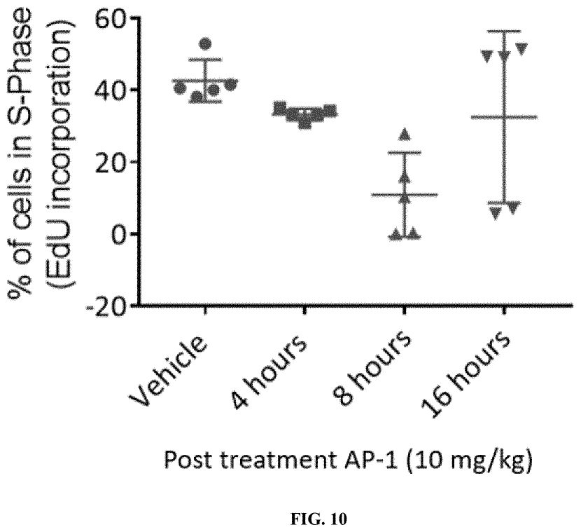

C57BL/6 mice were treated with 10 mg/kg AP-1. Bone marrow from EdU treated animal was collected at defined time points (4, 8, 16 hours) post administration of AP-1, followed by flow cytometric analysis of DNA synthesis in hematopoietic stem and progenitor cells (HSPC). As can be seen in FIG. 10, inhibition of DNA synthesis was observed in HSPCs within 4 hours post administration of AP-1 and reaching a nadir at 8 hours before returning to baseline.

Additional mice were treated with 5 mg/kg, 10 mg/kg, or 20 mg/kg AP-1. Cell cycle arrest in the bone marrow of mice was then measured by flow cytometry using EdU incorporation in lineage negative, Kit positive, hematopoietic stem and progenitor cells at pre-treatment (0 hours post treatment), and 4 hours, 8 hours, 16 hours, and 24 hours post treatment. As can be seen in FIG. 11, a decrease in the percentage of EdU+ cells from 0-8 hours post treatment was observed. The decrease reversed from 8-24 hours post treatment. These results indicate that AP-1 reversibly induced cell cycle arrest in mouse bone marrow cells in vivo.

### Example 24: MIC-1 Levels Following AP-1 Treatment

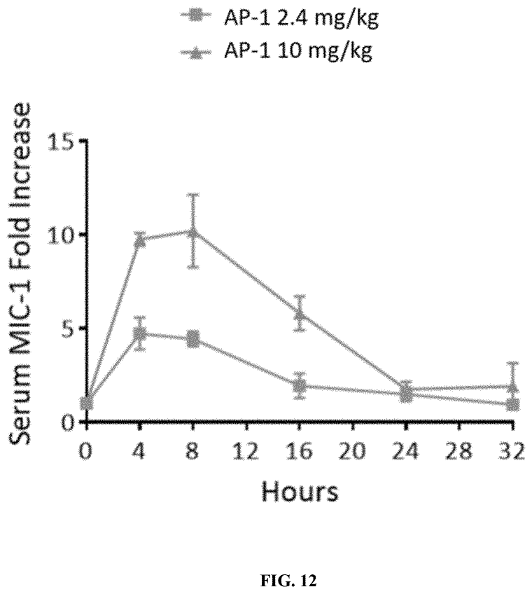

FIG. 12 shows the levels of MIC-1, a clinical biomarker of p53 activation, in the serum of mice before (time 0) or after treatment with AP-1 at a dose of 2.4 mg/kg or 10 mg/kg. Results show that murine MIC-1 levels, as measured by ELISA, peak between 4 and 8 hours post treatment with AP-1, correlating with cell cycle inhibition in vivo, as shown by FIG. 11.

### Example 25: Induction of Cell Cycle Arrest in MCF-Tumors

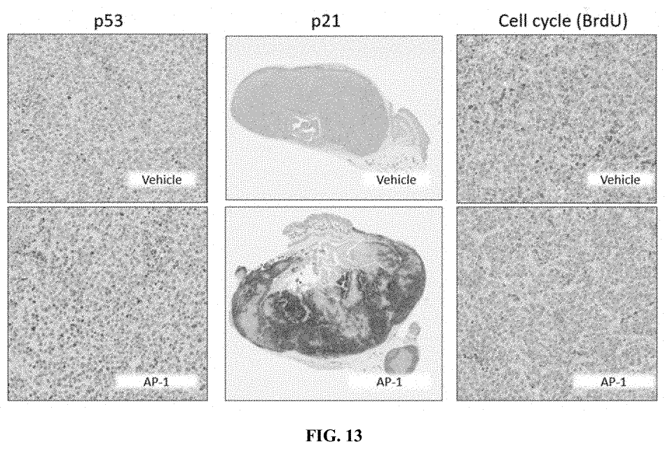

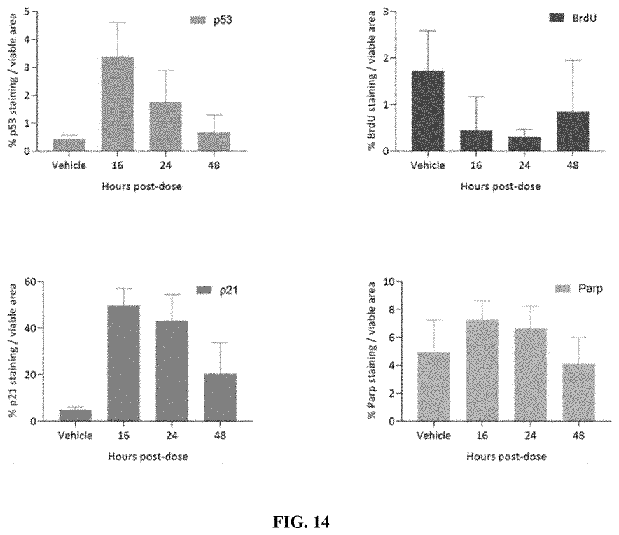

Mice bearing MCF-7 tumors were administered a single 20 mg/kg dose of AP-1 intravenously. Tumor samples were collected 16 hours post dose and stained for p⁵³, p²¹, PARP, and bromodeoxyuridine (BrdU), which is indicative of cell proliferation. As can be seen in FIG. 13, AP-1 treatment increased p53 and p21 expression while also causing cell cycle arrest as indicated by a decrease in BrdU staining. Furthermore, an increase in PARP staining was seen with AP-1 treatment. Quantification of p53, p21, PARP, and BrdU staining at 16, 24, and 48 hours post dose is shown in FIG. 14.

### Example 26: Combination Treatment of a MCF-7.1 Mouse Model with AP-1 and Abraxane®

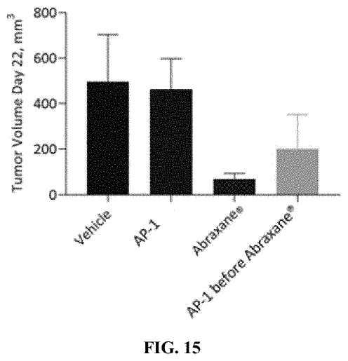

Female athymic nude mice bearing established, subcutaneous MCF-7.1 tumors were treated intravenously with 5 mg/kg AP-1, 15 mg/kg Abraxane®, 5 mg/kg AP-1 and 15 mg/kg Abraxane®, or vehicle control. Abraxane® was dosed once weekly (qwk), while vehicle and AP-1 was dosed twice weekly. In the combination treatment group, AP-1 was administered 24 hours prior to administration of Abraxane®. As can be seen in FIG. 15, the MCF-7.1 tumor volume decreased with Abraxane® or Abraxane® AP-1 combination treatment.

### Example 27: Protection Against Topotecan-Induced Neutrophil Depletion

Female C57BL/6 mice were treated with 1.5 mg/kg topotecan on days 1-5 of a treatment period. Twenty-four hours prior to first topotecan dose, mice were treated with 2.4 mg/kg AP-1 or vehicle control (n=5 mice per group). Mice were then treated with 2.4 mg/kg AP-1 or vehicle control 30 minutes before each subsequent dose of topotecan. As can be seen in FIG. 16A, mice that were pretreated with AP-1 had a higher neutrophil count compared to mice pretreated with vehicle controls. Similar results were achieved when the experiment was repeated with n=7 mice per group as shown in FIG. 16B. The result indicates a protective effect of AP-1 against topotecan induced neutrophil depletion.

In some embodiments, a preclinical or clinical conclusion can be drawn based upon analysis neutrophil levels of two treatment groups. For example, a preclinical conclusion can be based upon analysis of neutrophil levels in Group A and Group B as illustrated in FIG. 16C or FIG. 16D, where Group A consists of mice treated with 1.5 mg/kg topotecan on days 1-5 of a treatment period, and Group B consists of mice treated with topotecan on days 1-5 of a treatment period and AP-1 24 hours before the first topotecan dose and 30 minutes before each subsequent topotecan dose.

### Example 28: Effect of AP-1 on Anti-Tumor Activity of Topotecan in p53-Mutant Mouse Cancer Models

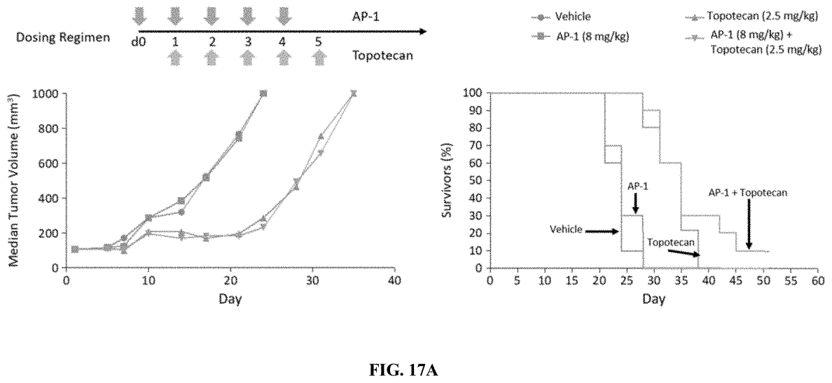

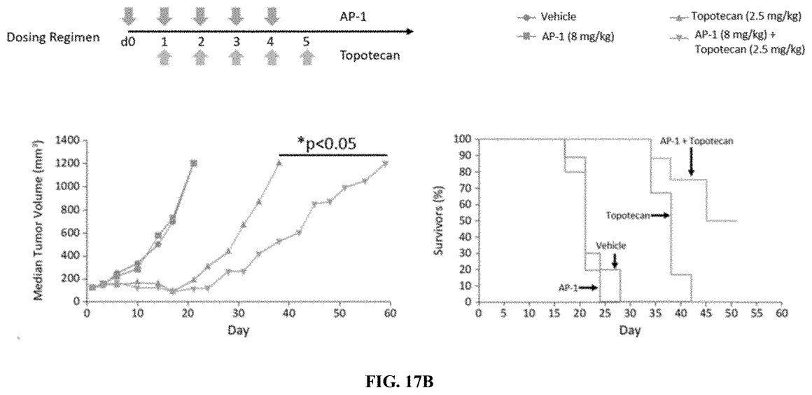

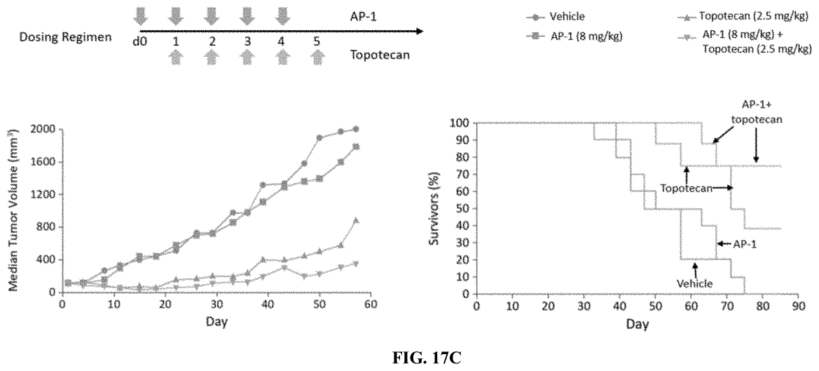

C57BL/6 mice bearing established syngeneic MC38 colon cancer tumors, or nu/nu mice bearing established H69 or H211 xenograft tumors were treated with topotecan on days 1-5 and either AP-1 or vehicle on days 0-4. Median tumor volume and mouse survival was assessed. As can be seen in FIG. 17A, FIG. 17B, and FIG. 17C, topotecan treatment led to decreased tumor volumes and increased survival. The anti-tumor activity of topotecan was not diminished by pretreatment with AP-1. In the MC38 and H69 models, pretreatment with AP-1 yielded a modest enhancement of mouse survival.

### Example 29: Protection Against Carboplatin/Paclitaxel-Induced Neutrophil Depletion

Mice were divided into six treatment groups and administered vehicle control, AP-1 alone, a combination of carboplatin and paclitaxel, or a combination of AP-1, carboplatin, and paclitaxel. The administration time(s) of AP-1 in relation to carboplatin and paclitaxel varied, with the time of carboplatin/paclitaxel administration being denoted as time 0 hours. Positive times (e.g. time+8 hours) indicate AP-1 treatments that occurred after treatment with carboplatin/paclitaxel and negative times (e.g., −1 hour) indicate AP-1 treatment before carboplatin/paclitaxel administration. AP-1 and paclitaxel were administered intravenously while carboplatin was administered via intraperitoneal injection.

Group 1 was treated with a vehicle control. Group 2 was treated with AP-1 (2.4 mg/kg) at times −8 hours, −1 hour and +8 hours (AP-1 @ −8 hr, −1 hr, +8 hr). Group 3 was treated with carboplatin (25 mg/kg) and paclitaxel (5 mg/kg) at time 0 hour (C+P). Group 4 was treated with AP-1 at times −24 hours and −1 hour and carboplatin (25 mg/kg) and paclitaxel (5 mg/kg) at time 0 hour (C+P+AP-1 @ −24 hr,−1 hr). Group 5 was treated with AP-1 at times −8 hours, −1 hours and +8 hours and carboplatin (25 mg/kg) and paclitaxel (5 mg/kg) at time 0 hour (C+P+AP-1 @ −8 hr,−1 hr, +8 hr). Group 6 was treated with AP-1 at times −8 hours and −1 hour and carboplatin (25 mg/kg) and paclitaxel (5 mg/kg) at time 0 hour (C+P+AP-1 @ −8 hr,−1 hr). Following treatment, blood was collected from mice and neutrophil levels in blood were determined. Results of neutrophil levels in mice from each treatment group 4 days after carboplatin and paclitaxel treatment are shown in FIG. 18A.

In some embodiments, a preclinical or clinical conclusion can be drawn based upon analysis of neutrophil levels in two treatment groups. For example, a preclinical conclusion can be based upon analysis of neutrophil levels in Group A and Group B as illustrated in FIG. 18B, where group A consists of mice treated with carboplatin (25 mg/kg) and paclitaxel (5 mg/kg) at time 0 hour, and Group B consists of mice treated with AP-1 at times −8 hours, −1 hours and +8 hours and carboplatin (25 mg/kg) and paclitaxel (5 mg/kg) at time 0 hour.

### Example 30: Effect of a Peptidomimetic Macrocycle on Docetaxel-Induced Neutrophil Depletion

Mice were divided into four treatment groups and administered vehicle control, AP-1 alone, docetaxel, or a combination of AP-1, and docetaxel. The administration time(s) of AP-1 in relation to docetaxel varied, with the time of docetaxel administration being denoted as time 0 hours. Positive times (e.g. time+8 hours) indicate AP-1 treatments that occurred after treatment with docetaxel and negative times (e.g., −1 hour) indicate AP-1 treatment before docetaxel administration. AP-1 and docetaxel were administered intravenously.

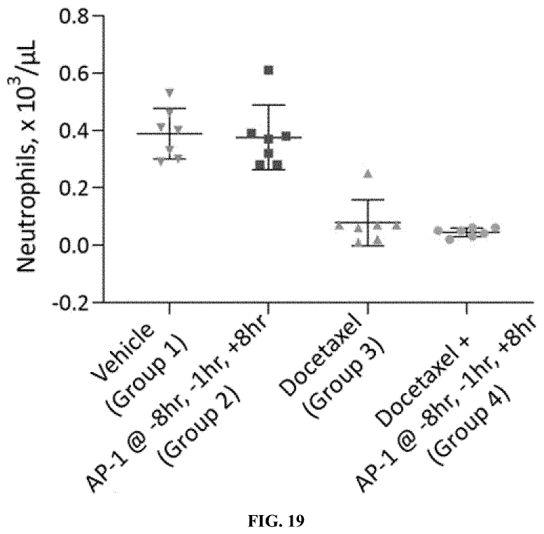

Group 1 was treated with a vehicle control. Group 2 was treated with AP-1 (2.4 mg/kg) at times −8 hours, −1 hour and +8 hours (AP-1 @ −8 hr,−1 hr, +8 hr). Group 3 was treated with docetaxel (10 mg/kg) at time 0 hour. Group 4 was treated with AP-1 at times −8 hours, −1 hour, and +8 hours and docetaxel (10 mg/kg) at time 0 hour (Docetaxel AP-1 @-h8r,−1 hr, +8 hr). Following treatment, blood was collected from mice and neutrophil levels in blood were determined. Results of neutrophil levels in mice from each treatment group 4 days after docetaxel treatment are shown in FIG. 19.

### Example 31: A Phase 1b/2 Trial of AP-1 for the Prevention of Chemotherapy-Induced Myelosuppression

### Phase 1b Study Objectives.

The primary objectives of the trial are:
To evaluate the safety and tolerability and determine the recommended Phase 2 dose (RP2D) of AP-1 when administered to patients with TP53-mutated extensive disease (ED) small cell lung cancer (SCLC) receiving topotecan as 2nd-line treatment.

The secondary objectives of the trial are:
To evaluate the preliminary myelopreservation effects of AP-1 when administered to patients with TP53-mutated ED SCLC undergoing 2nd-line treatment with topotecan.
To evaluate the efficacy of topotecan when administered as 2nd-line treatment for patients with TP53-mutated ED SCLC who are receiving supportive treatment with AP-1.
To evaluate the pharmacokinetic (PK) profile of AP-1 when administered with topotecan

An exploratory objective of the study is to assess pharmacodynamic (PD) biomarkers in blood and assess correlation with clinical response.

### Phase 1b Study Endpoints:

The primary phase 1b study endpoints include the proportion of patients with National Cancer Institute (NCI) Common Terminology Criteria for Adverse Events (CTCAE) Grade 3/4 treatment emergent adverse events (TEAEs). Secondary endpoints include:
The proportion of planned treatment cycles delayed due to toxicity.
Overall response rate (ORR) by Response Evaluation Criteria in Solid Tumors (RECIST) 1.1.
Progression free survival (PFS).
Overall survival (OS).
PK parameters (e.g., area-under-the-curve [AUC], maximum concentration [Cₘₐₓ], time of Cₘₐₓ [tₘₐₓ], half-life [t_{1/2}]) of AP-1.

Exploratory endpoints include:
Dose response curves of macrophage inhibitory cytokine-1 (MIC-1) levels in blood vs. myelopreservation parameters.
p21 RNA levels and other downstream indicators of p53-induced cellular arrest in peripheral blood mononuclear cells (PBMC).

### Phase 2 Study Objectives.

The primary phase 2 study objective is to evaluate the myelopreservation effects of AP-1 when administered at the RP2D to patients with TP53-mutated ED SCLC undergoing 2nd line treatment with topotecan.

Secondary phase 2 study objectives are to
To further evaluate the safety and tolerability of AP-1 as supportive care during treatment with topotecan.
To further evaluate the efficacy of topotecan when administered as 2nd-line treatment for patients with TP53-mutated ED SCLC who are receiving supportive treatment with AP-1.
To further evaluate the PK profile of AP-1 when administered with topotecan.

An exploratory phase 2 study objective is to assess PD biomarkers and assess correlation of PD biomarkers with clinical response.

### Phase 2 Study Endpoints.

The primary endpoint of the phase 2 study is the proportion of patients with Grade ≥3 neutropenia in Cycle 1. The secondary endpoints of the Phase 2 study are:
The proportion of patients with NCI CTCAE Grade 3/4 TEAEs
The proportion of planned treatment cycles delayed due to toxicity
ORR
PFS
OS
PK parameters (e.g., AUC, Cₘₐₓ, tₘₐₓ, t_{1/2}) of AP-1

Exploratory endpoints of the phase 2 study are:
Dose response curves of MIC-1 levels in blood vs. myelopreservation parameters.
p²¹ RNA levels and other downstream indicators of p53-induced cellular arrest in PBMCs.

### Study Design/Description:

Small Cell Lung Cancer:

During the Phase 1b dose optimization stage of the study, AP-1 and topotecan are administered as part of one or more treatment cycles. Treatment cycles are denoted to begin on Day 0 and end on Day 21. Topotecan is administered per standard practice on Days 1-5 of treatment cycles. Patients are randomized to receive 1 of 2 initial AP-1 dose levels, administered on Days 0-4 of each cycle, approximately 24 hours prior to each planned topotecan dose. On days when both drugs are administered (Days 1-4), AP-1 is administered after completion of topotecan infusion. On Days 6, 7, 8, 9, 10, 11, 12, 13, 14, 15, 16, 17, 18, 19, 20, and 21 of treatment cycles, neither topotecan nor AP-1 is administered.

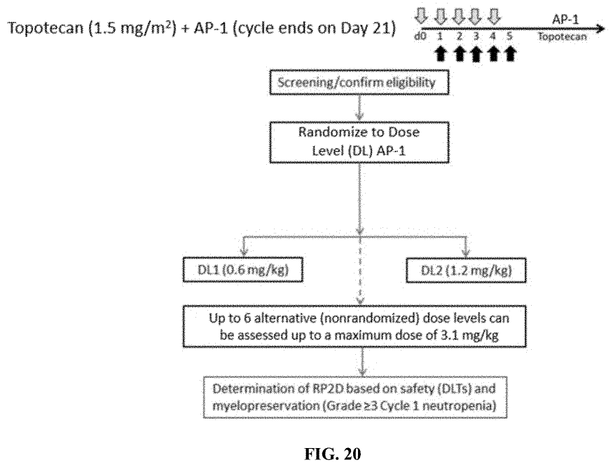

A schematic of the SCLC Phase 1b dose optimization study design is shown in FIG. 20.

A Phase 1b schedule optimization and expansion stage is also included, during which AP-1 is administered 6 hours prior to topotecan.

If pre-determined criteria for safety and myelopreservation activity are met and a RP2D of AP-1 is identified, the randomized Phase 1b expansion and the Phase 2 portion of the study in SCLC patients is triggered.

In the randomized Phase 1b expansion stage, 20 patients with ED SCLC harboring p53 loss of function mutations requiring 2nd-line treatment with topotecan are randomized 1:1 in a crossover fashion to one of two treatment sequences during Cycles 1 and 2:
Patients randomized to Sequence A receive treatment with topotecan+AP-1, as outlined above, during Cycle 1; and topotecan alone during Cycle 2. In all subsequent treatment cycles (Cycles ≥3), patients in Sequence A receive treatment with topotecan+AP-1.
Patients randomized to Sequence B receive treatment with topotecan alone during Cycle 1; and topotecan+AP-1 during Cycle 2. In all subsequent treatment cycles (Cycles ≥3), patients in Sequence B receive treatment with topotecan+AP-1.

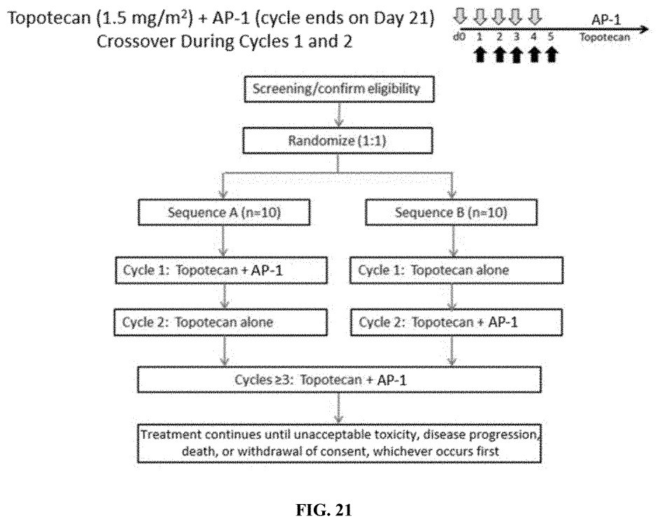

A schematic of the SCLC Phase 1b dose expansion study design is shown in FIG. 21.

Hematologic toxicities are monitored as described for Phase 1b dose optimization.

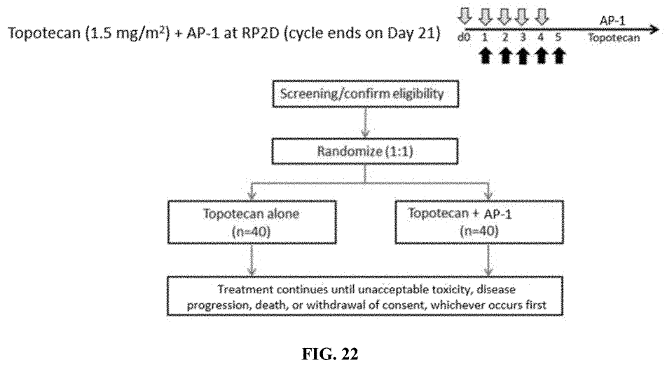

In Phase 2, patients with ED SCLC requiring 2nd line treatment with topotecan are randomized 1:1 to either receive topotecan alone (control arm) or topotecan with supportive AP-1 treatment (experimental arm). Monitoring of hematologic toxicities proceeds as in Phase 1b. A schematic of the SCLC Phase 2 study design is shown in FIG. 22.

### Study Population:

The SCLC inclusion criteria include:
1. Histopathological confirmation of ED SCLC that has recurred or been refractory to one line of treatment with standard platinum-based chemotherapy or immuno-chemotherapy. Patients who received immunotherapy after platinum-based chemotherapy remain eligible.

SCLC exclusion criteria include:

1. More than one line of prior chemotherapy for ED SCLC

### Treatment Regimens:

SCLC:

AP-1 and topotecan are administered as part of treatment cycles. Treatment cycles are denoted to begin on Day 0 and end on Day 21. Topotecan is given as an intravenous (IV) infusion over 30 minutes at a dose of 1.5 mg/m² on Days 1-5 of every treatment cycle. AP-1 is given as an IV infusion over 1 hour on Days 0-4 of every treatment cycle. The first dose of AP-1 (Day 0) is administered approximately 24 hours prior to the first topotecan dose (Day 1). On days when both drugs are administered (Days 1-4), AP-1 is administered after completion of the topotecan infusion. On Days 6, 7, 8, 9, 10, 11, 12, 13, 14, 15, 16, 17, 18, 19, 20, and 21 of treatment cycles, neither topotecan nor AP-1 is administered.

During Phase 1b dose optimization, patients are randomly assigned to initial AP-1 dose levels. Upon review of preliminary safety and efficacy data, alternative dose levels can also be evaluated. Alternative dose levels are determined based on emerging safety, tolerability, and myelopreservation activity as well as PK/PD data from previous dose levels.

During Phase 1b dose expansion, patients are randomly assigned to Sequence A or Sequence B. All patients in both treatment arms receive topotecan at a dose of 1.5 mg/m² on Days 1-5 of each treatment cycle. Patients randomized to Sequence A receive AP-1 as an IV infusion over 1 hour at the RP2D on Days 0-4 of Cycle 1, topotecan alone during Cycle 2, and AP-1+topotecan for all Cycles ≥3. Patients randomized to Sequence B receive topotecan alone during Cycle 1, AP-1 as an IV infusion over 1 hour at the RP2D on Days 0-4 of Cycle 2 and all subsequent cycles (Cycles ≥3). Neither topotecan nor AP-1 is administered on days 6-21 of treatment cycles.

During Phase 2, patients randomly assigned to the experimental arm receive AP-1 as an IV infusion over 1 hour at the RP2D on Days 0-4 of every cycle and topotecan at a dose of 1.5 mg/m² on Days 1-5 of each cycle. Neither topotecan nor AP-1 is administered on days 6-21 of treatment cycles. Patients randomly assigned to the control arm receive topotecan per the same dose and schedule but do not receive any administrations of AP-1.

### Myelopreservation Activity Assessments:

In both Phase 1b and Phase 2, the hematologic effects of treatment for individual patients are based on local laboratory results.

### Safety Assessments:

Other safety assessments include evaluation of adverse events (AEs) using NCI CTCAE (Version 5.0), clinical laboratory assessments (chemistry, hematology), vital sign measurements (blood pressure, heart rate, respiratory rate and body temperature), 12-lead electrocardiogram (ECG) measurements, and physical examination.

SCLC:

At each dose level in the Phase 1b dose optimization stage, 6 patients are first enrolled and evaluated, before additional patients are enrolled at that dose level to complete the cohort. Safety information from a lower dose level can be used to make a determination about further enrollment at a higher dose level, if safety or tolerability concerns arise. Alternative dose levels can be explored.

### PK/PD Assessments:

Blood samples for PK assessments of AP-1 are collected during Cycle 1 for patients enrolled in Phase 1b dose optimization and dose expansion (except for patients randomized to Sequence B of the SCLC cohort) and for patients randomized to the experimental arm in Phase 2. For patients randomized to Sequence B of the SCLC expansion cohort, blood samples for PK assessments of AP-1 are collected during Cycle 2.

### Evaluation of Tumor Response:

To assess for potential impact of AP-1 on the efficacy of topotecan and docetaxel, patients have radiographic response to treatment assessed after every 2 cycles. Response is based on investigator assessment according to RECIST (1.1).

### Treatment Duration:

Patients continue to receive topotecan or topotecan plus supportive AP-1 (SCLC) until unacceptable toxicity, disease progression, death, or withdrawal of consent, whichever occurs first.

### Example 32: Chemoprotection for Topotecan-Induce Mucositis

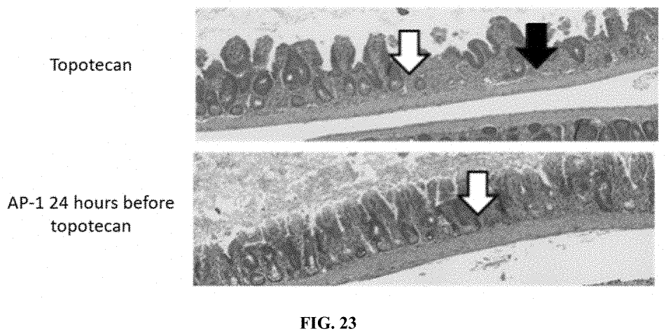

C57BL/6 mice (n=10 per group) were divided into four treatment groups. Group 1 was treated with a vehicle control. Group 2 was treated with 2.4 mg/kg AP-1 on days 0, 1, 2, 3, and 4 (0-4). Group 3 was treated with 1.5 mg/kg topotecan on days 1, 2, 3, 4, and 5 (1-5). Group 4 was treated with 2.4 mg/kg AP-1 on days 0-4 and 1.5 mg/kg topotecan on days 1-5. On days where topotecan and AP-1 were co-administered, AP-1 was given 30 minutes prior to topotecan. Gut samples were taken from mice at days 7 and 9 post treatment to assess hypertrophy and hyperplasia. Histopathology analysis of gut samples from mice treated with single agent topotecan showed marked epithelium hypertrophy/hyperplasia, moderate expansion of lamina propria (black arrow of FIG. 23) and moderate to severe crypt loss (white arrow of top panel FIG. 23). Gut tissue of animals treated with AP-1 prior to topotecan showed less extensive hypertrophy/hyperplasia; and mild crypt loss (white arrow of bottom panel of FIG. 23). Images and gut tissues and quantification of pathology scores can be seen in FIG. 24A.

In some embodiments, a preclinical or clinical conclusion can be drawn based upon analysis of quantified pathology scores in two treatment groups. For example, a preclinical conclusion can be based upon analysis of quantified pathology scores in Group A and Group B as illustrated in FIG. 24B, where Group A consists of mice treated with 1.5 mg/kg topotecan on days 1-5 of a treatment period, and Group B consists of mice treated with topotecan on days 1-5 of a treatment period and AP-1 24 hours before the first topotecan dose and 30 minutes before each subsequent topotecan dose.

### EMBODIMENTS

The following non-limiting embodiments provide illustrative examples of methods disclosed herein, but do not limit the scope of the disclosure.

### Embodiment 1

A method of treating a tumor in a subject in need thereof, the method comprising administering to the subject a therapeutically effective amount of a peptidomimetic macrocycle and a therapeutically effective amount of a first additional pharmaceutically-active agent, wherein:
the administration of the peptidomimetic macrocycle induces cell cycle arrest in a non-cancerous tissue in the subject;
the administration of the peptidomimetic macrocycle does not induce cell cycle arrest in the tumor; and
the administration of the peptidomimetic macrocycle does not induce apoptosis in the tumor.

### Embodiment 2

The method of embodiment 1, wherein the non-cancerous tissue is bone marrow.

### Embodiment 3

The method of embodiment 1, wherein the non-cancerous tissue is digestive tract tissue.

### Embodiment 4

The method of any one of embodiments 1-3, wherein the tumor has a p53 deactivating mutation.

### Embodiment 5

The method of embodiment 4, further comprising detecting the p53 deactivating mutation.

### Embodiment 6

The method of any one of embodiments 1-5, wherein the non-cancerous tissue comprises a functional p53 protein.

### Embodiment 7

The method of embodiment 6, further comprising confirming a presence of the functional p53 protein.

### Embodiment 8

The method of any one of embodiments 1-7, wherein administration of the peptidomimetic macrocycle reduces a likelihood of the subject developing a side effect associated with administration of the first additional pharmaceutically-active agent.

### Embodiment 9

The method of any one of embodiments 1-8, wherein administration of the peptidomimetic macrocycle reduces a level of a side effect in the subject, wherein the side effect is associated with administration of the first additional pharmaceutically-active agent.

### Embodiment 10

The method of embodiment 8, wherein the side effect is associated with myelosuppression.

### Embodiment 11

The method of embodiment 8, wherein the side effect is associated with digestive tissue.

### Embodiment 12

The method of embodiment 8, wherein the side effect is neutropenia.

### Embodiment 13

The method of embodiment 8, wherein the side effect is thrombocytopenia.

### Embodiment 14

The method of embodiment 8, wherein the side effect is mucositis.

### Embodiment 15

The method of embodiment 9, wherein the side effect is associated with myelosuppression.

### Embodiment 16

The method of embodiment 9, wherein the side effect is associated with digestive tissue.

### Embodiment 17

The method of embodiment 9, wherein the side effect is neutropenia.

### Embodiment 18

The method of embodiment 9, wherein the side effect is thrombocytopenia.

### Embodiment 19

The method of embodiment 9, wherein the side effect is mucositis.

### Embodiment 20

The method of embodiment 9, wherein administration of the peptidomimetic macrocycle increases a maximum tolerated dose of the first additional pharmaceutically-active agent.

### Embodiment 21

The method of any one of embodiments 1-20, wherein the first additional pharmaceutically-active agent is a topoisomerase inhibitor.

### Embodiment 22

The method of embodiment 21, wherein the topoisomerase inhibitor is a class I topoisomerase inhibitor.

### Embodiment 23

The method of embodiment 21, wherein the topoisomerase inhibitor is a class II topoisomerase inhibitor.

### Embodiment 24

The method of embodiment 21, wherein the topoisomerase inhibitor is topotecan.

### Embodiment 25

The method of embodiment 21, wherein the topoisomerase inhibitor is rubitecan.

### Embodiment 26

The method of embodiment 21, wherein the topoisomerase inhibitor is belotecan.

### Embodiment 27

The method of embodiment 21, wherein the topoisomerase inhibitor is etoposide.

### Embodiment 28

The method of embodiment 21, wherein the topoisomerase inhibitor is teniposide.

### Embodiment 29

The method of embodiment 21, wherein the therapeutically effective amount of the first additional pharmaceutically active agent is about 1.5 mg/m².

### Embodiment 30

The method of any one of embodiments 1-20, wherein the first additional pharmaceutically-active agent is a microtubule disassembly blocker.

### Embodiment 31

The method of embodiment 30, wherein the microtubule disassembly blocker is docetaxel.

### Embodiment 32

The method of embodiment 30, wherein the therapeutically effective amount of the first additional pharmaceutically active agent is about 75 mg/m².

### Embodiment 33

The method of any one of embodiments 1-20, wherein the first additional pharmaceutically-active agent is an alkylating-like agent.

### Embodiment 34

The method of embodiment 33, wherein the alkylating-like agent is carboplatin.

### Embodiment 35

The method of any one of embodiments 1-20, wherein the first additional pharmaceutically-active agent is a taxane.

### Embodiment 36

The method of embodiment 35, wherein the taxane is paclitaxel.

### Embodiment 37

The method of any one of embodiments 1-36, further comprising administering a therapeutically-effective amount of a second additional pharmaceutically-active agent.

### Embodiment 38

The method of embodiment 37, wherein the first additional pharmaceutically-active agent is a taxane and the second additional pharmaceutically-active agent is an alkylating-like agent.

### Embodiment 39

The method of embodiment 38, wherein the taxane is paclitaxel.

### Embodiment 40

The method of embodiment 38 or 39, wherein the alkylating-like agent is carboplatin.

### Embodiment 41

The method of embodiment 37, wherein the first additional pharmaceutically-active agent and the second additional pharmaceutically-active agent are administered concurrently.

### Embodiment 42

The method of embodiment 37, wherein the first additional pharmaceutically-active agent and the second additional pharmaceutically-active agent are administered sequentially.

### Embodiment 43

The method of any one of embodiments 1-42, wherein the peptidomimetic macrocycle and the first additional pharmaceutically-active agent are administered concurrently.

### Embodiment 44

The method of any one of embodiments 1-42, wherein the peptidomimetic macrocycle and the first additional pharmaceutically-active agent are administered sequentially.

### Embodiment 45

The method of any one of embodiments 1-42 or 44, wherein the peptidomimetic macrocycle is administered about 12 hours to about 36 hours before administration of the first additional pharmaceutically-active agent.

### Embodiment 46

The method of embodiment 45, wherein the peptidomimetic macrocycle is administered about 24 hours before administration of the first additional pharmaceutically-active agent.

### Embodiment 47

The method of any one of embodiments 1-46, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period; and
the first additional pharmaceutically-active agent is not administered on day 1 of the 6-day period.

### Embodiment 48

The method of embodiment 47, wherein each administration of the peptidomimetic macrocycle occurs about 12 hours to about 36 hours before each administration of the first additional pharmaceutically-active agent.

### Embodiment 49

The method of any one of embodiments 1-46, wherein:
the peptidomimetic macrocycle is administered on days 1, 2, and 3 of a 3-day period;
the first additional pharmaceutically-active agent is administered on day 2 of the 3-day period; and
the first additional pharmaceutically-active agent is not administered on day 1 or day 3 of the 3-day period.

### Embodiment 50

The method of any one of embodiments 1-49, wherein the peptidomimetic macrocycle binds to MDM2.

### Embodiment 51

The method of any one of embodiments 1-50, wherein the peptidomimetic macrocycle binds to MDMX.

### Embodiment 52

The method of any one of embodiments 1-49, wherein the peptidomimetic macrocycle binds to MDM2 and MDMX.

### Embodiment 53

The method of any one of embodiments 1-52, wherein the peptidomimetic macrocycle induces p53-dependent cell cycle arrest in the non-cancerous tissue.

### Embodiment 54

The method of any one of embodiments 1-53, wherein the therapeutically-effective amount of the peptidomimetic macrocycle is less than an amount of the peptidomimetic macrocycle that is needed to induce apoptosis in the non-cancerous tissue of the subject.

### Embodiment 55

The method of any one of embodiments 1-54, wherein the peptidomimetic macrocycle is of the formula:

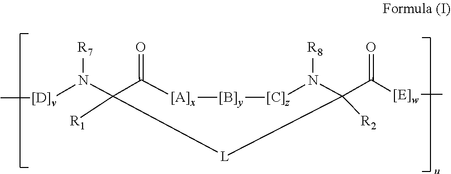

or a pharmaceutically acceptable salt thereof, wherein:
each A, C, D, and E is independently an amino acid;
each B is independently an amino acid,

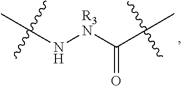

[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is 1, 2, 3, 4, 5, 6, 7, 8, 9, or 10;
each x, y and z is independently 1, 2, 3, 4, 5, 6, 7, 8, 9, or 10; and
each n is independently 1, 2, 3, 4, or 5.

### Embodiment 56

The method of embodiment 55, wherein v is 3-10.

### Embodiment 57

The method of embodiment 55, wherein v is 3.

### Embodiment 58

The method of any one of embodiments 55-57, wherein w is 3-10.

### Embodiment 59

The method of any one of embodiments 55-57, wherein w is 6.

### Embodiment 60

The method of any one of embodiments 55-59, wherein x+y+z=6.

### Embodiment 61

The method of any one of embodiments 55-60, wherein each L₁ and L₂ is independently alkylene, alkenylene, alkynylene, heteroalkylene, cycloalkylene, heterocycloalkylene, arylene, or heteroarylene.

### Embodiment 62

The method of any one of embodiments 55-60, wherein each L₁ and L₂ is independently alkylene or alkenylene.

### Embodiment 63

The method of any one of embodiments 55-62, wherein each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-.

### Embodiment 64

The method of any one of embodiments 55-62, wherein each R₁ and R₂ is independently hydrogen.

### Embodiment 65

The method of any one of embodiments 55-62, wherein each R₁ and R₂ is independently alkyl.

### Embodiment 66

The method of any one of embodiments 55-62, wherein each R₁ and R₂ is independently methyl.

### Embodiment 67

The method of any one of embodiments 55-65, wherein u is 1.

### Embodiment 68

The method of any one of embodiments 55-67, wherein each E is Ser or Ala, or d-Ala.

### Embodiment 69

The method of any one of embodiments 1-68, wherein the peptidomimetic macrocycle comprises an amino acid sequence that is at least 60% identical to an amino acid sequence listed Table 1, Table 1a, Table 1b, Table 1c, Table 2a, Table 2b, Table 3, or Table 3a.

### Embodiment 70

The method any one of embodiments 1-68, wherein the peptidomimetic macrocycle comprises an amino acid sequence that is at least 80% identical to an amino acid sequence listed in Table 1, Table 1a, Table 1b, Table 1c, Table 2a, Table 2b, Table 3, or Table 3a.

### Embodiment 71

The method of any one of embodiments 1-70, wherein the subject is a human.

### Embodiment 72

The method of any one of embodiments 1-71, wherein when, in a controlled study, 2.4 mg/kg of the peptidomimetic macrocycle is administered to a group of mice, changes in:

(i) an average p21 mRNA expression;

(ii) an average p53 upregulated modulator of apoptosis (PUMA) mRNA expression; and

(iii) an average Noxa mRNA expression;

in bone marrow of the group of mice occur with at most a 30% deviation from corresponding lines illustrated in FIG. 9.

### Embodiment 73

The method of any one of embodiments 1-71, wherein when, in a controlled study, 5 mg/kg of the peptidomimetic macrocycle is administered to a first group of 5-ethynyl-2′-deoxyuridine (EdU) treated mice, 10 mg/kg of the peptidomimetic macrocycle is administered to a second group of 5-ethynyl-2′-deoxyuridine (EdU) treated mice, and 20 mg/kg of the peptidomimetic macrocycle is administered to a third group of 5-ethynyl-2′-deoxyuridine (EdU) treated mice, a change in a percentage of lineage negative, Kit positive, hematopoietic stem and progenitor cells (HSPCs) that are EdU+ in the first group, the second group, and the third group occurs with at most a 30% deviation from corresponding lines illustrated in FIG. 11.

### Embodiment 74

The method of any one of embodiments 1-71, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a number of neutrophils present per μL of blood in mice of Group B is increased compared to a number of neutrophils present per μL of blood in mice of Group A as illustrated in FIG. 16D.

### Embodiment 75

The method of any one of embodiments 1-71, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a number of neutrophils present per μL of blood in mice of Group B is increased compared to a number of neutrophils present per μL of blood in mice of Group A as illustrated in FIG. 16C.

### Embodiment 76

The method of any one of embodiments 1-71, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a measure of hypertrophy/hyperplasia in digestive tract tissue in mice of Group B is modified compared to a measure of hypertrophy/hyperplasia in digestive tract tissue in mice of Group A as illustrated in FIG. 24B.

### Embodiment 77

The method of any one of embodiments 1-71, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

digestive tract tissue samples from about 80% of mice of Group B mice have a hypertrophy/hyperplasia score of 2, and digestive tract tissue samples from about 70% of mice of Group A have a hypertrophy/hyperplasia score of 3.

### Embodiment 78

The method of embodiment 37, wherein:
a first administration of the peptidomimetic macrocycle occurs 8 hours prior to the administration of the first additional pharmaceutically-active agent;
a second administration of the peptidomimetic macrocycle occurs 1 hour prior to the administration of the first additional pharmaceutically-active agent;
a third administration of the peptidomimetic macrocycle occurs 8 hours after the administration of the first additional pharmaceutically-active agent;
when, in a controlled study:
   (i) Group A consists of mice treated with 25 mg/kg carboplatin and 5 mg/kg paclitaxel at a first timepoint;
   (ii) Group B consists of mice treated with 25 mg/kg carboplatin and 5 mg/kg paclitaxel at the first timepoint and 2.4 mg/kg of the peptidomimetic macrocycle at a second timepoint, a third timepoint, and a fourth timepoint; and
   (iii) the second timepoint is about 8 hours prior to the first timepoint, the third timepoint is about 1 hour prior to the first timepoint, and the fourth timepoint is about 8 hours after the first timepoint;

a number of neutrophils present per μL of blood in mice of Group B is increased compared to a number of neutrophils present per μL of blood in mice of Group A as illustrated in FIG. 18B.

### Embodiment 79

A method of treating a cancer in a subject in need thereof, the method comprising administering to the subject a therapeutically effective amount of a peptidomimetic macrocycle and a therapeutically effective amount of a first additional pharmaceutically-active agent, wherein:
the cancer has a p53 deactivating mutation;
a non-cancerous tissue of the subject comprises a functional p53 protein; and
the non-cancerous tissue is bone marrow or digestive tract tissue.

### Embodiment 80

The method of embodiment 79, further comprising detecting the p53 deactivating mutation.

### Embodiment 81

The method of embodiment 79 or 80, wherein the non-cancerous tissue is bone marrow.

### Embodiment 82

The method of embodiment 79 or 80, wherein the non-cancerous tissue is digestive tract tissue.

### Embodiment 83

The method of any one of embodiments 79-82, wherein administration of the peptidomimetic macrocycle induces cell-cycle arrest in the non-cancerous tissue.

### Embodiment 84

The method of any one of embodiments 79-83 wherein administration of the peptidomimetic macrocycle does not induce cell cycle arrest or apoptosis in the cancer.

### Embodiment 85

The method of any one of embodiments 79-84, wherein administration of the peptidomimetic macrocycle reduces a likelihood of the subject developing a side effect associated with administration of the first additional pharmaceutically-active agent.

### Embodiment 86

The method of any one of embodiments 79-85, wherein administration of the peptidomimetic macrocycle reduces a level of a side effect in the subject, wherein the side effect is associated with administration of the first additional pharmaceutically-active agent.

### Embodiment 87

The method of embodiment 85, wherein the side effect is associated with myelosuppression.

### Embodiment 88

The method of embodiment 85, wherein the side effect is associated with digestive tissue.

### Embodiment 89

The method of embodiment 85, wherein the side effect is neutropenia.

### Embodiment 90

The method of embodiment 85, wherein the side effect is thrombocytopenia.

### Embodiment 91

The method of embodiment 85, wherein the side effect is mucositis.

### Embodiment 92

The method of embodiment 86, wherein the side effect is associated with myelosuppression.

### Embodiment 93

The method of embodiment 86, wherein the side effect is associated with digestive tissue.

### Embodiment 94

The method of embodiment 86, wherein the side effect is neutropenia.

### Embodiment 95

The method of embodiment 86, wherein the side effect is thrombocytopenia.

### Embodiment 96

The method of embodiment 86, wherein the side effect is mucositis.

### Embodiment 97

The method of any one of embodiments 79-96, wherein administration of the peptidomimetic macrocycle increases a maximum tolerated dose of the first additional pharmaceutically-active agent.

### Embodiment 98

The method of any one of embodiments 79-97, wherein the first additional pharmaceutically-active agent is a topoisomerase inhibitor.

### Embodiment 99

The method of embodiment 98, wherein the topoisomerase inhibitor is a class II topoisomerase inhibitor.

### Embodiment 100

The method of embodiment 98, wherein the topoisomerase inhibitor is a class I topoisomerase inhibitor.

### Embodiment 101

The method of embodiment 98, wherein the topoisomerase inhibitor is topotecan.

### Embodiment 102

The method of embodiment 98, wherein the topoisomerase inhibitor is rubitecan.

### Embodiment 103

The method of embodiment 98, wherein the topoisomerase inhibitor is belotecan.

### Embodiment 104

The method of embodiment 98, wherein the topoisomerase inhibitor is etoposide.

### Embodiment 105

The method of embodiment 98, wherein the topoisomerase inhibitor is teniposide.

### Embodiment 106

The method of embodiment 98, wherein the therapeutically effective amount of the first additional pharmaceutically active agent is about 1.5 mg/m².

### Embodiment 107

The method of any one of embodiments 79-97, wherein the first additional pharmaceutically-active agent is a microtubule disassembly blocker.

### Embodiment 108

The method of embodiment 107, wherein the microtubule disassembly blocker is docetaxel.

### Embodiment 109

The method of embodiment 107, wherein the therapeutically effective amount of the first additional pharmaceutically active agent is about 75 mg/m².

### Embodiment 110

The method of any one of embodiments 79-97, wherein the first additional pharmaceutically-active agent is an alkylating-like agent.

### Embodiment 111

The method of embodiment 110, wherein the alkylating-like agent is carboplatin.

### Embodiment 112

The method of any one of embodiments 79-97, wherein the first additional pharmaceutically-active agent is a taxane.

### Embodiment 113

The method of embodiment 112, wherein the taxane is paclitaxel.

### Embodiment 114

The method of any one of embodiments 79-113, further comprising administering a therapeutically-effective amount of a second additional pharmaceutically-active agent.

### Embodiment 115

The method of embodiment 114, wherein the first additional pharmaceutically-active agent is a taxane and the second additional pharmaceutically-active agent is an alkylating-like agent.

### Embodiment 116

The method of embodiment 115, wherein the taxane is paclitaxel.

### Embodiment 117

The method of embodiment 115 or 116, wherein the alkylating-like agent is carboplatin.

### Embodiment 118

The method of embodiment 114, wherein the first additional pharmaceutically-active agent and the second additional pharmaceutically-active agent are administered concurrently.

### Embodiment 119

The method of embodiment 114, wherein the first additional pharmaceutically-active agent and the second additional pharmaceutically-active agent are administered sequentially.

### Embodiment 120

The method of any one of embodiments 79-119, wherein the peptidomimetic macrocycle and the first additional pharmaceutically-active agent are administered concurrently.

### Embodiment 121

The method of any one of embodiments 79-119, wherein the peptidomimetic macrocycle and the first additional pharmaceutically-active agent are administered sequentially.

### Embodiment 122

The method of any one of embodiments 79-119 or 121, wherein the peptidomimetic macrocycle is administered about 12 hours to about 36 hours before administration of the first additional pharmaceutically-active agent.

### Embodiment 123

The method of embodiment 122, wherein peptidomimetic macrocycle is administered about 24 hours before administration of the first additional pharmaceutically-active agent.

### Embodiment 124

The method of any one of embodiments 79-123, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period; and
the first additional pharmaceutically-active agent is not administered on day 1 of the 6-day period.

### Embodiment 125

The method of embodiment 124, wherein each administration of the peptidomimetic macrocycle occurs about 12 hours to about 36 hours before each administration of the first additional pharmaceutically-active agent.

### Embodiment 126

The method of any one of embodiments 79-123, wherein:
the peptidomimetic macrocycle is administered on days 1, 2, and 3 of a 3-day period;
the first additional pharmaceutically-active agent is administered on day 2 of the 3-day period; and
the first additional pharmaceutically-active agent is not administered on day 1 or day 3 of the 3-day period.

### Embodiment 127

The method of any one of embodiments 79-126, wherein the peptidomimetic macrocycle binds to MDM2.

### Embodiment 128

The method of any one of embodiments 79-127, wherein the peptidomimetic macrocycle binds to MDMX.

### Embodiment 129

The method of any one of embodiments 79-126, wherein the peptidomimetic macrocycle binds to MDM2 and MDMX.

### Embodiment 130

The method of any one of embodiments 79-129, wherein the peptidomimetic macrocycle induces p53-dependent cell cycle arrest in the non-cancerous tissue.

### Embodiment 131

The method of any one of embodiments 79-130, wherein the therapeutically-effective amount of the peptidomimetic macrocycle is less than an amount of the peptidomimetic macrocycle that is needed to induce apoptosis in the non-cancerous tissue of the subject.

### Embodiment 132

The method of any one of embodiments 79-131, wherein the peptidomimetic macrocycle is of the formula:

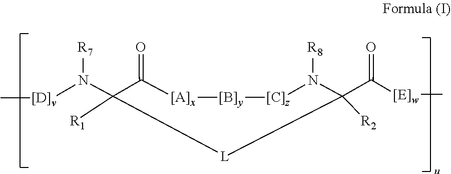

or a pharmaceutically acceptable salt thereof, wherein:
each A, C, D, and E is independently an amino acid;
each B is independently an amino acid,

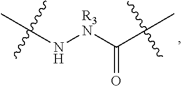

[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is 1, 2, 3, 4, 5, 6, 7, 8, 9, or 10;
each x, y and z is independently 1, 2, 3, 4, 5, 6, 7, 8, 9, or 10; and
each n is independently 1, 2, 3, 4, or 5.

### Embodiment 133

The method of embodiment 132, wherein v is 3-10.

### Embodiment 134

The method of embodiment 132, wherein v is 3.

### Embodiment 135

The method of any one of embodiments 132-134, wherein w is 3-10.

### Embodiment 136

The method of any one of embodiments 132-134, wherein w is 6.

### Embodiment 137

The method of any one of embodiments 132-136, wherein each L₁ and L₂ is independently alkylene, alkenylene, alkynylene, heteroalkylene, cycloalkylene, heterocycloalkylene, arylene, or heteroarylene.

### Embodiment 138

The method of any one of embodiments 132-136, wherein each L₁ and L₂ is independently alkylene or alkenylene.

### Embodiment 139

The method of any one of embodiments 132-136, wherein each L₁ and L₂ is independently alkylene or alkenylene.

### Embodiment 140

The method of any one of embodiments 132-139, wherein each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-.

### Embodiment 141

The method of any one of embodiments 132-139, wherein each R₁ and R₂ is independently hydrogen.

### Embodiment 142

The method of any one of embodiments 132-139, wherein each R₁ and R₂ is independently alkyl.

### Embodiment 143

The method of any one of embodiments 132-139, wherein each R₁ and R₂ is independently methyl.

### Embodiment 144

The method of any one of embodiments 132-143, wherein u is 1.

### Embodiment 145

The method of any one of embodiments 132-144, wherein each E is Ser or Ala, or d-Ala.

### Embodiment 146

The method of any one of embodiments 79-145, wherein the peptidomimetic macrocycle comprises an amino acid sequence that is at least 60% identical to an amino acid sequence listed Table 1, Table 1a, Table 1b, Table 1c, Table 2a, Table 2b, Table 3, or Table 3a.

### Embodiment 147

The method of any one of embodiments 79-145, wherein the peptidomimetic macrocycle comprises an amino acid sequence that is at least 80% identical to an amino acid sequence listed in Table 1, Table 1a, Table 1b, Table 1c, Table 2a, Table 2b, Table 3, or Table 3a.

### Embodiment 148

The method of any one of embodiments 79-147, wherein the subject is a human.

### Embodiment 149

The method of any one of embodiments 79-148, wherein when, in a controlled study, 2.4 mg/kg of the peptidomimetic macrocycle is administered to a group of mice, changes in:

(i) an average p21 mRNA expression;

(ii) an average p53 upregulated modulator of apoptosis (PUMA) mRNA expression; and

(iii) an average Noxa mRNA expression;

in bone marrow of the group of mice occur with at most a 30% deviation from corresponding lines illustrated in FIG. 9.

### Embodiment 150

The method of any one of embodiments 79-148, wherein when, in a controlled study, 5 mg/kg of the peptidomimetic macrocycle is administered to a first group of 5-ethynyl-2′-deoxyuridine (EdU) treated mice, 10 mg/kg of the peptidomimetic macrocycle is administered to a second group of 5-ethynyl-2′-deoxyuridine (EdU) treated mice, and 20 mg/kg of the peptidomimetic macrocycle is administered to a third group of 5-ethynyl-2′-deoxyuridine (EdU) treated mice, a change in a percentage of lineage negative, Kit positive, hematopoietic stem and progenitor cells (HSPCs) that are EdU+ in the first group, the second group, and the third group occurs with at most a 30% deviation from corresponding lines illustrated in FIG. 11.

### Embodiment 151

The method of any one of embodiments 79-148, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a number of neutrophils present per μL of blood in mice of Group B is increased compared to a number of neutrophils present per μL of blood in mice of Group A as illustrated in FIG. 16D.

### Embodiment 152

The method of any one of embodiments 79-148, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a number of neutrophils present per μL of blood in mice of Group B is increased compared to a number of neutrophils present per μL of blood in mice of Group A as illustrated in FIG. 16C.

### Embodiment 153

The method of any one of embodiments 79-148, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a measure of hypertrophy/hyperplasia in digestive tract tissue in mice of Group B is modified compared to a measure of hypertrophy/hyperplasia in digestive tract tissue in mice of Group A as illustrated in FIG. 24B.

### Embodiment 154

The method of any one of embodiments 79-148, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

digestive tract tissue samples from about 80% of mice of Group B mice have a hypertrophy/hyperplasia score of 2, and digestive tract tissue samples from about 70% of mice of Group A have a hypertrophy/hyperplasia score of 3.

### Embodiment 155

The method of embodiment 114, wherein:
a first administration of the peptidomimetic macrocycle occurs 8 hours prior to the administration of the first additional pharmaceutically-active agent;
a second administration of the peptidomimetic macrocycle occurs 1 hour prior to the administration of the first additional pharmaceutically-active agent;
a third administration of the peptidomimetic macrocycle occurs 8 hours after the administration of the first additional pharmaceutically-active agent;
when, in a controlled study:
   (i) Group A consists of mice treated with 25 mg/kg carboplatin and 5 mg/kg paclitaxel at a first timepoint;
   (ii) Group B consists of mice treated with 25 mg/kg carboplatin and 5 mg/kg paclitaxel at the first timepoint and 2.4 mg/kg of the peptidomimetic macrocycle at a second timepoint, a third timepoint, and a fourth timepoint; and
   (iii) the second timepoint is about 8 hours prior to the first timepoint, the third timepoint is about 1 hour prior to the first timepoint, and the fourth timepoint is about 8 hours after the first timepoint;

a number of neutrophils present per μL of blood in mice of Group B is increased compared to a number of neutrophils present per μL of blood in mice of Group A as illustrated in FIG. 18B.

### Embodiment 156

A method of treating a tumor in a subject in need thereof, the method comprising administering to the subject a therapeutically effective amount of a peptidomimetic macrocycle and a therapeutically effective amount of a first additional pharmaceutically-active agent, wherein:
the administration of the peptidomimetic macrocycle does not induce cell cycle arrest in the tumor;
the administration of the peptidomimetic macrocycle does not induce apoptosis in the tumor;
the therapeutically effective amount of the first additional pharmaceutically-active agent is associated with a side effect; and
the administration of the peptidomimetic macrocycle reduces a likelihood of the subject developing the side effect.

### Embodiment 157

The method of embodiment 156, wherein the subject is a human.

### Embodiment 158

The method of embodiment 156 or 157, wherein administration of the peptidomimetic macrocycle induces cell cycle arrest in a non-cancerous tissue.

### Embodiment 159

The method of any one of embodiments 156-158, wherein the tumor has a p53 deactivating mutation.

### Embodiment 160

The method of embodiment 159, further comprising detecting the p53 deactivating mutation.

### Embodiment 161

The method of any one of embodiments 156-160, wherein administration of the peptidomimetic macrocycle reduces a level of the side effect.

### Embodiment 162

The method of any one of embodiments 156-161, wherein the side effect is associated with myelosuppression.

### Embodiment 163

The method of any one of embodiments 156-161, wherein the side effect is associated with digestive tract tissue.

### Embodiment 164

The method of any one of embodiments 156-162, wherein the side effect is neutropenia.

### Embodiment 165

The method of any one of embodiments 156-162, wherein the side effect is thrombocytopenia.

### Embodiment 166

The method of any one of embodiments 156-161 or 163, wherein the side effect is mucositis.

### Embodiment 167

The method of any one of embodiments 156-166, wherein administration of the peptidomimetic macrocycle increases a maximum tolerated dose of the first additional pharmaceutically-active agent.

### Embodiment 168

The method of any one of embodiments 156-167, wherein the first additional pharmaceutically-active agent is a topoisomerase inhibitor.

### Embodiment 169

The method of embodiment 168, wherein the topoisomerase inhibitor is a class I topoisomerase inhibitor.

### Embodiment 170

The method of embodiment 168, wherein the topoisomerase inhibitor is a class II topoisomerase inhibitor.

### Embodiment 171

The method of embodiment 168, wherein the topoisomerase inhibitor is topotecan.

### Embodiment 172

The method of embodiment 168, wherein the topoisomerase inhibitor is rubitecan.

### Embodiment 173

The method of embodiment 168, wherein the topoisomerase inhibitor is belotecan.

### Embodiment 174

The method of embodiment 168, wherein the topoisomerase inhibitor is etoposide.

### Embodiment 175

The method of embodiment 168, wherein the topoisomerase inhibitor is teniposide.

### Embodiment 176

The method of embodiment 168, wherein the therapeutically effective amount of the first additional pharmaceutically active agent is about 1.5 mg/m².

### Embodiment 177

The method of any one of embodiments 156-167, wherein the first additional pharmaceutically-active agent is a microtubule disassembly blocker.

### Embodiment 178

The method of embodiment 177, wherein the microtubule disassembly blocker is docetaxel.

### Embodiment 179

The method of embodiment 177, wherein the therapeutically effective amount of the first additional pharmaceutically active agent is about 75 mg/m².

### Embodiment 180

The method of any one of embodiments 156-167, wherein the first additional pharmaceutically-active agent is an alkylating-like agent.

### Embodiment 181

The method of embodiment 180, wherein the alkylating-like agent is carboplatin.

### Embodiment 182

The method of any one of embodiments 156-167, wherein the first additional pharmaceutically-active agent is a taxane.

### Embodiment 183

The method of embodiment 182, wherein the taxane is paclitaxel.

### Embodiment 184

The method of any one of embodiments 156-183, further comprising administering a therapeutically-effective amount of a second additional pharmaceutically-active agent.

### Embodiment 185

The method of embodiment 184, wherein the first additional pharmaceutically-active agent is a taxane and the second additional pharmaceutically-active agent is an alkylating-like agent.

### Embodiment 186

The method of embodiment 185, wherein the taxane is paclitaxel.

### Embodiment 187

The method of embodiment 185 or 186, wherein the alkylating-like agent is carboplatin.

### Embodiment 188

The method of any one of embodiments 184-187, wherein the first additional pharmaceutically-active agent and the second additional pharmaceutically-active agent are administered concurrently.

### Embodiment 189

The method of any one of embodiments 184-187, wherein the first additional pharmaceutically-active agent and the second additional pharmaceutically-active agent are administered sequentially.

### Embodiment 190

The method of any one of embodiments 156-189, wherein the peptidomimetic macrocycle and the first additional pharmaceutically-active agent are administered concurrently.

### Embodiment 191

The method of any one of embodiments 156-189, wherein the peptidomimetic macrocycle and the first additional pharmaceutically-active agent are administered sequentially.

### Embodiment 192

The method of any one of embodiments 156-189 or 191, wherein the peptidomimetic macrocycle is administered about 12 hours to about 36 hours before administration of the first additional pharmaceutically-active agent.

### Embodiment 193

The method of embodiment 192, wherein the peptidomimetic macrocycle is administered about 24 hours before administration of the first additional pharmaceutically-active agent.

### Embodiment 194

The method of any one of embodiments 156-193, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period; and
the first additional pharmaceutically-active agent is not administered on day 1 of the 6-day period.

### Embodiment 195

The method of embodiment 194, wherein each administration of the peptidomimetic macrocycle occurs about 12 hours to about 36 hours before each administration of the first additional pharmaceutically-active agent.

### Embodiment 196

The method of any one of embodiments 156-193, wherein:
the peptidomimetic macrocycle is administered on days 1, 2, and 3 of a 3-day period;
the first additional pharmaceutically-active agent is administered on day 2 of the 3-day period; and
the first additional pharmaceutically-active agent is not administered on day 1 or day 3 of the 3-day period.

### Embodiment 197

The method of any one of embodiments 156-196, wherein the peptidomimetic macrocycle binds to MDM2.

### Embodiment 198

The method of any one of embodiments 156-197, wherein the peptidomimetic macrocycle binds to MDMX.

### Embodiment 199

The method of any one of embodiments 156-196, wherein the peptidomimetic macrocycle binds to MDM2 and MDMX.

### Embodiment 200

The method of any one of embodiments 156-199, wherein the peptidomimetic macrocycle induces p53-dependent cell cycle arrest in a non-cancerous tissue.

### Embodiment 201

The method of any one of embodiments 156-200, wherein the therapeutically-effective amount of the peptidomimetic macrocycle is less than an amount of the peptidomimetic macrocycle that is needed to induce apoptosis in the non-cancerous tissue of the subject.

### Embodiment 202

The method of any one of embodiments 156-201, wherein the peptidomimetic macrocycle is of the formula:

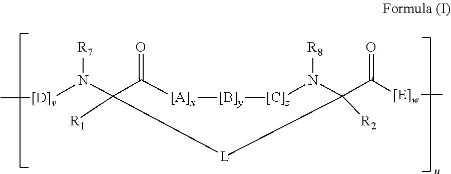

or a pharmaceutically acceptable salt thereof, wherein:
each A, C, D, and E is independently an amino acid;
each B is independently an amino acid,

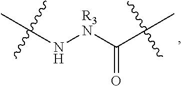

[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is 1, 2, 3, 4, 5, 6, 7, 8, 9, or 10;
each x, y and z is independently 1, 2, 3, 4, 5, 6, 7, 8, 9, or 10; and
each n is independently 1, 2, 3, 4, or 5.

### Embodiment 203

The method of embodiment 202, wherein v is 3-10.

### Embodiment 204

The method of embodiment 202, wherein v is 3.

### Embodiment 205

The method of any one of embodiments 202-204, wherein w is 3-10.

### Embodiment 206

The method of any one of embodiments 202-204, wherein w is 6.

### Embodiment 207

The method of any one of embodiments 202-206, wherein x+y+z=6.

### Embodiment 208

The method of any one of embodiments 202-207, wherein each L₁ and L₂ is independently alkylene, alkenylene, alkynylene, heteroalkylene, cycloalkylene, heterocycloalkylene, arylene, or heteroarylene.

### Embodiment 209

The method of any one of embodiments 202-207, wherein each L₁ and L₂ is independently alkylene or alkenylene.

### Embodiment 210

The method of any one of embodiments 202-209, wherein each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-.

### Embodiment 211

The method of any one of embodiments 202-209, wherein each R₁ and R₂ is independently hydrogen.

### Embodiment 212

The method of any one of embodiments 202-209, wherein each R₁ and R₂ is independently alkyl.

### Embodiment 213

The method of any one of embodiments 202-209, wherein each R₁ and R₂ is independently methyl.

### Embodiment 214

The method of any one of embodiments 202-213, wherein u is 1.

### Embodiment 215

The method of any one of embodiments 202-214, wherein each E is Ser or Ala, or d-Ala.

### Embodiment 216

The method of any one of embodiments 156-215, wherein the peptidomimetic macrocycle comprises an amino acid sequence that is at least 60% identical to an amino acid sequence listed Table 1, Table 1a, Table 1b, Table 1c, Table 2a, Table 2b, Table 3, or Table 3a.

### Embodiment 217

The method of any one of embodiments 156-215, wherein the peptidomimetic macrocycle comprises an amino acid sequence that is at least 80% identical to an amino acid sequence listed in Table 1, Table 1a, Table 1b, Table 1c, Table 2a, Table 2b, Table 3, or Table 3a.

### Embodiment 218

The method of any one of embodiments 156-217, wherein when, in a controlled study, 2.4 mg/kg of the peptidomimetic macrocycle is administered to a group of mice, changes in:

(i) an average p21 mRNA expression;

(ii) an average p53 upregulated modulator of apoptosis (PUMA) mRNA expression; and

(iii) an average Noxa mRNA expression;

in bone marrow of the group of mice occur with at most a 30% deviation from corresponding lines illustrated in FIG. 9.

### Embodiment 219

The method of any one of embodiments 156-217, wherein when, in a controlled study, 5 mg/kg of the peptidomimetic macrocycle is administered to a first group of 5-ethynyl-2′-deoxyuridine (EdU) treated mice, 10 mg/kg of the peptidomimetic macrocycle is administered to a second group of 5-ethynyl-2′-deoxyuridine (EdU) treated mice, and 20 mg/kg of the peptidomimetic macrocycle is administered to a third group of 5-ethynyl-2′-deoxyuridine (EdU) treated mice, a change in a percentage of lineage negative, Kit positive, hematopoietic stem and progenitor cells (HSPCs) that are EdU+ in the first group, the second group, and the third group occurs with at most a 30% deviation from corresponding lines illustrated in FIG. 11.

### Embodiment 220

The method of any one of embodiments 156-217, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a number of neutrophils present per μL of blood in mice of Group B is increased compared to a number of neutrophils present per μL of blood in mice of Group A as illustrated in FIG. 16D.

### Embodiment 221

The method of any one of embodiments 156-217, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a number of neutrophils present per μL of blood in mice of Group B is increased compared to a number of neutrophils present per μL of blood in mice of Group A as illustrated in FIG. 16C.

### Embodiment 222

The method of any one of embodiments 156-217, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

a measure of hypertrophy/hyperplasia in digestive tract tissue in mice of Group B is modified compared to a measure of hypertrophy/hyperplasia in digestive tract tissue in mice of Group A as illustrated in FIG. 24B.

### Embodiment 223

The method of any one of embodiments 156-217, wherein:
the peptidomimetic macrocycle is administered on days 1, 2, 3, 4, and 5 of a 6-day period;
the peptidomimetic macrocycle is not administered on day 6 of the 6-day period;
the first additional pharmaceutically-active agent is administered on days 2, 3, 4, 5, and 6 of the 6-day period;
the first additional pharmaceutically-active agent is not administered on day 1 of the 6-day period; and
when, in a controlled study:
   (i) Group A consists of mice treated with 1.5 mg/kg of topotecan on days 2, 3, 4, 5, and 6 of a 6-day study treatment period; and not treated with topotecan on day 1 of the 6-day study treatment period; and
   (ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;

digestive tract tissue samples from about 80% of mice of Group B mice have a hypertrophy/hyperplasia score of 2, and digestive tract tissue samples from about 70% of mice of Group A have a hypertrophy/hyperplasia score of 3.

### Embodiment 224

The method of any one of embodiments 156-217, wherein:
a first administration of the peptidomimetic macrocycle occurs 8 hours prior to the administration of the first additional pharmaceutically-active agent;
a second administration of the peptidomimetic macrocycle occurs 1 hour prior to the administration of the first additional pharmaceutically-active agent;
a third administration of the peptidomimetic macrocycle occurs 8 hours after the administration of the first additional pharmaceutically-active agent;
when, in a controlled study:
   (i) Group A consists of mice treated with 25 mg/kg carboplatin and 5 mg/kg paclitaxel at a first timepoint;

(ii) Group B consists of mice treated with 25 mg/kg carboplatin and 5 mg/kg paclitaxel at the first timepoint and 2.4 mg/kg of the peptidomimetic macrocycle at a second timepoint, a third timepoint, and a fourth timepoint; and

(iii) the second timepoint is about 8 hours prior to the first timepoint, the third timepoint is about 1 hour prior to the first timepoint, and the fourth timepoint is about 8 hours after the first timepoint;

a number of neutrophils present per μL of blood in mice of Group B is increased compared to a number of neutrophils present per μL of blood in mice of Group A as illustrated in FIG. 18B.

### Embodiment 225

The method of any one of embodiments 1-22 or 43-78, wherein the first additional pharmaceutically-active agent is a chemotherapeutic agent.

### Embodiment 226

The method of any one of embodiments 1-22 or 43-78, wherein the first additional pharmaceutically-active agent is an antineoplastic agent.

### Embodiment 227

The method of any one of embodiments 79-97, or 120-155, wherein the first additional pharmaceutically-active agent is a chemotherapeutic agent.

### Embodiment 228

The method of any one of embodiments 79-97, or 120-155, wherein the first additional pharmaceutically-active agent is an antineoplastic agent.

### Embodiment 229

The method of any one of embodiments 156-167, or 190-224, wherein the first additional pharmaceutically-active agent is a chemotherapeutic agent.

### Embodiment 230

The method of any one of embodiments 156-167, or 190-224, wherein the first additional pharmaceutically-active agent is an antineoplastic agent.

1. A method of treating a tumor in a subject in need thereof, the method comprising administering to the subject a therapeutically effective amount of a peptidomimetic macrocycle and a therapeutically effective amount of an additional pharmaceutically-active agent, wherein:
the administration of the peptidomimetic macrocycle induces cell cycle arrest in a non-cancerous tissue in the subject;
the administration of the peptidomimetic macrocycle does not induce cell cycle arrest in the tumor; and
the administration of the peptidomimetic macrocycle does not induce apoptosis in the tumor.

2. (canceled)

3. (canceled)

4. (canceled)

5. The method of claim 1, wherein administration of the peptidomimetic macrocycle reduces a level of a side effect in the subject, wherein the side effect is associated with administration of the additional pharmaceutically-active agent.

6-10. (canceled)

11. The method of claim 5, wherein the side effect is associated with myelosuppression.

12. The method of claim 5, wherein the side effect is associated with digestive tissue.

13. The method of claim 5, wherein the side effect is neutropenia.

14. The method of claim 5, wherein the side effect is thrombocytopenia.

15. The method of claim 5, wherein the side effect is mucositis.

16. The method of claim 1, wherein the additional pharmaceutically-active agent is a chemotherapeutic agent.

17. The method of claim 1, wherein the additional pharmaceutically-active agent is an antineoplastic agent.

18. The method of claim 1, wherein the additional pharmaceutically-active agent is a topoisomerase inhibitor.

19. (canceled)

20. The method of claim 1, wherein the additional pharmaceutically-active agent is an alkylating-like agent.

21. The method of claim 1, wherein the additional pharmaceutically-active agent is a taxane.

22-25. (canceled)

26. The method of claim 1, wherein:
the peptidomimetic macrocycle is administered on days 1, 2, 3, 4, and 5 of a 6-day period;
the peptidomimetic macrocycle is not administered on day 6 of the 6-day period;
the additional pharmaceutically-active agent is administered on days 2, 3, 4, 5, and 6 of the 6-day period; and
the additional pharmaceutically-active agent is not administered on day 1 of the 6-day period.

27. (canceled)

28. The method of claim 1, wherein the peptidomimetic macrocycle binds to MDM2.

29. The method of claim 1, wherein the peptidomimetic macrocycle binds to MDMX.

30. (canceled)

31. The method of claim 1, wherein the peptidomimetic macrocycle induces p53-dependent cell cycle arrest in the non-cancerous tissue.

32. (canceled)

33. The method of claim 1, wherein the peptidomimetic macrocycle is of the formula: 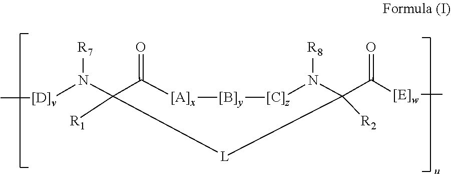
or a pharmaceutically acceptable salt thereof, wherein:
each A, C, D, and E is independently an amino acid;
each B is independently an amino acid, 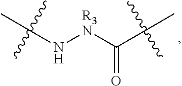
[—NH-L₃-CO—], [—NH-L₃-SO₂—], or [—NH-L₃-];
each R₁ and R₂ is independently hydrogen, alkyl, alkenyl, alkynyl, arylalkyl, cycloalkyl, cycloalkylalkyl, heteroalkyl, or heterocycloalkyl, unsubstituted or substituted with halo-; or
forms a macrocycle-forming linker L′ connected to the alpha position of one of said D or E amino acids;
each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-;
each L₁, L₂, and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently hydrogen, alkyl, alkenyl, alkynyl, arylalkyl, cycloalkylalkyl, heterocycloalkyl, a fluorescent moiety, a radioisotope or a therapeutic agent;
each R₇ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently hydrogen, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v is independently an integer from 1-1000;
each w is independently an integer from 1-1000;
u is 1, 2, 3, 4, 5, 6, 7, 8, 9, or 10;
each x, y and z is independently 1, 2, 3, 4, 5, 6, 7, 8, 9, or 10; and
each n is independently 1, 2, 3, 4, or 5.

34. (canceled)

35. The method of claim 1, wherein the peptidomimetic macrocycle comprises an amino acid sequence that is at least 80% identical to an amino acid sequence listed in Table 1, Table 1a, Table 1b, Table 1c, Table 2a, Table 2b, Table 3, or Table 3a.

36. The method of claim 1, wherein the subject is a human.

37-41. (canceled)

42. The method of claim 1, wherein:
the peptidomimetic macrocycle is administered on days 1, 2, 3, 4, and 5 of a 6-day period;
the peptidomimetic macrocycle is not administered on day 6 of the 6-day period;
the additional pharmaceutically-active agent is administered on days 2, 3, 4, 5, and 6 of the 6-day period;
the additional pharmaceutically-active agent is not administered on day 1 of the 6-day period; and
when, in a controlled study:
(i) Group A consists of mice treated with 1.5 mg/kg of topotecan on days 2, 3, 4, 5, and 6 of a 6-day study treatment period; and not treated with topotecan on day 1 of the 6-day study treatment period; and
(ii) Group B consists of mice treated with 2.4 mg/kg of the peptidomimetic macrocycle on days 1, 2, 3, 4, and 5 of the 6-day study treatment period and 1.5 mg/kg of topotecan on days 2, 3, 4, 5, and 6 of the 6-day study treatment period, wherein the mice of Group B are not treated with the peptidomimetic macrocycle on day 6 of the 6-day study treatment period and are not treated with topotecan on day 1 of the 6-day study treatment period;
digestive tract tissue samples from about 80% of mice of Group B mice have a hypertrophy/hyperplasia score of 2, and digestive tract tissue samples from about 70% of mice of Group A have a hypertrophy/hyperplasia score of 3.

43. (canceled)
